(19) United States
(12) Patent Application Publication
(10) Pub. No.: US 2011/0150757 A1
(43) Pub. Date: Jun. 23, 2011

(54) COMPOUNDS FOR INHIBITING KSP KINESIN ACTIVITY

(76) Inventors: M. Arshad Siddiqui, Newton, MA (US); Chaoyang Dai, Acton, MA (US); Umar Faruk Mansoor, Framingham, MA (US); Liping Yang, Arlington, MA (US); Lalalnthi Dilrukshi Vitharana, Somerville, MA (US); Angie R. Angeles, Boston, MA (US)

(21) Appl. No.: 12/738,529

(22) PCT Filed: Oct. 16, 2008
(86) PCT No.: PCT/US08/80179

§ 371 Date: Feb. 28, 2011

Related U.S. Application Data
(60) Provisional application No. 60/986,880, filed on Nov. 9, 2007.

(51) Int. Cl. A61K 31/403; C07D 209/96; C07D 491/107; C07D 401/12; C07D 471/10; A61K 31/407; A61K 31/454; A61K 31/438; A61K 31/7105; A61K 31/711; A61K 51/00; A61K 39/395; A61K 38/21; A61P 35/00; A61P 9/00; A61P 37/06; A61P 31/10; A61P 19/02; A61P 1/00; A61P 29/00; A61P 37/02; A61P 35/02

(52) U.S. Cl. 424/1.11; 548/411; 548/409; 546/15; 546/18; 514/409; 514/278; 514/44 A; 424/178.1; 424/85.4

(57) ABSTRACT

The present invention relates to compounds of Formula (I), below, (wherein R1, R2, R3, R27, R28, R29, p, E, ring A, and ring B are as defined herein). The present invention also relates to compositions (including pharmaceutically acceptable compositions) comprising these compounds, alone and in combination with one or more additional therapeutic agents, and to methods for their use in inhibiting KSP kinesin activity, and for treating cellular proliferative diseases or disorders associated with KSP kinesin activity.

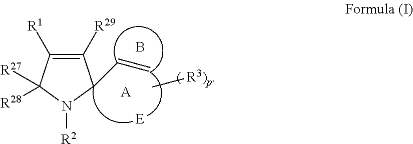

### PRIORITY

This application claims the benefit of priority of U.S. Provisional Application No. 60/986,880 filed, Nov. 9, 2007, the contents of which are incorporated in their entirety herein by reference.

### FIELD OF THE INVENTION

The present invention relates to compounds and compositions that are useful for treating cellular proliferative diseases or disorders associated with Kinesin Spindle Protein (“KSP”) kinesin activity and for inhibiting KSP kinesin activity.

### BACKGROUND OF THE INVENTION

Cancer is a leading cause of death in the United States and throughout the world. Cancer cells are often characterized by constitutive proliferative signals, defects in cell cycle checkpoints, as well as defects in apoptotic pathways. There is a great need for the development of new chemotherapeutic drugs that can block cell proliferation and enhance apoptosis of tumor cells.

Conventional therapeutic agents used to treat cancer include taxanes and vinca alkaloids, which target microtubules. Microtubules are an integral structural element of the mitotic spindle, which is responsible for the distribution of the duplicated sister chromatids to each of the daughter cells that result from cell division. Disruption of microtubules or interference with microtubule dynamics can inhibit cell division and induce apoptosis.

However, microtubules are also important structural elements in non-proliferative cells. For example, they are required for organelle and vesicle transport within the cell or along axons. Since microtubule-targeted drugs do not discriminate between these different structures, they can have undesirable side effects that limit usefulness and dosage. There is a need for chemotherapeutic agents with improved specificity to avoid side effects and improve efficacy.

Microtubules rely on two classes of motor proteins, the kinesins and dyneins, for their function. Kinesins are motor proteins that generate motion along microtubules. They are characterized by a conserved motor domain, which is approximately 320 amino acids in length. The motor domain binds and hydrolyses ATP as an energy source to drive directional movement of cellular cargo along microtubules and also contains the microtubule binding interface (Mandelkow and Mandelkow, Trends Cell Biol. 2002, 12:585-591).

Kinesins exhibit a high degree of functional diversity, and several kinesins are specifically required during mitosis and cell division. Different mitotic kinesins are involved in all aspects of mitosis, including the formation of a bipolar spindle, spindle dynamics, and chromosome movement. Thus, interference with the function of mitotic kinesins can disrupt normal mitosis and block cell division. Specifically, the mitotic kinesin KSP (also termed EG5), which is required for centrosome separation, was shown to have an essential function during mitosis. Cells in which KSP function is inhibited arrest in mitosis with unseparated centrosomes (Blangy at al., Cell 1995, 83:1159-1169). This leads to the formation of a monoastral array of microtubules, at the end of which the duplicated chromatids are attached in a rosette-like configuration. Further, this mitotic arrest leads to growth inhibition of tumor cells (Kaiser et al., J. Biol. Chem. 1999, 274:18925-18931). Inhibitors of KSP would be desirable for the treatment of proliferative diseases, such as cancer.

Kinesin inhibitors are known, and several molecules have recently been described in the literature. For example, adociasulfate-2 inhibits the microtubule-stimulated ATPase activity of several kinesins, including CENP-E (Sakowicz et al., Science 1998, 280:292-295). Rose Bengal lactone, another non-selective inhibitor, interferes with kinesin function by blocking the microtubule binding site (Hopkins at al., Biochemistry 2000, 39:2805-2814). Monastrol, a compound that has been isolated using a phenotypic screen, is a selective inhibitor of the KSP motor domain (Mayer et al., Science 1999, 286:971-974). Treatment of cells with monastrol arrests cells in mitosis with monopolar spindles.

KSP inhibitors have been disclosed in patents or publications, including: WO2006/031348, WO2006/110390, WO2006/068933, WO2006/023083, WO2006/007491, WO2006/086358, WO2003/105855, WO2006/023440, WO2003/079973, WO2004/087050, WO2004/111193, WO2004/112699, WO2006/007497, WO2006/101761, WO2006/007496, WO2005/017190, WO0224/037171, WO2005/019205, WO2005/019206, WO2005/102996, WO2006/101780, WO2006/007501, WO2005/018547, WO2004/058148, WO2004/058700, WO2005/018638, WO2007/054138, WO2006/133805, WO2006/002726, WO2006/133821, WO2005/108355, WO2006/094602, WO2005/092011, WO2006/031607, WO2004/111023, WO2006/137490, WO2006/101102, WO2006/101103, WO2006/101104, WO2006/101105, WO2004/092147, WO2005/035512, WO2006/044825, WO2006/044825, WO2006/119146, US2006/0247178, WO2006/098961, WO2006/098962, US2006/0258699, US2007/0213380, US2007/0112044, US2007/0155804, US2008/0194653, WO2008/042928. US2007/0249636, US2007/0287703, US2008/0153854, and US2007/0037853.

KSP, as well as other mitotic kinesins, are attractive targets for the discovery of novel chemotherapeutics with anti-proliferative activity. There is a need for compounds useful in the inhibition of KSP, and in the treatment of proliferative diseases, such as cancer.

### SUMMARY OF THE INVENTION

In one embodiment, the present invention provides a compound, or pharmaceutically acceptable salts, solvates, esters, prodrugs, or isomers of said compound, said compound having the general structure shown in Formula (I):

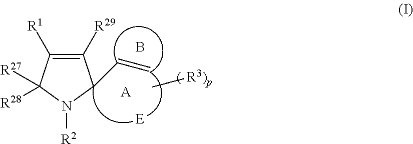

wherein R¹, R², R³, R²⁷, R²⁸, R²⁹, p, E, ring A, and ring B are selected independently of each other and wherein:
p is 0, 1, 2, 3, or 4;
ring A (including E and the unsaturation shown) is a 4-8 membered cycloalkenyl or heterocycloalkenyi ring;
E is selected from the group consisting of —O—, —S—, —S(O)—, —S(O)₂—, —C(R⁴)(R⁵)—, —N(R⁶)—, —N(C(Y)R⁷)—, —N(C(Y)OR⁸)—, —N(C(Y)N(R⁹)(R¹⁰))—, —C(O)—N(R¹¹)—, —N(R¹¹)—C(O)—, —S(O)₂—N(R¹¹)—, —N(R¹¹)—S(O)₂—, —C(O)—O—, —O—C(O)—, —O—N(R⁶)—, —N(R⁶)—O—, —N(R⁶)—N(R¹²)—, —C(R⁷)═N—, —C(O)—N═N—, —O—C(Y)—N(R¹¹)—, —N(R¹¹)—C(Y)—O—, —N(R¹¹)—C(Y)—N(R¹²)—, —C(Y)—N(R¹¹)—O—, —C(Y)—N(R¹¹)—N(R¹²)—, —O—N(R¹¹)—C(Y)—, and —N(R¹²)—N(R¹¹)—C(Y)—,
wherein each Y is independently selected from the group consisting of (═O), (═S), (═N(R¹³)), (═N(CN)), (═N(OR¹⁴)), (═N(R¹⁵)(R¹⁶)), and (═C(R¹⁷)(R¹⁸));
   ring B is an aromatic or heteroaromatic ring, or a partially unsaturated alicyclic ring, or a partially unsaturated heterocyclic ring,
wherein said ring is unsubstituted or optionally independently substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
   R¹ is selected from the group consisting of aryl, heteroaryl, cycloalkyl, cycloalkenyl, heterocycloalkyl, and heterocycloalkenyl,
wherein each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
   R² is selected from the group consisting of —C(Z)R⁷, —C(Z)NR⁹R¹⁰, —C(Z)OR⁸, —SO₂NR⁹R¹⁰, alkyl, heteroalkyl, aryl, heteroaryl, cycloalkyl, cycloalkenyl, heterocycloalkyl, and heterocycloalkenyl,
wherein each Z is independently selected from the group consisting of (═O), (═S), (═N(R¹³)), (═N(CN)), (═N(OR¹⁴)), (═N(R¹⁵)(R¹⁶)), and (═C(R¹⁷)(R¹⁸)), and
wherein each said alkyl, each said heteroalkyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group consisting of oxo (with the proviso that said aryl and said heteroaryl are not substituted with oxo), halogen, —CN, —NO₂, alkyl, heteroalkyl, haloalkyl, alkenyl, haloalkenyl, alkynyl, haloalkynyl, aryl, heteroaryl, aryl-alkyl, heteroaryl-alkyl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
   R²⁷ (when not joined with R²⁸) is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹—OC(O)R²⁴—NR²³C(N—CN)NR²⁵R²⁶ and —NR²³C(O)NR²⁵R²⁶,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰—NR²³SO₂R²⁴, —NR²³C(O)OR²⁰, —NR²³C(O)R²⁴, —SO₂NR²⁵R²⁶, —C(O)R²⁴—S(O)R¹⁹, —SO₂R¹⁹, —OC(O)R²⁴, —C(O)NR²⁵R²⁸, —NR²³C(N—CN)NR²⁵R²⁶ and —NR²³C(O)NR²⁵R²⁶;
   R²⁸ (when not joined with R²⁷) is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, R²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
   or, alternatively, R²⁷ and R²⁸, together with the carbon atom to which they are attached, form a cycloalkyl, a cycloalkenyl, a heterocycloalkyl ring containing from one to three heteroatoms selected from the group consisting of N, O, and S, or a heterocycloalkenyl ring containing from one to three heteroatoms selected from the group consisting of N, O, and S,
wherein said heterocycloalkyl ring and said heterocycloalkenyl ring are each unsubstituted or optionally independently substituted with one or more substituents, which can be the same or different, each substituent being independently selected from the group consisting of oxo, halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, NR²³C(N—CN)NR²⁵R²⁶ and —NR²³C(O)NR²⁵R²⁶:
   R²⁹ is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
   each R³ (when present) is independently selected from the group consisting of alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(S)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶, —NR²³C(O)NR²⁵R²⁶, and —NR²³—C(NH)—N(R²⁶)₂,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶,
or, alternatively, when p is 2, 3, or 4, any two R³ groups bound to the same ring carbon atom are taken together with the carbon atom to which they are attached to form a spirocycloalkyl, a spirocycloalkenyl, or a spiroheterocycloalkyl ring containing from one to three ring heteroatoms independently selected from the group consisting of —NH—, —NR⁶—, —S—, —S(O)—, —S(O)₂—, and —O—, or a spiroheterocycloalkenyl ring containing from one to three ring heteroatoms independently selected from the group consisting of —NH—, —NR⁶—, —S—, —S(O)—, —S(O)₂—, and —O—,
or, alternatively, R² and R³, together with the atom to which they are attached, are taken together with the carbon atom to which they are attached to form a cycloalkyl, a cycloalkenyl, a heterocycloalkyl ring containing from one to three ring heteroatoms independently selected from the group consisting of —NH—, —NR⁶—, —S—, —S(O)—, —S(O)₂—, and —O—, or a heterocycloalkenyl ring containing from one to three ring heteroatoms independently selected from the group consisting of —NH—, —NR⁶—, —S—. —S(O)—, —S(O)₂—, and —O—;
   each R⁴ (when not joined with R⁵) is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
   each R⁵ (when not joined with R⁴) is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
   or, alternatively, R⁴ and R⁵, together with the carbon atom to which they are attached, form a cycloalkyl, a cycloalkenyl, a heterocycloalkyl ring containing from one to three heteroatoms selected from the group consisting of N, O, and S, or a heterocycloalkenyl ring containing from one to three heteroatoms selected from the group consisting of N, O, and S,
wherein said heterocycloalkyl ring and said heterocycloalkenyl ring are each unsubstituted or optionally independently substituted with one or more substituents, which can be the same or different, each substituent being independently selected from the group consisting of oxo, halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
   each R⁶ is independently selected from the group consisting of H, alkyl, —C(O)R²⁴, —C(O)OR²⁰, —C(S)R²⁴, heteroalkyl, alkenyl, heteroalkenyl, alkynyl, heteroalkynyl, aryl, heteroaryl, cycloalkyl, cycloalkenyl, heterocycloalkyl, heterocycloalkenyl,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(S)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
   each R⁷ is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
   each R⁸ is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
   each R⁹ (when not joined with R¹⁰) is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
   each R¹⁹ (when not joined with R⁹) is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
   or, alternatively, R⁹ and R¹⁰, together with the N atom to which they are attached, form a heterocycloalkyl or a heterocycloalkenyl ring containing from one to three heteroatoms selected from the group consisting of N, O, and S,
wherein said heterocycloalkyl ring and said heterocycloalkenyl ring are each unsubstituted or optionally independently substituted with one or more substituents, which can be the same or different, each substituent being independently selected from the group consisting of oxo, halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
   each R¹¹ is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
   each R¹² is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
   each R¹³ is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²⁶C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
   each R¹⁴ is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
   each R¹⁵ (when not joined with R¹⁶) is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², NR²³SO₂R²⁴, —NR²³C(O)OR²⁰, —NR²³C(O)R²⁴, —SO₂NR²⁵R²⁶, —C(O)R²⁴, C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
   each R¹⁶ (when not joined with R¹⁶) is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
   or, alternatively, R¹⁵ and R¹⁶, together with the N atom to which they are attached, form a heterocycloalkyl or a heterocycloalkenyl ring containing from one to three heteroatoms selected from the group consisting of N, O, and S,
wherein said heterocycloalkyl ring and said heterocycloalkenyl ring are each unsubstituted or optionally independently substituted with one or more substituents, which can be the same or different, each substituent being independently selected from the group consisting of oxo, halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
   each R¹⁷ (when not joined with R¹⁸) is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, —CN, —OC(O)OR²⁰, —OR¹⁹, —NR²¹R²², —NR²³SO₂R²⁴, —NR²³C(O)OR²⁰, —NR²³C(O)R²⁴, —SO₂NR²⁵R²⁶, —(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰—NR²³SO₂R²⁴, —NR²³C(O)OR²⁰, —NR²³C(O)R²⁴, —SO₂NR²⁵R²⁶, —C(O)R²⁴, —C(O)OR²⁰, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
   each R¹⁸ (when not joined with R¹⁷) is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, —CN, —OC(O)OR²⁰, —OR¹⁹, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
   or, alternatively, R¹⁷ and R¹⁸, together with the carbon atom to which they are attached, form a cycloalkyl, a cycloalkenyl, a heterocycloalkyl ring containing from one to three heteroatoms selected from the group consisting of N, O, and S, or a heterocycloalkenyl ring containing from one to three heteroatoms selected from the group consisting of N, O, and S,
wherein said heterocycloalkyl ring and said heterocycloalkenyl ring are each unsubstituted or optionally independently substituted with one or more substituents, which can be the same or different, each substituent being independently selected from the group consisting of oxo, halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
   each R¹⁹ is independently selected from the group consisting of H, alkyl, haloalkyl, heteroalkyl, haloheteroalkyl, aryl, heteroaryl, cycloalkyl, halocycloalkyl;
   each R²⁰ is independently selected from the group consisting of H, alkyl, haloalkyl, heteroalkyl, haloheteroalkyl, aryl, heteroaryl, cycloalkyl, halocycloalkyl;
   each R²¹ (when not joined with R²²) is independently selected from the group consisting of H, alkyl, haloalkyl, heteroalkyl, haloheteroalkyl, aryl, heteroaryl, cycloalkyl, halocycloalkyl;
   each R²² (when not joined with R²¹) is independently selected from the group consisting of H, alkyl, haloalkyl, heteroalkyl, haloheteroalkyl, aryl, heteroaryl, cycloalkyl, halocycloalkyl;
   or, alternatively, R²¹ and R²², together with the N atom to which they are attached, form a heterocycloalkyl or a heterocycloalkenyl ring containing from one to three heteroatoms selected from the group consisting of N, O, and S;
   each R²³ is independently selected from the group consisting of H, alkyl, haloalkyl, heteroalkyl, haloheteroalkyl, aryl, heteroaryl, cycloalkyl, halocycloalkyl;
   each R²⁴ is independently selected from the group consisting of H, alkyl, haloalkyl, heteroalkyl, haloheteroalkyl, aryl, heteroaryl, cycloalkyl, halocycloalkyl;
   each R²⁵ (when not joined with R²⁶) is independently selected from the group consisting of H, alkyl, haloalkyl, heteroalkyl, haloheteroalkyl, aryl, heteroaryl, cycloalkyl, halocycloalkyl; and
   each R²⁶ (when not joined with R²⁵) is independently selected from the group consisting of H, alkyl, haloalkyl, heteroalkyl, haloheteroalkyl, aryl, heteroaryl, cycloalkyl, halocycloalkyl:
   or, alternatively, R²⁵ and R²⁶, together with the N atom to which they are attached, form a heterocycloalkyl or a heterocycloalkenyl ring containing from one to three heteroatoms selected from the group consisting of N, O, and S.

As explained in more detail below, it shall be understood that ring A can have unsaturation in addition to the unsaturation shown in the generic formulas provided herein.

Pharmaceutical formulations or compositions comprising a therapeutically effective amount of at least one of the inventive compounds, and/or pharmaceutically acceptable salts, solvates, esters, prodrugs, or isomers thereof and a pharmaceutically acceptable carrier also are provided. Pharmaceutical formulations or compositions comprising a therapeutically effective amount of at least one of the inventive compounds (and/or pharmaceutically acceptable salts, solvates, esters, prodrugs, or isomers thereof) and a pharmaceutically acceptable carrier together with one or more additional active ingredients are also contemplated.

Methods of treating cellular proliferative diseases, disorders associated with KSP kinesin activity and/or for inhibiting KSP kinesin activity in a subject comprising administering to a subject in need of such treatment an effective amount of at least one of the inventive compounds or formulations or compositions according to the invention are also are provided. The methods according to the invention may be used in a single agent regimen or as part of a multiple agent regimen as is determined appropriate by those skilled in the art.

Other than in the operating examples, or where otherwise indicated, all numbers expressing quantities of ingredients, reaction conditions, and so forth used in the specification and claims are to be understood as being modified in all instances by the term “about.”

### DETAILED DESCRIPTION

In one embodiment, the compounds of the invention have a structure shown in Formula (I) and include pharmaceutically acceptable salts, solvates, esters, prodrugs, or isomers of said compounds.

As stated in Formula (I) (and in other formulas described herein depicting various embodiments of the compounds of the invention), ring A is a 4-8 membered cycloalkenyl or heterocycloalkenyl ring. It shall be understood that such cycloalkenyl or heterocycloalkenyl rings of ring A can have unsaturation that is in addition to the unsaturation shown in the generic formulas provided herein. For purposes of illustration only, non-limiting examples of such additional unsaturation in ring A include:

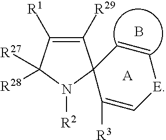

Additional non-limiting examples include:

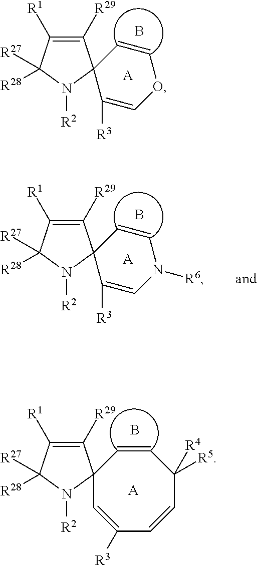

In one embodiment, in Formula (I), ring A is a cycloalkenyl ring.

In one embodiment, in Formula (I), ring A is a heterocycloalkenyl ring.

In one embodiment, in Formula (I), ring A is a 4-membered ring.

In one embodiment, in Formula (I), ring A is a 5-membered ring.

In one embodiment, in Formula (I), ring A is a 6-membered ring.

In one embodiment, in Formula (I), ring A is a 7-membered ring.

In one embodiment, in Formula (I), ring A is an 8-membered ring.

In one embodiment, in Formula (I), ring A (including the unsaturation shown) is mono-unsaturated.

In one embodiment, in Formula (I), ring A (including the unsaturation shown) is poly-unsaturated.

In one embodiment, in Formula (I), E is —C(R⁴)(R⁵)—.

In one embodiment, in Formula (I), E is selected from the group consisting of —O—, —S—, —S(O)—, —S(O)₂—, —N(R⁶)—, —N(C(Y)R⁷)—, —N(C(Y)OR⁸)—, —N(C(Y)N(R⁹)(R¹⁰))—, —C(O)—N(R¹¹)—, —N(R¹¹)—C(O)—, —S(O)₂—N(R¹¹)—, —N(R¹¹)—S(O)₂—, —C(O)—O—, —O—C(O)—, —O—N(R⁶)—, —N(R⁶)—O—, —N(R⁶)—N(R¹²)—, —C(R⁷)═N—, —C(O)—C(R⁷)═N—, —C(O)—N═N—, —O—C(Y)—N(R¹¹)—, —N(R¹¹)—C(Y)—O—, —N(R¹¹)—C(Y)—N(R¹²)—, —C(Y)—N(R¹¹)—O—, —C(Y)—N(R¹¹)—N(R¹²)—, —O—N(R¹¹)—C(Y)—, and —N(R¹²)—N(R¹¹)—C(Y)—.

In one embodiment, in Formula (I), E is selected from the group consisting of —O—, —S—, —S(O)—, —S(O)₂—, and —N(R⁶)—.

In one embodiment, in Formula (I), E is selected from the group consisting of —O—, —S—, —S(O)—, —S(O)₂—, and —N(R⁶)—, wherein R⁶ is selected from the group consisting of H, alkyl, —C(O)R²⁴, —C(O)OR²⁰, and —C(S)R²⁴.

In one embodiment, in Formula (I), E is selected from the group consisting of —O— and —N(R⁶)—, wherein R⁶ is selected from the group consisting of H, alkyl, —C(O)R²⁴, —C(O)OR²⁰, and —C(S)R²⁴.

In one embodiment, in Formula (I), when E is —N(R⁶)—, then p is 0 and R³ is absent. In such embodiments, non-limiting examples of R⁶ include H, alkyl, —C(O)R²⁴, —C(O)OR²⁰, and —C(S)R²⁴.

In one embodiment, in Formula (I), E is —O—.

In one embodiment, in Formula (I), E is —S—.

In one embodiment, in Formula (I), E is —S(O)—.

In one embodiment, in Formula (I), E is —S(O)₂—.

In one embodiment, in Formula (I), E is —CH₂—.

In one embodiment, in Formula (I), E is —CHR⁴—.

In one embodiment, in Formula (I), E is —CR⁴R⁶—.

In one embodiment, in Formula (I), E is —N(R⁶)—.

In one embodiment, in Formula (I), E is —N(C(Y)R⁷)—.

In one embodiment, in Formula (I), E is —N(C(Y)OR⁸)—.

In one embodiment, in Formula (I), E is —N(C(Y)N(R⁹)(R¹⁰))—.

In one embodiment, in Formula (I), E is —C(O)—N(R¹¹)—.

In one embodiment, in Formula (I), E is —N(R¹¹)—C(O)—.

In one embodiment, in Formula (I), E is —S(O)₂—N(R¹¹)—.

In one embodiment, in Formula (I), E is —N(R¹¹)—S(O)₂—.

In one embodiment, in Formula (I), E is —C(O)—O—.

In one embodiment, in Formula (I), E is —O—C(O)—.

In one embodiment, in Formula (I), E is —O—N(R⁶)—.

In one embodiment, in Formula (I), E is —N(R⁶)—O—.

In one embodiment, in Formula (I), E is —N(R⁶)—N(R¹²)

In one embodiment, in Formula (I), E is —N═N—.

In one embodiment, in Formula (I), E is —C(R⁷)═N—.

In one embodiment, in Formula (I), E is —C(O)—C(R⁷)═N—.

In one embodiment, in Formula (I), E is —C(O)—N═N—.

In one embodiment, in Formula (I), E is —O—C(Y)—N(R¹¹)—.

In one embodiment, in Formula (I), E is —N(R¹¹)—C(Y)—O—.

In one embodiment, in Formula (I), E is —N(R¹¹)—C(Y)—N(R¹²)—.

In one embodiment, in Formula (I), E is —C(Y)—N(R¹¹)—O—.

In one embodiment, in Formula (I), E is —C(Y)—N(R¹¹)—N(R¹²)—.

In one embodiment, in Formula (I), E is —O—N(R¹¹)—C(Y)—.

In one embodiment, in Formula (I), E is —N(R¹²)—N(R¹¹)—C(Y)—.

In one embodiment, in Formula (I), Y is (═O).

In one embodiment, in Formula (I), Y is (═S).

In one embodiment, in Formula (I), Y is (═N(R¹³)).

In one embodiment, in Formula (I), Y is (═N(CN)).

In one embodiment, in Formula (I), Y is (═N(OR¹⁴)).

In one embodiment, in Formula (I), Y is (═N(R¹⁵)(R¹⁶)).

In one embodiment, in Formula (I), Y is (═C(R¹⁷)(R¹⁸)).

In one embodiment, in Formula (I), ring A is a 4-7-membered cycloalkylene ring and E is —C(R⁴)(R⁵)—.

In one embodiment, in Formula (I), ring A is a 5-7-membered heterocycloalkylene ring and E is selected from the group consisting of —O—, —S—, —S(O)—, —S(O)₂—, —N(R⁶)—, —N(C(Y)R⁷)—, —N(C(Y)OR⁸)—, —N(C(Y)N(R⁹)(R¹⁰))—, —C(O)—N(R¹¹)—, —N(R¹¹)—C(O)—, —S(O)₂—N(R¹¹)—, —N(R¹¹)—S(O)₂—, —C(O)—O—, —O—C(O)—, —O—N(R⁶)—, —N(R⁶)—O—, —N(R⁶)—N(R¹²)—, —C(R⁷)═N—, —C(O)—C(R⁷)═N—, —C(O)—N═N—, —O—C(Y)—N(R¹¹)—, —N(R¹¹)—C(Y)—O—, —N(R¹¹)—C(Y)—N(R¹²)—, —C(Y)—N(R¹¹)—O—, —C(Y)—N(R¹¹)—N(R¹²)—, —O—N(R¹¹)—C(Y)—, and —N(R¹²)—N(R¹¹)—C(Y)—.

In one embodiment, in Formula (I), ring A is a 5-6-membered heterocycloalkylene ring and E is selected from the group consisting of —O—, —S—, —S(O)—, —S(O)₂—, —N(R⁶)—, —C(O)—N(R¹¹)—, and —N(R¹¹)—C(O)—.

In one embodiment, in Formula (I), ring A is a 5-6-membered heterocycloalkylene ring and E is selected from the group consisting of —O—, —S—, —S(O)—, —S(O)₂—, and —N(R⁶)—. In one such embodiment, in Formula (I), R⁶ is selected from the group consisting of H, alkyl, —C(O)R²⁴, —C(O)OR²⁰, and —C(S)R²⁴.

In one embodiment, in Formula (I), ring A is a 5-6-membered heterocycloalkylene ring and E is selected from the group consisting of —O— and —N(R⁶)—. In one such embodiment, in Formula (I), R⁶ is selected from the group consisting of H, alkyl, —C(O)R²⁴, —C(O)OR²⁰, and —C(S)R²⁴. In one such embodiment, in Formula (I), ring A is a 5-membered heterocycloalkylene ring. In another such embodiment, in Formula (I), ring A is a 6-membered heterocycloalkylene ring.

In one embodiment, in Formula (I), ring A is a 4-membered ring and E is —C(R⁴)(R⁵)—.

In one embodiment, in Formula (I), ring A is a 4-membered ring and E is selected from the group consisting of —O—, —S—, —S(O)—, —S(O)₂—, —N(R⁶)—, —N(C(Y)R⁷)—, —N(C(Y)OR⁸)—, —N(C(Y)N(R⁹)(R¹⁰))—, —C(O)—N(R¹¹)—, —N(R¹¹)—C(O)—, —S(O)₂—N(R¹¹)—, —N(R¹¹)—S(O)₂—, —C(O)—O—, —O—C(O)—, —O—N(R⁶)—, —N(R⁶)—O—, —N(R⁶)—N(R¹²)—, —C(R⁷)═N—, —C(O)—C(R⁷)═N—, —C(O)—N═N—, —O—C(Y)—N(R¹¹)—, —N(R¹¹)—C(Y)—O—, —N(R¹¹)—C(Y)—N(R¹²)—, —C(Y)—N(R¹¹)—O—, —C(Y)—N(R¹¹)—N(R¹²)—, —O—N(R¹¹)—C(Y)—, and —N(R¹²)—N(R¹¹)—C(Y)—.

In one embodiment, in Formula (I), ring A is a 4-membered ring and E is selected from the group consisting of —O—, —S—, —S(O)—, —S(O)₂—, and —N(R⁶)—.

In one embodiment, in Formula (I), ring A is a 4-membered ring and E is selected from the group consisting of —O—, —S—, —S(O)—, —S(O)₂—, and —N(R⁶)—, wherein R⁶ is selected from the group consisting of H, alkyl, —C(O)R²⁴, —C(O)OR²⁰, and —C(S)R²⁴.

In one embodiment, in Formula (I), ring A is a 4-membered ring and E is selected from the group consisting of —O— and —N(R⁶)—, wherein R⁶ is selected from the group consisting of H, alkyl, —C(O)R²⁴, —C(O)OR²⁰, and —C(S)R²⁴.

In one embodiment, in Formula (I), ring A is a 4-membered ring and E is selected from the group consisting of —O—, —S—, —S(O)—, —S(O)₂—, —C(R⁴)(R⁵)—, —N(R⁶)—, —N(C(Y)R⁷)—, —N(C(Y)OR⁸)—, —N(C(Y)N(R⁹)(R¹⁰))—.

In one embodiment, in Formula (I), A is a 4-membered ring and E is selected from the group consisting of —CH₂—, —CH(R⁴)—, —C(R⁴)(R⁵)—.

In one embodiment, in Formula (I), ring A is a 5-membered ring and E is —C(R⁴)(R⁵)—.

In one embodiment, in Formula (I), ring A is a 5-membered ring and E is selected from the group consisting of —O—, —S—, —S(O)—, —S(O)₂—, —N(R⁶)—, —N(C(Y)R⁷)—, —N(C(Y)OR⁸)—, —N(C(Y)N(R⁹)(R¹⁰))—, —C(O)—N(R¹¹)—, —N(R¹¹)—C(O)—, —S(O)₂—N(R¹¹)—, —N(R¹¹)—S(O)₂—, —C(O)—O—, —O—C(O)—, —O—N(R⁶)—, —N(R⁶)—O—, —N(R⁶)—N(R¹²)—, —C(R⁷)═N—, —C(O)—C(R⁷)═N—, —C(O)—N═N—, —O—C(Y)—N(R¹¹)—, —N(R¹¹)—C(Y)—O—, —N(R¹¹)—C(Y)—N(R¹²)—, —C(Y)—N(R¹¹)—O—, —C(Y)—N(R¹¹)—N(R¹²)—, —O—N(R¹¹)—C(Y)—, and —N(R¹²)—N(R¹¹)—C(Y)—.

In one embodiment, in Formula (I), ring A is a 5-membered ring and E is selected from the group consisting of —O—, —S—, —S(O)—, —S(O)₂—, and —N(R⁶)—.

In one embodiment, in Formula (I), ring A is a 5-membered ring and E is selected from the group consisting of —O—, —S—, —S(O)—, —S(O)₂—, and —N(R⁶)—, wherein R⁶ is selected from the group consisting of H, alkyl, —C(O)R²⁴, —C(O)OR²⁰, and C(S)R²⁴.

In one embodiment, in Formula (I), ring A is a 5-membered ring and E is selected from the group consisting of —O— and —N(R⁶)—, wherein R⁶ is selected from the group consisting of H, alkyl, —C(O)R²⁴, —C(O)OR²⁰, and —C(S)R²⁴.

In one embodiment, in Formula (I), ring A is a 5-membered ring and E is selected from the group consisting of —O—, —S—, —S(O)—, —S(O)₂—, —C(R⁴)(R⁵)—, —N(R⁶)—, —N(C(Y)R⁷)—, —N(C(Y)OR⁸)—, —N(C(Y)N(R⁹)(R¹⁰))—, —C(O)—N(R¹¹)—, —N(R¹¹)—C(O)—, —S(O)₂—N(R¹¹)—, —N(R¹¹)—S(O)₂—, —C(O)—O—, —O—C(O)—, —O—N(R⁶)—, —N(R⁶)—O—, —N(R⁶)—N(R¹²)—, and —C(R⁷)═N—.

In one embodiment, in Formula (I), A is a 5-membered ring and E is —O—.

In one embodiment, in Formula (I), A is a 5-membered ring and E is —S—.

In one embodiment, in Formula (I), A is a 5-membered ring and E is —S(O)—.

In one embodiment, in Formula (I), A is a 5-membered ring and E is —S(O)₂—.

In one embodiment, in Formula (I), A is a 5-membered ring and E is —C(R⁴)(R⁵)—.

In one embodiment, in Formula (I), A is a 5-membered ring and E is —N(R⁶)—.

In one embodiment, in Formula (I), A is a 5-membered ring and E is —N(C(Y)R⁷)—.

In one embodiment, in Formula (I), A is a 5-membered ring and E is —N(C(Y)OR⁸)—.

In one embodiment, in Formula (I), A is a 5-membered ring and E is —N(C(Y)N(R⁹)(R¹⁰))—.

In one embodiment, in Formula (I), A is a 5-membered ring and E is —C(O)—N(R¹¹)—.

In one embodiment, in Formula (I), A is a 5-membered ring and E is —N(R¹¹)—C(O)—.

In one embodiment, in Formula (I), A is a 5-membered ring and E is —S(O)₂—N(R¹¹)—.

In one embodiment, in Formula (I), A is a 5-membered ring and E is —N(R¹¹)—S(O)₂—.

In one embodiment, in Formula (I), A is a 5-membered ring and E is —C(O)—O—.

In one embodiment, in Formula (I), A is a 5-membered ring and E is —O—C(O)—.

In one embodiment, in Formula (I), A is a 5-membered ring and E is —O—N(R⁶)—.

In one embodiment, in Formula (I), A is a 5-membered ring and E is —N(R⁶)—O—.

In one embodiment, in Formula (I), A is a 5-membered ring and E is —N(R⁶)—N(R¹²)—.

In one embodiment, in Formula (I), A is a 5-membered ring and E is —N═N—.

In one embodiment, in Formula (I), A is a 5-membered ring and E is —C(R⁷)═N—.

In one embodiment, in Formula (I), ring A is a 6-membered ring and E is —C(R⁴)(R⁵)—.

In one embodiment, in Formula (I), ring A is a 6-membered ring and E is selected from the group consisting of —O—, —S—, —S(O)—, —S(O)₂—, —N(R⁶)—, —N(C(Y)R⁷)—, —N(C(Y)OR⁸)—, —N(C(Y)N(R⁹)(R¹⁰))—, —C(O)—N(R¹¹)—, —N(R¹¹)—C(O)—, —S(O)₂—N(R¹¹)—, —N(R¹¹)—S(O)₂—, —C(O)—O—, —O—C(O)—, —O—N(R⁶)—, —N(R⁸)—O—, —N(R⁶)—N(R¹²)—, —N═N—, —C(R⁷)═N—, —C(O)—C(R⁷)═N—, —C(O)—N═N—, —O—C(Y)—N(R¹¹)—, —N(R¹¹)—C(Y)—O—, —N(R¹¹)—C(Y)—N(R¹²)—, —C(Y)—N(R¹¹)—O—, —C(Y)—N(R¹¹)—N(R¹²)—, —O—N(R¹¹)—C(Y)—, and —N(R¹²)—N(R¹¹)—C(Y)—.

In one embodiment, in Formula (I), ring A is a 6-membered ring and E is selected from the group consisting of —O—, —S—, —S(O)—, —S(O)₂—, and —N(R⁶)—.

In one embodiment, in Formula (I), ring A is a 6-membered ring and E is selected from the group consisting of —O—, —S—, —S(O)—, —S(O)₂—, and —N(R⁶)—, wherein R⁶ is selected from the group consisting of H, alkyl, —C(O)R²⁴, —C(O)OR²⁰, and —C(S)R²⁴.

In one embodiment, in Formula (I), ring A is a 6-membered ring and E is selected from the group consisting of —O— and —N(R⁶)—, wherein R⁶ is selected from the group consisting of H, alkyl, —C(O)R²⁴, —C(O)OR²⁰, and —C(S)R²⁴.

In one embodiment, in Formula (I), A is a 6-membered ring and E is selected from the group consisting of —O—, —S—, —S(O)—, —S(O)₂—, —C(R⁴)(R⁵)—, —N(R⁶)—, —N(C(Y)R⁷)—, —N(C(Y)OR⁸)—, —N(C(Y)N(R⁹)(R¹⁰))—, —C(O)—N(R¹¹)—, —N(R¹¹)—C(O)—, —S(O)₂—N(R¹¹)—, —N(R¹¹)—S(O)₂—, —C(O)—O—, —O—C(O)—, —O—N(R6)-, —N(R6)-O—, —N(R6)-N(R12)-, —N═N—, —C(R7)=N—, —C(O)—C(R7)=N—, —C(O)—N═N—, —O—C(Y)—N(R¹¹)—, —N(R¹¹)—C(Y)—O—, —N(R¹¹)—C(Y)—N(R¹²)—, —C(Y)—N(R¹¹)—O—, —C(Y)—N(R¹¹)—N(R¹²)—, —O—N(R¹¹)—C(Y)—, and —N(R¹²)—N(R¹¹)—C(Y)—.

In one embodiment, in Formula (I), ring A is a 6-membered ring and E is —O—.

In one embodiment, in Formula (I), ring A is a 6-membered ring and E is —S—.

In one embodiment, in Formula (I), ring A is a 6-membered ring and E is —S(O)—.

In one embodiment, in Formula (I), ring A is a 6-membered ring and E is —S(O)₂—.

In one embodiment, in Formula (I), ring A is a 6-membered ring and E is —C(R⁴)(R⁵)—.

In one embodiment, in Formula (I), ring A is a 6-membered ring and E is —N(R⁶)—.

In one embodiment, in Formula (I), ring A is a 6-membered ring and E is —N(C(Y)R⁷)—.

In one embodiment, in Formula (I), ring A is a 6-membered ring and E is —N(C(Y)OR⁸)—.

In one embodiment, in Formula (I), ring A is a 6-membered ring and E is —N(C(Y)N(R⁹)(R¹⁰))—.

In one embodiment, in Formula (I), ring A is a 6-membered ring and E is —C(O)—N(R¹¹)—.

In one embodiment, in Formula (I), ring A is a 6-membered ring and E is —N(R¹¹)—C(O)—.

In one embodiment, in Formula (I), ring A is a 6-membered ring and E is —S(O)₂—N(R¹¹)—.

In one embodiment, in Formula (I), ring A is a 6-membered ring and E is —N(R¹¹)—S(O)₂—.

In one embodiment, in Formula (I), ring A is a 6-membered ring and E is —C(O)—O—.

In one embodiment, in Formula (I), ring A is a 6-membered ring and E is —O—C(O)—.

In one embodiment, in Formula (I), ring A is a 6-membered ring and E is —O—N(R⁶)—,

In one embodiment, in Formula (I), ring A is a 6-membered ring and E is —N(R⁶)—O—.

In one embodiment, in Formula (I), ring A is a 6-membered ring and E is —N(R6)-N(R12)-.

In one embodiment, in Formula (I), ring A is a 6-membered ring and E is —N═N—.

In one embodiment, in Formula (I), ring A is a 6-membered ring and E is —C(R7)=N—.

In one embodiment, in Formula (I), ring A is a 6-membered ring and E is —C(O)—C(R7)=N—.

In one embodiment, in Formula (I), ring A is a 6-membered ring and E is —C(O)—N═N—.

In one embodiment, in Formula (I), ring A is a 6-membered ring and E is —O—C(Y)—N(R¹¹)—.

In one embodiment, in Formula (I), ring A is a 6-membered ring and E is —N(R¹¹)—C(Y)—O—.

In one embodiment, in Formula (I), ring A is a 6-membered ring and E is —N(R¹¹)—C(Y)—N(R¹²)—.

In one embodiment, in Formula (I), ring A is a 6-membered ring and E is —C(Y)—N(R¹¹)—O—.

In one embodiment, in Formula (I), ring A is a 6-membered ring and E is —C(Y)—N(R¹¹)—N(R¹²)—.

In one embodiment, in Formula (I), ring A is a 6-membered ring and E is —O—N(R¹¹)—C(Y)—.

In one embodiment, in Formula (I), ring A is a 6-membered ring and E is —N(R¹²)—N(R¹¹)—C(Y)—.

In one embodiment, in Formula (I), ring A is a 7-membered ring and E is —C(R⁴)(R⁵)—.

In one embodiment, in Formula (I), ring A is a 7-membered ring and E is selected from the group consisting of —O—, —S—, —S(O)—, —S(O)₂—, —N(R⁶)—, —N(C(Y)R⁷)—, —N(C(Y)OR⁸)—, —N(C(Y)N(R⁹)(R¹⁰))—, —C(O)—N(R¹¹)—, —N(R¹¹)—C(O)—, —S(O)₂—N(R¹¹)—, —N(R¹¹)—S(O)₂—, —C(O)—O—, —O—C(O)—, —O—N(R⁶)—, —N(R⁶)—O—, —N(R⁶)—N(R¹²)—, —C(R⁷)═N—, —C(O)—C(R⁷)═N—, —C(O)—N═N—, —O—C(Y)—N(R¹¹)—, —N(R¹¹)—C(Y)—O—, —N(R¹¹)—C(Y)—N(R¹²)—, —C(Y)—N(R¹¹)—O—, —C(Y)—N(R¹¹)—N(R¹²)—, —O—N(R¹¹)—C(Y)—, and —N(R¹²)—N(R¹¹)—C(Y)—.

In one embodiment, in Formula (I), ring A is a 7-membered ring and E is selected from the group consisting of —O—, —S—, —S(O)—, —S(O)₂—, and —N(R⁶)—.

In one embodiment, in Formula (I), ring A is a 7-membered ring and E is selected from the group consisting of —O—, —S—, —S(O)—, —S(O)₂—, and —N(R⁶)—, wherein R⁶ is selected from the group consisting of H, alkyl, —C(O)R²⁴, —C(O)OR²⁰, and —C(S)R²⁴.

In one embodiment, in Formula (I), ring A is a 7-membered ring and E is selected from the group consisting of —O— and —N(R⁶)—, wherein R⁶ is selected from the group consisting of H, alkyl, —C(O)R²⁴, —C(O)OR²⁰, and —C(S)R²⁴.

In one embodiment, in Formula (I), ring A is a 7-membered ring and E is selected from the group consisting of —O—, —S—, —S(O)—, —S(O)₂—, —C(R⁴)(R⁵)—, —N(R⁶)—, —N(C(Y)R⁷)—, —N(C(Y)OR8)-, —N(C(Y)N(R9)(R10))-, —C(O)—N(R11)-, —N(R11)-C(O)—, —S(O)₂—N(R11)-, —N(R11)-S(O)₂—, —C(O)—O—, —O—C(O)—, —O—N(R6)-, —N(R6)-O—, —N(R6)-N(R12)-, —N═N—, —C(R7)=N—, —C(O)—C(R7)=N—, —C(O)—N═N—, —O—C(Y)—N(R11)-, —N(R11)-C(Y)—O—, —N(R11)-C(Y)—N(R12)-, —C(Y)—N(R11)-O—, —C(Y)—N(R11)-N(R12)-, —O—N(R11)-C(Y)—, and —N(R12)-N(R11)-C(Y)—.

In one embodiment, in Formula (I), A is a 7-membered ring and E is —O—.

In one embodiment, in Formula (I), A is a 7-membered ring and E is —S—.

In one embodiment, in Formula (I), A is a 7-membered ring and E is —S(O)—.

In one embodiment, in Formula (I), A is a 7-membered ring and E is —S(O)₂—.

In one embodiment, in Formula (I), A is a 7-membered ring and E is —C(R⁴)(R⁵)—.

In one embodiment, in Formula (I), A is a 7-membered ring and E is —N(R⁶)—.

In one embodiment, in Formula (I), A is a 7-membered ring and E is —N(C(Y)R⁷)—.

In one embodiment, in Formula (I), A is a 7-membered ring and E is —N(C(Y)OR⁸)—.

In one embodiment, in Formula (I), A is a 7-membered ring and E is —N(C(Y)N(R⁹)(R¹⁰))—.

In one embodiment, in Formula (I), A is a 7-membered ring and E is —C(O)—N(R¹¹)—.

In one embodiment, in Formula (I), A is a 7-membered ring and E is —N(R¹¹)—C(O)—.

In one embodiment, in Formula (I), A is a 7-membered ring and E is —S(O)₂—N(R¹¹)—.

In one embodiment, in Formula (I), A is a 7-membered ring and E is —N(R¹¹)—S(O)₂—.

In one embodiment, in Formula (I), A is a 7-membered ring and E is —C(O)—O—.

In one embodiment, in Formula (I), A is a 7-membered ring and E is —O—C(O)—.

In one embodiment, in Formula (I), A is a 7-membered ring and E is —O—N(R⁶)—.

In one embodiment, in Formula (I), A is a 7-membered ring and E is —N(R⁶)—O—.

In one embodiment, in Formula (I), A is a 7-membered ring and E is —N(R⁶)—N(R¹²)—.

In one embodiment, in Formula (I), A is a 7-membered ring and E is —N═N—.

In one embodiment, in Formula (I), A is a 7-membered ring and E is —C(R⁷)═N—.

In one embodiment, in Formula (I), A is a 7-membered ring and E is —C(O)—C(R⁷)═N—.

In one embodiment, in Formula (I), A is a 7-membered ring and E is —C(O)—N═N—.

In one embodiment, in Formula (I), A is a 7-membered ring and E is —O—C(Y)—N(R¹¹)—.

In one embodiment, in Formula (I), A is a 7-membered ring and E is —N(R¹¹)—C(Y)—O—.

In one embodiment, in Formula (I), A is a 7-membered ring and E is —N(R¹¹)—C(Y)—N(R¹²)—.

In one embodiment, in Formula (I), A is a 7-membered ring and E is —C(Y)—N(R¹¹)—O—.

In one embodiment, in Formula (I), A is a 7-membered ring and E is —C(Y)—N(R¹¹)—N(R¹²)—.

In one embodiment, in Formula (I), A is a 7-membered ring and E is —O—N(R¹¹)—C(Y)—.

In one embodiment, in Formula (I), A is a 7-membered ring and E is —N(R¹²)—N(R¹¹)—C(Y)—.

In one embodiment, in Formula (I), ring A is a 8-membered ring and E is —C(R⁴)(R⁵)—.

In one embodiment, in Formula (I), ring A is a 8-membered ring and E is selected from the group consisting of —O—, —S—, —S(O)—, —S(O)₂—, —N(R⁶)—, —N(C(Y)R⁷)—, —N(C(Y)OR⁸)—, —N(C(Y)N(R⁹)(R¹⁰))—, —C(O)—N(R¹¹)—, —N(R¹¹)—C(O)—, —S(O)₂—N(R¹¹)—, —N(R¹¹)—S(O)₂—, —C(O)—O—, —O—C(O)—, —O—N(R⁶)—, —N(R⁶)—O—, —N(R⁶)—N(R¹²)—, —N═N—, —C(R⁷)═N—, —C(O)—C(R⁷)═N—, —C(O)—N═N—, —O—C(Y)—N(R¹¹)—, —N(R¹¹)—C(Y)—O—, —N(R¹¹)—C(Y)—N(R¹²)—, —C(Y)—N(R¹¹)—O—, —C(Y)—N(R¹¹)—N(R¹²)—, —O—N(R¹¹)—C(Y)—, and —N(R¹²)—N(R¹¹)—C(Y)—.

In one embodiment, in Formula (I), ring A is a 8-membered ring and E is selected from the group consisting of —O—, —S—, —S(O)—, —S(O)₂—, and —N(R⁶)—.

In one embodiment, in Formula (I), ring A is a 8-membered ring and E is selected from the group consisting of —O—, —S—, —S(O)—, —S(O)₂—, and —N(R⁶)—, wherein R⁶ is selected from the group consisting of H, alkyl, —C(O)R²⁴, —C(O)OR²⁰, and —C(S)R²⁴.

In one embodiment, in Formula (I), ring A is a 8-membered ring and E is selected from the group consisting of —O— and —N(R⁶)—, wherein R⁶ is selected from the group consisting of H, alkyl, —C(O)R²⁴, —C(O)OR^{2D}, and —C(S)R²⁴.

In one embodiment, in Formula (I), ring A is a 8-membered ring and E is selected from the group consisting of —O—, —S—, —S(O)—, —S(O)₂—, —C(R⁴)(R⁶)—, —N(R⁶)—, —N(C(Y)R⁷)—, —N(C(Y)OR⁸)—, —N(C(Y)N(R⁹)(R¹⁰))—, —C(O)—N(R¹¹)—, —N(R¹¹)—C(O)—, —S(O)₂—N(R¹¹)—, —N(R¹¹)—S(O)₂—, —C(O)—O—, —O—C(O)—, —O—N(R⁶)—, —N(R⁶)—O—, —N(R⁶)—N(R¹²)—, —N═N—, —C(R⁷)═N—, —C(O)—C(R⁷)═N—, —C(O)—N═N—, —O—C(Y)—N(R¹¹)—, —N(R¹¹)—C(Y)—O—, —N(R¹¹)—C(Y)—N(R¹²)—, —C(Y)—N(R¹¹)—O—, —C(Y)—N(R¹¹)—N(R¹²)—, —O—N(R¹¹)—C(Y)—, and —N(R¹²)—N(R¹¹)—C(Y)—.

In one embodiment, in Formula (I), A is a 8-membered ring and E is —O—.

In one embodiment, in Formula (I), A is a 8-membered ring and E is —S—.

In one embodiment, in Formula (I), A is a 8-membered ring and E is —S(O)—.

In one embodiment, in Formula (I), A is a 8-membered ring and E is —S(O)₂—.

In one embodiment, in Formula (I), A is a 8-membered ring and E is —C(R⁴)(R⁶)—.

In one embodiment, in Formula (I), A is a 8-membered ring and E is —N(R⁶)—.

In one embodiment, in Formula (I), A is a 8-membered ring and E is —N(C(Y)R⁷)—.

In one embodiment, in Formula (I), A is a 8-membered ring and E is —N(C(Y)OR⁸)—.

In one embodiment, in Formula (I), A is a 8-membered ring and E is —N(C(Y)N(R⁸)(R¹⁰))—.

In one embodiment, in Formula (I), A is a 8-membered ring and E is —C(O)—N(R¹¹)—.

In one embodiment, in Formula (I), A is a 8-membered ring and E is —N(R¹¹)—C(O)—.

In one embodiment, in Formula (I), A is a 8-membered ring and E is —S(O)₂—N(R¹¹)—.

In one embodiment, in Formula (I), A is a 8-membered ring and E is —N(R¹¹)—S(O)₂—.

In one embodiment, in Formula (I), A is a 8-membered ring and E is —C(O)—O—.

In one embodiment, in Formula (I), A is a 8-membered ring and E is —O—C(O)—.

In one embodiment, in Formula (I), A is a 8-membered ring and E is —O—N(R⁶)—.

In one embodiment, in Formula (I), A is a 8-membered ring and E is —N(R⁶)—O—.

In one embodiment, in Formula (I), A is a 8-membered ring and E is —N(R⁶)—N(R¹²)—.

In one embodiment, in Formula (I), A is a 8-membered ring and E is —N═N—.

In one embodiment, in Formula (I), A is a 8-membered ring and E is —C(R⁷)═N—.

In one embodiment, in Formula (I), A is a 8-membered ring and E is —C(O)—C(R⁷)═N—.

In one embodiment, in Formula (I), A is a 8-membered ring and E is —C(O)—N═N—.

In one embodiment, in Formula (I), A is a 8-membered ring and E is —O—C(Y)—N(R¹¹)—.

In one embodiment, in Formula (I), A is a 8-membered ring and E is —N(R¹¹)—C(Y)—O—.

in one embodiment, in Formula (I), A is a 8-membered ring and E is —N(R¹¹)—C(Y)—N(R¹²)—.

In one embodiment, in Formula (I), A is a 8-membered ring and E is —C(Y)—N(R¹¹)—O—.

In one embodiment, in Formula (I), A is a 8-membered ring and E is —C(Y)—N(R¹¹)—N(R¹²)—.

In one embodiment, in Formula (I), A is a 8-membered ring and E is —O—N(R¹¹)—C(Y)—.

In one embodiment, in Formula (I), A is a 8-membered ring and E is —N(R¹²)—N(R¹¹)—C(Y)—.

In one embodiment, in Formula (I), ring B is an unsubstituted or substituted benzo or an unsubstituted or substituted thiophenyl ring.

In one embodiment, in Formula (I), ring B is an unsubstituted benzo or an unsubstituted thiophenyl ring.

In one embodiment, in Formula (I), ring B is an unsubstituted aromatic ring or an aromatic ring which is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴—, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (I), ring B is an unsubstituted benzo ring or a benzo ring which is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (I), ring B is an unsubstituted or substituted heteroaromatic ring or a substituted heteroaromatic ring which is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶. In one such embodiment, in Formula (I), ring B is a 5-6-membered heteroaromatic ring having from 1-3 ring heteroatoms, which can be the same or different, each hetero ring atom being independently selected from the group consisting of N, S, O, S(O), and S(O)₂.

In one embodiment, in Formula (I), ring B is an unsubstituted or substituted moiety selected from the group consisting of benzo, furanyl, thiophenyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, and triazinyl.

In one embodiment, in Formula (I), ring B is an unsubstituted aromatic ring.

In one embodiment, in Formula (I), ring B is an unsubstituted benzo ring, and Formula (I) has the general structure:

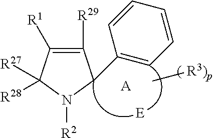

In one embodiment, in Formula (I), B is an aromatic ring which is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (I), B is a benzo ring which is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (I), B is an unsubstituted heteroaromatic ring.

In one embodiment, in Formula (I), B is an unsubstituted 5-6-membered heteroaromatic ring having from 1-3 ring heteroatoms, which can be the same or different, each hetero ring atom being independently selected from the group consisting of N, S, O, S(O), and S(O)₂.

In one embodiment, in Formula (I), B is a heteroaromatic ring which is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (I), B is a 5-6-membered heteroaromatic ring having from 1-3 ring heteroatoms, which can be the same or different, each hetero ring atom being independently selected from the group consisting of N, S, O, S(O), and S(O)₂, which heteroaromatic ring is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (I), B is an unsubstituted 6-membered heteroaromatic ring having from 1-3 ring heteroatoms, which can be the same or different, each hetero ring atom being independently selected from the group consisting of N, S, and O.

In one embodiment, in Formula (I), B is a 6-membered heteroaromatic ring having from 1-3 ring heteroatoms, which can be the same or different, each hetero ring atom being independently selected from the group consisting of N, S, and O, which heteroaromatic ring is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (I), B is an unsubstituted 6-membered heteroaromatic ring having 2 ring heteroatoms, each ring heteroatom being independently selected from of N, S, and O.

In one embodiment, in Formula (I), B is a 6-membered heteroaromatic ring having 2 ring heteroatoms, each ring heteroatom being independently selected from of N, S, and O, which heteroaromatic ring is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (I), B is an unsubstituted 5-membered heteroaromatic ring having from 1-2 ring heteroatoms, which can be the same or different, each hetero ring atom being independently selected from the group consisting of N, S, and O.

In one embodiment, in Formula (I), B is a 5-membered heteroaromatic ring having from 1-2 ring heteroatoms, which can be the same or different, each hetero ring atom being independently selected from the group consisting of N, S, and O, which heteroaromatic ring is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (I), B is an unsubstituted 5-membered heteroaromatic ring having 1 ring heteroatom selected from of N, S, and O.

In one embodiment, in Formula (I), B is a 5-membered heteroaromatic ring having 1 ring heteroatom selected from of N, S, and O, which heteroaromatic ring is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (I), B is a 5-membered heteroaromatic ring having S as the ring heteroatom, which heteroaromatic ring is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (I), B is an unsubstituted 5-membered heteroaromatic ring having S as the ring heteroatom.

In one embodiment, in Formula (I), B is a thiophenyl group.

In one embodiment, in Formula (I), B is selected from the group consisting of

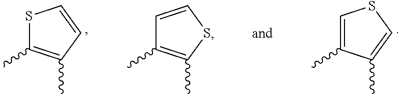

In one embodiment, in Formula (I), B is a pyridine.

In one embodiment, in Formula (I), B is a partially unsaturated alicyclic ring, which ring is unsubstituted.

In one embodiment, in Formula (I), B is a partially unsaturated alicyclic ring which is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (I), B is a partially unsaturated heterocyclic ring, which ring is unsubstituted.

In one embodiment, in Formula (I), B is a partially unsaturated heterocyclic ring which is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (I), R^{t} is unsubstituted aryl or aryl substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (I), R¹ is phenyl substituted with one to four substituents, which can be the same or different, each substituent being independently selected from the group consisting of halo, —OH, —CN, —NO₂, —NR²¹R²², and haloalkyl.

In one embodiment, in Formula (I), R¹ is unsubstituted aryl.

In one embodiment, in Formula (I), R¹ is unsubstituted phenyl.

In one embodiment, in Formula (I), R¹ unsubstituted naphthyl.

In one embodiment, in Formula (I), R¹ is substituted aryl.

In one embodiment, in Formula (I), R¹ is substituted phenyl.

In one embodiment, in Formula (I), R¹ is substituted naphthyl.

In one embodiment, in Formula (I). R¹ is aryl substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (I), R¹ is phenyl substituted with one or more substituents, which can be the same or different, each substituent being independently selected from the group consisting halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl, cycloalkyl, cycloalkenyl, heterocycloalkyl, heterocycloalkenyl, azido, —OR¹⁹, —OC(O)OR²⁰, —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴—NR²³C(N—CN)NR²⁵R²⁶ and —NR²³C(O)NR²⁵R²⁶.

In one embodiment, in Formula (I), R¹ is phenyl substituted with one to four substituents, which can be the same or different, each substituent being independently selected from the group consisting of halo, —OH, —CN, —NO₂, —NR²¹R²², and haloalkyl.

In one embodiment, in Formula (I), R¹ is selected from the group consisting of:

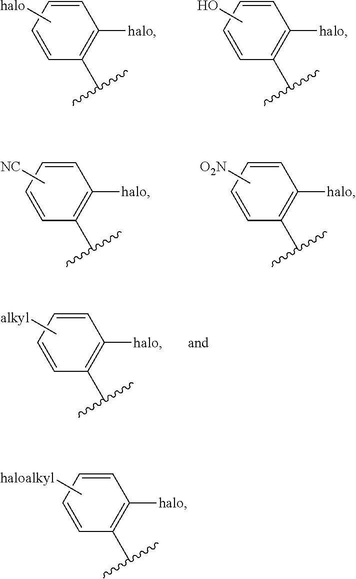

In one embodiment, in Formula (I), R¹ is:

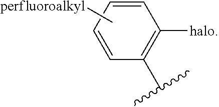

In one embodiment, in Formula (I), R¹ is phenyl substituted with one to three fluoro groups.

In one embodiment, in Formula (I), R¹ is phenyl substituted with two fluoro groups.

In one embodiment, in Formula (I), R¹ is phenyl substituted with one fluoro group.

In one embodiment, in Formula (I), R¹ is:

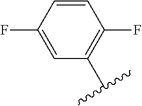

In one embodiment, in Formula (I), R²⁷, R²⁸, and R²⁹ are each independently selected from the group consisting of H and alkyl.

In one embodiment, in Formula (I), R² is selected from the group consisting of —C(O)R⁷, —C(O)NR⁹R¹⁰, and —C(O)OR⁸.

In one embodiment, in Formula (I), R² is —C(Z)R⁷.

In one embodiment, in Formula (I), R² is —C(Z)NR⁹R¹⁰.

In one embodiment, in Formula (I), R² is —C(Z)OR⁸.

In one embodiment, in Formula (I), R² is —SO₂NR⁹R¹⁰.

In one embodiment, in Formula (I), R² is alkyl.

In one embodiment, in Formula (I), R² is heteroalkyl.

In one embodiment, in Formula (I), R² is aryl.

In one embodiment, in Formula (I), R² is heteroaryl.

In one embodiment, in Formula (I), R² is cycloalkyl.

In one embodiment, in Formula (I), R² is cycloalkenyl.

In one embodiment, in Formula (I), R² is heterocycloalkyl.

In one embodiment, in Formula (I), R² is heterocycloalkenyl.

In one embodiment, in Formula (I), Z is (═O).

In one embodiment, in Formula (I), Z is (═S).

In one embodiment, in Formula (I), Z is (═N(R¹³)).

In one embodiment, in Formula (I), Z is (═N(CN)).

In one embodiment, in Formula (I), Z is (═N(OR¹⁴)).

In one embodiment, in Formula (I), Z is (═N(R¹⁵)(R¹⁶)).

In one embodiment, in Formula (I), Z is (═C(R¹⁷)(R¹⁸)).

In one embodiment, in Formula (I), R² is —C(Z)R⁷, and Z is (═O).

In one embodiment, in Formula (I), R² is —C(O)H.

In one embodiment, in Formula (I), R² is —C(O)alkyl.

In one embodiment, in Formula (I), R² is —C(O)CH₃.

In one embodiment, in Formula (I), R² is —C(O)R⁷, wherein said R⁷ is alkyl substituted with one or more substituents, which can be the same or different, each substituent being independently selected from the group consisting of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (I), R² is —C(O)R⁷, wherein said R⁷ is alkyl substituted with one to three substituents, which can be the same or different, each substituent being independently selected from the group consisting of —OR¹⁹, —NR²¹R²², and cycloalkyl.

In one embodiment, in Formula (I), R² is —C(O)R⁷, wherein said R⁷ is alkyl, wherein said alkyl is substituted with alkyl and —OH.

In one embodiment, in Formula (I), R² is —C(O)R⁷, wherein said R⁷ is alkyl substituted with one to three substituents, which can be the same or different, each substituent being independently selected from the group consisting of —OH, —NH₂, and cyclopropyl.

In one embodiment, in Formula (I), R² is —C(O)R⁷, wherein said R⁷ is alkyl substituted with one to two substituents, which can be the same or different, each substituent being independently selected from the group consisting of —NH₂, and cyclopropyl.

In one embodiment, in Formula (I), R² is —C(O)R⁷, wherein said R⁷ is alkyl substituted with —OH.

In one embodiment, in Formula (I), R² is —C(O)R⁷, wherein said R⁷ is unsubstituted heterocycloalkyl.

In one embodiment, in Formula (I), R² is —C(O)R⁷, wherein said R⁷ is substituted heterocycloalkyl.

In one embodiment, in Formula (I), R² is —C(O)R⁷, wherein said R⁷ is heterocycloalkyl substituted with one or more substituents, which can be the same or different, each substituent being independently selected from the group consisting of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (I), R² is —C(O)R⁷, wherein said R⁷ is selected from the group consisting of substituted piperidine, substituted piperazine, substituted morpholine, substituted pyrrolidine, and substituted azetidine.

In one embodiment, in Formula (I), R² is a moiety selected from the group consisting of:

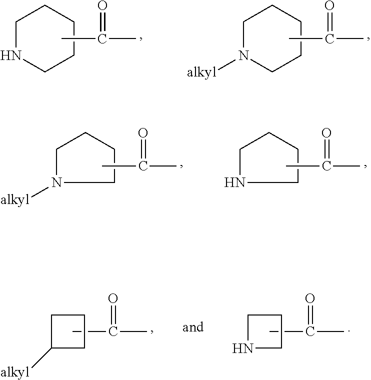

In one embodiment, in Formula (I), R² is —C(O)NR⁹R¹⁰.

In one embodiment, in Formula (I), R² is —C(O)NH₂.

In one embodiment, in Formula (I), R² is —C(O)NR⁹R¹⁰, wherein R⁹ and R¹⁰ can be the same or different, each being independently selected from alkyl.

In one embodiment, in Formula (I), R² is —C(O)NR⁹R¹⁰, wherein R⁹ is unsubstituted heterocycloalkyl and R¹⁰ is selected from the group consisting of H and alkyl.

In one embodiment, in Formula (I), R² is —C(O)NR⁹R¹⁰, wherein R⁹ is substituted heterocycloalkyl and R¹⁰ is selected from the group consisting of H and alkyl.

In one embodiment, in Formula (I), R² is —C(O)NR⁹R¹⁰, wherein R⁹ is heterocycloalkyl substituted with from one to three substituents, which can be the same or different, each substituent being independently selected from alkyl, and R¹⁰ is selected from the group consisting of H and alkyl.

In one embodiment, in Formula (I), R² is selected from the group consisting of: alkyl, haloalkyl, heteroalkyl, heterohaloalkyl, —C(O)R⁷. —C(O)OR⁸, and —C(O)NR⁹R¹⁰.

Non-limiting examples of R² include the following moieties:

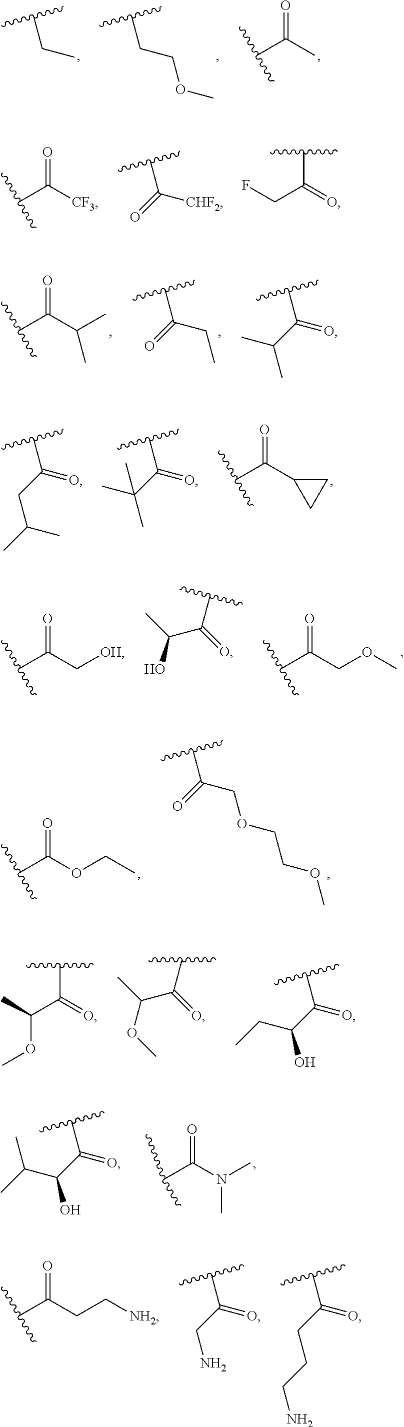

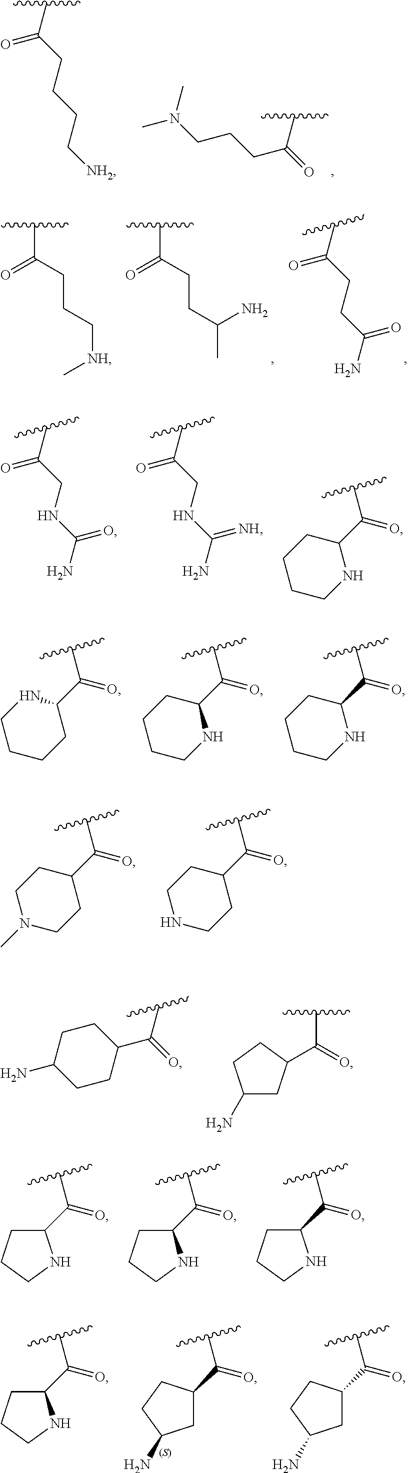

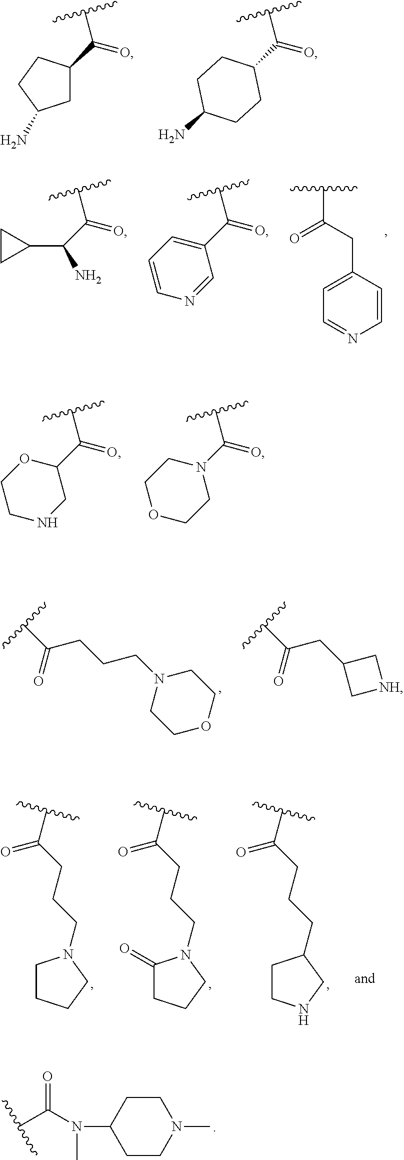

In one embodiment, in Formula (I), R² is

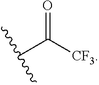

In one embodiment, in Formula (I), R² is

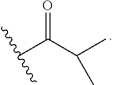

In one embodiment, in Formula (I), R² is

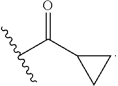

In one embodiment, in Formula (I), R² is

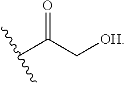

In one embodiment, in Formula (I), R² is

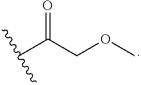

In one embodiment, in Formula (I), R² is

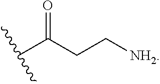

In one embodiment, in Formula (I), R² is

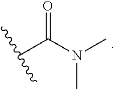

In one embodiment, in Formula (I), R² is

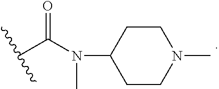

In one embodiment, in Formula (I), p is 0 and R³ is not present.

In one embodiment, in Formula (I), p is 1.

In one embodiment, in Formula (I), p is 2.

In one embodiment, in Formula (I), p is 3.

In one embodiment, in Formula (I), p is 4.

In one embodiment, in Formula (I), p is 2, 3, or 4, and at least two groups R³ are attached to the same ring atom.

In one embodiment, in Formula (I), p is 1, 2, 3, or 4 and each R³ is independently selected from the group consisting of alkyl, heteroalkyl, alkenyl, heteroalkenyl, —CN, —NO₂, —OR¹⁹, —OC(O)OR²⁰, —NR²¹R²², —C(O)R²⁴, —C(S)R²⁴, —O(O)OR²⁰, and —C(O)NR²⁵R²⁶,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁵, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (I), p is 1 and R³ is independently selected from the group consisting of alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (I), p is 2, 3, or 4 and each R³ is independently selected from the group consisting of alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (I), p is 2, 3, or 4 and at least two groups R³ are bound to the same ring carbon atom, wherein each R³, which may be the same or different, is independently selected from the group consisting of alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (I), p is 2, 3, or 4 and at least two groups R³ are bound to the same ring carbon atom, wherein two R³ groups, which may be the same or different, together with the carbon atom to which they are attached, form a cycloalkyl, a cycloalkenyl, a heterocycloalkyl ring containing from one to three heteroatoms selected from the group consisting of N, O, and S, or a heterocycloalkenyl ring containing from one to three heteroatoms selected from the group consisting of N, O, and S.

In one embodiment, in Formula (I), each R³ (when present) is independently selected from the group consisting of alkyl, heteroalkyl, alkenyl, heteroalkenyl, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(S)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶, —NR²³C(O)NR²⁵R²⁶, and —NR²³—C(NH)—NR²⁵R²⁶,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (I), each R³ (when present) is independently selected from the group consisting of alkyl, heteroalkyl, alkenyl, heteroalkenyl, —CN, —NO₂, —OR¹⁹, —OC(O)OR²⁰, —NR²¹R²², —C(O)R²⁴—C(O)OR²⁰, and —C(O)NR²⁵R²⁶,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (I), p is 1 and R³ is selected from the group consisting of alkyl, heteroalkyl, alkenyl, and heteroalkenyl,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (I), p is 2, 3, or 4, and any two R³ groups bound to the same ring A atom are taken together with the carbon atom to which they are attached to form a spirocycloalkyl, a spirocycloalkenyl, a spiroheterocycloalkyl ring containing from one to three ring heteroatoms independently selected from the group consisting of —NH—, —NR⁶—, —S—, —S(O)—, —S(O)₂—, and —O—, or a spiroheterocycloalkenyl ring containing from one to three ring heteroatoms independently selected from the group consisting of —NH—, —NR⁶—, —S—, —S(O)—, —S(O)₂—, and —O—. Non-limiting examples of compounds of the invention in which two R³ groups are thus taken together include:

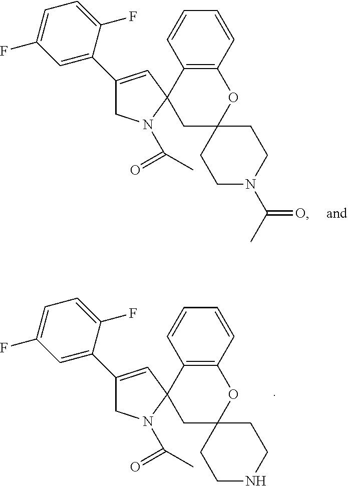

In one embodiment, in Formula (I), R² and R³ are taken together with the carbon atom to which they are attached to form a cycloalkyl, a cycloalkenyl, a heterocycloalkyl ring containing from one to three ring heteroatoms independently selected from the group consisting of —NH—, —NR⁶—, —S—, —S(O)—, —S(O)₂—, and —O—, or a heterocycloalkenyl ring containing from one to three ring heteroatoms independently selected from the group consisting of —NH—, —NR⁶—, —S—, —S(O)—, —S(O)₂—, and —O—. Non-limiting examples of a compound of the invention in which R² and R³ are thus taken together include the following compound:

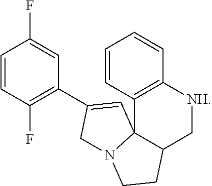

In one embodiment, in Formula (I), R³ is alkyl.

In one embodiment, in Formula (I), R³ is heteroalkyl.

In one embodiment, in Formula (I), R³ is alkenyl.

In one embodiment, in Formula (I), R³ is heteroalkenyl.

In one embodiment, in Formula (I), R³ is alkynyl.

In one embodiment, in Formula (I), R³ is heteroalkynyl.

In one embodiment, in Formula (I), R³ is aryl.

In one embodiment, in Formula (I), R³ is heteroaryl.

In one embodiment, in Formula (I), R³ is cycloalkyl.

In one embodiment, in Formula (I), R³ is cycloalkenyl.

In one embodiment, in Formula (I), R³ is heterocycloalkyl.

In one embodiment, in Formula (I), R³ is heterocycloalkenyl.

In one embodiment, in Formula (I), R³ is halogen.

In one embodiment, in Formula (I), R³ is —CN.

In one embodiment, in Formula (I), R³ is —NO₂.

In one embodiment, in Formula (I), R³ is —OR¹⁹.

In one embodiment, in Formula (I), R³ is —OC(O)OR²⁰.

In one embodiment, in Formula (I), R³ is —NR²¹R²².

In one embodiment, in Formula (I), R³ is —NR²³SO₂R²⁴.

In one embodiment, in Formula (I), R³ is —NR²³C(O)OR²⁰.

In one embodiment, in Formula (I), R³ is —NR²³C(O)R²⁴.

In one embodiment, in Formula (I), R³ is —SO₂NR²⁵R²⁶

In one embodiment, in Formula (I), R³ is —C(O)R²⁴.

In one embodiment, in Formula (I), R³ is —C(S)R²⁴.

In one embodiment, in Formula (I), R³ is —C(O)OR²⁰.

In one embodiment, in Formula (I), R³ is —SR¹⁹.

In one embodiment, in Formula (I), R³ is —S(O)R¹⁹.

In one embodiment, in Formula (I), R³ is —SO₂R¹⁹.

In one embodiment, in Formula (I), R³ is —OC(O)R²⁴.

In one embodiment, in Formula (I), R³ is —C(O)NR²⁵R²⁶.

In one embodiment, in Formula (I), R³ is —NR²³C(N—CN)NR²⁵R²⁶.

In one embodiment, in Formula (I). R³ is —NR²³C(O)NR²⁵R²⁶.

Non-limiting examples of R³ include the following: methyl, ethyl, propyl (straight or branched), butyl (straight or branched), pentyl (straight or branched), phenyl,

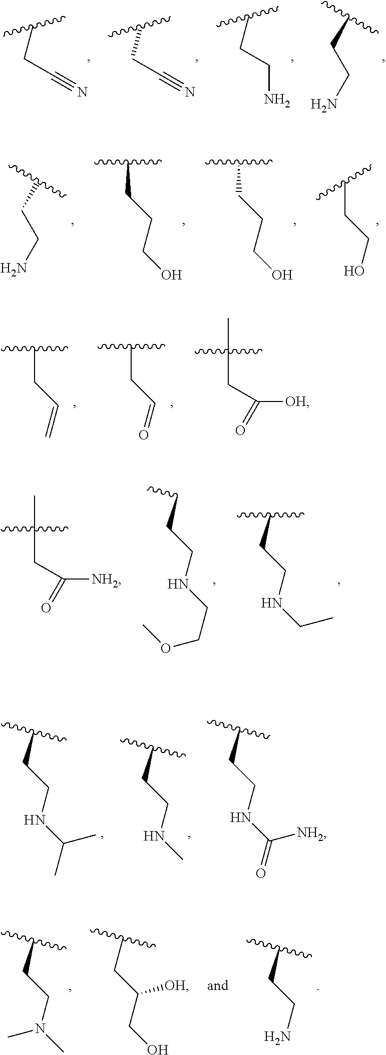

In one embodiment, in Formula (I), when E is —NR⁶—, R³ is absent.

In one embodiment, Formula (I) has the general structure shown in Formula (I.a):

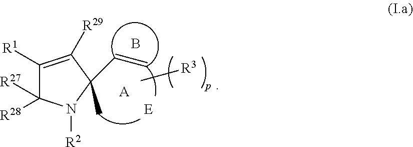

In one embodiment, Formula (I) has the general structure shown in Formula (I.b):

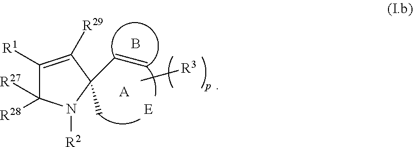

In one embodiment, Formula (I) has the general structure shown in Formula (I.c):

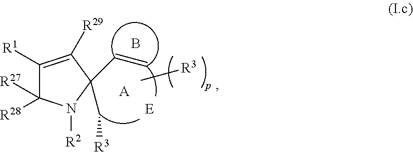

wherein p is 0, 1, 2, or 3.

In one embodiment, Formula (I) has the general structure shown in Formula (I.d):

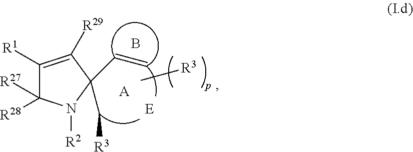

wherein p is 0, 1, 2, or 3.

In one embodiment, Formula (I) has the general structure shown in Formula (I.e):

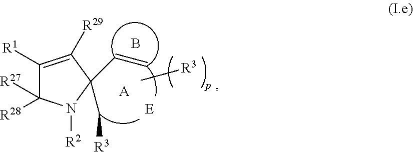

wherein p is 0, 1, 2, or 3.

In one embodiment, Formula (I) has the general structure shown in Formula (I.f):

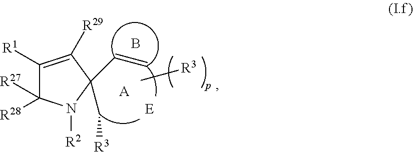

wherein p is 0, 1, 2, or 3.

In one embodiment, Formula (I) has the general structure shown in Formula (I.g):

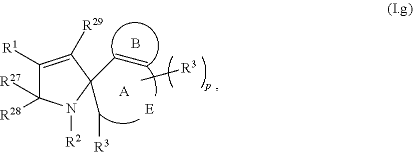

wherein p is 0, 1, 2, or 3.

In some embodiments, in each of formulas (I), (I.a), (I.b), (I.c), (I.d), (I.e), (I.f), and (I.g), R¹ is

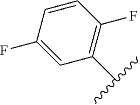

and the compounds of the invention have the general structure shown in Formula (I.h):

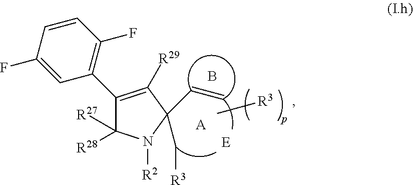

wherein p is 0, 1, 2, or 3.

In some embodiments, in each of Formulas (I), (I.a), (I.b), (I.c), (I.d), (I.e), (I.f), (I.g), and (I.h), p is 0.

For the various embodiments of the present invention described herein, it shall be understood that any variable of a structural formula not explicitly defined therein is as defined in the formula to which the embodiment refers. It shall also be understood that each R³, when present, is attached to a ring atom or ring heteroatom of ring A by replacement of an available hydrogen atom.

In other embodiments, in each of Formulas (I), (I.a), (I.b), (I.c), (I.d), (I.e), (I.f), (I.g), and (I.h):

ring A is a 4-7 membered cycloalkenyl ring;

### E is —C(R⁴)(R⁵)—; and

ring B is a benzo ring or a 5-6 membered heteroaromatic ring,
wherein said ring is unsubstituted or optionally independently substituted with from 1 to 3 substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In other embodiments, in each of Formulas (I), (I.a), (I.b), (I.c), (I.d), (I.e), (I.f), and (I.g):

ring A is a 4-7 membered cycloalkenyl ring;

### E is —C(R⁴)(R⁵)—; and

ring B is a benzo ring or a 5-6 membered heteroaromatic ring,
wherein said ring is unsubstituted or optionally independently substituted with from 1 to 3 substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
   R¹ is phenyl substituted with one to four substituents, which can be the same or different, each substituent being independently selected from the group consisting of halo, —OH, —NR²¹R²², and haloalkyl;
   R² is selected from the group consisting of: alkyl, haloalkyl, heteroalkyl, heterohaloalkyl, —C(O)R⁷. —C(O)OR⁸, and —C(O)NR⁹R¹⁰; and
   each R³ (when present) is independently selected from the group consisting of alkyl, heteroalkyl, alkenyl, heteroalkenyl, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —NR²¹R²², —NR²³SO₂R²⁴, —NR²³C(O)OR²⁰, —NR²³C(O)R²⁴, —SO₂NR²⁵R²⁶, —C(O)R²⁴, —C(S)R²⁴, —C(O)OR²⁰, —SR¹⁹, —S(O)R¹⁹, —OC(O)R²⁴, —C(O)NR²⁵R²⁶, —NR²³C(N—CN)NR²⁵R²⁶, —NR²³C(O)NR²⁵R²⁶, and —NR²³—C(NH)—NR²⁵R²⁶,
wherein each said alkyl, each said heteroalkyl, each said alkenyl, and each said hateroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In other embodiments, in each of Formulas (I), (I.a), (I.b), (I.c), (I.d), (I.e), (I.f), and (I.g):

ring A is a 4-7 membered cycloalkenyl ring;

### E is —C(R⁴)(R⁵)—;

ring B is a benzo ring or a 5-6 membered heteroaromatic ring,
wherein said ring is unsubstituted or optionally independently substituted with from 1 to 3 substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
   R¹ is phenyl substituted with one to four substituents, which can be the same or different, each substituent being independently selected from the group consisting of halo, —OH, —CN, —NO₂, —NR²¹R²², haloalkyl;
   R² is selected from the group consisting of: alkyl, haloalkyl, heteroalkyl, heterohaloalkyl, —C(O)R⁷. —C(O)OR⁸, and —C(O)NR⁹R¹⁰; and
   each R³ (when present) is independently selected from the group consisting of alkyl, heteroalkyl, alkenyl, heteroalkenyl, —CN, —NO₂, —OR¹⁹, —OC(O)OR²⁰, —NR²¹R²², —C(O)R²⁴, —C(S)R²⁴, —C(O)OR²⁰, and —C(O)NR²⁵R²⁶,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶

In other embodiments, in each of Formulas (I), (I.a), (I.b), (I.c), (I.d), (I.e), (I.f), and (I.g):

ring A is a 4-7 membered cycloalkenyl ring;

### E is —C(R⁴)(R⁵)—; and

ring B is a benzo ring or a 5-6 membered heteroaromatic ring,
wherein said ring is unsubstituted or optionally independently substituted with from 1 to 3 substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁸, —NR²³C(N—CN)NR²⁵R²⁶ and —NR²³C(O)NR²⁵R²⁶;
   R¹ is phenyl substituted with one to four substituents, which can be the same or different, each substituent being independently selected from the group consisting of halo, —OH, —CN, —NO₂, —NR²¹R²², and haloalkyl;
   R²⁷, R²⁸, and R²⁹ are each independently selected from the group consisting of H and alkyl;
   R² is selected from the group consisting of: alkyl, haloalkyl, heteroalkyl, heterohaloalkyl, —C(O)R⁷. —C(O)OR⁸, and —C(O)NR⁹R¹⁰; and
   p is 1 and R³ is selected from the group consisting of alkyl, heteroalkyl, alkenyl, and heteroalkenyl,
wherein each said alkyl, each said heteroalkyl, each said alkenyl, and each said heteroalkenyl, is unsubstituted or optionally independently substituted with from 1 to 3 substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In other embodiments, in each of Formulas (I), (I.a), (I.b), (I.c), (I.d), (I.e), (I.f), (I.g), and (I.h):

ring A is a 5-6 membered heterocycloalkenyl ring;
E is selected from the group consisting of —O—, —S—, —S(O)—, —S(O)₂—, —N(R⁶)—, —N(C(Y)R⁷)—, —N(C(Y)OR⁸)—, —N(C(Y)N(R⁹)(R¹⁰))—, —C(O)—N(R¹¹)—, —N(R¹¹)—C(O)—, —S(O)₂—N(R¹¹)—, —N(R¹¹)—S(O)₂—, —C(O)—O—, —O—C(O)—, —O—N(R⁶)—, —N(R⁶)—O—, —N(R⁶)—N(R¹²)—, —N═N—, —C(R⁷)═N—, —C(O)—C(R⁷)═N—, —C(O)—N═N—, —O—C(Y)—N(R¹¹)—, —N(R¹¹)—C(Y)—O—, —N(R¹¹)—C(Y)—N(R¹²)—, —C(Y)—N(R¹¹)—O—, —C(Y)—N(R¹¹)—N(R¹²)—, —O—N(R¹¹)—C(Y)—, and —N(R¹²)—N(R¹¹)—C(Y)—; and
ring B is a benzo ring or a 5-6 membered heteroaromatic ring,
wherein said ring is unsubstituted or optionally independently substituted with from 1 to 3 substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In other embodiments, in each of Formulas (I), (I.a), (I.b), (I.c), (I.d), (I.e), (I.f), and (I.g):

ring A is a 5-6 membered heterocycloalkenyl ring;
E is selected from the group consisting —O—, —S—, —S(O)—, —S(O)₂—, and —N(R⁶)—, wherein R⁶ is selected from the group consisting of H, alkyl, —C(O)R²⁴, —C(O)OR²⁰, and —C(S)R²⁴;
ring B is a benzo ring or a 5-6 membered heteroaromatic ring,
wherein said ring is unsubstituted or optionally independently substituted with from 1 to 3 substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
   R¹ is phenyl substituted with one to four substituents, which can be the same or different, each substituent being independently selected from the group consisting of halo, —OH, —CN, —NO₂, —NR²¹R²², and haloalkyl;
   R² is selected from the group consisting of: alkyl, haloalkyl, heteroalkyl, heterohaloalkyl, —C(O)R⁷. —C(O)OR⁸, and —C(O)NR⁹R¹⁰; and
   each R³ (when present) is independently selected from the group consisting of alkyl, heteroalkyl, alkenyl, heteroalkenyl, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(S)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶, —NR²³C(O)NR²⁵R²⁶, and —NR²³—C(NH)—NR²⁸R²⁶,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In other embodiments, in each of Formulas (I), (I.a), (I.b), (I.c), (I.d), (I.e), (I.f), and (I.g):

ring A is a 5-6 membered heterocycloalkenyl ring;
E is selected from the group consisting —O—, —S—, —S(O)—, —S(O)₂—, and —N(R⁶)—, wherein R⁶ is selected from the group consisting of H, alkyl, —C(O)R²⁴, —C(O)OR²⁰, and —C(S)R²⁴;
ring B is a benzo ring or a 5-6 membered heteroaromatic ring,
wherein said ring is unsubstituted or optionally independently substituted with from 1 to 3 substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁶, —OC(O)R²⁴, —C(O)NR²⁵R²⁶, —NR²³C(N—CN)NR²⁵R²⁶ and —NR²³C(O)NR²⁵R²⁶;
   R¹ is phenyl substituted with one to four substituents, which can be the same or different, each substituent being independently selected from the group consisting of halo, —OH, —CN, —NO₂, —NR²¹R²², and haloalkyl;
   R² is selected from the group consisting of: alkyl, haloalkyl, heteroalkyl, heterohaloalkyl, —C(O)R⁷. —C(O)OR⁸, and —C(O)NR⁹R¹⁰; and
   each R³ (when present) is independently selected from the group consisting of alkyl, heteroalkyl, alkenyl, heteroalkenyl, —CN, —NO₂, —OR¹⁹, —OC(O)OR²⁰, —NR²¹R²², —C(O)R²⁴, —C(S)R²⁴, —C(O)OR²⁰, and —C(O)NR²⁵R²⁶,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In other embodiments, in each of Formulas (I), (I.a), (I.b), (I.c), (I.d), (I.e), (I.f), and (I.g):

ring A is a 5-6 membered heterocycloalkenyl ring;
E is selected from the group consisting —O—, —S—, —S(O)—, —S(O)₂—, and —N(R⁶)—, wherein R⁶ is selected from the group consisting of H, alkyl, —C(O)R²⁴, —C(O)OR²⁰ and —C(S)R²⁴;
ring B is a benzo ring or a 5-6 membered heteroaromatic ring,
wherein said ring is unsubstituted or optionally independently substituted with from 1 to 3 substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹—S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
   R¹ is phenyl substituted with one to four substituents, which can be the same or different, each substituent being independently selected from the group consisting of halo, —OH, —CN, —NO₂, —NR²¹R²², and haloalkyl;
   R² is selected from the group consisting of: alkyl, haloalkyl, heteroalkyl, heterohaloalkyl, —C(O)R⁷. —C(O)OR⁸, and —C(O)NR⁹R¹⁰; and
   R²⁷, R²⁸, and R²⁹ are each independently selected from the group consisting of H and alkyl;
   p is 1 and R³ is selected from the group consisting of alkyl, heteroalkyl, alkenyl, and heteroalkenyl,
wherein each said alkyl, each said heteroalkyl, each said alkenyl, and each said heteroalkenyl, is unsubstituted or optionally independently substituted with from 1 to 3 substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, the compounds of the invention have a structure shown in Formula (II) and include pharmaceutically acceptable salts, solvates, esters, prodrugs, or isomers of said compounds:

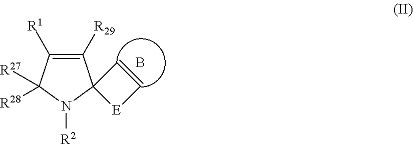

wherein R¹, R², R²⁷, R²⁸, R²⁹, E, and ring B are selected independently of each other and wherein
E is selected from the group consisting of —O—, —S—, —S(O)—, —S(O)₂—, —C(R⁴)(R⁵)—, —N(R⁶)—, —N(C(Y)R⁷)—, —N(C(Y)OR⁸)—, —N(C(Y)N(R⁹)(R¹⁰))—;
and ring B, R¹, R², R⁴, R⁵, R⁶, R⁷, R⁸, R⁹, R¹⁰, R²⁷, R²⁸, R²⁹, Y, and the optional substituents on ring B are as defined in any of the embodiments described above in Formula (I).

In one embodiment, in Formula (If):

E is selected from the group consisting of —O—, —S—, —S(O)—, —S(O)₂—, —C(R⁴)(R⁵)—, and —N(R⁶)—;
ring B is an unsubstituted or substituted moiety selected from the group consisting of benzo, furanyl, thiophenyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, and triazinyl;
R¹ is phenyl substituted with one to four substituents, which can be the same or different, each substituent being independently selected from the group consisting of halo, —OH, —CN, —NO₂, —NR²¹R²², and haloalkyl; and
R² is selected from the group consisting of —C(O)R⁷, —C(O)NR⁹R¹⁰, and —C(O)OR⁸.

In one embodiment, in Formula (II):

### R¹ is:

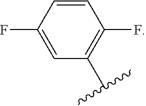

in one embodiment, in Formula (II):

### R¹ is:

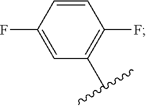

and R²⁷. R²⁸, and R²⁹ are each independently selected from the group consisting of H and alkyl.

In one embodiment, the compounds of the invention have a structure shown in Formula (II.a) and include pharmaceutically acceptable salts, solvates, esters, prodrugs, or isomers of said compounds:

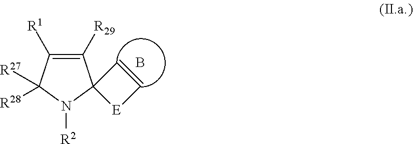

wherein R¹, R², R²⁷, R²⁸, R²⁹, E, and ring B are selected independently of each other and wherein:
E is selected from the group consisting of —O—, —S—, —S(O)—, —S(O)₂—, —C(R⁴)(R⁶)—, —N(R⁶)—, —N(C(Y)R⁷)—, —N(C(Y)OR⁸)—, —N(C(Y)N(R⁹)(R¹⁰))—.
ring B is a substituted or unsubstituted aromatic ring;
and R¹, R², R⁴, R⁵, R⁶, R⁷, R⁸, R⁹, R¹⁰, R²⁷, R²⁸, R²⁹, Y, and the optional substituents on ring B are as defined in any of the embodiments described above in Formula (I).

In one embodiment, Formula (II.a.) has the general structure shown in Formula (II.a.1):

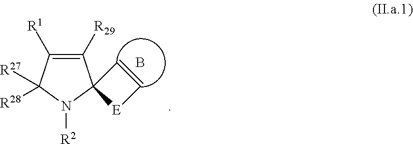

In one embodiment, Formula (II.a.) has the general structure shown in Formula (II.a.2):

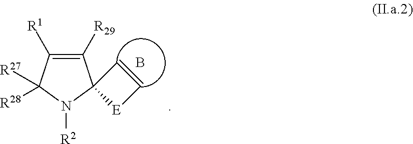

In some embodiments, in each of Formulas (II.a.), (II.a.1), and (II.a.2), E is —C(R⁴)(R⁵)—.

In some embodiments, in each of Formulas (II.a.1), and (II.a.2), E is selected from the group consisting of —O—, —S—, —S(O)—, —S(O)₂—, and —N(R⁶)—.

In some embodiments, in each of Formulas (II.a.), (II.a.1), and (II.a.2), E is selected from the group consisting of —O—, —S—, —S(O)—, —S(O)₂—, and —N(R⁶)—, wherein R⁶ is selected from the group consisting of H, alkyl, —C(O)R²⁴, and —C(S)R²⁴.

In some embodiments, in each of Formulas (II.a.), (II.a.1), and (II.a.2), E is selected from the group consisting of —O— and —N(R⁶)—, wherein R⁶ is selected from the group consisting of H, alkyl, —C(O)R²⁴, and —C(S)R²⁴.

In some embodiments, in each of Formulas (II.a.), (II.a.1), and (II.a.2), E is —O—.

In some embodiments, in each of Formulas (II.a.), (II.a.1), and (II.a.2), E is —S—.

In some embodiments, in each of Formulas (II.a.), (II.a.1), and (II.a.2), E is —S(O)—.

In some embodiments, in each of Formulas (II.a.), (II.a.1), and (II.a.2), E is —S(O)₂—.

In some embodiments, in each of Formulas (II.a.), (II.a.1), and (II.a.2), E is —C(R⁴)(R⁵)—.

In some embodiments, in each of Formulas (II.a.), (II.a.1), and (II.a.2), E is —N(R⁶)—.

In some embodiments, in each of Formulas (II.a.), (II.a.1), and (II.a.2), E is —N(C(Y)R⁷)—.

In some embodiments, in each of Formulas (II.a.), (II.a.1), and (II.a.2), E is —N(C(Y)OR⁸)—.

In some embodiments, in each of Formulas (II.a.), (II.a.1), and (II.a.2), E is —N(C(Y)N(R⁹)(R¹⁰))—.

In some embodiments, in each of Formulas (II.a.), (II.a.1), and (II.a.2), Y is (═O).

In some embodiments, in each of Formulas (II.a.), (II.a.1), and (II.a.2), Y is (═S).

In some embodiments, in each of Formulas (II.a.), (II.a.1), and (II.a.2), Y is (═N(R¹³)).

In some embodiments, in each of Formulas (II.a.), (II.a.1), and (II.a.2), Y is (═N(CN)).

In some embodiments, in each of Formulas (II.a.), (II.a.1), and (II.a.2), Y is (═N(OR¹⁴)).

In some embodiments, in each of Formulas (II.a.), (II.a.1), and (II.a.2), Y is (═N(R¹⁵)(R¹⁶)).

In some embodiments, in each of Formulas (II.a.), (II.a.1), and (II.a.2), Y is (═C(R¹⁷)(R¹⁸)).

In some embodiments, in each of Formulas (II.a.), (II.a.1), and (II.a.2), B is an unsubstituted aromatic ring.

In some embodiments, in each of Formulas (II.a.), (II.a.1), and (II.a.2), B is an unsubstituted benzo ring, and Formula (II.a.) has the general structure:

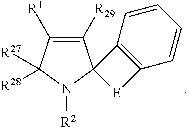

In some embodiments, in each of Formulas (II.a.), (II.a.1), and (II.a.2), B is an unsubstituted benzo ring, and Formula (II.a.) has the general structure:

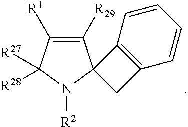

In some embodiments, in each of Formulas (II.a.), (II.a.1), and (II.a.2), B is an aromatic ring which is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, NR²³C(N—CN)NR²⁵R²⁶ and —NR²³C(O)NR²⁵R²⁶,

In some embodiments, in each of Formulas (II.a.), (II.a.1), and (II.a.2), B is a benzo ring which is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR¹⁴R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (II.a.), (II.a.1), and (II.a.2), R¹ is unsubstituted aryl.

In some embodiments, in each of Formulas (II.a.), (II.a.1), and (II.a.2), R¹ is unsubstituted phenyl.

In some embodiments, in each of Formulas (II.a.), (II.a.1), and (II.a.2), R¹ is unsubstituted naphthyl.

In some embodiments, in each of Formulas (II.a.), (II.a.1), and (II.a.2), R¹ is substituted aryl.

In some embodiments, in each of Formulas (II.a.), (II.a.1), and (II.a.2), R¹ is substituted phenyl.

In some embodiments, in each of Formulas (II.a.), (II.a.1), and (II.a.2), R¹ is substituted naphthyl.

In some embodiments, in each of Formulas (II.a.), (II.a.1), and (II.a.2), R¹ is aryl substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl, cycloalkyl, cycloalkenyl, heterocycloalkyl, heterocycloalkenyl, azido, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (II.a.), (II.a.1), and (II.a.2), R¹ is phenyl substituted with one or more substituents, which can be the same or different, each substituent being independently selected from the group consisting halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (II.a.), (II.a.1), and (II.a.2), R¹ is phenyl substituted with one to four substituents, which can be the same or different, each substituent being independently selected from the group consisting of halo, —OH, —CN, —NO₂, —NR²¹R²², and haloalkyl.

In some embodiments, in each of Formulas (II.a.), (II.a.1), and (II.a.2), R¹ is selected from the group consisting of:

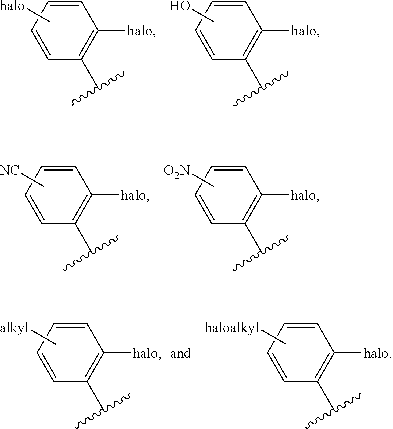

In some embodiments, in each of Formulas (II.a.), (II.a.1), and (II.a.2), R¹ is:

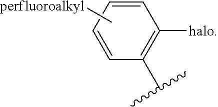

In some embodiments, in each of Formulas (II.a.), (II.a.1), and (II.a.2), R¹ is phenyl substituted with one to three fluoro groups.

In some embodiments, in each of Formulas (II.a.), (II.a.1), and (II.a.2), R¹ is phenyl substituted with two fluoro groups.

In some embodiments, in each of Formulas (II.a.), (II.a.1), and (II.a.2), R¹ is phenyl substituted with one fluoro group.

In some embodiments, in each of Formulas (II.a.), (II.a.1), and (II.a.2), R¹ is:

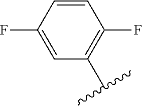

In some embodiments, in each of Formulas (II.a.), (II.a.1), and (II.a.2), R²⁷, R²⁸ and R²⁹ are each independently selected from the group consisting of H and alkyl.

In some embodiments, in each of Formulas (II.a.), (II.a.) and (II.a.2), R² is —C(Z)R⁷.

In some embodiments, in each of Formulas (II.a.), (II.a.1), and (II.a.2). R² is —C(Z)NR⁹R¹⁰.

In some embodiments, in each of Formulas (II.a.), (II.a.1), and (II.a.2), R² is —C(Z)OR⁸.

In some embodiments, in each of Formulas (II.a.), (II.a.1), and (II.a.2), R² is —SO₂NR⁹R¹⁰.

In some embodiments, in each of Formulas (II.a.), (II.a.1), and (II.a.2), R² is alkyl.

In some embodiments, in each of Formulas (II.a.), (II.a.1), and (II.a.2), R² is heteroalkyl.

In some embodiments, in each of Formulas (II.a.), (II.a.1), and (II.a.2), R² is aryl.

In some embodiments, in each of Formulas (II.a.), (II.a.1), and (II.a.2), R² is heteroaryl.

In some embodiments, in each of Formulas (II.a.), (II.a.1), and (II.a.2), R² is cycloalkyl.

In some embodiments, in each of Formulas (II.a.), (II.a.1), and (II.a.2), R² is cycloalkenyl.

In some embodiments, in each of Formulas (II.a.), (II.a.1), and (II.a.2), R² is heterocycloalkyl.

In some embodiments, in each of Formulas (II.a.), (II.a.1), and (II.a.2), R² is heterocycloalkenyl.

In some embodiments, in each of Formulas (II.a.), (II.a.1), and (II.a.2), Z is (═O).

In some embodiments, in each of Formulas (II.a.), (II.a.1), and (II.a.2), Z is (═S).

In some embodiments, in each of Formulas (II.a.), (II.a.1), and (II.a.2), Z is (═N(R¹³)).

In some embodiments, in each of Formulas (II.a.), (II.a.1), and (II.a.2), Z is (═N(CN)).

In some embodiments, in each of Formulas (II.a.), (II.a.1), and (II.a.2), Z is (═N(OR¹⁴)).

In some embodiments, in each of Formulas (II.a.), (II.a.1), and (II.a.2), Z is (═N(R¹⁵)(R¹⁶)).

In some embodiments, in each of Formulas (II.a.), (II.a.1), and (II.a.2), Z is (═C(R¹⁷)(R¹⁸)).

In some embodiments, in each of Formulas (II.a.), (II.a.1), and (II.a.2), R² is —C(Z)R⁷, and Z is (═O).

In some embodiments, in each of Formulas (II.a.), (II.a.1), and (II.a.2), R² is —C(O)H.

In some embodiments, in each of Formulas (II.a.), (II.a.1), and (II.a.2), R² is —C(O)alkyl.

In some embodiments, in each of Formulas (II.a.), (II.a.1), and (II.a.2), R² is —C(O)CH₃.

In some embodiments, in each of Formulas (II.a.), (II.a.1), and (II.a.2), R² is —C(O)R⁷, wherein said R⁷ is alkyl substituted with one or more substituents, which can be the same or different, each substituent being independently selected from the group consisting of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR^{2G}, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (II.a.), (II.a.1), and (II.a.2), R² is —C(O)R⁷, wherein said R⁷ is alkyl substituted with one to three substituents, which can be the same or different, each substituent being independently selected from the group consisting of —OR¹⁹, —NR²¹R²², and cycloalkyl.

In some embodiments, in each of Formulas (II.a.), (II.a.1), and (II.a.2), R² is —C(O)R⁷, wherein said R⁷ is alkyl, wherein said alkyl is substituted with alkyl and —OH.

In some embodiments, in each of Formulas (II.a.), (II.a.1), and (II.a.2), R² is —C(O)R⁷, wherein said R⁷ is alkyl substituted with one to three substituents, which can be the same or different, each substituent being independently selected from the group consisting of —OH, —NH₂, and cyclopropyl.

In some embodiments, in each of Formulas (II.a.), (II.a.1), and (II.a.2), R² is —C(O)R⁷, wherein said R⁷ is alkyl substituted with one to two substituents, which can be the same or different, each substituent being independently selected from the group consisting of —NH₂, and cyclopropyl.

In some embodiments, in each of Formulas (II.a.), (II.a.1), and (II.a.2), R² is —C(O)R⁷, wherein said R⁷ is alkyl substituted with —OH.

In some embodiments, in each of Formulas (II.a.), (II.a.1), and (II.a.2), R² is —C(O)R⁷, wherein said R⁷ is unsubstituted heterocycloalkyl.

In some embodiments, in each of Formulas (II.a.), (II.a.1), and (II.a.2), R² is —C(O)R⁷, wherein said R⁷ is substituted heterocycloalkyl.

In some embodiments, in each of Formulas (II.a.), (II.a.1), and (II.a.2), R² is —C(O)R⁷, wherein said R⁷ is heterocycloalkyl substituted with one or more substituents, which can be the same or different, each substituent being independently selected from the group consisting of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (II.a.), (II.a.1), and (II.a.2), R² is —C(O)R⁷, wherein said R⁷ is selected from the group consisting of substituted piperidine, substituted piperazine, substituted morpholine, substituted pyrrolidine, and substituted azetidine.

In some embodiments, in each of Formulas (II.a.), (II.a.1), and (II.a.2), R² is selected from:

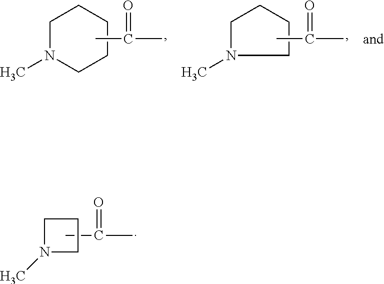

In some embodiments, in each of Formulas (II.a.), (II.a.1), and (II.a.2), R² is —C(O)NR⁹R¹⁰.

In some embodiments, in each of Formulas (II.a.), (II.a.1), and (II.a.2), R² is —C(O)NH₂.

In some embodiments, in each of Formulas (II.a.), (II.a.1), and (II.a.2), R² is —C(O)NR⁹R¹⁰, wherein R⁹ and R¹⁰ can be the same or different, each being independently selected from alkyl.

In some embodiments, in each of Formulas (II.a.), (II.a.1), and (II.a.2), R² is —C(O)NR⁹R¹⁰, wherein R⁹ is unsubstituted heterocycloalkyl and R¹⁰ is selected from the group consisting of H and alkyl.

In some embodiments, in each of Formulas (II.a.), (II.a.1) and (II.a.2), R² is —C(O)NR⁹R¹⁰, wherein R⁹ is substituted heterocycloalkyl and R¹⁰ is selected from the group consisting of H and alkyl.

In some embodiments, in each of Formulas (II.a.), (II.a.1), and (II.a.2), R² is —C(O)NR⁹R¹⁰, wherein R⁹ is heterocycloalkyl substituted with from one to three substituents, which can be the same or different, each substituent being independently selected from alkyl, and R¹⁰ is selected from the group consisting of H and alkyl.

In some embodiments, in each of Formulas (II.a.), (II.a.1), and (II.a.2), R² is selected from the group consisting of: alkyl, haloalkyl, heteroalkyl, heterohaloalkyl, —C(O)R⁷. —C(O)OR⁸, and —C(O)NR⁹R¹⁰.

Non-limiting examples of R² include the following moieties:

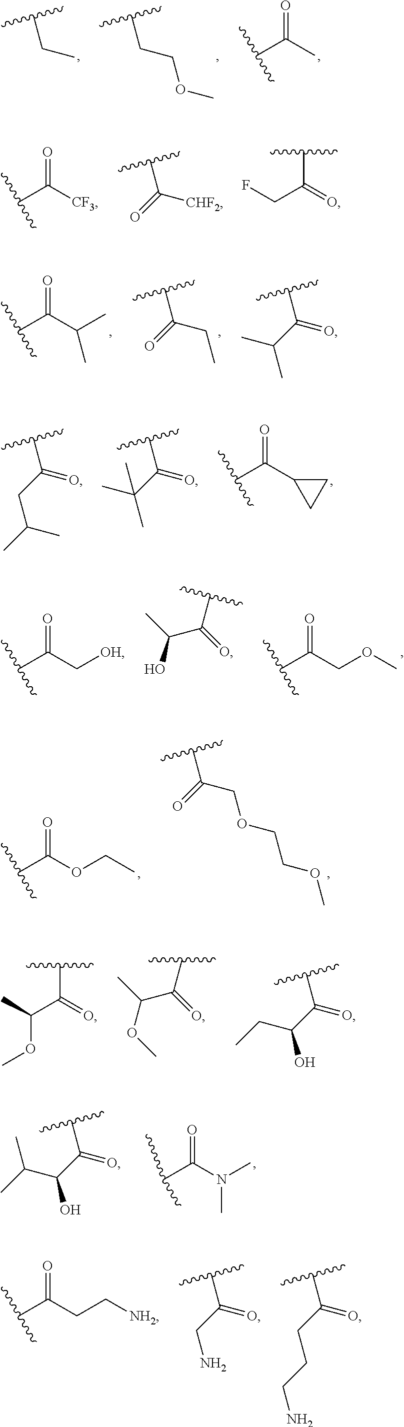

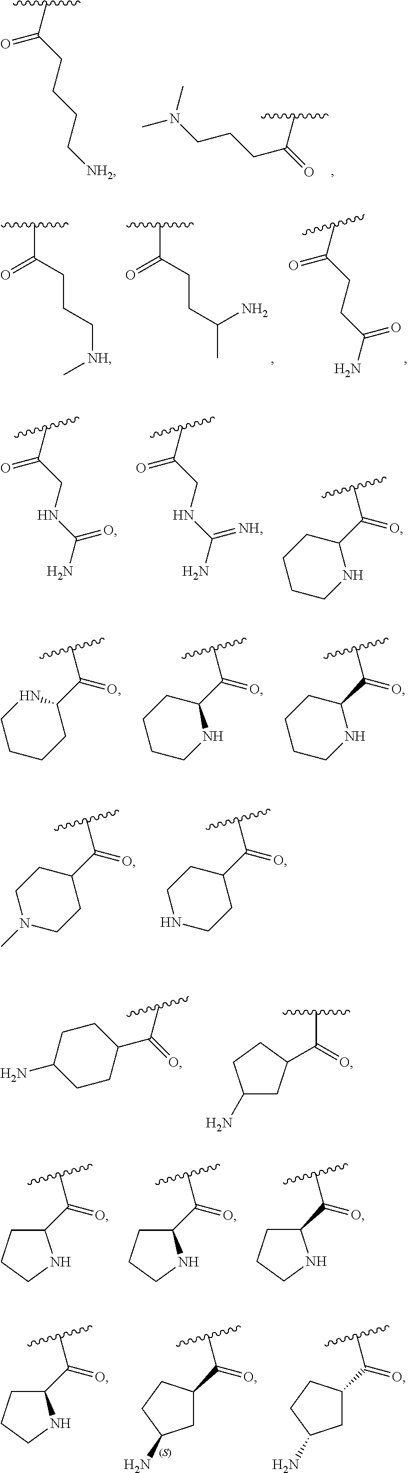

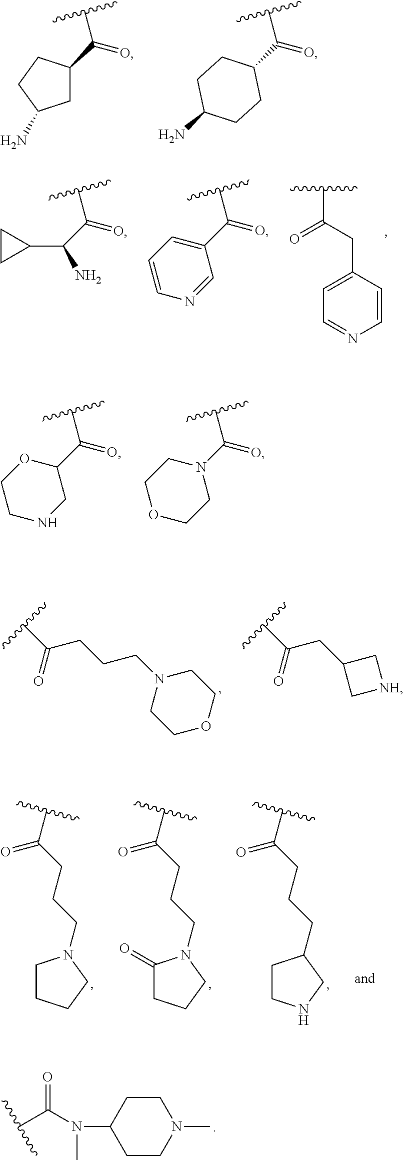

In some embodiments, in each of Formulas (II.a.), (II.a.1), and (II.a2), R² is

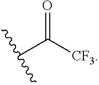

In some embodiments, in each of Formulas (II.a.), (II.a.1), and (II.a.2), R² is

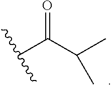

In some embodiments, in each of Formulas (II.a.), (II.a.1), and (II.a.2), R² is

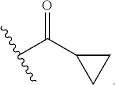

In some embodiments, in each of Formulas (II.a.), (II.a.1), and (II.a.2), R² is

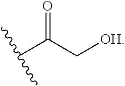

In some embodiments, in each of Formulas (II.a.), (II.a.1), and (II.a.2), R² is

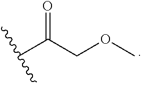

In some embodiments, in each of Formulas (II.a.), (II.a.1), and (II.a.2), R² is

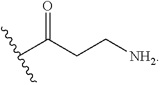

In some embodiments, in each of Formulas (II.a.), (II.a.1), and (II.a.2), R² is

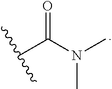

In some embodiments, in each of Formulas (II.a.), (II.a.1), and (II.a.2), R² is

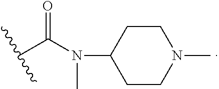

In one embodiment, the compounds of the invention have a structure shown in Formula (II.b) and include pharmaceutically acceptable salts, solvates, esters, prodrugs, or isomers of said compounds:

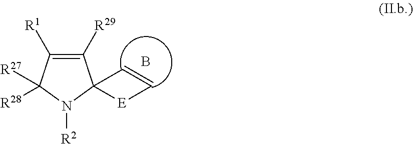

wherein R¹, R², R²⁷, R²⁸, R²⁹, E, and ring B are selected independently of each other and wherein:
E is selected from the group consisting of —O—, —S—, —S(O)—, —S(O)₂—, —C(R⁴)(R⁵)—, —N(R⁶)—, —N(C(Y)R⁷)—, —N(C(Y)OR⁸)—, —N(C(Y)N(R⁹)(R¹⁰))—,
ring B is a substituted or unsubstituted heteroaromatic ring;
and R¹, R², R⁴, R⁵, R⁶, R⁷, R⁸, R⁹, R¹⁰, R²⁷, R²⁸, R²⁹, Y, and the optional substituents on ring B are as defined in any of the embodiments described above in Formula (I).

In one embodiment, Formula (II.b.) has the general structure shown in Formula (II.b.1):

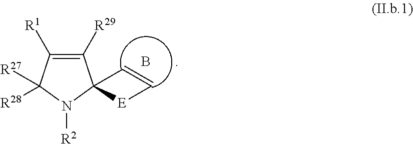

In one embodiment, Formula (II.b.) has the general structure shown in Formula (II.b.2):

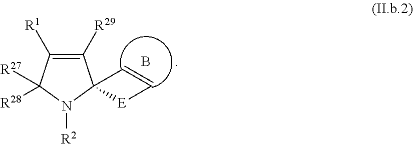

In some embodiments, in each of Formulas (II.b), (II.b.1), and (II.b.2), E is —C(R⁴)(R⁵)—.

In some embodiments, in each of Formulas (II.b), (II.b.1), and (II.b.2), E is selected from the group consisting of —O—, —S—, —S(O)—, —S(O)₂—, and —N(R⁶)—.

In some embodiments, in each of Formulas (II.b), (II.b.1), and (II.b.2), E is selected from the group consisting of —O—, —S—, —S(O)—, —S(O)₂—, and —N(R⁶)—, wherein R⁶ is selected from the group consisting of H, alkyl, —C(O)R²⁴, and —C(S)R²⁴.

In some embodiments, in each of Formulas (II.b), (II.b.1), and (II.b.2), E is selected from the group consisting of —O— and —N(R⁶)—, wherein R⁶ is selected from the group consisting of H, alkyl, —C(O)R²⁴, and —C(S)R²⁴.

In some embodiments, in each of Formulas (II.b), (II.b.1), and (II.b.2), E is —O—.

In some embodiments, in each of Formulas (II.b), (II.b.1), and (II.b.2), E is —S—.

In some embodiments, in each of Formulas (II.b), (II.b.1), and (II.b.2), E is —S(O)—.

In some embodiments, in each of Formulas (II.b), (II.b.1), and (II.b.2), E is —S(O)₂—.

In some embodiments, in each of Formulas (II.b), (II.b.1), and (II.b.2), E is —C(R⁴)(R⁵)—.

In some embodiments, in each of Formulas (II.b), (II.b.1), and (II.b2), E is —N(R⁶)—.

In some embodiments, in each of Formulas (II.b), (II.b.1), and (II.b.2), E is —N(C(Y)R⁷)—.

In some embodiments, in each of Formulas (II.b), (II.b.1), and (II.b.2), E is —N(C(Y)OR⁸)—.

In some embodiments, in each of Formulas (II.b), (II.b.1), and (II.b.2), E is —N(C(Y)N(R⁹)(R¹⁰))—.

In some embodiments, in each of Formulas (II.b), (II.b.1), and (II.b.2), Y is (═O).

In some embodiments, in each of Formulas (II.b), (II.b.1), and (II.b.2), Y is (═S).

In some embodiments, in each of Formulas (II.b), (II.b.1), and (II.b.2), Y is (═N(R¹³)).

In some embodiments, in each of Formulas (II.b), (II.b.1), and (II.b.2), Y is (═N(CN)).

In some embodiments, in each of Formulas (II.b), (II.b.1), and (II.b.2), Y is (═N(OR¹⁴)).

In some embodiments, in each of Formulas (II.b), (II.b.1), and (II.b.2), Y is (═N(R¹⁵)(R¹⁶)).

In some embodiments, in each of Formulas (II.b), (II.b.1), and (II.b.2), Y is (═C(R¹⁷)(R¹⁸)).

In some embodiments, in each of Formulas (II.b), (II.b.1), and (II.b.2), B is an unsubstituted heteroaromatic ring.

In some embodiments, in each of Formulas (II.b), (II.b.1), and (II.b.2), B is an unsubstituted 5-6-membered heteroaromatic ring having from 1-3 ring heteroatoms, which can be the same or different, each hetero ring atom being independently selected from the group consisting of N, S, O, S(O), and S(O)₂.

In some embodiments, in each of Formulas (III), (II.b.1), and (II.b.2), B is a heteroaromatic ring which is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁹, —NR²³C(O)R²⁴, —SO₂NR²⁵R²⁶, —O(O)R²⁴, —C(O)OR²⁰, —S(O)R¹⁹, —SO₂R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (II.b), (II.b.1), and (II.b.2), B is a 5-6-membered heteroaromatic ring having from 1-3 ring heteroatoms, which can be the same or different, each hetero ring atom being independently selected from the group consisting of N, S, O, S(O), and S(O)₂, which heteroaromatic ring is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (II.b), (II.b.1), and (II.b.2), B is an unsubstituted 6-membered heteroaromatic ring having from 1-3 ring heteroatoms, which can be the same or different, each hetero ring atom being independently selected from the group consisting of N, S, and O.

In some embodiments, in each of Formulas (II.b), (II.b.1), and (II.b.2), B is a 6-membered heteroaromatic ring having from 1-3 ring heteroatoms, which can be the same or different, each hetero ring atom being independently selected from the group consisting of N, S, and O, which heteroaromatic ring is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (II.b), (II.b.1), and (II.b.2), B is an unsubstituted 6-membered heteroaromatic ring having 2 ring heteroatoms, each ring heteroatom being independently selected from of N, S, and O.

In some embodiments, in each of Formulas (II.b), (II.b.1), and (II.b.2), B is a 6-membered heteroaromatic ring having 2 ring heteroatoms, each ring heteroatom being independently selected from of N, S, and O, which heteroaromatic ring is substituted with one or more substituents, which can be the same or different, each substituent being independently selected from the group consisting of halogen, —CN, —NO₂, alkyl, heteroalkyl, haloalkyl, —OR¹⁹, —NR²¹R²², —C(O)R²⁴, —C(O)OR²⁰, —SR¹⁹, —S(O)R¹⁹, —SO₂R¹⁹, —OC(O)R²⁴, and —C(O)NR²⁵R²⁶.

In some embodiments, in each of Formulas (II.b), (II.b.1), and (II.b.2), B is an unsubstituted 5-membered heteroaromatic ring having from 1-2 ring heteroatoms, which can be the same or different, each hetero ring atom being independently selected from the group consisting of N, S, and O.

In some embodiments, in each of Formulas (II.b), (II.b.1), and (II.b.2), B is a 5-membered heteroaromatic ring having from 1-2 ring heteroatoms, which can be the same or different, each hetero ring atom being independently selected from the group consisting of N, S, and O, which heteroaromatic ring is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (II.b), (II.b.1), and (II.b.2), B is an unsubstituted 5-membered heteroaromatic ring having 1 ring heteroatom selected from of N, S, and O.

In some embodiments, in each of Formulas (II.b), (II.b.1), and (II.b.2), B is a 5-membered heteroaromatic ring having 1 ring heteroatom selected from of N, S, and O, which heteroaromatic ring is substituted with one or more substituents, which can be the same or different, each substituent being independently selected from the group consisting of halogen, —CN, —NO₂, alkyl, heteroalkyl, haloalkyl, —OR¹⁹, —NR²¹R²², —C(O)R²⁴, —C(O)OR²⁰, —S(O)R¹⁹, —SO₂R¹⁹, —OC(O)R²⁴, and —C(O)NR²⁵R²⁶.

In some embodiments, in each of Formulas (II.b), (II.b.1), and (II.b.2), B is a 5-membered heteroaromatic ring having S as the ring heteroatom, which heteroaromatic ring is substituted with one or more substituents, which can be the same or different, each substituent being independently selected from the group consisting of halogen, —CN, —NO₂, alkyl, heteroalkyl, haloalkyl, —OR¹⁹, —NR²¹R²², —C(O)R²⁴, —C(O)OR²⁰, —SR¹⁹, —S(O)R¹⁹, —SO₂R¹⁹, —OC(O)R²⁴, and —C(O)NR²⁵R²⁶.

In some embodiments, in each of Formulas (II.b), (II.b.1), and (II.b.2), B is an unsubstituted 5-membered heteroaromatic ring having S as the ring heteroatom.

In one embodiment, Formula (II.b.) has the general structure:

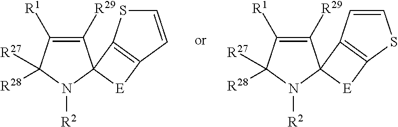

In one embodiment, Formula (II.b.) has the general structure:

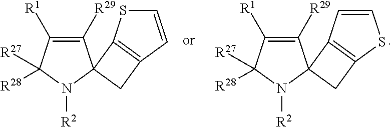

In some embodiments, in each of Formulas (II.b), (II.b.1), and (II.b.2), R¹ is unsubstituted aryl.

In some embodiments, in each of Formulas (II.b), (II.b.1), and (II.b.2), R¹ is unsubstituted phenyl.

In some embodiments, in each of Formulas (II.b), (II.b.1), and (II.b.2), R¹ is unsubstituted naphthyl.

In some embodiments, in each of Formulas (II.b), (II.b.1), and (II.b.2), R¹ is substituted aryl.

In some embodiments, in each of Formulas (II.b), (II.b.1), and (II.b.2), R¹ is substituted phenyl.

In some embodiments, in each of Formulas (II.b), (II.b.1), and (II.b.2), R¹ is substituted naphthyl.

In some embodiments, in each of Formulas (II.b), (II.b.1), and (II.b.2), R¹ is aryl substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl, cycloalkyl, cycloalkenyl, heterocycloalkyl, heterocycloalkenyl, azide, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (II.b), (II.b.1), and (II.b.2), R¹ is phenyl substituted with one or more substituents, which can be the same or different, each substituent being independently selected from the group consisting halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (II.b), (II.b.1), and (II.b.2), R¹ is phenyl substituted with one to four substituents, which can be the same or different, each substituent being independently selected from the group consisting of halo, —OH, —CN, —NO₂, —NR²¹R²², and haloalkyl.

In some embodiments, in each of Formulas (II.b), (II.b.1), and (II.b.2), R¹ is selected from the group consisting of:

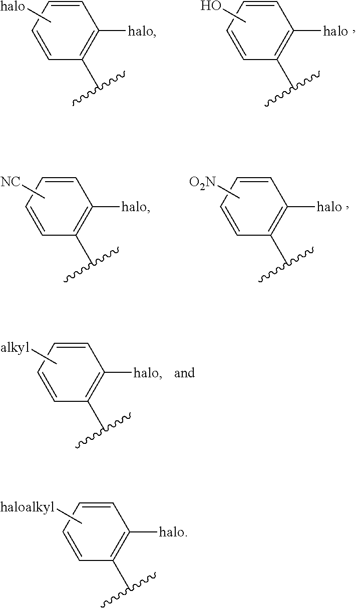

In some embodiments, in each of Formulas (II.b), (II.b.1), and (II.b.2), R¹ is:

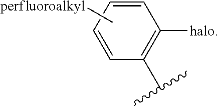

In some embodiments, in each of Formulas (II.b), (II.b.1), and (II.b.2), R¹ is phenyl substituted with one to three fluoro groups.

In some embodiments, in each of Formulas (II.b), (II.b.1), and (II.b.2), R¹ is phenyl substituted with two fluoro groups.

In some embodiments, in each of Formulas (II.b), (II.b.1), and (II.b.2), R¹ is phenyl substituted with one fluoro group.

In some embodiments, in each of Formulas (II.b), (II.b.1), and (II.b.2), R¹ is:

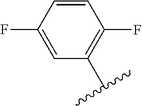

In some embodiments, in each of Formulas (II.b), (II.b.1), and (II.b.2), R²⁷, R²⁸, and R²⁹ are each independently selected from the group consisting of H and alkyl.

In some embodiments, in each of Formulas (II.b), (II.b.1), and (II.b.2), R² is —C(Z)R⁷.

In some embodiments, in each of Formulas (II.b), (II.b.1), and (II.b.2), R² is —C(Z)NR⁹R¹⁰.

In some embodiments, in each of Formulas (II.b), (II.b.1), and (II.b.2), R² is —C(Z)OR⁸.

In some embodiments, in each of Formulas (II.b), (II.b.1), and (II.b.2), R² is —SO₂NR⁹R¹⁰.

In some embodiments, in each of Formulas (II.b), (II.b.1), and (II.b.2), R² is alkyl.

In some embodiments, in each of Formulas (II.b), (II.b.1), and (II.b.2), R² is heteroalkyl.

In some embodiments, in each of Formulas (II.b), (II.b.1), and (II.b.2), R² is aryl.

In some embodiments, in each of Formulas (II.b), (II.b.1), and (II.b.2), R² is heteroaryl.

In some embodiments, in each of Formulas (II.b), (II.b.1), and (II.b.2), R² is cycloalkyl.

In some embodiments, in each of Formulas (II.b), (II.b.1), and (II.b.2), R² is cycloalkenyl.

In some embodiments, in each of Formulas (II.b), (II.b.1), and (II.b.2), R² is heterocycloalkyl.

In some embodiments, in each of Formulas (II.b), (II.b.1), and (II.b.2), R² is heterocycloalkenyl.

In some embodiments, in each of Formulas (II.b), (II.b.1), and (II.b.2), Z is (═O).

In some embodiments, in each of Formulas (II.b), (II.b.1), and (II.b.2), Z is (═S).

In some embodiments, in each of Formulas (II.b), (II.b.1), and (II.b.2), Z is (═N(R¹³)).

In some embodiments, in each of Formulas (II.b), (II.b.1), and (II.b.2), Z is (═N(CN)).

In some embodiments, in each of Formulas (II.b), (II.b.1), and (II.b.2), Z is (═N(OR¹⁴)).

In some embodiments, in each of Formulas (II.b), (II.b.1), and (II.b.2), Z is (═N(R¹⁵)(R¹⁶)).

In some embodiments, in each of Formulas (II.b), (II.b.1), and (II.b.2), Z is (═O(R¹⁷)(R¹⁸)).

In some embodiments, in each of Formulas (II.b), (II.b.1), and (II.b.2), R² is —C(Z)R⁷, and Z is (═O).

In some embodiments, in each of Formulas (II.b), (II.b.1), and (II.b.2), R² is —C(O)H.

In some embodiments, in each of Formulas (II.b), (II.b.1), and (II.b.2), R² is —C(O)alkyl.

In some embodiments, in each of Formulas (II.b), (II.b.1), and (II.b.2), R² is —C(O)CH₃.

In some embodiments, in each of Formulas (II.b), (II.b.1), and (II.b.2), R² is —C(O)R⁷, wherein said R⁷ is alkyl substituted with one or more substituents, which can be the same or different, each substituent being independently selected from the group consisting of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁵, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (II.b), (II.b.1), and (II.b.2), R² is —C(O)R⁷, wherein said R⁷ is alkyl substituted with one to three substituents, which can be the same or different, each substituent being independently selected from the group consisting of —OR¹⁹, —NR²¹R²², and cycloalkyl.

In some embodiments, in each of Formulas (II.b), (II.b.1), and (II.b.2), R² is —C(O)R⁷, wherein said R⁷ is alkyl, wherein said alkyl is substituted with alkyl and —OH.

In some embodiments, in each of Formulas (II.b), (II.b.1), and (II.b.2), R² is —C(O)R⁷, wherein said R⁷ is alkyl substituted with one to three substituents, which can be the same or different, each substituent being independently selected from the group consisting of —OH, —NH₂, and cyclopropyl.

In some embodiments, in each of Formulas (II.b), (II.b.1), and (II.b.2), R² is —C(O)R⁷, wherein said R⁷ is alkyl substituted with one to two substituents, which can be the same or different, each substituent being independently selected from the group consisting of —NH₂, and cyclopropyl.

In some embodiments, in each of Formulas (II.b), (II.b.1), and (II.b.2), R² is —C(O)R⁷, wherein said R⁷ is alkyl substituted with —OH.

In some embodiments, in each of Formulas (II.b), (II.b.1), and (II.b.2), R² is —C(O)R⁷, wherein said R⁷ is unsubstituted heterocycloalkyl.

In some embodiments, in each of Formulas (II.b), (II.b.1), and (II.b.2), R² is —C(O)R⁷, wherein said R⁷ is substituted heterocycloalkyl.

In some embodiments, in each of Formulas (II.b), (II.b.1), and (II.b.2), R² is —C(O)R⁷, wherein said R⁷ is heterocycloalkyl substituted with one or more substituents, which can be the same or different, each substituent being independently selected from the group consisting of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (II.b), (II.b.1), and (II.b.2), R² is —C(O)R⁷, wherein said R⁷ is selected from the group consisting of substituted piperidine, substituted piperazine, substituted morpholine, substituted pyrrolidine, and substituted azetidine.

In some embodiments, in each of Formulas (II.b), (II.b.1), and (II.b.2), R² is selected from:

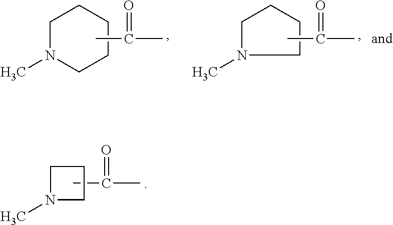

In some embodiments, in each of Formulas (II.b), (II.b.1), and (II.b.2), R² is —C(O)NR⁹R¹⁰.

In some embodiments, in each of Formulas (II.b), (II.b.1), and (II.b.2), R² is —C(O)NH₂.

In some embodiments, in each of Formulas (II.b), (II.b.1), and (II.b.2), R² is —C(O)NR⁹R¹⁰, wherein R⁹ and R¹⁰ can be the same or different, each being independently selected from alkyl.

In some embodiments, in each of Formulas (II.b), (II.b.1), and (II.b.2), R² is —C(O)NR⁹R¹⁰, wherein R⁹ is unsubstituted heterocycloalkyl and R¹⁰ is selected from the group consisting of H and alkyl.

In some embodiments, in each of Formulas (II.b), (II.b.1), and (II.b.2), R² is —C(O)NR⁹R¹⁰, wherein R⁹ is substituted heterocycloalkyl and R¹⁰ is selected from the group consisting of H and alkyl.

In some embodiments, in each of Formulas (II.b), (II.b.1), and (II.b.2), R² is —C(O)NR⁹R¹⁰, wherein R⁹ is heterocycloalkyl substituted with from one to three substituents, which can be the same or different, each substituent being independently selected from alkyl, and R¹⁰ is selected from the group consisting of H and alkyl.

In some embodiments, in each of Formulas (II.b), (II.b.1), and (II.b.2), R² is selected from the group consisting of: alkyl, haloalkyl, heteroalkyl, heterohaloalkyl, —C(O)R⁷. —C(O)OR⁸, and —C(O)NR⁹R¹⁰.

Non-limiting examples of R² include the following moieties:

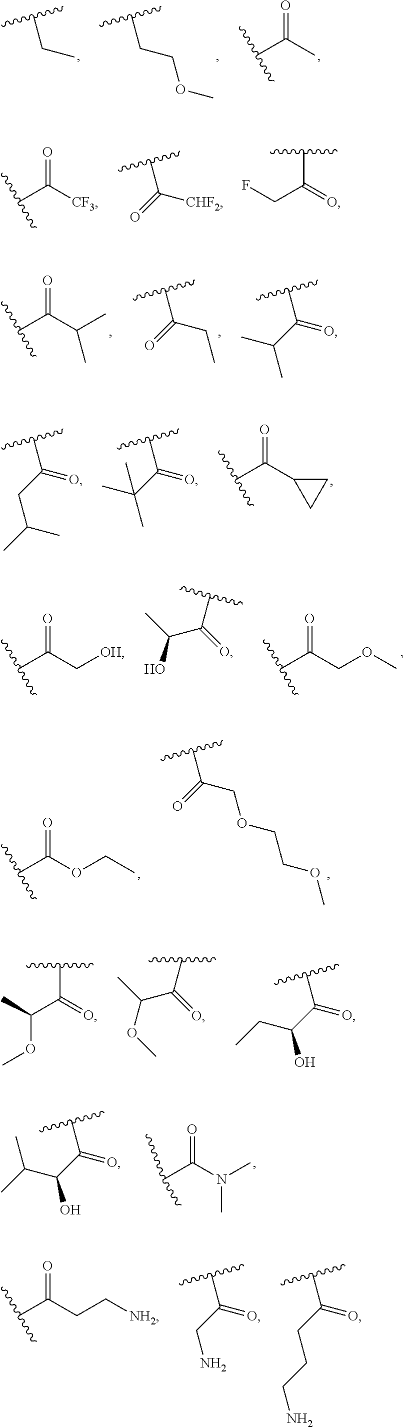

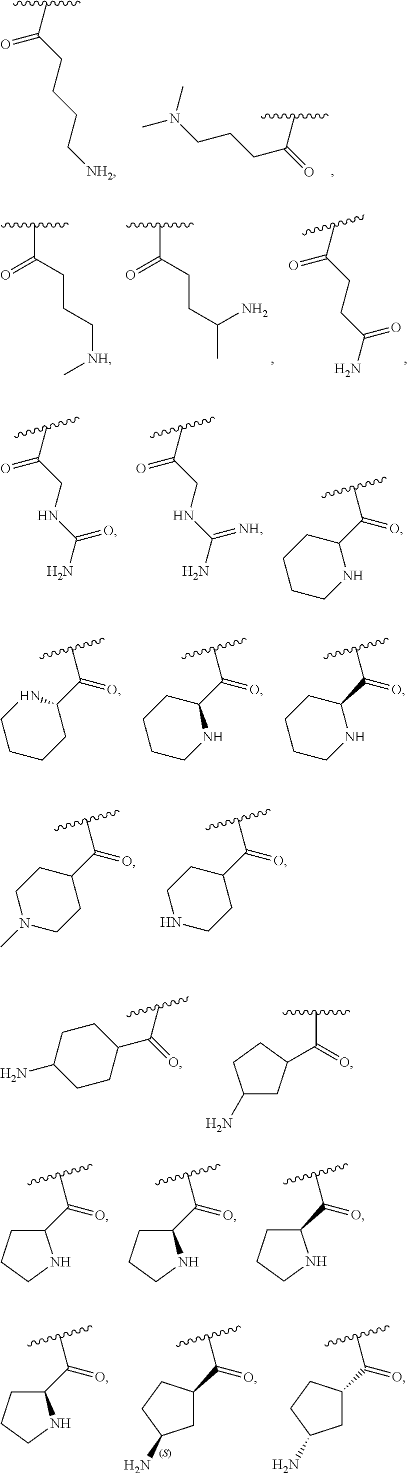

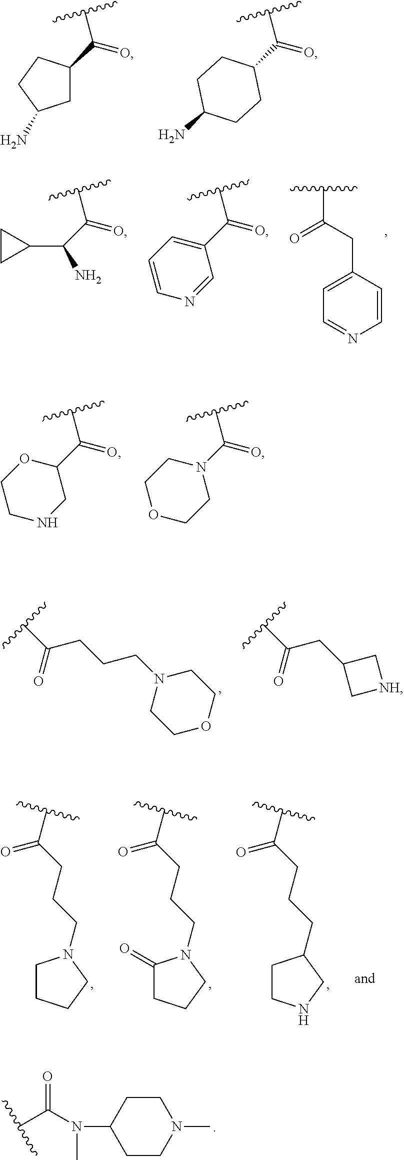

In some embodiments, in each of Formulas (II.b), (II.b.1), and (II.b.2), R² is

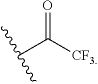

In some embodiments, in each of Formulas (II.b), (II.b.1), and (II.b.2), R² is

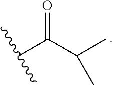

In some embodiments, in each of Formulas (II.b), (II.b.1), and (II.b.2), R² is

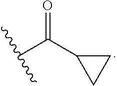

In some embodiments, in each of Formulas (II.b), (II.b.1), and (II.b.2), R² is

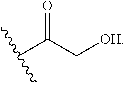

In some embodiments, in each of Formulas (II.b), (II.b.1), and (II.b.2), R² is

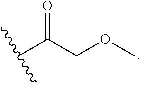

In some embodiments, in each of Formulas (II.b), (II.b.1), and (II.b.2), R² is

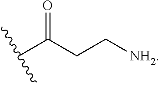

In some embodiments, in each of Formulas (II.b), (II.b.1), and (II.b.2), R² is

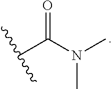

In some embodiments, in each of Formulas (II.b), (II.b.1), and (II.b.2), R² is

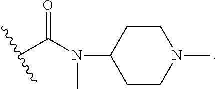

In one embodiment, the compounds of the invention have a structure shown in Formula (III.1) and include pharmaceutically acceptable salts, solvates, esters, prodrugs, or isomers of said compounds:

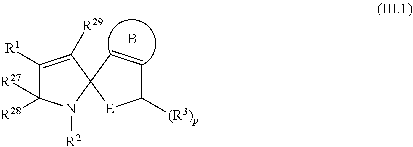

wherein R¹, R², R³, R²⁷, R²⁸, R²⁹, p, E, and ring B are selected independently of each other and wherein:
E is selected from the group consisting of —O—, —S—, —S(O)—, —S(O)₂—, —C(R⁴)(R⁵)—, —N(R⁶)—, —N(C(Y)R⁷)—, —N(C(Y)OR⁸)—, and —N(C(Y)N(R⁹)(R¹⁰))—; and
p is 0, 1, or 2; and
ring B, R¹, R², R⁴, R⁵, R⁶, R⁷, R⁸, R⁹, R¹⁰, R²⁷, R²⁸, R²⁹, Y, and the optional substituents on ring B are as defined in any of the embodiments described above in Formula (I).

In one embodiment, in Formula (III.1):

E is selected from the group consisting of —C(R⁴)(R⁵)—, —O—, —S—, —S(O)—, —S(O)₂—, and —N(R⁶)—;
ring B is an unsubstituted or substituted aromatic ring or an unsubstituted or substituted 5-6-membered heteroaromatic ring having from 1-3 ring heteroatoms, which ring heteroatoms can be the same or different, each ring heteroatom being independently selected from the group consisting of N, S, O, S(O), and S(O)₂, said substituents on said aromatic ring or said heteroaromatic ring (when present) being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
R¹ is unsubstituted aryl or aryl substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
R² is selected from the group consisting of —C(O)R⁷, —C(O)NR⁹R¹⁰, and —C(O)OR⁸;
p is 0 or 1; and
each R³ (when present) is independently selected from the group consisting of alkyl, heteroalkyl, alkenyl, heteroalkenyl, —CN, —NO₂, —OR¹⁹, —OC(O)OR²⁰, —NR²¹R²², —C(O)R²⁴, —C(S)R²⁴, —C(O)OR²⁰, and —C(O)NR²⁵R²⁶,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁶, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (III.1):

ring B is an unsubstituted or substituted moiety selected from the group consisting of benzo, (uranyl, thiophenyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, and triazinyl;
R¹ is phenyl substituted with one to four substituents, which can be the same or different, each substituent being independently selected from the group consisting of halo, —OH, —CN, —NO₂, —NR²¹R²², and haloalkyl;
R² is selected from the group consisting of —C(O)R⁷, —C(O)NR⁹R¹⁰, and —C(O)OR⁸;
p is 0 or 1; and
each R³ (when present) is independently selected from the group consisting of alkyl, heteroalkyl, alkenyl, heteroalkenyl,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one such embodiment, in Formula (III.1):

### R¹ is:

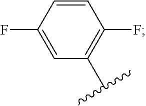

R⁶ is selected from the group consisting of H, alkyl, —C(O)R²⁴, —C(O)OR²⁰, and —C(S)R²⁴; and
R²⁷, R²⁸, and R²⁹ are each independently selected from the group consisting of H and alkyl.

In one embodiment, the compounds of the invention have a structure shown in Formula (III.2) and include pharmaceutically acceptable salts, solvates, esters, prodrugs, or isomers of said compounds:

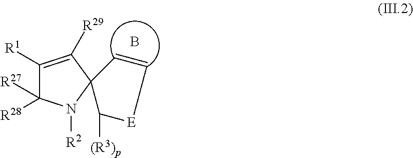

wherein R¹, R², R³, R²⁷, R²⁸, R²⁹, p, E, and ring B are selected independently of each other and wherein:
E is selected from the group consisting of —O—, —S—, —S(O)—, —S(O)₂—, —C(R⁴)(R⁵)—, —N(R⁶)—, —N(C(Y)R⁷)—, —N(C(Y)OR⁸)—, and —N(C(Y)N(R⁹)(R¹⁰))—; and
p is 0, 1, or 2, and
ring B, R¹, R², R⁴, R⁵, R⁶, R⁷, R⁸, R⁹, R¹⁰, R²⁷, R²⁸, R²⁹, Y, and the optional substituents on ring B are as defined in any of the embodiments described above in Formula (I).

In one embodiment, in Formula (III.2):

E is selected from the group consisting of —C(R⁴)(R⁵)—, —O—, —S—, —S(O)—, —S(O)₂—, and —N(R⁶)—;
ring B is an unsubstituted or substituted aromatic ring or an unsubstituted or substituted 5-6-membered heteroaromatic ring having from 1-3 ring heteroatoms, which ring heteroatoms can be the same or different, each ring heteroatom being independently selected from the group consisting of N, S, O, S(O), and S(O)₂, said substituents on said aromatic ring or said heteroaromatic ring (when present) being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
R¹ is unsubstituted aryl or aryl substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
R² is selected from the group consisting of —C(O)R⁷, —O(O)NR⁹R¹⁰, and —C(O)OR⁸;
p is 0 or 1; and
each R³ (when present) is independently selected from the group consisting of alkyl, heteroalkyl, alkenyl, heteroalkenyl, —CN, —NO₂, —OR¹⁹, —OC(O)OR²⁰, —NR²¹R²², —C(O)R²⁴, —C(S)R²⁴, —C(O)OR²⁰, and —C(O)NR²⁵R²⁶,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (III.2):

ring B is an unsubstituted or substituted moiety selected from the group consisting of benzo, (uranyl, thiophenyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, and triazinyl;
R¹ is phenyl substituted with one to four substituents, which can be the same or different, each substituent being independently selected from the group consisting of halo, —OH, —CN, —NO₂, —NR²¹R²², and haloalkyl;
R² is selected from the group consisting of —C(O)R⁷, —C(O)NR⁹R¹⁰, and —C(O)OR⁸;
p is 0 or 1; and
each R³ (when present) is independently selected from the group consisting of alkyl, heteroalkyl, alkenyl, heteroalkenyl,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one such embodiment, in Formula (III.2):

### R¹ is:

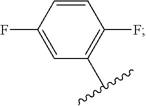

R⁶ is selected from the group consisting of H, alkyl, —C(O)R²⁴, —C(O)OR²⁰, and —C(S)R²⁴; and
R²⁷, R²⁸, and R²⁹ are each independently selected from the group consisting of H and alkyl.

In one embodiment, the compounds of the invention have a structure shown in Formula (III.a) and include pharmaceutically acceptable salts, solvates, esters, prodrugs, or isomers of said compounds:

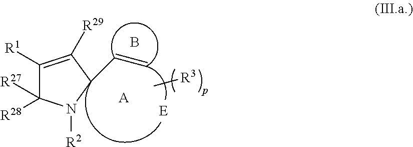

wherein R¹, R², R³, R²⁷, R²⁸, R²⁹, p, E, ring A, and ring B are selected independently of each other and wherein:
ring A (including E and the unsaturation shown) is a 5-membered cycloalkenyl or heterocycloalkenyl ring;
E is selected from the group consisting of —O—, —S—, —S(O)₂—, —C(R⁴)(R⁵)—. —N(R⁶)—, —N(C(Y)R⁷)—, —N(C(Y)OR⁸)—, —N(C(Y)N(R⁹)(R¹⁰))—, —C(O)—N(R¹¹)—, —N(R¹¹)—C(O)—, —S(O)₂—N(R¹¹)—, —N(R¹¹)—S(O)₂—, —C(O)—O—, —O—C(O)—, —O—N(R⁶)—, —N(R⁶)—O—, —N(R⁶)—N(R¹²)—, —N═N—, and —C(R⁷)═N—;
ring B is a substituted or unsubstituted aromatic ring;
p R¹, R², R³, R⁴, R⁵, R⁶, R⁷, R⁸, R⁹, R¹⁰, R¹¹, R¹², R²⁷, R²⁸, R²⁹, Y, and the optional substituents on ring B are as defined in any of the embodiments described above in Formula (I).

In one embodiment, Formula (III.a) has the general structure:

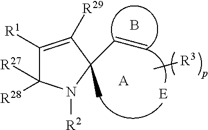

In one embodiment, Formula (III.a) has the general structure:

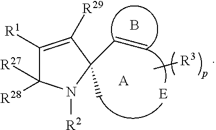

In one embodiment, in Formula (III.a.), p is 0, 1, or 2;

In one embodiment, in Formula (III.a.), ring A is a cycloalkenyl ring and E is —C(R⁴)(R⁵)—.

In one embodiment, in Formula (III.a.), ring A is a heterocycloalkenyl ring and E is selected from the group consisting of —C(O)—N(R¹¹)—, —N(R¹¹)—C(O)—, —S(O)₂—N(R¹¹)—, —N(R¹¹)—S(O)₂—, —C(O)—O—, —O—C(O)—, —O—N(R⁶)—, —N(R⁶)—O—, —N(R⁶)—N(R¹²)—, —N═N—, and —C(R⁷)═N—. By way of non-limiting illustration, an example of a compound of Formula (III.a.) wherein E is —C(O)—N(R¹¹)—includes:

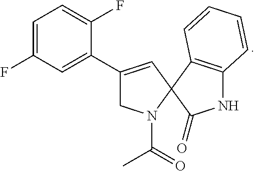

In one embodiment, in Formula (III.a.), ring A is a heterocycloalkenyl ring and E is selected from the group consisting of —O—, —S—, —S(O)—, —S(O)₂—, and —N(R⁶)—.

In one embodiment, in Formula (III.a.), E is selected from the group consisting of —O—, —S—, —S(O)—, —S(O)₂—, and —N(R⁶)—, wherein R⁶ is selected from the group consisting of H, alkyl, —C(O)R²⁴, and —C(S)R²⁴.

In one embodiment, in Formula (III.a.), E is selected from the group consisting of —O— and —N(R⁶)—, wherein R⁶ is selected from the group consisting of H, alkyl, —C(O)R²⁴, and —C(S)R²⁴.

In one embodiment, in Formula (III.a.), E is —O—.

In one embodiment, in Formula (III.a.), E is —S—.

In one embodiment, in Formula (III.a.), E is —S(O)—.

In one embodiment, in Formula (III.a.), E is —S(O)₂—.

In one embodiment, in Formula (III.a.), E is —C(R⁴)(R⁵)—.

In one embodiment, in Formula (III.a.), E is —N(R⁶)—.

In one embodiment, in Formula (III.a.), E is —N(C(Y)R⁷)—.

In one embodiment, in Formula (III.a.), E is —N(C(Y)OR⁸)—.

In one embodiment, in Formula (III.a.), E is —N(C(Y)N(R⁹)(R¹⁰))—.

In one embodiment, in Formula (III.a.), E is —C(O)—N(R¹¹)—.

In one embodiment, in Formula (III.a.), E is —N(R¹¹)—C(O)—.

In one embodiment, in Formula (III.a.), E is —S(O)₂—N(R¹¹)—.

In one embodiment, in Formula (III.a.), E is —N(R¹¹)—S(O)₂—.

In one embodiment, in Formula (III.a.), E is —C(O)—O—.

In one embodiment, in Formula (III.a.), E is —O—C(O)—.

In one embodiment, in Formula (III.a.), E is —O—N(R⁶)—.

In one embodiment, in Formula (III.a.), E is —N(R⁶)—O—.

In one embodiment, in Formula (III.a.), E is —N(R⁶)—N(R¹²)—.

In one embodiment, in Formula (III.a.), E is —N═N—.

In one embodiment, in Formula (III.a.), E is —C(R⁷)═N—.

In one embodiment, in Formula (III.a.), Y is (═O).

In one embodiment, in Formula (III.a.), Y is (═S).

In one embodiment, in Formula (III.a.), Y is (═N(R¹³)).

In one embodiment, in Formula (III.a.), Y is (═N(CN)).

In one embodiment, in Formula (III.a.), Y is (═N(OR¹⁴)).

In one embodiment, in Formula (III.a.), Y is (═N(R¹⁵)(R¹⁶).

In one embodiment, in Formula (III.a.), Y is (═C(R¹⁷)(R¹⁸)).

In one embodiment, in Formula (III.a.), B is an unsubstituted aromatic ring.

In one embodiment, in Formula (III.a.), B is an unsubstituted benzo ring, and Formula (III.a.) has the general structure:

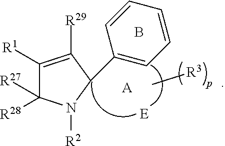

In one embodiment, in Formula (III.a.), B is an aromatic ring which is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (III.a.), B is a benzo ring which is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (III.a.), R¹ is unsubstituted aryl.

In one embodiment, in Formula (III.a.), R¹ is unsubstituted phenyl.

In one embodiment, in Formula (III.a.), R¹ is unsubstituted naphthyl.

In one embodiment, in Formula (III.a.), R¹ is substituted aryl.

In one embodiment, in Formula (III.a.), R¹ is substituted phenyl.

In one embodiment, in Formula (III.a.), R¹ is substituted naphthyl.

In one embodiment, in Formula (III.a.), R¹ is aryl substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (III.a.), R¹ is phenyl substituted with one or more substituents, which can be the same or different, each substituent being independently selected from the group consisting halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (III.a.), R¹ is phenyl substituted with one to four substituents, which can be the same or different, each substituent being independently selected from the group consisting of halo, —OH, —CN, —NO₂, —NR²¹R²², and haloalkyl.

In one embodiment, in Formula (III.a.), R¹ is selected from the group consisting of:

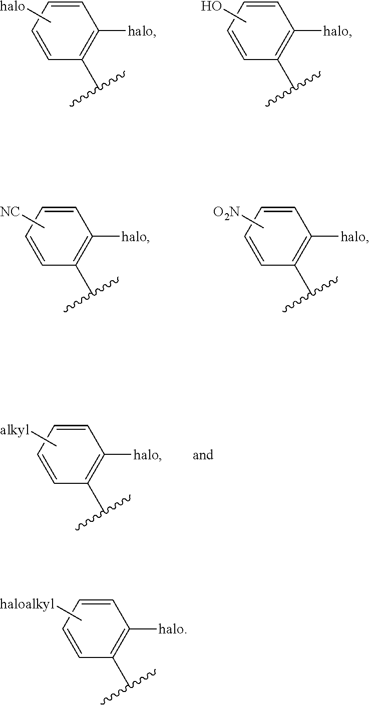

In one embodiment, in Formula (III.a.), R¹ is:

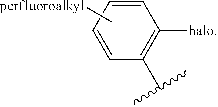

In one embodiment, in Formula (III.a.), R¹ is phenyl substituted with one to three fluoro groups.

In one embodiment, in Formula (III.a.), R¹ is phenyl substituted with two fluoro groups.

In one embodiment, in Formula (III.a.), R¹ is phenyl substituted with one fluoro group.

In one embodiment, in Formula (III.a.), R¹ is:

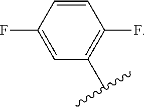

In one embodiment, in Formula (III.a.), R²⁷, R²⁸, and R²⁹ are each independently selected from the group consisting of H and alkyl.

In one embodiment, in Formula (III.a.), R¹ is:

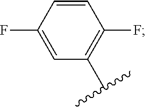

and R²⁷, R²⁸, and R²⁹ are each independently selected from the group consisting of H and alkyl.

In one embodiment, in Formula (III.a.), R² is —C(Z)R⁷.

In one embodiment, in Formula (III.a.), R² is —C(Z)NR⁹R¹⁰.

In one embodiment, in Formula (III.a.), R² is —C(Z)OR⁸.

In one embodiment, in Formula (III.a.), R² is —SO₂NR⁹R¹⁰.

In one embodiment, in Formula (III.a.), R² is alkyl.

In one embodiment, in Formula (III.a.), R² is heteroalkyl.

In one embodiment, in Formula (III.a.), R² is aryl.

In one embodiment, in Formula (III.a.), R² is heteroaryl.

In one embodiment, in Formula (III.a.), R² is cycloalkyl.

In one embodiment, in Formula (III.a.), R² is cycloalkenyl.

In one embodiment, in Formula (III.a.), R² is heterocycloalkyl.

In one embodiment, in Formula (III.a.), R² is heterocycloalkenyl.

In one embodiment, in Formula (III.a.), Z is (═O).

In one embodiment, in Formula (III.a.), Z is (═S).

In one embodiment, in Formula (III.a.), Z is (═N(R¹³)).

In one embodiment, in Formula (III.a.), Z is (═N(CN)).

In one embodiment, in Formula (III.a.), Z is (═N(OR¹⁴)).

In one embodiment, in Formula (III.a.), Z is (═N(R¹⁵)(R¹⁶)).

In one embodiment, in Formula (III.a.), Z is (═C(R¹⁷)(R¹⁸)).

In one embodiment, in Formula (III.a.), R² is —C(Z)R⁷, and Z is (═O).

In one embodiment, in Formula (III.a.), R² is —C(O)H.

In one embodiment, in Formula (III.a.), R² is —C(O)alkyl.

In one embodiment, in Formula (III.a.), R² is —C(O)CH₃.

In one embodiment, in Formula (III.a.), R² is —C(O)R⁷, wherein said R⁷ is alkyl substituted with one or more substituents, which can be the same or different, each substituent being independently selected from the group consisting of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (III.a.), R² is —C(O)R⁷, wherein said R⁷ is alkyl substituted with one to three substituents, which can be the same or different, each substituent being independently selected from the group consisting of —OR¹⁹, —NR²¹R²², and cycloalkyl.

In one embodiment, in Formula (III.a.), R² is —C(O)R⁷, wherein said R⁷ is alkyl, wherein said alkyl is substituted with alkyl and —OH.

In one embodiment, in Formula (III.a.), R² is —C(O)R⁷, wherein said R⁷ is alkyl substituted with one to three substituents, which can be the same or different, each substituent being independently selected from the group consisting of —OH, —NH₂, and cyclopropyl.

In one embodiment, in Formula (III.a.), R² is —C(O)R⁷, wherein said R⁷ is alkyl substituted with one to two substituents, which can be the same or different, each substituent being independently selected from the group consisting of —NH₂, and cyclopropyl.

In one embodiment, in Formula (III.a.), R² is —C(O)R⁷, wherein said R⁷ is alkyl substituted with —OH.

In one embodiment, in Formula (III.a.), R² is —C(O)R⁷, wherein said R⁷ is unsubstituted heterocycloalkyl.

In one embodiment, in Formula (III.a.), R² is —C(O)R⁷, wherein said R⁷ is substituted heterocycloalkyl.

In one embodiment, in Formula (III.a.), R² is —C(O)R⁷, wherein said R⁷ is heterocycloalkyl substituted with one or more substituents, which can be the same or different, each substituent being independently selected from the group consisting of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (III.a.), R² is —C(O)R⁷, wherein said R⁷ is selected from the group consisting of substituted piperidine, substituted piperazine, substituted morpholine, substituted pyrrolidine, and substituted azetidine.

In one embodiment, in Formula (III.a.), R² is selected from:

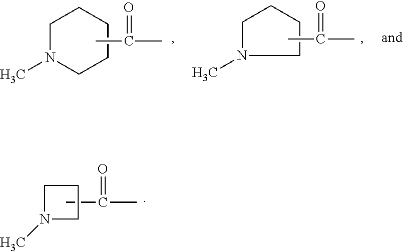

In one embodiment, in Formula (III.a.), R² is —C(O)NR⁹R¹⁰.

In one embodiment, in Formula (III.a.), R² is —C(O)NH₂.

In one embodiment, in Formula (III.a.), R² is —C(O)NR⁹R¹⁰, wherein R⁹ and R¹⁰ can be the same or different, each being independently selected from alkyl.

In one embodiment, in Formula (III.a.), R² is —C(O)NR⁹R¹⁰, wherein R⁹ is unsubstituted heterocycloalkyl and R¹⁰ is selected from the group consisting of H and alkyl.

In one embodiment, in Formula (III.a.), R² is —C(O)NR⁹R¹⁰, wherein R⁹ is substituted heterocycloalkyl and R¹⁰ is selected from the group consisting of H and alkyl.

In one embodiment, in Formula (III.a.), R² is —C(O)NR⁹R¹⁰, wherein R⁹ is heterocycloalkyl substituted with from one to three substituents, which can be the same or different, each substituent being independently selected from alkyl, and R¹⁰ is selected from the group consisting of H and alkyl.

In one embodiment, in Formula (III.a.), R² is selected from the group consisting of: alkyl, haloalkyl, heteroalkyl, heterohaloalkyl, —C(O)R⁷. —C(O)OR⁸, and —C(O)NR⁹R¹⁰.

In one embodiment, in Formula (III.a.), R² is selected from the group consisting of

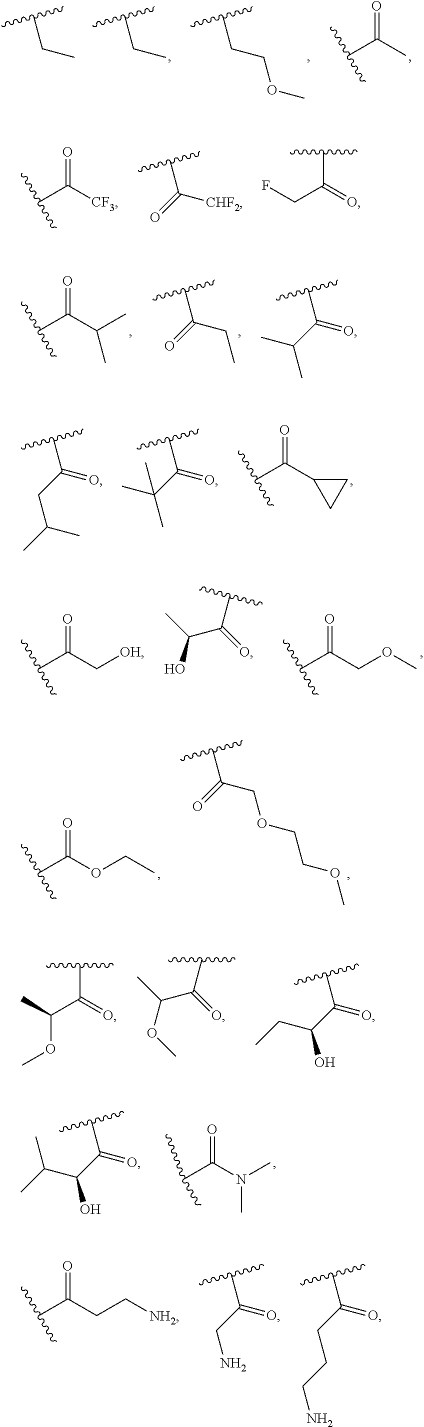

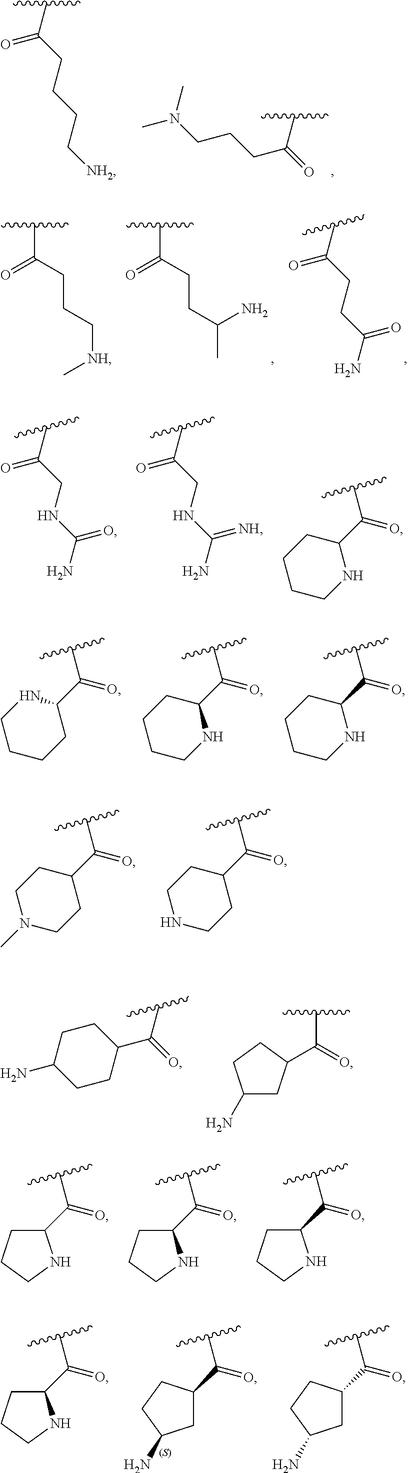

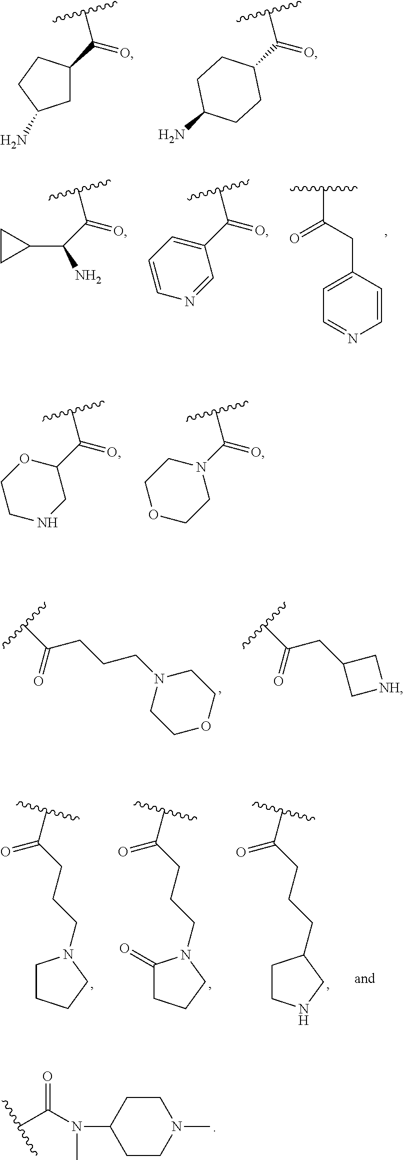

In one embodiment, in Formula (III.a.), R² is

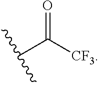

In one embodiment, in Formula (III.a.), R² is

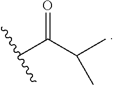

In one embodiment, in Formula (III.a.), R² is

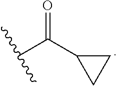

In one embodiment, in Formula (III.a.), R² is

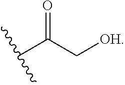

In one embodiment, in Formula (III.a.), R² is

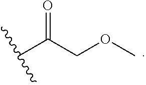

In one embodiment, in Formula (III.a.), R² is

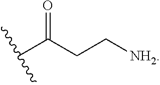

In one embodiment, in Formula (III.a.), R² is

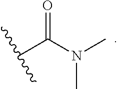

In one embodiment, in Formula (III.a.), R² is

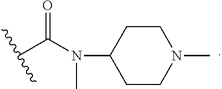

In one embodiment, in Formula (III.a.), p is 0 and R³ is not present.

In one embodiment, in Formula (III.a.), p is 1.

In one embodiment, in Formula (III.a.), p is 2.

In one embodiment, in Formula (III.a.), p is 3.

In one embodiment, in Formula (III.a.), p is 4.

In one embodiment, in Formula (III.a.), p is ≧2 and at least two groups R³ are attached to the same ring atom.

In one embodiment, in Formula (III.a.), p is 1 and R³ is independently selected from the group consisting of alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹—, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (III.a.), p is 2, 3, or 4 and each R³ is independently selected from the group consisting of alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)OR²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (III.a.), p is 2, 3, or 4 and at least two groups R³ are bound to the same ring carbon atom, wherein each R³, which may be the same or different, is independently selected from the group consisting of alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (III.a.), p is 2, 3, or 4 and at least two groups R³ are bound to the same ring carbon atom, wherein two R³ groups, which may be the same or different, together with the carbon atom to which they are attached, form a cycloalkyl, a cycloalkenyl, a heterocycloalkyl ring containing from one to three heteroatoms selected from the group consisting of N, O, and S, or a heterocycloalkenyl ring containing from one to three heteroatoms selected from the group consisting of N, O, and S.

In one embodiment, in Formula (III.a.), p is 1 or 2 and each R³ is independently selected from the group consisting of alkyl, heteroalkyl, alkenyl, heteroalkenyl, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(S)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶, —NR²³C(O)NR²⁵R²⁶, and —NR²³—C(NH)—NR²⁵R²⁶,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (III.a.), p is 1 and R³ is selected from the group consisting of alkyl, heteroalkyl, alkenyl, and heteroalkenyl,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (III.a.), p is 2 and any two R³ groups bound to the same ring A atom are taken together to form a —C(O)— group.

In one embodiment, in Formula (III.a.), p is 2 and any two R³ groups bound to the same ring A atom are taken together to form a spiroheterocycloalkyl group having from 1 to 3 ring heteroatoms independently selected from the group consisting of —NH—, —NR⁶—, O, S, S(O), and S(O)₂, or spiroheterocycloalkenyl group having from 1 to 3 ring heteroatoms independently selected from the group consisting of —NH—, —NR⁶—, O, S, S(O), and S(O)₂.

In one embodiment, in Formula (III.a.), R³ is alkyl.

In one embodiment, in Formula (III.a.), R³ is heteroalkyl.

In one embodiment, in Formula (III.a.), R³ is alkenyl.

In one embodiment, in Formula (III.a.), R³ is heteroalkenyl.

In one embodiment, in Formula (III.a.), R³ is alkynyl.

In one embodiment, in Formula (III.a.), R³ is heteroalkynyl.

In one embodiment, in Formula (III.a.), R³ is aryl.

In one embodiment, in Formula (III.a.), R³ is heteroaryl.

In one embodiment, in Formula (III.a.), R³ is cycloalkyl.

In one embodiment, in Formula (III.a.), R³ is cycloalkenyl.

In one embodiment, in Formula (III.a.), R³ is heterocycloalkyl.

In one embodiment, in Formula (III.a.), R³ is heterocycloalkenyl.

In one embodiment, in Formula (III.a.), R³ is halogen.

In one embodiment, in Formula (III.a.), R³ is —CN.

In one embodiment, in Formula (III.a.), R³ is —NO₂.

In one embodiment, in Formula (III.a.), R³ is —OR¹⁹.

In one embodiment, in Formula (III.a.), R³ is —OC(O)OR²⁰.

In one embodiment, in Formula (III.a.), R³ is —NR²¹R²².

In one embodiment, in Formula (III.a.), R³ is —NR²³SO₂R²⁴.

In one embodiment, in Formula (III.a.), R³ is —NR²³C(O)OR²⁰.

In one embodiment, in Formula (III.a.), R³ is —NR²³C(O)R²⁴.

In one embodiment, in Formula (III.a.), R³ is —SO₂NR²⁵R²⁶.

In one embodiment, in Formula (III.a.), R³ is —C(O)R²⁴.

In one embodiment, in Formula (III.a.), R³ is —C(O)OR²⁰.

In one embodiment, in Formula (III.a.), R³ is —SR¹⁹.

In one embodiment, in Formula (III.a.), R³ is —S(O)R¹⁹.

In one embodiment, in Formula (III.a.), R³ is —SO₂R¹⁹.

In one embodiment, in Formula (III.a.), R³ is —OC(O)R²⁴.

In one embodiment, in Formula (III.a.), R³ is —C(O)NR²⁵R²⁶.

In one embodiment, in Formula (III.a.), R³ is —NR²³C(N—CN)NR²⁵R²⁶.

In one embodiment, in Formula (III.a.), R³ is —NR²³C(O)NR²⁵R²⁶.

In one embodiment, in Formula (III.a.), R³ is selected from the group consisting of: methyl, ethyl, propyl (straight or branched), butyl (straight or branched), pentyl (straight or branched), phenyl,

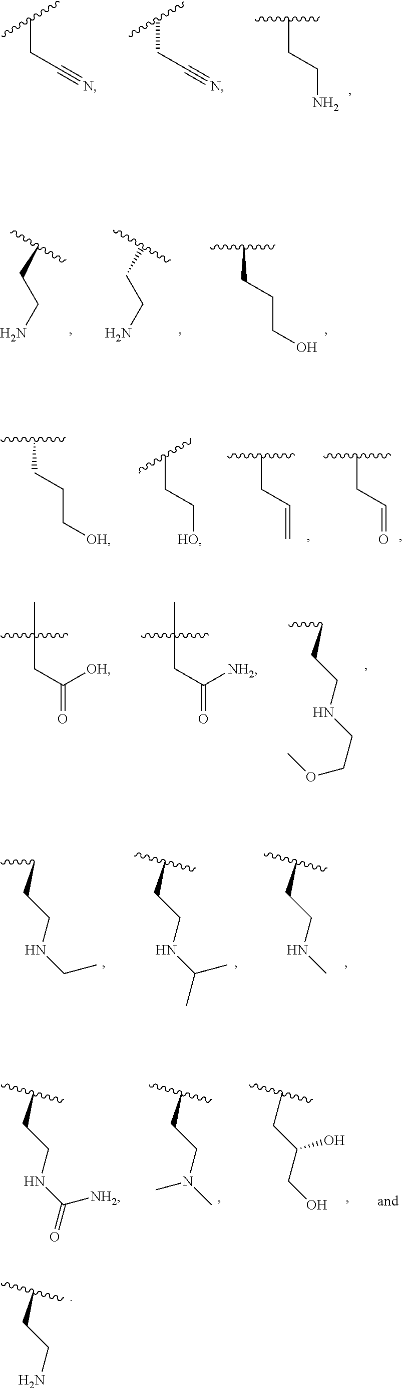

In one embodiment, in Formula (III.a.), when E is —NR⁶, R³ is absent.

In one embodiment, Formula (III.a.) has the general structure (III.a.1):

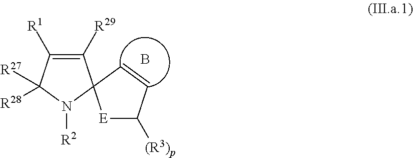

wherein R¹, R², R³, R²⁷, R²⁸, R²⁹, p, E, and ring B are selected independently of each other and wherein:
E is selected from the group consisting of —O—, —S—, —S(O)—, —S(O)₂—, —C(R⁴)(R⁵)—, —N(R⁶)—, —N(C(Y)R⁷)—, —N(C(Y)OR⁸)—, and —N(C(Y)N(R⁹)(R¹⁰))—;
and p, R¹, R², R³, R⁴, R⁵, R⁶, R⁷, R⁸, R⁹, R¹⁰, R²⁷, R²⁸, R²⁹, Y, and the optional substituents on ring B are as defined in any of the embodiments described above in Formula (III.a.).

In one embodiment, Formula (III.a.1) has the general structure shown in Formula (III.a.1.1):

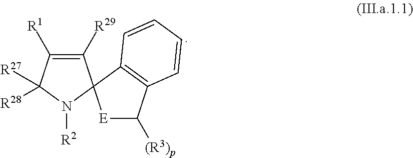

In one embodiment, Formula (III.a.) has the general structure III.a.2:

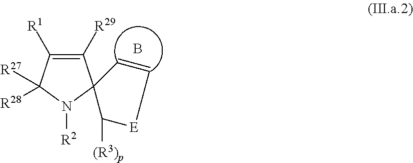

wherein R¹, R², R³, R²⁷, R²⁸, R²⁹, p, E, and ring B are selected independently of each other and wherein:
E is selected from the group consisting of —O—, —S—, —S(O)—, —S(O)₂—, —C(R⁴)(R⁵)—, —N(R⁶)—, —N(C(Y)R⁷)—, —N(C(Y)OR⁸)—, and —N(C(Y)N(R⁹)(R¹⁰))—;
and p, R¹, R², R³, R⁴, R⁵, R⁶, R⁷, R⁸, R⁹, R¹⁰, R²⁷, R²⁸, Y, and the optional substituents on ring B are as defined in any of the embodiments described above in Formula (III.a.).

In one embodiment, Formula (III.a.2) has the general structure shown in Formula (III.a.2.1):

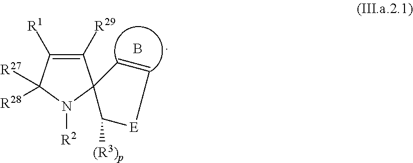

In one embodiment, Formula (III.a.2) has the general structure shown in Formula (III.a.2.2):

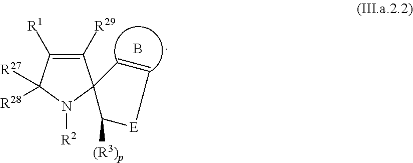

In one embodiment, Formula (III.a.2) has the general structure shown in Formula (III.a.2.3):

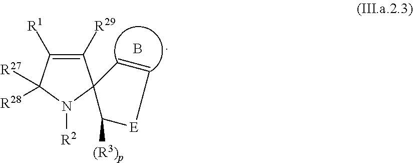

In one embodiment, Formula (III.a.2) has the general structure shown in Formula (III.a.2.4):

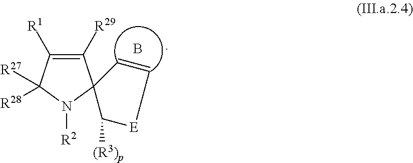

In some embodiments, in each of Formulas (III.a.1), (III.a.1.1), (III.a.2), (III.a.2.1), (III.a.2.2), (III.a.2.3), and (III.a.2.4), p is 0.

In some embodiments, in each of Formulas (III.a.1), (III.a.1.1), (III.a.2), (III.a.2.1), (III.a.2.2), (III.a.2.3), and (III.a.2.4), p is 1.

In some embodiments, in each of Formulas (III.a.1), (III.a.1.1), (III.a.2), (III.a.2.1), (III.a.2.2), (III.a.2.3), and (III.a.2.4), p is 2.

In some embodiments, in each of Formulas (III.a.1), (III.a.1.1), (III.a.2), (III.a.2.1), (III.a.2.2), (III.a.2.3), and (III.a.2.4), E is —C(R⁴)(R⁵)—.

In some embodiments, in each of Formulas (III.a.1), (III.a.1.1), (III.a.2), (III.a.2.1), (III.a.2.2), (III.a.2.3), and (III.a.2.4), E is selected from the group consisting of —O—, —S—, —S(O)—, —S(O)₂—, and —N(R⁶)—.

In some embodiments, in each of Formulas (III.a.1), (III.a.1.1), (III.a.2), (III.a.2.1), (III.a.2.2), (III.a.2.3), and (III.a.2.4), E is selected from the group consisting of —O—, —S—, —S(O)—, —S(O)₂—, and —N(R⁶)—, wherein R⁶ is selected from the group consisting of H, alkyl, —C(O)R²⁴, and —C(S)R²⁴.

In some embodiments, in each of Formulas (III.a.1), (III.a.1.1), (III.a.2), (III.a.2.1), (III.a.2.2), (III.a.2.3), and (III.a.2.4), E is selected from the group consisting of —O— and —N(R⁶)—, wherein R⁶ is selected from the group consisting of H, alkyl, —C(O)R²⁴, and —C(S)R²⁴.

In some embodiments, in each of Formulas (III.a.1), (III.a.1.1), (III.a.2), (III.a.2.1), (III.a.2.2), (III.a.2.3), and (III.a.2.4), E is —O—.

In some embodiments, in each of Formulas (III.a.1), (III.a.1.1), (III.a.2), (III.a.2.2), (III.a.2.3), and (III.a.2.4), E is —S—.

In some embodiments, in each of Formulas (III.a.1), (III.a.1.1), (III.a.2.1), (III.a.2.2), (III.a.2.3), and (III.a.2.4), E is —S(O)—.

In some embodiments, in each of Formulas (III.a.1), (III.a.1.1), (III.a.2), (III.a.2.1), (III.a.2.2), (III.a.2.3), and (III.a.2.4), E is —S(O)₂—.

In some embodiments, in each of Formulas (III.a.1), (III.a.1.1), (III.a.2), (III.a.2.1), (III.a.2.2), (III.a.2.3), and (III.a.2.4), E is —C(R⁴)(R⁵)—.

In some embodiments, in each of Formulas (III.a.1), (III.a.1.1), (III.a.2), (III.a.2.1), (III.a.2.2), (III.a.2.3), and (III.a.2.4), E is —N(R⁶)—.

In some embodiments, in each of Formulas (III.a.1), (III.a.1.1), (III.a.2), (III.a.2.1), (III.a.2.2), (III.a.2.3), and (III.a.2.4), E is —N(C(Y)R⁷)—.

In some embodiments, in each of Formulas (III.a.1), (III.a.1.1), (III.a.2), (III.a.21), (III.a.2.2), (III.a.2.3), and (III.a.2.4), E is —N(C(Y)OR⁸)—.

In some embodiments, in each of Formulas (III.a.1), (III.a.1.1), (III.a.2), (III.a.2.1), (III.a.2.2), (III.a.2.3), and (III.a.2.4), E is —N(C(Y)N(R⁹)(R¹⁰))—.

In some embodiments, in each of Formulas (III.a.1), (III.a.1.1), (III.a.2), (III.a.2.1), (III.a.2.2), (III.a.2.3), and (III.a.2.4), Y is (═O).

In some embodiments, in each of Formulas (III.a.1), (III.a.1.1), (III.a.2), (III.a.2.1), (III.a.2.2), (III.a.2.3), and (III.a.2.4), Y is (═S).

In some embodiments, in each of Formulas (III.a.1), (III.a.2), (III.a.2.1), (III.a.2.2), (III.a.2.3), and (III.a.2.4), Y is (═N(R¹³)).

In some embodiments, in each of Formulas (III.a.1), (III.a.1.1), (III.a.2), (III.a.2.1), (III.a.2.2), (III.a.2.3), and (III.a.2.4), Y is (═N(CN)).

In some embodiments, in each of Formulas (III.a.1), (III.a.2), (III.a.2.1), (III.a.2.2), (III.a.2.3), and (III.a.2.4), Y is (═N(OR¹⁴)).

In some embodiments, in each of Formulas (III.a.1), (III.a.1.1), (III.a.2), (III.a.2.1), (III.a.2.2), (III.a.2.3), and (III.a.2.4), Y is (═N(R¹⁵)(R¹⁶)).

In some embodiments, in each of Formulas (III.a.1), (III.a.2), (III.a.2.1), (III.a.2.2), (III.a.2.3), and (III.a.2.4), Y is (═C(R¹⁷)(R¹⁸)).

In some embodiments, in each of Formulas (III.a.1), (III.a.1.1), (III.a.2), (III.a.2.1), (III.a.2.2), (III.a.2.3), and (III.a.2.4), R¹ is phenyl substituted with one to four substituents, which can be the same or different, each substituent being independently selected from the group consisting of halo, —OH, —CN, —NO₂, —NR²¹R²², and haloalkyl.

In some embodiments, in each of Formulas (III.a.1), (III.a.1.1), (III.a.2), (III.a.2.1), (III.a.2.2), (III.a.2.3), and (III.a.2.4), R¹ is selected from the group consisting of:

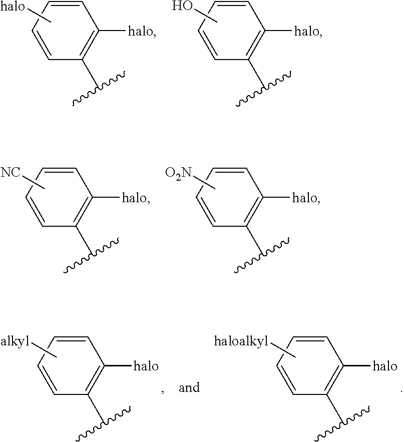

In one embodiment, in Formula (I), R¹ is:

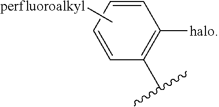

In some embodiments, in each of Formulas (III.a.1), (III.a.1.1), (III.a.2), (III.a.2.1), (III.a.2.2), (III.a.2.3), and (III.a.2.4), R¹ is phenyl substituted with one to three fluoro groups.

In some embodiments, in each of Formulas (III.a.1), (III.a.2), (III.a.2.1), (III.a.2.2), (III.a.2.3), and (III.a.2.4), R¹ is phenyl substituted with two fluoro groups.

In some embodiments, in each of Formulas (III.a.1), (III.a.1.1), (III.a.2), (III.a.2.1), (III.a.2.3), and (III.a.2.4), R¹ is phenyl substituted with one fluoro group.

In some embodiments, in each of Formulas (III.a.1), (III.a.1.1), (III.a.2), (III.a.2.1), (III.a.2.2), (III.a.2.3), and (III.a.2.4), R¹ is:

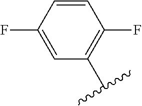

In some embodiments, in each of Formulas (III.a.1), (III.a.1.1), (III.a.2), (III.a.2.1), (III.a.2.2), (III.a.2.3), and (III.a.2.4), R²⁷, R²⁸, and R²⁹ are each independently selected from the group consisting of H and alkyl.

In some embodiments, in each of Formulas (III.a.1), (III.a.1.1), (III.a.2), (III.a.2.1), (III.a.2.2), (III.a.2.3), and (III.a.2.4), R² is selected from the group consisting of: alkyl, haloalkyl, heteroalkyl, heterohaloalkyl, —C(O)R⁷. —C(O)OR⁸, and —C(O)NR⁹R¹⁰.

In some embodiments, in each of Formulas (III.a.1), (III.a.2), (III.a.2.1), (III.a.2.2), (III.a.2.3), and (III.a.2.4), R² is selected from the group consisting of:

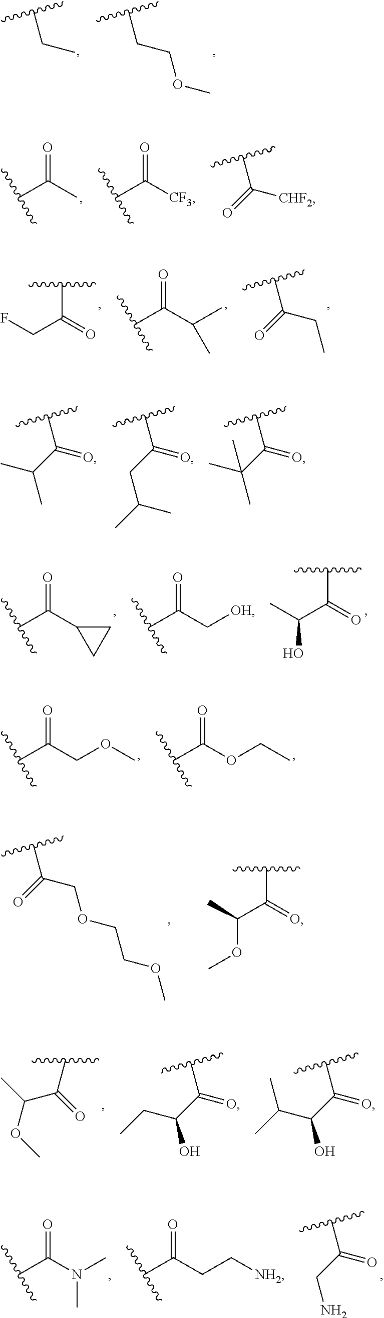

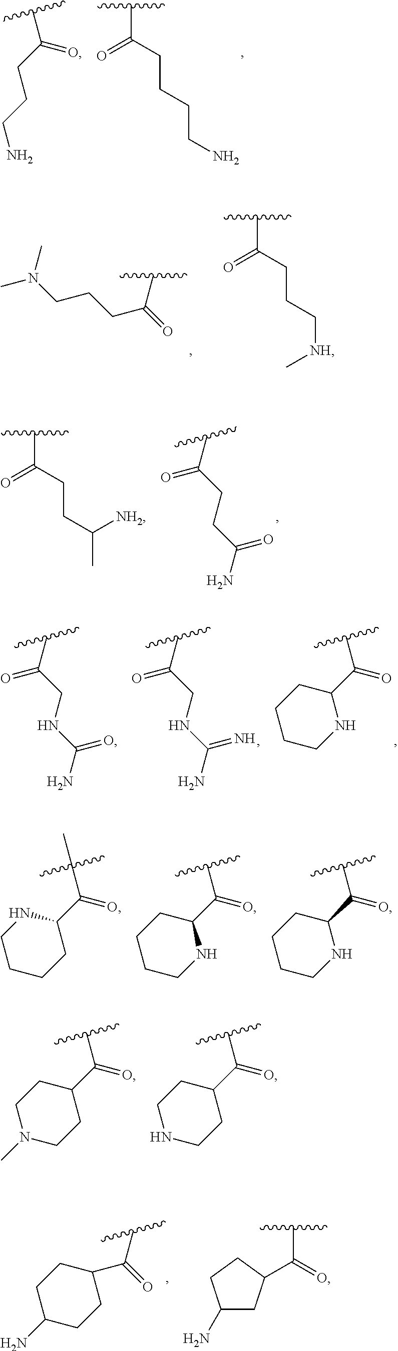

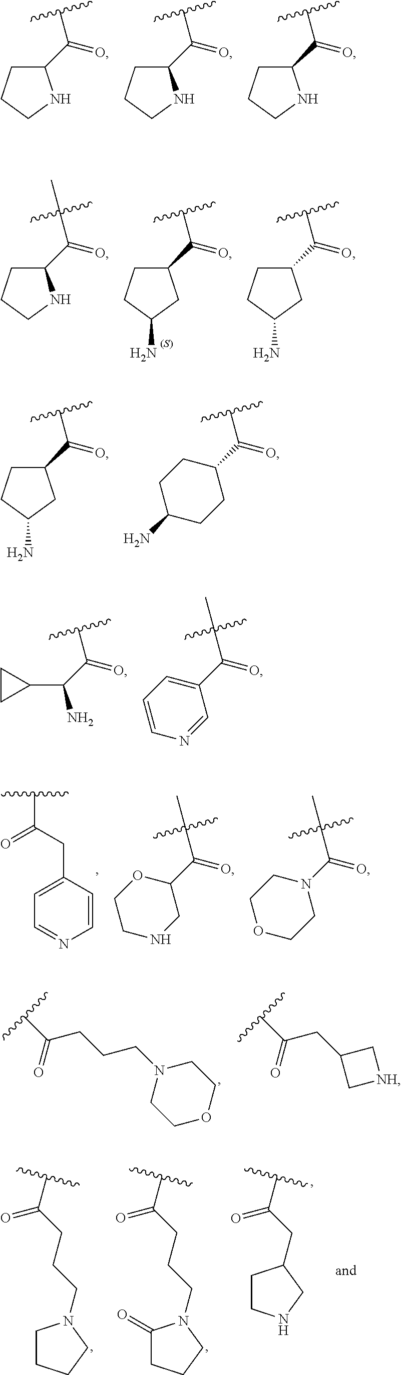

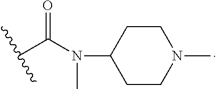

In some embodiments, in each of Formulas (III.a.1), (III.a.1.1), (III.a.2), (III.a.2.1), (III.a.2.2), (III.a.2.3), and (III.a.2.4), p is 1 or 2 and each R³ is independently selected from the group consisting of alkyl, heteroalkyl, alkenyl, heteroalkenyl, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴—, —C(S)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶, —NR C(O)NR²⁵R²⁶, and —NR²³—C(NH)—NR²⁵R²⁶,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²³R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶

In some embodiments, in each of Formulas (III.a.1), (III.a.1.1), (III.a.2), (III.a.2.2), (III.a.2.3), and (III.a.2.4), p is 1 and R³ is selected from the group consisting of alkyl, heteroalkyl, alkenyl, and heteroalkenyl,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶

In some embodiments, in each of Formulas (III.a.1), (III.a.1.1), (III.a.2), (III.a.2.1), (III.a.2.2), (III.a.2.3), and (III.a.2.4), p is 2 and any two R³ groups bound to the same ring A atom are taken together to form a —C(O)— group.

In some embodiments, in each of Formulas (III.a.1), (III.a.1.1), (III.a.2), (III.a.2.1), (III.a.2.2), (III.a.2.3), and (III.a.2.4), p is 2 and any two R³ groups bound to the same ring A atom are taken together to form a spiroheterocycloalkyl group having from 1 to 3 ring heteroatoms independently selected from the group consisting of —NH—, —NR⁶—, O, S, S(O), and S(O)₂, or spiroheterocycloalkenyl group having from 1 to 3 ring heteroatoms independently selected from the group consisting of —NH—, —NR⁶—, O, S, S(O), and S(O)₂.

In one embodiment, the compounds of the invention have a structure shown in Formula (III.b) and include pharmaceutically acceptable salts, solvates, esters, prodrugs, or isomers of said compounds:

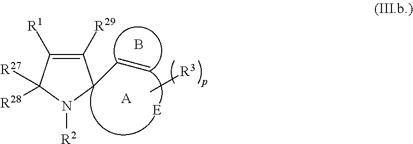

wherein R¹, R², R³, R²⁷, R²⁸, R²⁹, p, E, ring A, and ring B are selected independently of each other and wherein:
ring A (including E and the unsaturation shown) is a 5-membered cycloalkenyl or heterocycloalkenyl ring;
E is selected from the group consisting of —O—, —S—, —S(O)—, —S(O)₂—, —C(R⁴)(R⁵)—, —N(R⁶)—, —N(C(Y)R⁷)—, —N(C(Y)OR⁸)—, —N(C(Y)N(R⁹)(R¹⁰))—, —C(O)—N(R¹¹)—, —N(R¹¹)—C(O)—, —S(O)₂—N(R¹¹)—, —N(R¹¹)—S(O)₂—, —C(O)—O—, —O—C(O)—, —O—N(R⁶)—, —N(R⁶)—O—, —N(R⁶)—N(R¹²)—, —N═N—, and —C(R⁷)═N—;
ring B is a substituted or unsubstituted heteroaromatic ring;
and p, R¹, R², R³, R⁴, R⁵, R⁶, R⁷, R⁸, R⁹, R¹⁰, R¹¹, R¹², R²⁷, R²⁸, R²⁹, Y, and the optional substituents on ring B are as defined in any of the embodiments described above in Formula (I).

In one embodiment, Formula (III.b) has the general structure:

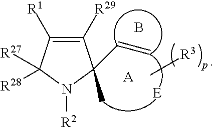

In one embodiment, Formula (III.b) has the general structure:

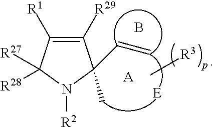

In one embodiment, in Formula (III.b.), p is 0, 1, or 2.

In one embodiment, in Formula (III.b.), ring A is a cycloalkenyl ring and E is —C(R⁴)(R⁵)—.

In one embodiment, in Formula (III.b.), ring A is a heterocycloalkenyl ring and E is selected from the group consisting of —C(O)—N(R¹¹)—, —N(R¹¹)—C(O)—, —S(O)₂—N(R¹)—, —N(R¹¹)—S(O)₂—, —C(O)—O—, —O—C(O)—, —O—N(R⁶)—, —N(R⁶)—O—, —N(R⁶)—N(R¹²)—, and —C(R⁷)═N—. By way of non-limiting illustration, an example of a compound of Formula (III.a.) wherein E is —C(O)—N(R¹¹)—includes:

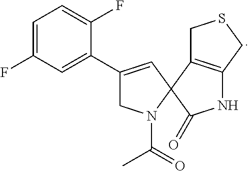

In one embodiment, in Formula (III.b.), ring A is a heterocycloalkenyl ring and E is selected from the group consisting of —O—, —S—, —S(O)—, —S(O)₂—, and —N(R⁶)—.

In one embodiment, in Formula (III.b.), E is selected from the group consisting of —O—, —S—, —S(O)—, —S(O)₂—, and —N(R⁶)—, wherein R⁶ is selected from the group consisting of H, alkyl, —C(O)R²⁴, and —C(S)R²⁴.

In one embodiment, in Formula (III.b.), E is selected from the group consisting of —O— and —N(R⁶)—, wherein R⁶ is selected from the group consisting of H, alkyl, —C(O)R²⁴, and —C(S)R²⁴.

In one embodiment, in Formula (III.b.), E is —O—.

In one embodiment, in Formula (III.b.), E is —S—.

In one embodiment, in Formula (III.b.), E is —S(O)—.

In one embodiment, in Formula (III.b.), E is —S(O)₂—.

In one embodiment, in Formula (III.b.), E is —C(R⁴)(R⁵)—.

In one embodiment, in Formula (III.b.), E is —N(R⁶)—.

In one embodiment, in Formula (III.b.), E is —N(C(Y)R⁷)—.

In one embodiment, in Formula (III.b.), E is —N(C(Y)OR⁸)—.

In one embodiment, in Formula (III.b.), E is —N(C(Y)N(R⁹)(R¹⁰))—.

In one embodiment, in Formula (III.b.), E is —C(O)—N(R¹¹)—.

In one embodiment, in Formula (III.b.), E is —N(R¹¹)—C(O)—.

In one embodiment, in Formula (III.b.), E is —S(O)₂—N(R¹¹)—.

In one embodiment, in Formula (III.b.), E is —N(R¹¹)—S(O)₂—.

In one embodiment, in Formula (III.b.), E is —C(O)—O—.

In one embodiment, in Formula (III.b.), E is —O—C(O)—.

In one embodiment, in Formula (III.b.), E is —O—N(R⁸)—.

In one embodiment, in Formula (III.b.), E is —N(R⁶)—O—.

In one embodiment, in Formula (III.b.), E is —N(R⁶)—N(R¹²)—.

In one embodiment, in Formula (III.b.), E is —N═N—.

In one embodiment, in Formula (III.b.), E is —C(R⁷)═N—.

In one embodiment, in Formula (III.b.), Y is (═O).

In one embodiment, in Formula (III.b.), Y is (═S).

In one embodiment, in Formula (III.b.), Y is (═N(R¹³)).

In one embodiment, in Formula (III.b.), Y is (═N(CN)).

In one embodiment, in Formula Y is (═N(OR¹⁴)).

In one embodiment, in Formula (III.b.), Y is (═N(R¹⁵)(R¹⁶)).

In one embodiment, in Formula Y is (═C(R¹⁷)(R¹⁸)).

In one embodiment, in Formula (III.b.), B is an unsubstituted heteroaromatic ring.

In one embodiment, in Formula (III.b.), B is an unsubstituted 5-6-membered heteroaromatic ring having from 1-3 ring heteroatoms, which can be the same or different, each hetero ring atom being independently selected from the group consisting of N, S, O, S(O), and S(O)₂.

In one embodiment, in Formula (III.b.), B is a heteroaromatic ring which is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —O(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (III.b.), B is a 5-6-membered heteroaromatic ring having from 1-3 ring heteroatoms, which can be the same or different, each hetero ring atom being independently selected from the group consisting of N, S, O, S(O), and S(O)₂, which heteroaromatic ring is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (III.b.), B is an unsubstituted 6-membered heteroaromatic ring having from 1-3 ring heteroatoms, which can be the same or different, each hetero ring atom being independently selected from the group consisting of N, S, and O.

In one embodiment, in Formula (III.b.), B is a 6-membered heteroaromatic ring having from 1-3 ring heteroatoms, which can be the same or different, each hetero ring atom being independently selected from the group consisting of N, S, and O, which heteroaromatic ring is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (III.b.), B is an unsubstituted 6-membered heteroaromatic ring having 2 ring heteroatoms, each ring heteroatom being independently selected from of N, S, and O.

In one embodiment, in Formula (III.b.), B is a 6-membered heteroaromatic ring having 2 ring heteroatoms, each ring heteroatom being independently selected from of N, S, and O, which heteroaromatic ring is substituted with one or more substituents, which can be the same or different, each substituent being independently selected from the group consisting of halogen, —CN, —NO₂, alkyl, heteroalkyl, haloalkyl, —OR¹⁹, —NR²¹R²², —C(O)R²⁴, —C(O)OR²⁰, —SR¹⁹, —S(O)R¹⁹, —SO₂R¹⁹, —OC(O)R²⁴, and —C(O)NR²⁵R²⁶.

In one embodiment, in Formula (III.b.), B is an unsubstituted 5-membered heteroaromatic ring having from 1-2 ring heteroatoms, which can be the same or different, each hetero ring atom being independently selected from the group consisting of N, S, and O.

In one embodiment, in Formula (III.b.), B is a 5-membered heteroaromatic ring having from 1-2 ring heteroatoms, which can be the same or different, each hetero ring atom being independently selected from the group consisting of N, S, and O, which heteroaromatic ring is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (III.b.), B is an unsubstituted 5-membered heteroaromatic ring having 1 ring heteroatom selected from of N, S, and O.

In one embodiment, in Formula (III.b.), B is a 5-membered heteroaromatic ring having 1 ring heteroatom selected from of N, S, and O, which heteroaromatic ring is substituted with one or more substituents, which can be the same or different, each substituent being independently selected from the group consisting of halogen, —CN, —NO₂, alkyl, heteroalkyl, haloalkyl, —OR¹⁹, —NR²¹R²², —C(O)R²⁴, —C(O)OR²⁰, —SR¹⁹, —S(O)R¹⁹, —SO₂R¹⁹, —OC(O)R²⁴, and —C(O)NR²⁵R²⁶.

In one embodiment, in Formula (III.b.), B is a 5-membered heteroaromatic ring having S as the ring heteroatom, which heteroaromatic ring is substituted with one or more substituents, which can be the same or different, each substituent being independently selected from the group consisting halogen, —CN, —NO₂, alkyl, heteroalkyl, haloalkyl, —OR¹⁹, —NR²¹R²², —C(O)R²⁴, —C(O)OR²⁰, —SR¹⁹, —S(O)R¹⁹, —SO₂R¹⁹, —OC(O)R²⁴, and —C(O)NR²⁵R²⁶.

In one embodiment, in Formula (III.b.), B is an unsubstituted 5-membered heteroaromatic ring having S as the ring heteroatom.

In one embodiment, in Formula (III.b.), B is selected from the group consisting of

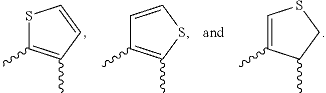

In one embodiment, in Formula (III.b.), R¹ is unsubstituted aryl.

In one embodiment, in Formula (III.b.), R¹ is unsubstituted phenyl.

In one embodiment, in Formula (III.b.), R¹ is unsubstituted naphthyl.

In one embodiment, in Formula (III.b.), R¹ is substituted aryl.

In one embodiment, in Formula (III.b.), R¹ is substituted phenyl.

In one embodiment, in Formula (III.b.), R¹ is substituted naphthyl.

In one embodiment, in Formula (III.b.), R¹ is aryl substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (III.b.), R¹ is phenyl substituted with one or more substituents, which can be the same or different, each substituent being independently selected from the group consisting halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (III.b.), R¹ is phenyl substituted with one to four substituents, which can be the same or different, each substituent being independently selected from the group consisting of halo, —OH, —CN, —NO₂, —NR²¹R²², and haloalkyl.

In one embodiment, in Formula (III.b.), R¹ is selected from the group consisting of:

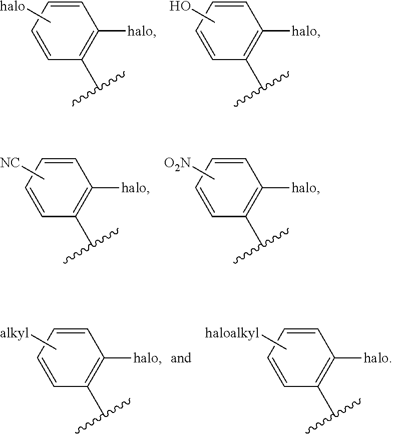

In one embodiment, in Formula (III.b.), R¹ is:

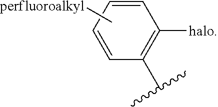

In one embodiment, in Formula (III.b.), R¹ is phenyl substituted with one to three fluoro groups.

In one embodiment, in Formula (III.b.), R¹ is phenyl substituted with two fluoro groups.

In one embodiment, in Formula (III.b.), R¹ is phenyl substituted with one fluoro group.

In one embodiment, in Formula (III.b.), R¹ is:

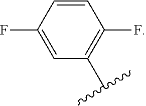

In one embodiment, in Formula (III.b.), R²⁷, R²⁸, and R²⁹ are each independently selected from the group consisting of H and alkyl.

In one embodiment, in Formula R¹ is:

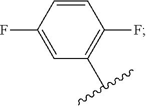

and R²⁷, R²⁸, and R²⁹ are each independently selected from the group consisting of H and alkyl.

In one embodiment, in Formula (III.b.), R² is —C(Z)R⁷.

In one embodiment, in Formula (III.b.), R² is —C(Z)NR⁹R¹⁰.

In one embodiment, in Formula (III.b.), R² is —C(Z)OR⁸.

In one embodiment, in Formula (III.b.), R² is —SO₂NR⁹R¹⁰.

In one embodiment, in Formula (III.b.), R² is alkyl.

In one embodiment, in Formula (III.b.), R² is heteroalkyl.

In one embodiment, in Formula (III.b.), R² is aryl.

In one embodiment, in Formula (III.b.), R² is heteroaryl.

In one embodiment, in Formula (III.b.), R² is cycloalkyl.

In one embodiment, in Formula (III.b.), R² is cycloalkenyl.

In one embodiment, in Formula (III.b.), R² is heterocycloalkyl.

In one embodiment, in Formula (III.b.), R² is heterocycloalkenyl.

In one embodiment, in Formula (III.b.), Z is (═O).

In one embodiment, in Formula (III.b.), Z is (═S).

In one embodiment, in Formula (III.b.), Z is (═N(R¹³)).

In one embodiment, in Formula (III.b.), Z is (═N(CN)).

In one embodiment, in Formula (III.b.), Z is (═N(OR¹⁴)).

In one embodiment, in Formula (III.b.), Z is (═N(R¹⁵)(R¹⁶)).

In one embodiment, in Formula (III.b.), Z is (═C(R¹⁷)(R¹⁸)).

In one embodiment, in Formula (III.b.), R² is —C(Z)R⁷, and Z is (═O).

in one embodiment, in Formula (III.b.), R² is —C(O)H.

In one embodiment, in Formula (III.b.), R² is —C(O)alkyl.

In one embodiment, in Formula (III.b.), R² is —C(O)CH₃.

In one embodiment, in Formula (III.b.), R² is —C(O)R⁷, wherein said R⁷ is alkyl substituted with one or more substituents, which can be the same or different, each substituent being independently selected from the group consisting of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁸, —SR¹⁹, —S(O)R¹⁹, —SO₂R¹⁵, —OC(O)R²⁴, —C(O)NR²⁵R²⁶, —NR²³C(N—CN)NR²⁵R²⁶ and —NR²³C(O)NR²⁵R²⁶.

In one embodiment, in Formula (III.b.), R² is —C(O)R⁷, wherein said R⁷ is alkyl substituted with one to three substituents, which can be the same or different, each substituent being independently selected from the group consisting of —OR¹⁹, —NR²¹R²², and cycloalkyl.

In one embodiment, in Formula (III.b.), R² is —C(O)R⁷, wherein said R⁷ is alkyl, wherein said alkyl is substituted with alkyl and —OH.

In one embodiment, in Formula (III.b.), R² is —C(O)R⁷, wherein said R⁷ is alkyl substituted with one to three substituents, which can be the same or different, each substituent being independently selected from the group consisting of —OH, —NH₂, and cyclopropyl.

In one embodiment, in Formula (III.b.), R² is —C(O)R⁷, wherein said R⁷ is alkyl substituted with one to two substituents, which can be the same or different, each substituent being independently selected from the group consisting of —NH₂, and cyclopropyl.

In one embodiment, in Formula (III.b.), R² is —C(O)R⁷, wherein said R⁷ is alkyl substituted with —OH.

In one embodiment, in Formula (III.b.), R² is —C(O)R⁷, wherein said R⁷ is unsubstituted heterocycloalkyl.

In one embodiment, in Formula (III.b.), R² is —C(O)R⁷, wherein said R⁷ is substituted heterocycloalkyl.

In one embodiment, in Formula (III.b.), R² is —C(O)R⁷, wherein said R⁷ is heterocycloalkyl substituted with one or more substituents, which can be the same or different, each substituent being independently selected from the group consisting of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (III.b.), R² is —C(O)R⁷, wherein said R⁷ is selected from the group consisting of substituted piperidine, substituted piperazine, substituted morpholine, substituted pyrrolidine, and substituted azetidine.

In one embodiment, in Formula (III.b.), R² is selected from:

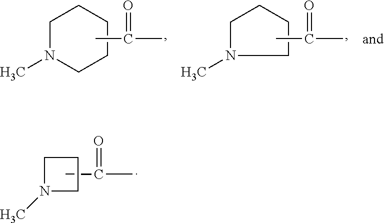

In one embodiment, in Formula (III.b.), R² is —C(O)NR⁹R¹⁰.

In one embodiment, in Formula (III.b.), R² is —C(O)NH₂.

In one embodiment, in Formula (III.b.), R² is —C(O)NR⁹R¹⁰, wherein R⁹ and R¹⁰ can be the same or different, each being independently selected from alkyl.

In one embodiment, in Formula (III.b.), R² is —C(O)NR⁹R¹⁰, wherein R⁹ is unsubstituted heterocycloalkyl and R¹⁰ is selected from the group consisting of H and alkyl.

In one embodiment, in Formula (III.b.), R² is —C(O)NR⁹R¹⁰, wherein R⁹ is substituted heterocycloalkyl and R¹⁰ is selected from the group consisting of H and alkyl.

In one embodiment, in Formula (III.b), R² is —C(O)NR⁹R¹⁰, wherein R⁹ is heterocycloalkyl substituted with from one to three substituents, which can be the same or different, each substituent being independently selected from alkyl, and R¹⁰ is selected from the group consisting of H and alkyl.

In one embodiment, in Formula (III.b.), R² is selected from the group consisting of: alkyl, haloalkyl, heteroalkyl, heterohaloalkyl, —C(O)R⁷. —C(O)OR⁸, and —C(O)NR⁹R¹⁰.

In one embodiment, in Formula (III.b.), R² is selected from the group consisting of

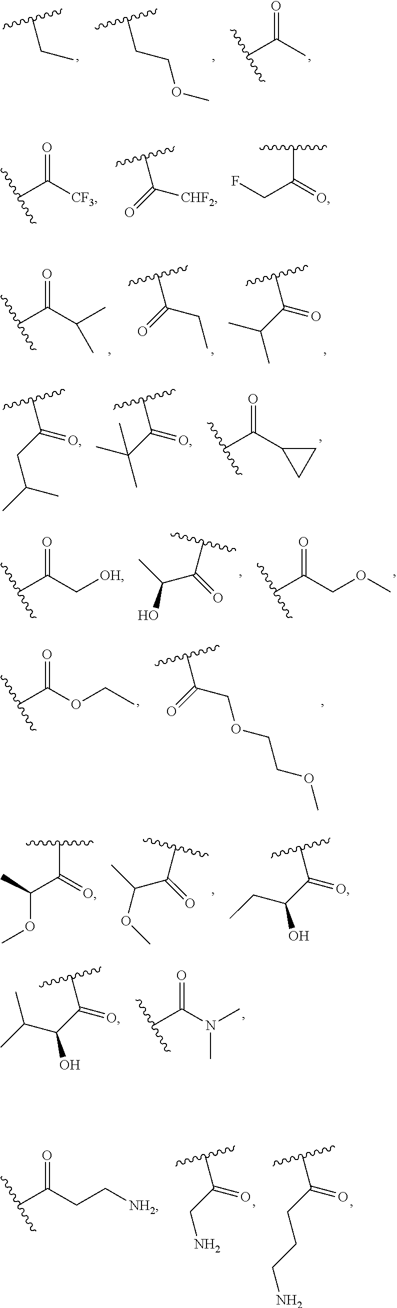

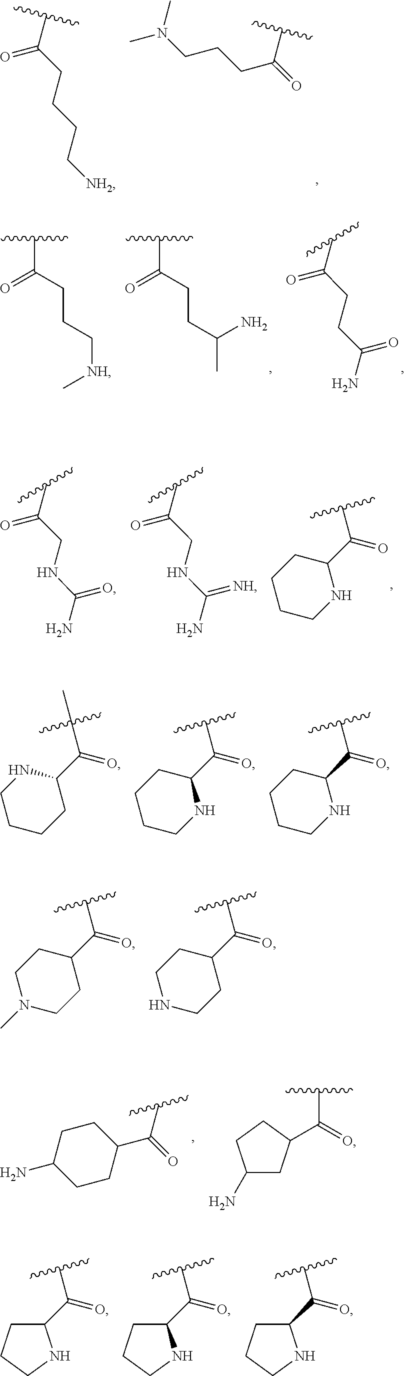

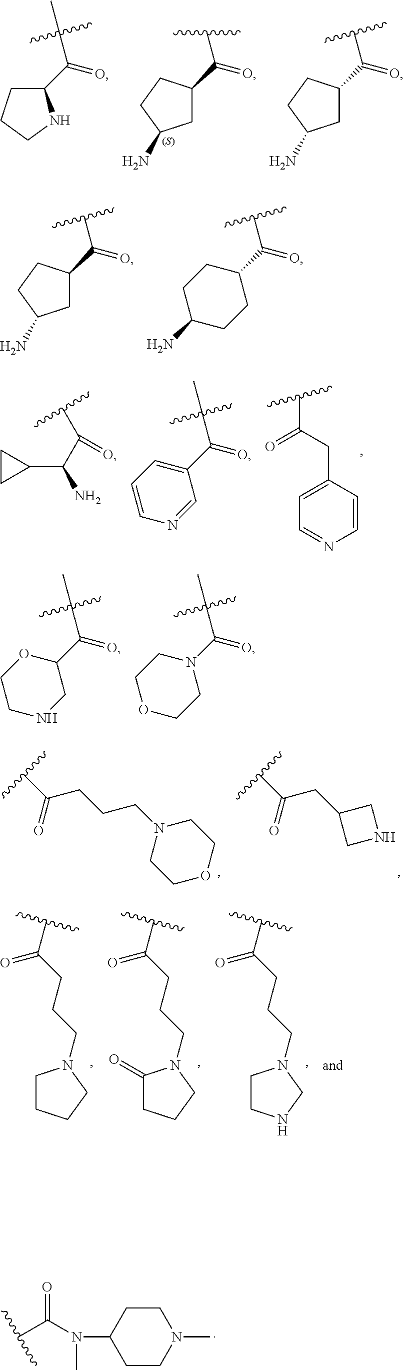

in one embodiment, in Formula (III.b.), R² is

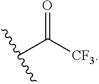

In one embodiment, in Formula (III.b.), R² is

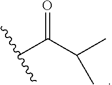

In one embodiment, in Formula (III.b.), R² is

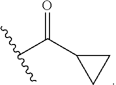

In one embodiment, in Formula (III.b.), R² is

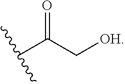

In one embodiment, in Formula (III.b.), R² is

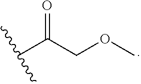

In one embodiment, in Formula (III.b.), R² is

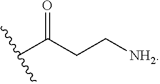

In one embodiment, in Formula (III.b.), R² is

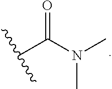

In one embodiment, in Formula (III.b.), R² is

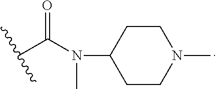

In one embodiment, in Formula (III.b.), p is 0 and R³ is not present.

In one embodiment, in Formula (III.b.), p is 1.

In one embodiment, in Formula (III.b.), p is 2.

In one embodiment, in Formula p is 3.

In one embodiment, in Formula (III.b.), p is 4.

In one embodiment, in Formula (III.b.), p is ≧2 and at least two groups R³ are attached to the same ring atom.

In one embodiment, in Formula (III.b.), p is 1 and R³ is independently selected from the group consisting of alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (III.b.), p is 2, 3, or 4 and each R³ is independently selected from the group consisting of alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (Int.), p is 2, 3, or 4 and at least two groups R³ are bound to the same ring carbon atom, wherein each R³, which may be the same or different, is independently selected from the group consisting of alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (III.b.), p is 2, 3, or 4 and at least two groups R³ are bound to the same ring carbon atom, wherein two R³ groups, which may be the same or different, together with the carbon atom to which they are attached, form a cycloalkyl, a cycloalkenyl, a heterocycloalkyl ring containing from one to three heteroatoms selected from the group consisting of N, O, and S, or a heterocycloalkenyl ring containing from one to three heteroatoms selected from the group consisting of N, O, and S.

In one embodiment, in Formula (III.b.), p is 1 or 2 and each R³ is independently selected from the group consisting of alkyl, heteroalkyl, alkenyl, heteroalkenyl, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(S)R²⁴, C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶, —NR²³C(O)NR²⁵R²⁶, and —NR²³—C(NH)—NR²⁵R²⁶,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶

In one embodiment, in Formula (III.b.), p is 1 and R³ is selected from the group consisting of alkyl, heteroalkyl, alkenyl, and heteroalkenyl,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (III.b.), p is 2 and any two R³ groups bound to the same ring A atom are taken together to form a —C(O)— group.

In one embodiment, in Formula (III.b.), p is 2 and any two R³ groups bound to the same ring A atom are taken together to form a spiroheterocycloalkyl group having from 1 to 3 ring heteroatoms independently selected from the group consisting of —NH—, —NR⁶—, O, S, S(O), and S(O)₂, or spiroheterocycloalkenyl group having from 1 to 3 ring heteroatoms independently selected from the group consisting of —NH—, —NR⁶—, O, S, S(O), and S(O)₂.

In one embodiment, in Formula (III.b.), R³ is alkyl.

In one embodiment, in Formula (III.b.), R³ is heteroalkyl.

In one embodiment, in Formula (III.b.), R³ is alkenyl.

In one embodiment, in Formula (III.b.), R³ is heteroalkenyl.

In one embodiment, in Formula (III.b.), R³ is alkynyl.

In one embodiment, in Formula (III.b.), R³ is heteroalkynyl.

In one embodiment, in Formula (III.b.), R³ is aryl.

In one embodiment, in Formula (III.b.), R³ is heteroaryl.

In one embodiment, in Formula (III.b.), R³ is cycloalkyl.

In one embodiment, in Formula (III.b.), R³ is cycloalkenyl.

In one embodiment, in Formula (III.b.), R³ is heterocycloalkyl.

In one embodiment, in Formula (III.b.), R³ is heterocycloalkenyl.

In one embodiment, in Formula (III.b.), R³ is halogen.

In one embodiment, in Formula (III.b.), R³ is —CN.

In one embodiment, in Formula (III.b.), R³ is —NO₂.

In one embodiment, in Formula (III.b.), R³ is —OR¹⁹.

In one embodiment, in Formula (III.b.), R³ is —OC(O)OR²⁰.

In one embodiment, in Formula (III.b.), R³ is —NR²¹R²².

In one embodiment, in Formula (III.b.), R³ is —NR²³SO₂R²⁴.

In one embodiment, in Formula (III.b.), R³ is —NR²³C(O)OR²⁰.

In one embodiment, in Formula (III.b.), R³ is —NR²³C(O)R²⁴.

In one embodiment, in Formula (III.b.), R³ is —SO₂NR²⁵R²⁶.

In one embodiment, in Formula (III.b.), R³ is —C(O)R²⁴.

In one embodiment, in Formula (III.b.), R³ is —C(O)OR²⁰.

In one embodiment, in Formula (III.b.), R³ is —SR¹⁹.

In one embodiment, in Formula (III.b.), R³ is —S(O)R¹⁹.

In one embodiment, in Formula (III.b.), R³ is —SO₂R¹⁹.

In one embodiment, in Formula (III.b.), R³ is —OC(O)R²⁴.

In one embodiment, in Formula (III.b.), R³ is —C(O)NR²⁵R²⁶.

In one embodiment, in Formula (III.b.), R³ is —NR²³C(N—CN)NR²⁵R²⁶.

In one embodiment, in Formula (III.b.), R³ is —NR²³C(O)NR²⁵R²⁶.

In one embodiment, in Formula (III.b.), R³ is selected from the group consisting of: methyl, ethyl, propyl (straight or branched), butyl (straight or branched), pentyl (straight or branched), phenyl,

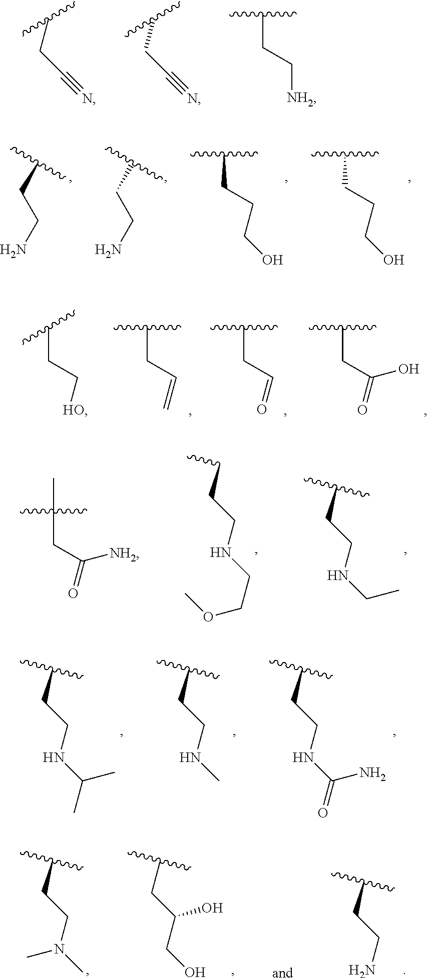

In one embodiment, in Formula (III.b.), when E is —NR⁶—, R³ is absent.

In one embodiment, Formula (III.b.) has the general structure (III.b.1):

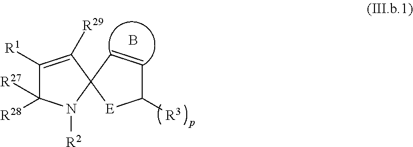

wherein R¹, R², R³, R²⁷, R²⁸, R²⁹, p, E, and ring B are selected independently of each other and wherein:
E is selected from the group consisting of —O—, —S—, —S(O)—, —S(O)₂—, —C(R⁴)(R⁵)—, —N(R⁶)—, —N(C(Y)R⁷)—, —N(C(Y)OR⁸)—, and —N(C(Y)N(R⁹)(R¹⁰))—;
and p, R¹, R², R³, R⁴, R⁵, R⁶, R⁷, R⁸, R⁹, R¹⁰, R²⁷, R²⁸, R²⁹, Y, and the optional substituents on ring B are as defined in any of the embodiments described above in Formula (III.b.).

In one embodiment, Formula (III.b) has the general structure shown in Formula (III.b.2):

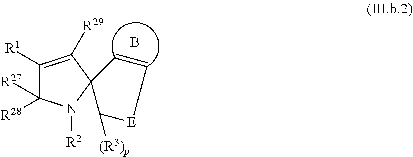

In one embodiment, Formula (III.b) has the general structure shown in Formula (III.b.2.1):

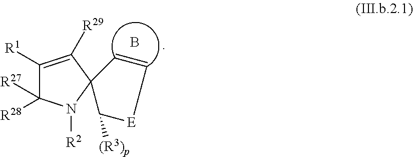

In one embodiment, Formula (III.b) has the general structure shown in Figure (III.b.2.2):

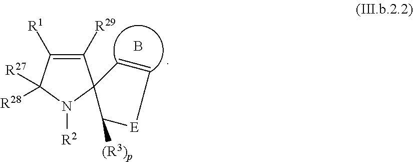

In one embodiment, Formula (III.b) has the general structure shown in Formula (III.b.2.3):

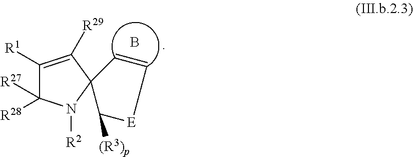

In one embodiment, Formula (III.b) has the general structure shown in Formula (III.b.2.4):

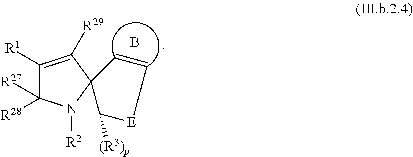

In some embodiments, in each of Formulas (III.b.1), (III.b.2), (III.b.2.1), (III.b.2.2), (III.b.2.3), and (III.b.2.4), p is 0.

In some embodiments, in each of Formulas (III.b.1), (III.b.2), (III.b.2.1), (III.b.2.2), (III.b.2.3), and (III.b.2.4), p is 1.

In some embodiments, in each of Formulas (III.b.1), (III.b.2), (III.b.2.1), (III.b.2.2), (III.b.2.3), and (III.b.2.4), p is 2.

In some embodiments, in each of Formulas (III.b.1), (III.b2), (III.b.2.1), (III.b.2.2), (III.b.2.3), and (III.b.2.4), E is —C(R⁴)(R⁶)—.

In some embodiments, in each of Formulas (III.b.1), (III.b.2), (III.b.2.1), (III.b.2.2), (III.b.2.3), and (III.b.2.4), E is selected from the group consisting of —O—, —S—, —S(O)—, —S(O)₂—, and —N(R⁶)—.

In some embodiments, in each of Formulas (III.b.1), (III.b.2), (III.b.2.1), (III.b.2.2), (III.b.2.3), and (III.b.2.4), E is selected from the group consisting of —O—, —S—, —S(O)—, —S(O)₂—, and —N(R⁶)—, wherein R⁶ is selected from the group consisting of H, alkyl, —C(O)R²⁴, and —C(S)R²⁴.

In some embodiments, in each of Formulas (III.b.1), (III.b.2), (III.b.2.1), (III.b.2.2), (III.b.2.3), and (III.b.2.4), E is selected from the group consisting of —O— and —N(R⁶)—, wherein R⁶ is selected from the group consisting of H, alkyl, —C(O)R²⁴, and —C(S)R²⁴.

In some embodiments, in each of Formulas (III.b.1), (III.b.2), (III.b.2.1), (III.b.2.2), (III.b.2.3), and (III.b.2.4), E is —O—.

In some embodiments, in each of Formulas (III).1), (III.b.2), (III.b.2.1), (III.b2.2), (III.b.2.3), and (III.b.2.4), E is —S—.

In some embodiments, in each of Formulas (III.b.1), (III.b.2), (III.b.2.1), (III.b.2.2), (III.b.2.3), and (III.b.2.4), E is —S(O)—.

In some embodiments, in each of Formulas (III.b.1), (III.b.2), III.b.2.1), (III.b.2.2), (III.b.2.3), and (III.b.2.4), E is —S(O)₂—.

In some embodiments, in each of Formulas (III.b.1), (III.b.2), (III.b.2.1), (III.b.2.2), (III.b.2.3), and (III.b.2.4), E is —C(R⁴)(R⁵)—.

In some embodiments, in each of Formulas (III.b.1), (III.b.2), (III.b.2.1), (III.b.2.2), (III.b.2.3), and (III.b.2.4), E is —N(R⁶)—.

In some embodiments, in each of Formulas (III.b.1), (III.b.2), (III.b.2.1), (III.b.2.2), (III.b.2.3), and (III.b.2.4), E is —N(C(Y)R⁷)—.

In some embodiments, in each of Formulas (III.b.1), (III.b.2), (III.b.2.1), (III.b.2.2), (III.b.2.3), and (III.b.2.4), E is —N(C(Y)OR⁸)—.

In some embodiments, in each of Formulas (III.b.1), (III.b.2), (III.b.2.1), (III.b.2.2), (III.b.2.3), and (III.b.2.4), E is —N(C(Y)N(R⁹)(R¹⁰))—.

In some embodiments, in each of Formulas (III.b.1), (III.b.2), (III.b.2.1), (III.b.2.2), (III.b.2.3), and (III.b.2.4), Y is (═O).

In some embodiments, in each of Formulas (III.b.1), (III.b.2), (III.b2.1), (III.b.2.2), (III.b.2.3), and (III.b.2.4), Y is (═S).

In some embodiments, in each of Formulas (III.b.1), (III.b.2), (III.b.2.1), (III.b.2.2), (III.b.2.3), and (III.b.2.4), Y is (═N(R¹³)).

In some embodiments, in each of Formulas (III.b.1), (III.b.2), (III.b.21), (III.b.2.2), (III.b.2.3), and (III.b.2.4), Y is (═N(CN)).

In some embodiments, in each of Formulas (III.b.1), (III.b.2), (III.b2.1), (III.b.2.2), (III.b.2.3), and (III.b.2.4), Y is (═N(OR¹⁴)).

In some embodiments, in each of Formulas (III.b.1), (III.b.2), (III.b.2.1), (III.b.2.2), (III.b.2.3), and (III.b.2.4), Y is (═N(R¹⁵)(R¹⁶)).

In some embodiments, in each of Formulas (III.b.1), (III.b.2), (III.b.2.1), (III.b.2.2), (III.b.2.3), and (III.b.2.4), Y is (═C(R¹⁷)(R¹⁸)).

In some embodiments, in each of Formulas (III.b.1), (III.b.2), (III.b.2.1), (III.b.2.2), (III.b.2.3), and (III.b.2.4), R¹ is phenyl substituted with one to four substituents, which can be the same or different, each substituent being independently selected from the group consisting of halo, —OH, —CN, —NO₂, —NR²¹R²², and haloalkyl.

In some embodiments, in each of Formulas (III.b.1), (III.b.2), (III.b.2.1), (III.b.2.2), (III.b.2.3), and (III.b.2.4), R¹ is selected from the group consisting of

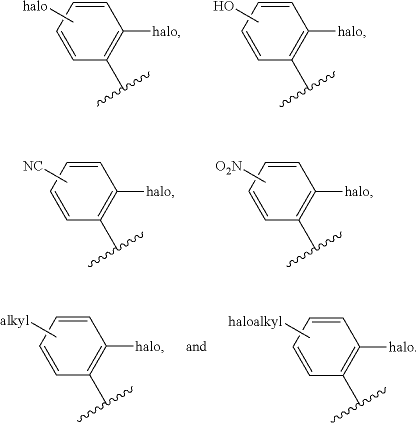

In one embodiment, in Formula (I), R¹ is:

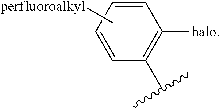

In some embodiments, in each of Formulas (III.b.1), (III.b.2), (III.b.2.1), (III.b.2.2), (III.b.2.3), and (III.b.2.4), R¹ is phenyl substituted with one to three fluoro groups.

In some embodiments, in each of Formulas (III.b.1), (III.b.2), (III.b.2.1), (III.b.2.2), (III.b.2.3), and (III.b.2.4), R¹ is phenyl substituted with two fluoro groups.

In some embodiments, in each of Formulas (III.b.1), (III.b.2), (III.b.2.1), (III.b.2.2), (III.b.2.3), and (III.b.2.4), R¹ is phenyl substituted with one fluoro group.

In some embodiments, in each of Formulas (III.b.1), (III.b.2), (III.b.2.1), (III.b.2.2), (III.b.2.3), and (III.b.2.4), R¹ is:

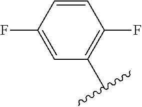

In some embodiments, in each of Formulas (III.b.1), (III.b.2), (III.b.2.1), (III.b.2.2), (III.b.2.3), and (III.b.2.4), R²⁷, R²⁸, and R²⁹ are each independently selected from the group consisting of H and alkyl.

In some embodiments, in each of Formulas (III.b.1), (III.b.2), (III.b.2.1), (III.b.2.2), (III.b.2.3), and (III.b.2.4), R² is selected from the group consisting of: alkyl, haloalkyl, heteroalkyl, heterohaloalkyl, —C(O)R⁷. —C(O)OR⁸, and —C(O)NR⁹R¹⁰.

In some embodiments, in each of Formulas (III.b.1), (III.b.2), (III.b.2.1), (III.b.2.2), (III.b.2.3), and (III.b.2.4), R² is selected from the group consisting of:

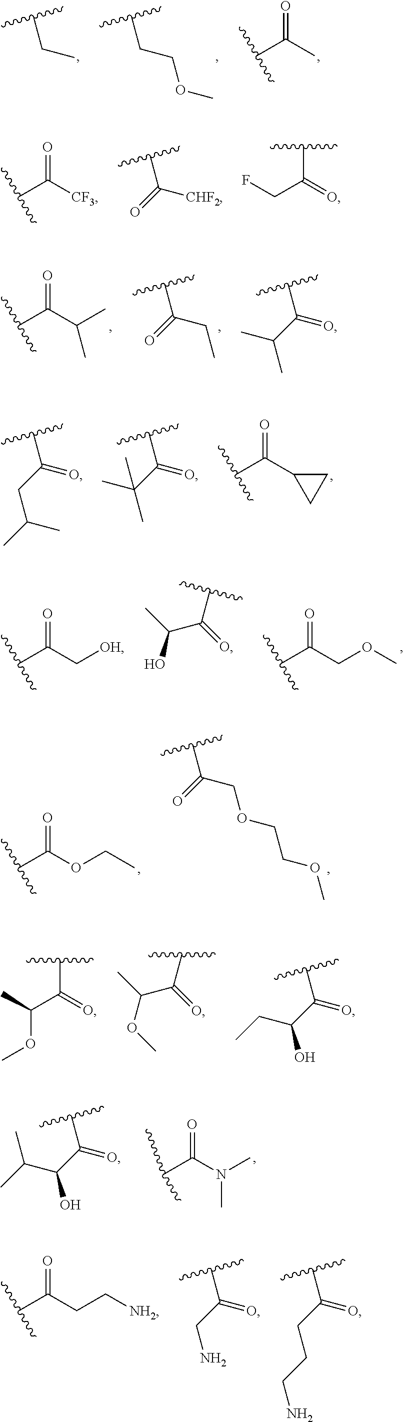

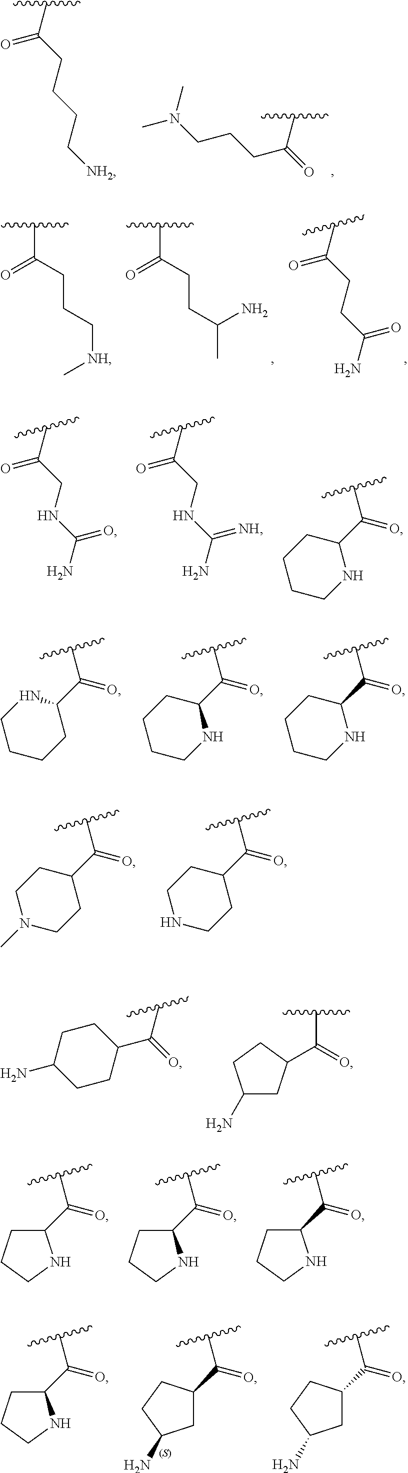

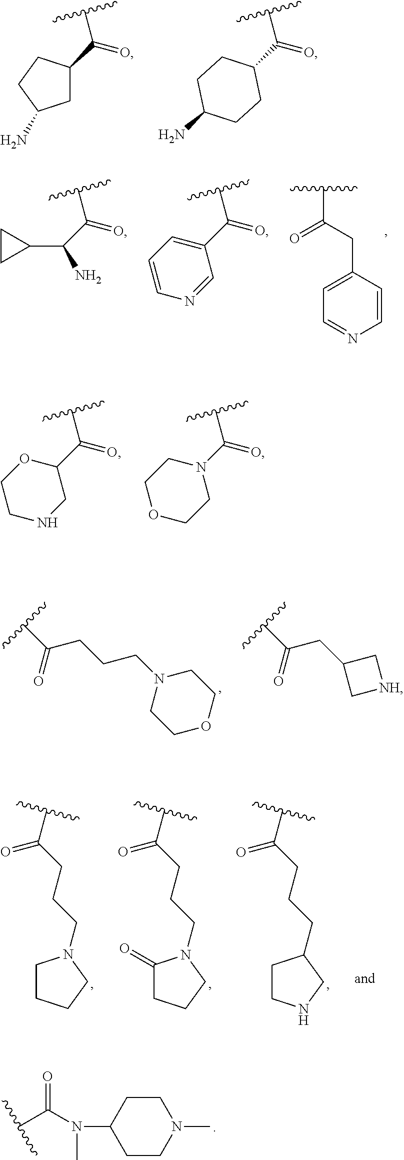

In some embodiments, in each of Formulas (III.b.1), (III.b.2), (III.b.2.1), (III.b.2.2), (III.b.2.3), and (III.b.2.4), p is 1 or 2 and each R³ is independently selected from the group consisting of alkyl, heteroalkyl, alkenyl, heteroalkenyl, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(S)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶, —NR²³C(O)NR²⁵R²⁶, and —NR²³—C(NH)—NR²⁵R²⁶,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶

In some embodiments, in each of Formulas (III.b.1), (III.b.2), (III.b.2.1), (III.b.2.2), (III.b.2.3), and (III.b.2.4), p is 1 and R³ is selected from the group consisting of alkyl, heteroalkyl, alkenyl, and heteroalkenyl,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (III.b.1), (III.b.2.1), (III.b.2.2), (III.b.2.3), and (III.b.2.4), p is 2 and any two R³ groups bound to the same ring A atom are taken together to form a —C(O)— group.

In some embodiments, in each of Formulas (III.b.1), (III.b.2), (III.b.2.1), (III.b.2.2), (III.b.2.3), and (III.b.2.4), p is 2 and any two R³ groups bound to the same ring A atom are taken together to form a spiroheterocycloalkyl group having from 1 to 3 ring heteroatoms independently selected from the group consisting of —NH—, —NR⁶—, O, S, S(O), and S(O)₂, or spiroheterocycloalkenyl group having from 1 to 3 ring heteroatoms independently selected from the group consisting of —NH—, —NR⁶—, O, S, S(O), and S(O)₂.

In one embodiment, the compounds of the invention have a structure shown in Formula (IV) and include pharmaceutically acceptable salts, solvates, esters, prodrugs, or isomers of said compounds:

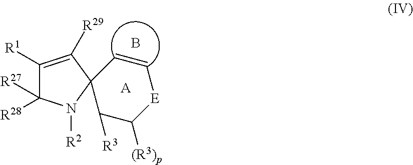

wherein R¹, R², R³, R²⁷, R²⁸, R²⁹, p, E, ring A, and ring B and the optional groups attached to ring B are each selected independently of each other and wherein:
E is selected from the group consisting of —C(R⁴)(R⁵)—, —O—, —S—, —S(O)—, —S(O)₂—, and —N(R⁶)—;
ring B is an unsubstituted or substituted aromatic ring or an unsubstituted or substituted 5-6-membered heteroaromatic ring having from 1-3 ring heteroatoms, which ring heteroatoms can be the same or different, each ring heteroatom being independently selected from the group consisting of N, S, O, S(O), and S(O)₂, said substituents on said aromatic ring or said heteroaromatic ring (when present) being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
R¹ is unsubstituted aryl or aryl substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
R² is selected from the group consisting of —C(O)R⁷, —C(O)NR⁹R¹⁰, and —C(O)OR⁸;
p is 0, 1, or 2; and
each R³ (when present) is independently selected from the group consisting of alkyl, heteroalkyl, alkenyl, heteroalkenyl, —CN, —NO₂, —OR¹⁹, —OC(O)OR²⁰, —NR²¹R²², —C(O)R²⁴, —C(S)R²⁴, —C(O)OR²⁰, and —C(O)NR²⁵R²⁶,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶; and
all remaining variables are as defined in each of the embodiments described above in Formula (I).

In one such embodiment, in Formula (IV):

E is selected from the group consisting of —O— and —N(R⁶)—;
ring B is an unsubstituted or substituted moiety selected from the group consisting of benzo, furanyl, thiophenyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, and triazinyl;
R¹ is phenyl substituted with one to four substituents, which can be the same or different, each substituent being independently selected from the group consisting of halo, —OH, —CN, —NO₂, —NR²¹R²², and haloalkyl;
R² is selected from the group consisting of —C(O)R⁷, —C(O)NR⁹R¹⁰, and —C(O)OR⁸;
p is 0 or 1; and
each R³ (when present) is independently selected from the group consisting of alkyl, heteroalkyl, alkenyl, heteroalkenyl,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶,

In one such embodiment, in Formula (IV):

### R¹ is:

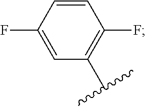

and
R⁶ is selected from the group consisting of H, alkyl, —C(O)R²⁴, —C(O)OR²⁰, and —C(S)R²⁴.

In one such embodiment, in Formula (IV):

### R¹ is:

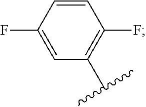

R⁶ is selected from the group consisting of H, alkyl, —C(O)R²⁴ and —C(S)R²⁴; and
R²⁷, R²⁸, and R²⁹ are each independently selected from the group consisting of H and alkyl.

In one embodiment, the compounds of the invention have a structure shown in Formula (IV.a) and include pharmaceutically acceptable salts, solvates, esters, prodrugs, or isomers of said compounds:

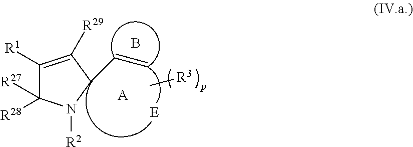

wherein R¹, R², R³, R²⁷, R²⁸, R²⁹, p, E, ring A, and ring B are selected independently of each other and wherein:
ring A (including E and the unsaturation shown) is a 6-membered cycloalkenyl or heterocycloalkenyl ring;
E is selected from the group consisting of —O—, —S—, —S(O)—, —S(O)₂—, —C(R⁴)(R⁵)—, —N(R⁶)—, —N(C(Y)R⁷)—, —N(C(Y)OR⁸)—, —N(C(Y)N(R⁹)(R¹⁰))—, —C(O)—N(R¹¹)—, —N(R¹¹)—C(O)—, —S(O)₂—N(R¹¹)—, —N(R¹¹)—S(O)₂—, —C(O)—O—, —O—C(O)—, —O—N(R⁶)—, —N(R⁶)—O—, —N(R⁶)—N(R¹²)—, —N═N—, —C(R⁷)═N—, —C(O)—C(R⁷)═N—, —C(O)—N═N—, —O—C(Y)—N(R¹¹)—, —N(R¹¹)—C(Y)—O—, —N(R¹¹)—C(Y)—N(R¹²)—, —C(Y)—N(R¹¹)—O—, —C(Y)—N(R¹¹)—N(R¹²)—, —O—N(R¹¹)—C(Y)—, and —N(R¹²)—N(R¹¹)—C(Y)—,
ring B is a substituted or unsubstituted aromatic ring;
and p, R¹, R², R³, R⁴, R⁵, R⁶, R⁷, R⁸, R⁹, R¹⁰, R¹¹, R¹², R²⁷, R²⁸, R²⁹, Y, and the optional substituents on ring B are as defined in each of the embodiments described above in Formula (I).

In one embodiment, Formula (IV.a) has the general structure shown in Formula (IV.a.1):

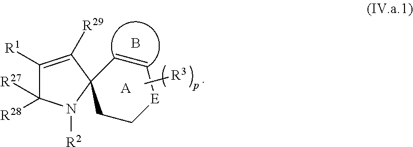

In one embodiment, Formula (IV.a) has the general structure shown in Formula (IV.a.2):

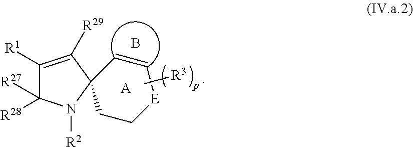

In one embodiment, Formula (IV.a) has the general structure shown in Formula (IV.a.3):

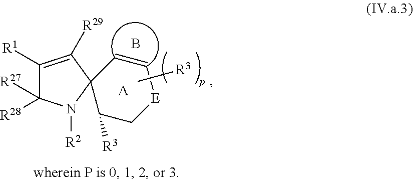

In one embodiment, Formula (IV.a) has the general structure shown in Formula (IV.a.4):

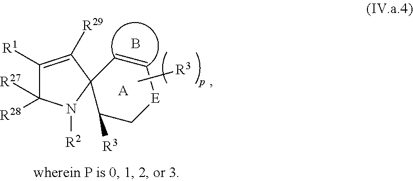

In one embodiment, Formula (IV.a) has the general structure shown in Formula (IV.a.5):

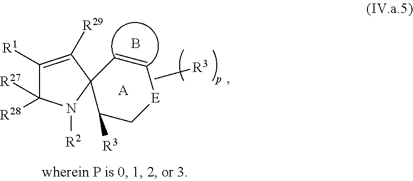

In one embodiment, Formula (IV.a) has the general structure shown in Formula (IV.a.6):

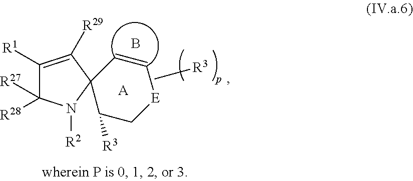

In some embodiments, in each of Formulas (IV.a), (IV.a.2), (IV.a.3), (IV.a.4), (IV.a.5), and (IV.a.6), ring A is a cycloalkenyl ring and E is —C(R⁴)(R⁵)—.

In some embodiments, in each of Formulas (IV.a), (IV.a.1), (IV.a.2), (IV.a.3), (IV.a.4), (IV.a.5), and (IV.a.6), ring A is a heterocycloalkenyl ring and E is selected from the group consisting of —O—, —S—, —S(O)—, —S(O)₂—, and —N(R⁶)—.

In some embodiments, in each of Formulas (IV.a), (IV.a.2), (IV.a.3), (IV.a.4), (IV.a.5), and (IV.a.6), ring A is a heterocycloalkenyl ring and E is selected from the group consisting of —O—, —S—, —S(O)—, —S(O)₂—, and —N(R⁶)—, wherein R⁶ is selected from the group consisting of 1-1, alkyl, —C(O)R²⁴, and —C(S)R²⁴.

In some embodiments, in each of Formulas (IV.a), (IV.a.1), (IV.a.2), (IV.a.3), (IV.a.4), (IV.a.5), and (IV.a.6), ring A is a heterocycloalkenyl ring and E is selected from the group consisting of —O— and —N(R⁶)—, wherein R⁶ is selected from the group consisting of H, alkyl, —C(O)R²⁴, and —C(S)R²⁴.

In some embodiments, in each of Formulas (IV.a), (IV.a.1), (IV.a.2), (IV.a.3), (IV.a.4), (IV.a.5), and (IV.a.6), E is —O—.

In some embodiments, in each of Formulas (IV.a), (IV.a.1), (IV.a.2), (IV.a.3), (IV.a.4), (IV.a.5), and (IV.a.6), E is —S—.

In some embodiments, in each of Formulas (IV.a), (IV.a.1), (IV.a.2), (IV.a.3), (IV.a.4), (IV.a.5), and (IV.a.6), E is —S(O)—.

In some embodiments, in each of Formulas (IV.a), (IV.a.1), (IV.a.2), (IV.a.3), (IV.a.4), (IV.a.5), and (IV.a.6), E is —S(O)₂—.

In some embodiments, in each of Formulas (IV.a), (IV.a.1), (IV.a.2), (IV.a.3), (IV.a.4), (IV.a.5), and (IV.a.6), E is —C(R⁴)(R⁵)—.

In some embodiments, in each of Formulas (IV.a), (IV.a.1), (IV.a.2), (IV.a.3), (IV.a.4), (IV.a.5), and (IV.a.6), E is —N(R⁶)—.

In some embodiments, in each of Formulas (IV.a), (IV.a.1), (IV.a.2), (IV.a.3), (IV.a.4), (IV.a.5), and (IV.a.6), E is —N(C(Y)R⁷)—.

In some embodiments, in each of Formulas (IV.a), (IV.a.1), (IV.a.2), (IV.a.3), (IV.a.4), (IV.a.5), and (IV.a.6), E is —N(C(Y)OR⁸)—.

In some embodiments, in each of Formulas (IV.a), (IV.a.1), (IV.a.2), (IV.a.3), (IV.a.4), (IV.a.5), and (IV.a.6), E is —N(C(Y)N(R⁹)(R¹⁰))—.

In some embodiments, in each of Formulas (IV.a), (IV.a.1), (IV.a.2), (IV.a.3), (IV.a.4), (IV.a.5), and (IV.a.6), E is —C(O)—N(R¹¹)—.

In some embodiments, in each of Formulas (IV.a), (IV.a.1), (IV.a.2), (IV.a.3), (IV.a.4), (IV.a.5), and (IV.a.6), E is —N(R¹¹)—C(O)—.

In some embodiments, in each of Formulas (IV.a), (IV.a.1), (IV.a.2), (IV.a.3), (IV.a.4), (IV.a.5), and (IV.a.6), E is —S(O)₂—N(R¹¹)—.

In some embodiments, in each of Formulas (IV.a), (IV.a.1), (IV.a.2), (IV.a.3), (IV.a.4), (IV.a.5), and (IV.a.6), E is —N(R¹¹)—S(O)₂—.

In some embodiments, in each of Formulas (IV.a), (IV.a.1), (IV.a.2), (IV.a.3), (IV.a.4), (IV.a.5), and (IV.a.6), E is —C(O)—O—.

In some embodiments, in each of Formulas (IV.a), (IV.a.1), (IV.a.2), (IV.a.3), (IV.a.4), (IV.a.5), and (IV.a.6), E is —O—C(O)—.

In some embodiments, in each of Formulas (IV.a), (IV.a.1), (IV.a.2), (IV.a.3), (IV.a.4), (IV.a.5), and (IV.a.6), E is —O—N(R⁶)—.

In some embodiments, in each of Formulas (IV.a), (IV.a.1), (IV.a.2), (IV.a.3), (IV.a.4), (IV.a.5), and (IV.a.6), E is —N(R⁶)—O—.

In some embodiments, in each of Formulas (IV.a), (IV.a.1), (IV.a.2), (IV.a.3), (IV.a.4), (IV.a.5), and (IV.a.6), E is —N(R⁶)—N(R¹²)—.

In some embodiments, in each of Formulas (IV.a), (IV.a.1), (IV.a.3), (IV.a.4), (IV.a.5), and (IV.a.6), E is —N═N—.

In some embodiments, in each of Formulas (IV.a), (IV.a.1), (IV.a.2), (IV.a.3), (IV.a.4), (IV.a.5), and (IV.a.6), E is —C(R⁷)═N—.

In some embodiments, in each of Formulas (IV.a), (IV.a.1), (IV.a.2), (IV.a.3), (IV.a.4), (IV.a.5), and (IV.a.6), E is —C(O)—C(R⁷)═N—.

In some embodiments, in each of Formulas (IV.a), (IV.a.1), (IV.a.2), (IV.a.3), (IV.a.4), (IV.a.5), and (IV.a.6), E is —C(O)—N═N—.

In some embodiments, in each of Formulas (IV.a), (IV.a.1), (IV.a.2), (IV.a.3), (IV.a.4), (IV.a.5), and (IV.a.6), E is —O—C(Y)—N(R¹¹)—.

In some embodiments, in each of Formulas (IV.a), (IV.a.1), (IV.a.2), (IV.a.3), (IV.a.4), (IV.a.5), and (IV.a.6), E is —N(R¹¹)—C(Y)—O—.

In some embodiments, in each of Formulas (IV.a), (IV.a.1), (IV.a.2), (IV.a.3), (IV.a.4), (IV.a.5), and (IV.a.6), E is —N(R¹¹)—C(Y)—N(R¹²)—.

In some embodiments, in each of Formulas (IV.a), (IV.a.1), (IV.a.2), (IV.a.3), (IV.a.4), (IV.a.5), and (IV.a.6), E is —C(Y)—N(R¹¹)—O—.

In some embodiments, in each of Formulas (IV.a), (IV.a.1), (IV.a.2), (IV.a.3), (IV.a.4), (IV.a.5), and (IV.a.6), E is —C(Y)—N(R¹¹)—N(R¹²)—.

In some embodiments, in each of Formulas (IV.a), (IV.a.1), (IV.a.2), (IV.a.3), (IV.a.4), (IV.a.5), and (IV.a.6), E is —O—N(R¹¹)—C(Y)—.

In some embodiments, in each of Formulas (IV.a), (IV.a.1), (IV.a.2), (IV.a.3), (IV.a.4), (IV.a.5), and (IV.a.6), E is —N(R¹²)—N(R¹¹)—C(Y)—.

In some embodiments, in each of Formulas (IV.a), (IV.a.1), (IV.a.2), (IV.a.3), (IV.a.4), (IV.a.5), and (IV.a.6), Y is (═O).

In some embodiments, in each of Formulas (IV.a), (IV.a.1), (IV.a.2), (IV.a.3), (IV.a.4), (IV.a.5), and (IV.a.6), Y is (═S).

In some embodiments, in each of Formulas (IV.a), (IV.a.1), (IV.a.2), (IV.a.3), (IV.a.4), (IV.a.5), and (IV.a.6), Y is (═N(R¹³)).

In some embodiments, in each of Formulas (IV.a), (IV.a.1), (IV.a.2), (IV.a.3), (IV.a.4), (IV.a.5), and (IV.a.6), Y is (═N(CN)).

In some embodiments, in each of Formulas (IV.a), (IV.a.1), (IV.a.2), (IV.a.3), (IV.a.4), (IV.a.5), and (IV.a.6), Y is (═N(OR¹⁴)).

In some embodiments, in each of Formulas (IV.a), (IV.a.1), (IV.a.2), (IV.a.3), (IV.a.4), (IV.a.5), and (IV.a.6), Y is (═N(R¹⁵)(R¹⁶)).

In some embodiments, in each of Formulas (IV.a), (IV.a.1), (IV.a.2), (IV.a.3), (IV.a.4), (IV.a.5), and (IV.a.6), Y is (═C(R¹⁷)(R¹⁸)).

In some embodiments, in each of Formulas (IV.a), (IV.a.1), (IV.a.2), (IV.a.3), (IV.a.4), (IV.a.5), and (IV.a.6), B is an unsubstituted aromatic ring.

In some embodiments, in each of Formulas (IV.a), (IV.a.1), (IV.a.2), (IV.a.3), (IV.a.4), (IV.a.5), and (IV.a.6), B is an unsubstituted benzo ring, and Formula (IV.a.) has the general structure:

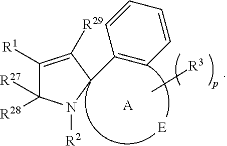

In some embodiments, in each of Formulas (IV.a), (IV.a.1), (IV.a.2), (IV.a.3), (IV.a.4), (IV.a.5), and (IV.a.6), B is an aromatic ring which is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (IV.a), (IV.a.1), (IV.a.2), (IV.a.3), (IV.a.4), (IV.a.5), and (IV.a.6), B is a benzo ring which is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (IV.a), (IV.a.1), (IV.a.2), (IV.a.3), (IV.a.4), (IV.a.5), and (IV.a.6), R¹ is unsubstituted aryl.

In some embodiments, in each of Formulas (IV.a), (IV.a.1), (IV.a.2), (IV.a.3), (IV.a.4), (IV.a.5), and (IV.a.6), R¹ is unsubstituted phenyl.

In some embodiments, in each of Formulas (IV.a), (IV.a.1), (IV.a.2), (IV.a.4), (IV.a.5), and (IV.a.6), R¹ is unsubstituted naphthyl.

In some embodiments, in each of Formulas (IV.a), (IV.a.1), (IV.a.2), (IV.a.3), (IV.a.4), (IV.a.5), and (IV.a.6), R¹ is substituted aryl.

In some embodiments, in each of Formulas (IV.a), (IV.a.1), (IV.a.2), (IV.a.3), (IV.a.4), (IV.a.5), and (IV.a.6), R¹ is substituted phenyl.

In some embodiments, in each of Formulas (IV.a), (IV.a.1), (IV.a.2), (IV.a.3), (IV.a.4), (IV.a.5), and (IV.a.6), R¹ is substituted naphthyl.

In some embodiments, in each of Formulas (IV.a), (IV.a.1), (IV.a.2), (IV.a.3), (IV.a.4), (IV.a.5), and (IV.a.6), R¹ is aryl substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (IV.a), (IV.a.1), (IV.a.2), (IV.a.3), (IV.a.5), and (IV.a.6), R¹ is phenyl substituted with one or more substituents, which can be the same or different, each substituent being independently selected from the group consisting halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶

In some embodiments, in each of Formulas (IV.a), (IV.a.1), (IV.a.2), (IV.a.3), (IV.a.4), (IV.a.5), and (IV.a.6), R¹ is phenyl substituted with one to four substituents, which can be the same or different, each substituent being independently selected from the group consisting of halo, —OH, —CN, —NO₂, —NR²¹R²², and haloalkyl.

In some embodiments, in each of Formulas (IV.a), (IV.a.1), (IV.a.2), (IV.a.3), (IV.a.4), (IV.a.5), and (IV.a.6), R¹ is selected from the group consisting of:

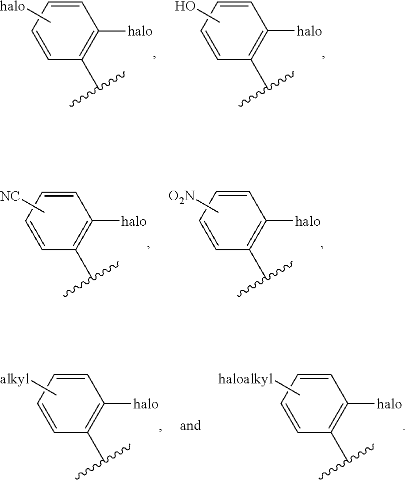

In some embodiments, in each of Formulas (IV.a), (IV.a.1), (IV.a.2), (IV.a.3), (IV.a.4), (IV.a.5), and (IV.a.6), R¹ is:

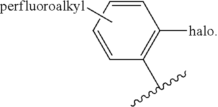

In some embodiments, in each of Formulas (IV.a), (IV.a.1), (IV.a.2), (IV.a.3), (IV.a.4), (IV.a.5), and (IV.a.6), R¹ is phenyl substituted with one to three fluoro groups.

In some embodiments, in each of Formulas (IV.a), (IV.a.1), (IV.a.2), (IV.a.3), (IV.a.4), (IV.a.5), and (IV.a.6), R¹ is phenyl substituted with two fluoro groups.

In some embodiments, in each of Formulas (IV.a), (IV.a.1), (IV.a.2), (IV.a.3), (IV.a.4), (IV.a.5), and (IV.a.6), R¹ is phenyl substituted with one fluoro group.

In some embodiments, in each of Formulas (IV.a), (IV.a.1), (IV.a.2), (IV.a.3), (IV.a.4), (IV.a.5), and (IV.a.6), R¹ is:

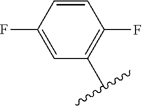

In some embodiments, in each of Formulas (IV.a), (IV.a.1), (IV.a.2), (IV.a.3), (IV.a.4), (IV.a.5), and (IV.a.6), R²⁷, R²⁸, and R²⁹ are each independently selected from the group consisting of H and alkyl.

In some embodiments, in each of Formulas (IV.a), (IV.a.1), (IV.a.2), (IV.a.3), (IV.a.4), (IV.a.5), and (IV.a.6), R² is —C(Z)R⁷.

In some embodiments, in each of Formulas (IV.a), (IV.a.1), (IV.a.2), (IV.a.3), (IV.a.4), (IV.a.5), and (IV.a.6), R² is —C(Z)NR⁹R¹⁰.

In some embodiments, in each of Formulas (IV.a), (IV.a.1), (IV.a.2), (IV.a.3), (IV.a.4), (IV.a.5), and (IV.a.6), R² is —C(Z)OR⁸.

In some embodiments, in each of Formulas (IV.a), (IV.a.1), (IV.a.2), (IV.a.3), (IV.a.4), (IV.a.5), and (IV.a.6), R² is —SO₂NR⁹R¹⁰.

In some embodiments, in each of Formulas (IV.a), (IV.a.1), (IV.a.2), (IV.a.3), (IV.a.4), (IV.a.5), and (IV.a.6), R² is alkyl.

In some embodiments, in each of Formulas (IV.a), (IV.a.1), (IV.a.2), (IV.a.3), (IV.a.4), (IV.a.5), and (IV.a.6), R² is heteroalkyl.

In some embodiments, in each of Formulas (IV.a), (IV.a.1), (IV.a.2), (IV.a.3), (IV.a.4), (IV.a.5), and (IV.a.6), R² is aryl.

In some embodiments, in each of Formulas (IV.a), (IV.a.1), (IV.a.2), (IV.a.3), (IV.a.4), (IV.a.5), and (IV.a.6), R² is heteroaryl.

In some embodiments, in each of Formulas (IV.a), (IV.a.1), (IV.a.2), (IV.a.3), (IV.a.4), (IV.a.5), and (IV.a.6), R² is cycloalkyl.

In some embodiments, in each of Formulas (IV.a), (IV.a.1), (IV.a.2), (IV.a.3), (IV.a.4), (IV.a.5), and (IV.a.6), R² is cycloalkenyl.

In some embodiments, in each of Formulas (IV.a), (IV.a.1), (IV.a.2), (IV.a.3), (IV.a.4), (IV.a.5), and (IV.a.6), R² is heterocycloalkyl.

In some embodiments, in each of Formulas (IV.a), (IV.a.1), (IV.a.2), (IV.a.3), (IV.a.4), (IV.a.5), and (IV.a.6), R² is heterocycloalkenyl.

In some embodiments, in each of Formulas (IV.a), (IV.a.1), (IV.a.2), (IV.a.3), (IV.a.4), (IV.a.5), and (IV.a.6), Z is (═O).

In some embodiments, in each of Formulas (IV.a), (IV.a.1), (IV.a.2), (IV.a.3), (IV.a.4), (IV.a.5), and (IV.a.6), Z is (═S).

In some embodiments, in each of Formulas (IV.a), (IV.a.1), (IV.a.2), (IV.a.3), (IV.a.4), (IV.a.5), and (IV.a.6), Z is (═N(R¹³)).

In some embodiments, in each of Formulas (IV.a), (IV.a.1), (IV.a.2), (IV.a.3), (IV.a.4), (IV.a.5), and (IV.a.6), Z is (═N(CN)).

In some embodiments, in each of Formulas (IV.a), (IV.a.1), (IV.a.2), (IV.a.3), (IV.a.4), (IV.a.5), and (IV.a.6), Z is (═N(OR¹⁴)).

In some embodiments, in each of Formulas (IV.a), (IV.a.1), (IV.a.2), (IV.a.3), (IV.a.4), (IV.a.5), and (IV.a.6), Z is (═N(R¹⁵)(R¹⁶)).

In some embodiments, in each of Formulas (IV.a), (IV.a.1), (IV.a.2), (IV.a.3), (IV.a.4), (IV.a.5), and (IV.a.6), Z is (═C(R¹⁷)(R¹⁸)).

In some embodiments, in each of Formulas (IV.a), (IV.a.1), (IV.a.2), (IV.a.3), (IV.a.4), (IV.a.5), and (IV.a.6), R² is —C(Z)R⁷, and Z is (═O).

In some embodiments, in each of Formulas (IV.a), (IV.a.1), (IV.a.2), (IV.a.3), (IV.a.4), (IV.a.5), and (IV.a.6), R² is —C(O)H.

In some embodiments, in each of Formulas (IV.a), (IV.a.1), (IV.a.2), (IV.a.3), (IV.a.4), (IV.a.5), and (IV.a.6), R² is —C(O)alkyl.

In some embodiments, in each of Formulas (IV.a), (IV.a.1), (IV.a.2), (IV.a.3), (IV.a.4), (IV.a.5), and (IV.a.6), R² is —C(O)CH₃.

In some embodiments, in each of Formulas (IV.a), (IV.a.1), (IV.a.2), (IV.a.3), (IV.a.4), (IV.a.5), and (IV.a.6), R² is —C(O)R⁷, wherein said R⁷ is alkyl substituted with one or more substituents, which can be the same or different, each substituent being independently selected from the group consisting of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (IV.a), (IV.a.1), (IV.a.2), (IV.a.3), (IV.a.4), (IV.a.5), and (IV.a.6), R² is —C(O)R⁷, wherein said R⁷ is alkyl substituted with one to three substituents, which can be the same or different, each substituent being independently selected from the group consisting of —OR¹⁹, —NR²¹R²², and cycloalkyl.

In some embodiments, in each of Formulas (IV.a), (IV.a.1), (IV.a.2), (IV.a.3), (IV.a.4), (IV.a.5), and (IV.a.6), R² is —C(O)R⁷, wherein said R⁷ is alkyl, wherein said alkyl is substituted with alkyl and —OH.

In some embodiments, in each of Formulas (IV.a), (IV.a.1), (IV.a.2), (IV.a.3), (IV.a.4), (IV.a.5), and (IV.a.6), R² is —C(O)R⁷, wherein said R⁷ is alkyl substituted with one to three substituents, which can be the same or different, each substituent being independently selected from the group consisting of —OH, —NH₂, and cyclopropyl.

In some embodiments, in each of Formulas (IV.a), (IV.a.1), (IV.a.2), (IV.a.3), (IV.a.4), (IV.a.5), and (IV.a.6), R² is —C(O)R⁷, wherein said R⁷ is alkyl substituted with one to two substituents, which can be the same or different, each substituent being independently selected from the group consisting of —NH₂, and cyclopropyl.

In some embodiments, in each of Formulas (IV.a), (IV.a.1), (IV.a.2), (IV.a.3), (IV.a.4), (IV.a.5), and (IV.a.6), R² is —C(O)R⁷, wherein said R⁷ is alkyl substituted with —OH.

In some embodiments, in each of Formulas (IV.a), (IV.a.1), (IV.a.2), (IV.a.3), (IV.a.5), and (IV.a.6), R² is —C(O)R⁷, wherein said R¹ is unsubstituted heterocycloalkyl.

In some embodiments, in each of Formulas (IV.a), (IV.a.1), (IV.a.2), (IV.a.3), (IV.a.4), (IV.a.5), and (IV.a.6), R² is —C(O)R⁷, wherein said R⁷ is substituted heterocycloalkyl.

In some embodiments, in each of Formulas (IV.a), (IV.a.1), (IV.a.2), (IV.a.4), (IV.a.5), and (IV.a.6), R² is —C(O)R⁷, wherein said R⁷ is heterocycloalkyl substituted with one or more substituents, which can be the same or different, each substituent being independently selected from the group consisting of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (IV.a), (IV.a.1), (IV.a.2), (IV.a.3), (IV.a.5), and (IV.a.6), R² is —C(O)R⁷, wherein said R⁷ is selected from the group consisting of substituted piperidine, substituted piperazine, substituted morpholine, substituted pyrrolidine, and substituted azetidine.

In some embodiments, in each of Formulas (IV.a), (IV.a.1), (IV.a.2), (IV.a.3), (IV.a.5), and (IV.a.6), R² is selected from:

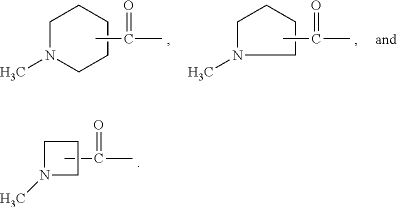

In some embodiments, in each of Formulas (IV.a), (IV.a.1), (IV.a.2), (IV.a.3), (IV.a.5), and (IV.a.6), R² is —C(O)NR⁹R¹⁰.

In some embodiments, in each of Formulas (IV.a), (IV.a.1), (IV.a.2), (IV.a.3), (IV.a.4), (IV.a.5), and (IV.a.6), R² is —C(O)NH₂.

In some embodiments, in each of Formulas (IV.a), (IV.a.1), (IV.a.2), (IV.a.3), (IV.a.4), (IV.a.5), and (IV.a.6), R² is —C(O)NR⁹R¹⁰, wherein R⁹ and R¹⁰ can be the same or different, each being independently selected from alkyl.

In some embodiments, in each of Formulas (IV.a), (IV.a.1), (IV.a.2), (IV.a.3), (IV.a.4), (IV.a.5), and (IV.a.6), R² is —C(O)NR⁹R¹⁰, wherein R⁹ is unsubstituted heterocycloalkyl and R¹⁹ is selected from the group consisting of H and alkyl.

In some embodiments, in each of Formulas (IV.a), (IV.a.1), (IV.a.2), (IV.a.3), (IV.a.4), (IV.a.5), and (IV.a.6), R² is —C(O)NR⁹R¹⁰, wherein R⁹ is substituted heterocycloalkyl and R¹⁰ is selected from the group consisting of H and alkyl.

In some embodiments, in each of Formulas (IV.a), (IV.a.1), (IV.a.2), (IV.a.3), (IV.a.4), (IV.a.5), and (IV.a.6), R² is —C(O)NR⁹R¹⁰, wherein R⁹ is heterocycloalkyl substituted with from one to three substituents, which can be the same or different, each substituent being independently selected from alkyl, and R¹⁰ is selected from the group consisting of H and alkyl.

In some embodiments, in each of Formulas (IV.a), (IV.a.1), (IV.a.2), (IV.a.3), (IV.a.4), (IV.a.5), and (IV.a.6), R² is selected from the group consisting of: alkyl, haloalkyl, heteroalkyl, heterohaloalkyl, —C(O)R⁷. —C(O)OR⁸, and —C(O)NR⁹R¹⁰.

In some embodiments, in each of Formulas (IV.a), (IV.a.1), (IV.a.2), (IV.a.3), (IV.a.4), (IV.a.5), and (IV.a.6), R² is selected from the group consisting of

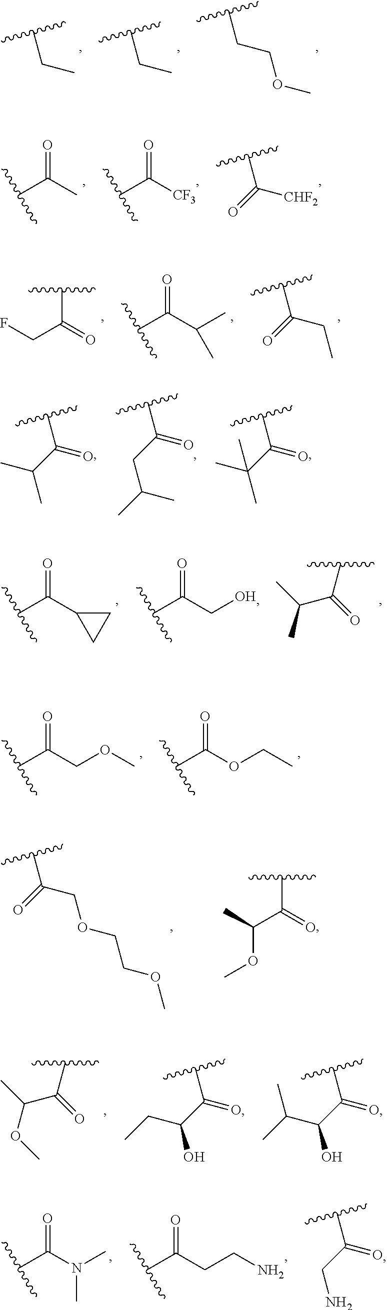

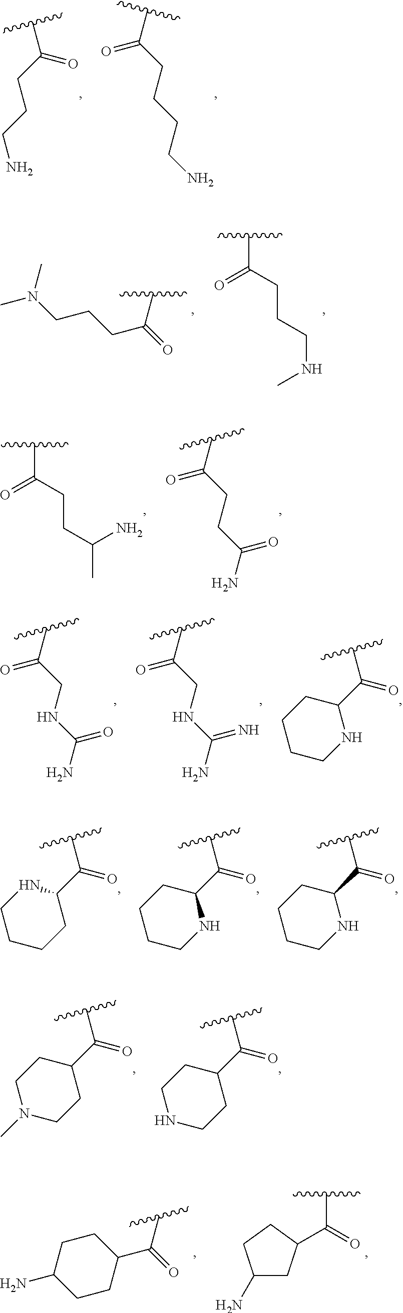

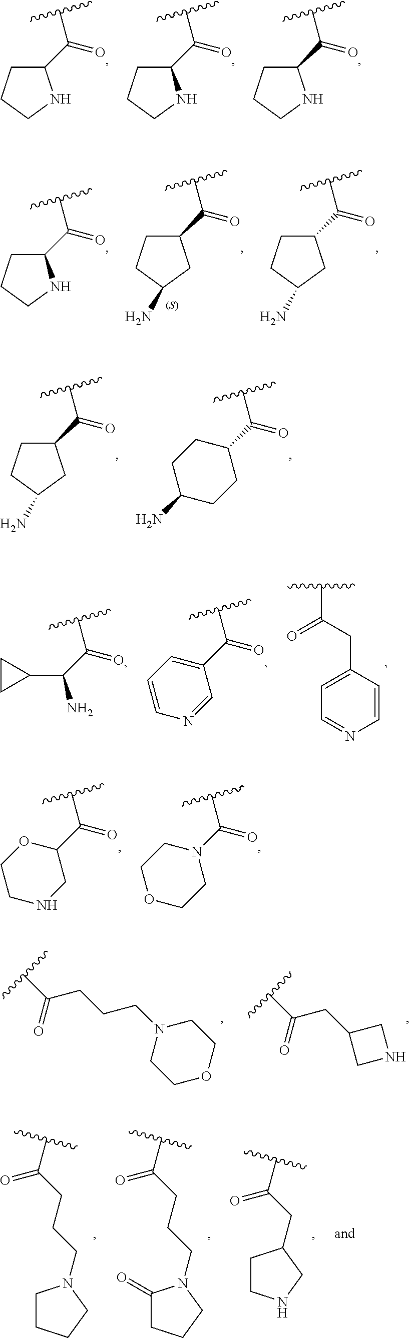

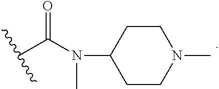

In some embodiments, in each of Formulas (IV.a), (IV.a.1), (IV.a.2), (IV.a.3), (IV.a.4), (IV.a.5), and (IV.a.6), R² is

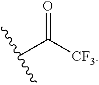

In some embodiments, in each of Formulas (IV.a), (IV.a.1), (IV.a.2), (IV.a.3), (IV.a.4), (IV.a.5), and (IV.a.6), R² is

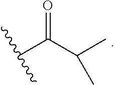

In some embodiments, in each of Formulas (IV.a), (IV.a.1), (IV.a.2), (IV.a.3), (IV.a.4), (IV.a.5), and (IV.a.6), R² is

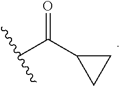

In some embodiments, in each of Formulas (IV.a), (IV.a.1), (IV.a.2), (IV.a.3), (IV.a.4), (IV.a.5), and (IV.a.6), R² is

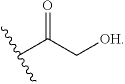

In some embodiments, in each of Formulas (IV.a), (IV.a.1), (IV.a.2), (IV.a.3), (IV.a.4), (IV.a.5), and (IV.a.6), R² is

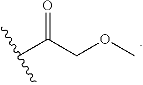

In some embodiments, in each of Formulas (IV.a), (IV.a.1), (IV.a.2), (IV.a.3), (IV.a.4), (IV.a.5), and (IV.a.6), R² is

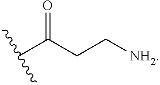

In some embodiments, in each of Formulas (IV.a), (IV.a.1), (IV.a.2), (IV.a.3), (IV.a.4), (IV.a.5), and (IV.a.6), R² is

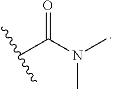

In some embodiments, in each of Formulas (IV.a), (IV.a.1), (IV.a.2), (IV.a.3), (IV.a.4), (IV.a.5), and (IV.a.6), R² is

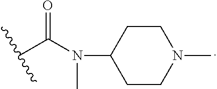

In some embodiments, in each of Formulas (IV.a), (IV.a.1), (IV.a.2), (IV.a.3), (IV.a.4), (IV.a.5), and (IV.a.6), p is 0 and R³ is not present.

In some embodiments, in each of Formulas (IV.a), (IV.a.1), (IV.a.2), (IV.a.3), (IV.a.4), (IV.a.5), and (IV.a.6), p is 1.

In some embodiments, in each of Formulas (IV.a), (IV.a.1), (IV.a.2), (IV.a.3), (IV.a.4), (IV.a.5), and (IV.a.6), p is 2.

In some embodiments, in each of Formulas (IV.a), (IV.a.1), (IV.a.2), (IV.a.3), (IV.a.4), (IV.a.5), and (IV.a.6), p is 3.

In some embodiments, in each of Formulas (IV.a), (IV.a.1), (IV.a.2), (IV.a.3), (IV.a.4), (IV.a.5), and (IV.a.6), p is 4.

In some embodiments, in each of Formulas (IV.a), (IV.a.1), (IV.a.2), (IV.a.3), (IV.a.4), (IV.a.5), and (IV.a.6), p is ≧2 and at least two groups R³ are attached to the same ring atom.

In some embodiments, in each of Formulas (IV.a), (IV.a.1), (IV.a.2), (IV.a.3), (IV.a.5), and (IV.a.6), p is 1 and R³ is independently selected from the group consisting of alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (IV.a), (IV.a.1), (IV.a.2), (IV.a.3), (IV.a.4), (IV.a.5), and (IV.a.6), p is 2, 3, or 4 and each R³ is independently selected from the group consisting of alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (IV.a), (IV.a.1), (IV.a.2), (IV.a.3), (IV.a.4), (IV.a.5), and (IV.a.6), p is 2, 3, or 4 and at least two groups R³ are bound to the same ring carbon atom, wherein each R³, which may be the same or different, is independently selected from the group consisting of alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (IV.a), (IV.a.1), (IV.a.3), (IV.a.4), (IV.a.5), and (IV.a.6), p is 2, 3, or 4 and at least two groups R³ are bound to the same ring carbon atom, wherein two R³ groups, which may be the same or different, together with the carbon atom to which they are attached, form a cycloalkyl, a cycloalkenyl, a heterocycloalkyl ring containing from one to three heteroatoms selected from the group consisting of N, O, and S, or a heterocycloalkenyl ring containing from one to three heteroatoms selected from the group consisting of N, O, and S.

In one embodiment, in Formula (IV.a), p is 1, 2, 3, or 4 and each R³ is independently selected from the group consisting of alkyl, heteroalkyl, alkenyl, heteroalkenyl, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(S)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶, —NR²³C(O)NR²⁵R²⁶, and —NR²³—C(NH)—NR²⁶,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (IV.a), p is 1 and R³ is selected from the group consisting of alkyl, heteroalkyl, alkenyl, and heteroalkenyl,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (IV.a), p is 2, 3, or 4 and any two R³ groups bound to the same ring A atom are taken together to form a —C(O)— group.

In one embodiment, in Formula (IV.a), p is 2, 3, or 4 and any two R³ groups bound to the same ring A atom are taken together to form a spiroheterocycloalkyl group having from 1 to 3 ring heteroatoms independently selected from the group consisting of —NH—, —NR⁶—, O, S, S(O), and S(O)₂, or spiroheterocycloalkenyl group having from 1 to 3 ring heteroatoms independently selected from the group consisting of —NH—, —NR⁶—, O, S, S(O), and S(O)₂.

In some embodiments, in each of Formulas (IV.a), (IV.a.1), (IV.a.2), (IV.a.3), (IV.a.4), (IV.a.5), and (IV.a.6), R³ is alkyl.

In some embodiments, in each of Formulas (IV.a), (IV.a.1), (IV.a.2), (IV.a.3), (IV.a.4), (IV.a.5), and (IV.a.6), R³ is heteroalkyl.

In some embodiments, in each of Formulas (IV.a), (IV.a.1), (IV.a.2), (IV.a.3), (IV.a.4), (IV.a.5), and (IV.a.6), R³ is alkenyl.

In some embodiments, in each of Formulas (IV.a), (IV.a.1), (IV.a.2), (IV.a.3), (IV.a.4), (IV.a.5), and (IV.a.6), R³ is heteroalkenyl.

In some embodiments, in each of Formulas (IV.a), (IV.a.1), (IV.a.2), (IV.a.3), (IV.a.4), (IV.a.5), and (IV.a.6), R³ is alkynyl.

In some embodiments, in each of Formulas (IV.a), (IV.a.1), (IV.a.2), (IV.a.3), (IV.a.4), (IV.a.5), and (IV.a.6), R³ is heteroalkynyl.

In some embodiments, in each of Formulas (IV.a), (IV.a.1), (IV.a.3), (IV.a.4), (IV.a.5), and (IV.a.6), R³ is aryl.

In some embodiments, in each of Formulas (IV.a), (IV.a.1), (IV.a.2), (IV.a.3), (IV.a.4), (IV.a.5), and (IV.a.6), R³ is heteroaryl.

In some embodiments, in each of Formulas (IV.a), (IV.a.1), (IV.a.2), (IV.a.3), (IV.a.4), (IV.a.5), and (IV.a.6), R³ is cycloalkyl.

In some embodiments, in each of Formulas (IV.a), (IV.a.1), (IV.a.2), (IV.a.3), (IV.a.4), (IV.a.5), and (IV.a.6), R³ is cycloalkenyl.

In some embodiments, in each of Formulas (IV.a), (IV.a.1), (IV.a.2), (IV.a.3), (IV.a.4), (IV.a.5), and (IV.a.6), R³ is heterocycloalkyl.

In some embodiments, in each of Formulas (IV.a), (IV.a.1), (IV.a.2), (IV.a.3), (IV.a.4), (IV.a.5), and (IV.a.6), R³ is heterocycloalkonyl.

In some embodiments, in each of Formulas (IV.a), (IV.a.1), (IV.a.2), (IV.a.3), (IV.a.4), (IV.a.5), and (IV.a.6), R³ is halogen.

In some embodiments, in each of Formulas (IV.a), (IV.a.1), (IV.a.2), (IV.a.3), (IV.a.4), (IV.a.5), and (IV.a.6), R³ is —CN.

In some embodiments, in each of Formulas (IV.a), (IV.a.1), (IV.a.2), (IV.a.3), (IV.a.4), (IV.a.5), and (IV.a.6), R³ is —NO₂.

In some embodiments, in each of Formulas (IV.a), (IV.a.1), (IV.a.2), (IV.a.3), (IV.a.4), (IV.a.5), and (IV.a.6), R³ is —OR¹⁹.

In some embodiments, in each of Formulas (IV.a), (IV.a.1), (IV.a.2), (IV.a.3), (IV.a.4), (IV.a.5), and (IV.a.6), R³ is —OC(O)OR²⁰.

In some embodiments, in each of Formulas (IV.a), (IV.a.1), (IV.a.2), (IV.a.3), (IV.a.4), (IV.a.5), and (IV.a.6), R³ is —NR²¹R²².

In some embodiments, in each of Formulas (IV.a), (IV.a.1), (IV.a.2), (IV.a.3), (IV.a.4), (IV.a.5), and (IV.a.6), R³ is —NR²³SO₂R²⁴.

In some embodiments, in each of Formulas (IV.a), (IV.a.1), (IV.a.2), (IV.a.3), (IV.a.4), (IV.a.5), and (IV.a.6), R³ is —NR²³C(O)OR²⁰.

In some embodiments, in each of Formulas (IV.a), (IV.a.1), (IV.a.2), (IV.a.3), (IV.a.4), (IV.a.5), and (IV.a.6), R³ is —NR²³C(O)R²⁴

In some embodiments, in each of Formulas (IV.a), (IV.a.1), (IV.a.2), (IV.a.3), (IV.a.4), (IV.a.5), and (IV.a.6), R³ is —SO₂NR²⁵R²⁶.

In some embodiments, in each of Formulas (IV.a), (IV.a.1), (IV.a.2), (IV.a.3), (IV.a.4), (IV.a.5), and (IV.a.6), R³ is —C(O)R²⁴.

In some embodiments, in each of Formulas (IV.a), (IV.a.1), (IV.a.2), (IV.a.3), (IV.a.5), and (IV.a.6), R³ is —C(O)OR²⁰.

In some embodiments, in each of Formulas (IV.a), (IV.a.1), (IV.a.2), (IV.a.3), (IV.a.4), (IV.a.5), and (IV.a.6), R³ is —SR¹⁹.

In some embodiments, in each of Formulas (IV.a), (IV.a.1), (IV.a.2), (IV.a.3), (IV.a.4), (IV.a.5), and (IV.a.6), R³ is —S(O)R¹⁹.

In some embodiments, in each of Formulas (IV.a), (IV.a.1), (IV.a.2), (IV.a.3), (IV.a.4), (IV.a.5), and (IV.a.6), R³ is —SO₂R¹⁹.

In some embodiments, in each of Formulas (IV.a), (IV.a.1), (IV.a.2), (IV.a.3), (IV.a.4), (IV.a.5), and (IV.a.6), R³ is —OC(O)R²⁴.

In some embodiments, in each of Formulas (IV.a), (IV.a.1), (IV.a.2), (IV.a.3), (IV.a.4), (IV.a.5), and (IV.a.6), R³ is —C(O)NR²⁵R²⁶.

In some embodiments, in each of Formulas (IV.a), (IV.a.1), (IV.a.2), (IV.a.3), (IV.a.4), (IV.a.5), and (IV.a.6), R³ is —NR²³C(N—CN)NR²⁵R²⁶.

In some embodiments, in each of Formulas (IV.a), (IV.a.1), (IV.a.2), (IV.a.3), (IV.a.4), (IV.a.5), and (IV.a.6), R³ is —NR²³C(O)NR²⁵R²⁶

In some embodiments, in each of Formulas (IV.a), (IV.a.1), (IV.a.2), (IV.a.3), (IV.a.4), (IV.a.5), and (IV.a.6), R³ is selected from the group consisting of: methyl, ethyl, propyl (straight or branched), butyl (straight or branched), pentyl (straight or branched), phenyl,

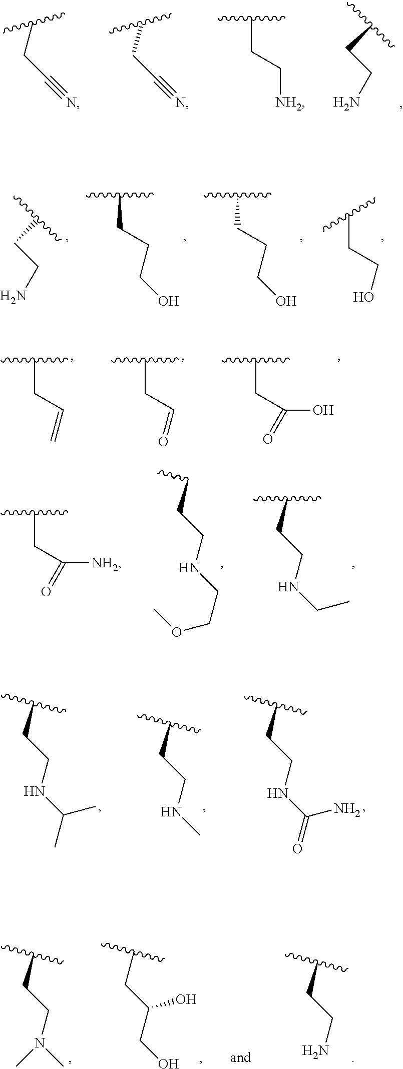

In some embodiments, in each of Formulas (IV.a), (IV.a.1), (IV.a.2), (IV.a.3), (IV.a.4), (IV.a.5), and (IV.a.6), when E is —NR⁶—, R³ is absent.

In one embodiment, the compounds of the invention have a structure shown in Formula (IV.b) and include pharmaceutically acceptable salts, solvates, esters, prodrugs, or isomers of said compounds:

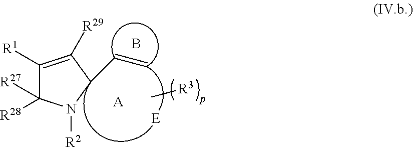

wherein R¹, R², R³, R²⁷, R²⁸, R²⁹, p, E, ring A, and ring B are selected independently of each other and wherein:
ring A (including E and the unsaturation shown) is a 6-membered cycloalkenyl or heterocycloalkenyl ring:
E is selected from the group consisting of —S—, —S(O)—, —S(O)₂—, —C(R⁴)(R⁵)—, —N(R⁶)—, —N(C(Y)R⁷)—, —N(C(Y)OR⁸)—, —N(C(Y)N(R⁹)(R¹⁰))—, —C(O)—N(R¹¹)—, —N(R¹¹)—C(O)—, —S(O)₂—N(R¹¹)—, —N(R¹¹)—S(O)₂—, —C(O)—O—, —O—C(O)—, —O—N(R⁶)—, —N(R⁶)—O—, —N(R⁶)—N(R¹²)—, —N═—, —C(R⁷)═N—, —C(O)—C(R⁷)═N—, —C(O)—N═N—, —O—C(Y)—N(R¹¹)—, —N(R¹¹)—C(Y)—O—, —N(R¹¹)—C(Y)—N(R¹²)—, —C(Y)—N(R¹¹)—O—, —C(Y)—N(R¹¹)—N(R¹²)—, —O—N(R¹¹)—C(Y)—, and —N(R¹²)—N(R¹¹)—C(Y)—;
ring B is a substituted or unsubstituted heteroaromatic ring;
and p, R¹, R², R³, R⁴, R⁵, R⁶, R⁷, R⁸, R⁹, R¹⁰, R¹¹, R¹², R²⁷, R²⁸, R²⁹, Y, and the optional substituents on ring B are as defined in any of the embodiments described above in Formula (I).

In one embodiment, Formula (IV.b) has the general structure shown in Formula (IV.b.1):

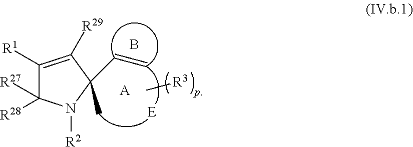

In one embodiment, Formula (Mb) has the general structure shown in Formula (IV.b.2):

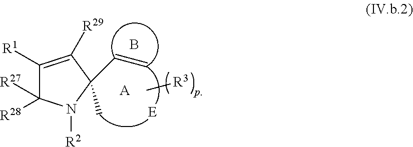

In one embodiment, Formula (IV.b) has the general structure shown in Formula (IV.b.3):

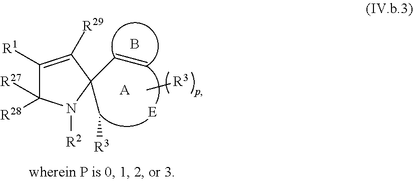

In one embodiment, Formula (IV.b) has the general structure shown in Formula (IV.b.4):

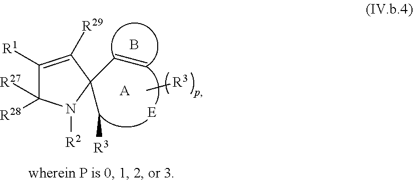

In one embodiment, Formula (IV.b) has the general structure shown in Formula (IV.b.5):

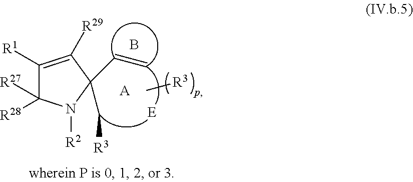

In one embodiment, Formula (IV.b) has the general structure shown in Formula (IV.b.6):

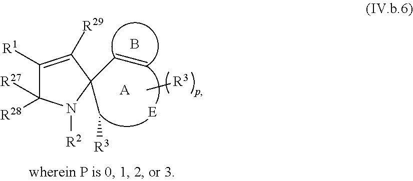

In some embodiments, in each of Formulas (IV.b), (IV.b.1), (IV.b.2), (IV.b.3), (IV.b.4), (IV.b.5), and (IV.b.6), ring A is a cycloalkenyl ring and E is —C(R⁴)(R⁶)—.

In some embodiments, in each of Formulas (IV.b), (IV.b.1), (IV.b.2), (IV.b.3), (IV.b.4), (IV.b.5), and (IV.b.6), E is selected from the group consisting of —O—, —S—, —S(O)—, —S(O)₂—, and —N(R⁵)—.

In some embodiments, in each of Formulas (IV.b), (IV.b.1), (IV.b.2), (IV.b.3), (IV.b.4), (IV.b.5), and (IV.b.6), E is selected from the group consisting of —O—, —S—, —S(O)—, —S(O)₂—, and —N(R⁶)—, wherein R⁶ is selected from the group consisting of H, alkyl, —C(O)R²⁴, and —C(S)R²⁴.

In some embodiments, in each of Formulas (IV.b), (IV.b.1), (IV.b.2), (IV.b.3), (IV.b.4), (IV.b.5), and (IV.b.6), E is selected from the group consisting of —O— and —N(R⁶)—, wherein R⁶ is selected from the group consisting of H, alkyl, —C(O)R²⁴, and —C(S)R²⁴.

In some embodiments, in each of Formulas (IV.b), (IV.b.1), (IV.b.2), (IV.b.3), (IV.b.4), (IV.b.5), and (IV.b.6), E is —O—.

In some embodiments, in each of Formulas (IV.b), (IV.b.1), (IV.b.2), (IV.b.3), (IV.b.4), (IV.b.5), and (IV.b.6), E is —S—.

In some embodiments, in each of Formulas (IV.b), (IV.b.1), (IV.b.2), (IV.b.3), (IV.b.4), (IV.b.5), and (IV.b.6), E is —S(O)—.

In some embodiments, in each of Formulas (IV.b), (IV.b.1), (IV.b.2), (IV.b.3), (IV.b.4), (IV.b.5), and (IV.b.6), E is —S(O)₂—.

In some embodiments, in each of Formulas (IV.b), (IV.b.1), (IV.b.2), (IV.b.3), (IV.b.4), (IV.b.5), and (IV.b.6), E is —C(R⁴)(R⁵)—.

In some embodiments, in each of Formulas (IV.b), (IV.b.1), (IV.b.2), (IV.b.3), (IV.b.4), (IV.b.5), and (IV.b.6), E is —N(R⁶)—.

In some embodiments, in each of Formulas (IV.b), (IV.b.1), (IV.b.2), (IV.b.3), (IV.b.4), (IV.b.5), and (IV.b.6), E is —N(C(Y)R⁷)—.

In some embodiments, in each of Formulas (IV.b), (IV.b.1), (IV.b.2), (IV.b.3), (IV.b.4), (IV.b.5), and (IV.b.6), E is —N(C(Y)OR⁸)—.

In some embodiments, in each of Formulas (IV.b), (IV.b.1), (IV.b.2), (IV.b.3), (IV.b.4), (IV.b.5), and (IV.b.6), E is —N(C(Y)N(R⁹)(R¹⁰))—.

In some embodiments, in each of Formulas (IV.b), (IV.b.1), (IV.b.2), (IV.b.3), (IV.b.4), (IV.b.5), and (IV.b.6), E is —C(O)—N(R¹¹)—.

In some embodiments, in each of Formulas (IV.b), (IV.b.1), (IV.b.2), (IV.b.3), (IV.b.4), (IV.b.5), and (IV.b.6), E is —N(R¹¹)—C(O)—.

In some embodiments, in each of Formulas (IV.b), (IV.b.1), (IV.b.2), (IV.b.3), (IV.b.4), (IV.b.5), and (IV.b.6), E is —S(O)₂—N(R¹¹)—.

In some embodiments, in each of Formulas (IV.b), (IV.b.1), (IV.b.2), (IV.b.3), (IV.b.4), (IV.b.5), and (IV.b.6), E is —N(R¹¹)—S(O)₂—.

In some embodiments, in each of Formulas (IV.b), (IV.b.1), (IV.b.2), (IV.b.3), (IV.b.4), (IV.b.5), and (IV.b.6), E is —C(O)—O—.

In some embodiments, in each of Formulas (IV.b), (IV.b.1), (IV.b.2), (IV.b.3), (IV.b.4), (IV.b.5), and (IV.b.6), E is —O—C(O)—.

In some embodiments, in each of Formulas (IV.b), (IV.b.1), (IV.b.2), (IV.b.3), (IV.b.4), (IV.b.5), and (IV.b.6), E is —O—N(R⁶)—.

In some embodiments, in each of Formulas (IV.b), (IV.b.1), (IV.b.3), (IV.b.4), (IV.b.5), and (IV.b.6), E is —N(R⁶)—O—.

In some embodiments, in each of Formulas (IV.b), (IV.b.1), (IV.b.2), (IV.b.3), (IV.b.4), (IV.b.5), and (IV.b.6), E is —N(R⁶)—N(R¹²)—.

In some embodiments, in each of Formulas (IV.b), (IV.b.1), (IV.b.2), (IV.b.3), (IV.b.4), (IV.b.5), and (IV.b.6), E is —N═N—.

In some embodiments, in each of Formulas (IV.b), (IV.b.1), (IV.b.2), (IV.b.3), (IV.b.4), (IV.b.5), and (IV.b.6), E is —C(R⁷)═N—.

In some embodiments, in each of Formulas (IV.b), (IV.b.1), (IV.b.2), (IV.b.3), (IV.b.4), (IV.b.5), and (IV.b.6), E is —C(O)—C(R⁷)═N—.

In some embodiments, in each of Formulas (IV.b), (IV.b.1), (IV.b.2), (IV.b.3), (IV.b.4), (IV.b.5), and (IV.b.6), E is —C(O)—N═N—.

In some embodiments, in each of Formulas (IV.b), (IV.b.1), (IV.b.2), (IV.b.3), (IV.b.4), (IV.b.5), and (IV.b.6), E is —O—C(Y)—N(R¹¹)—.

In some embodiments, in each of Formulas (IV.b), (IV.b.1), (IV.b.2), (IV.b.3), (IV.b.4), (IV.b.5), and (IV.b.6), E is —N(R¹¹)—C(Y)—O—.

In some embodiments, in each of Formulas (IV.b), (IV.b.1), (IV.b.3), (IV.b.4), (IV.b.5), and (IV.b.6), E is —N(R¹¹)—C(Y)—N(R¹²)—.

In some embodiments, in each of Formulas (IV.b), (IV.b.1), (IV.b.2), (IV.b.3), (IV.b.4), (IV.b.5), and (IV.b.6), E is —C(Y)—N(R¹¹)—O—.

In some embodiments, in each of Formulas (IV.b), (IV.b.1), (IV.b.2), (IV.b.3), (IV.b.4), (IV.b.5), and (IV.b.6), E is —C(Y)—N(R¹¹)—N(R¹²)—.

In some embodiments, in each of Formulas (IV.b), (IV.b.1), (IV.b.2), (IV.b.3), (IV.b.4), (IV.b.5), and (IV.b.6). E is —O—N(R¹¹)—C(Y)—.

In some embodiments, in each of Formulas (IV.b), (IV.b.1), (IV.b.2), (IV.b.3), (IV.b.4), (IV.b.5), and (IV.b.6), E is —N(R¹²)—N(R¹¹)—C(Y)—.

In some embodiments, in each of Formulas (IV.b), (IV.b.1), (IV.b.2), (IV.b.3), (IV.b.4), (IV.b.5), and (IV.b.6), Y is (═O).

In some embodiments, in each of Formulas (IV.b), (IV.b.1), (IV.b.2), (IV.b.4), (IV.b.5), and (IV.b.6), Y is (═S).

In some embodiments, in each of Formulas (IV.b), (IV.b.1), (IV.b.2), (IV.b.3), (IV.b.4), (IV.b.5), and (IV.b.6), Y is (═N(R¹³)).

In some embodiments, in each of Formulas (IV.b), (IV.b.1), (IV.b.2), (IV.b.3), (IV.b.4), (IV.b.5), and (IV.b.6), Y is (═N(CN)).

In some embodiments, in each of Formulas (IV.b), (IV.b.1), (IV.b.2), (IV.b.3), (IV.b.4), (IV.b.5), and (IV.b.6), Y is (═N(OR¹⁴)).

In some embodiments, in each of Formulas (IV.b), (IV.b.1), (IV.b.2), (IV.b.4), (IV.b.5), and (IV.b.6), Y is (═N(R¹⁵)(R¹⁶)).

In some embodiments, in each of Formulas (IV.b), (IV.b.1), (IV.b.2), (IV.b.3), (IV.b.4), (IV.b.5), and (IV.b.6), Y is (═C(R¹⁷)(R¹⁸)).

In some embodiments, in each of Formulas (IV.b), (IV.b.1), (IV.b.2), (IV.b.4), (IV.b.5), and (IV.b.6), B is an unsubstituted heteroaromatic ring.

In some embodiments, in each of Formulas (IV.b), (IV.b.1), (IV.b.2), (IV.b.3), (IV.b.4), (IV.b.5), and (IV.b.6), B is an unsubstituted 5-6-membered heteroaromatic ring having from 1-3 ring heteroatoms, which can be the same or different, each hetero ring atom being independently selected from the group consisting of N, S, O, S(O), and S(O)₂.

In some embodiments, in each of Formulas (IV.b), (IV.b.1), (IV.b.2), (IV.b.3), (IV.b.4), (IV.b.5), and (IV.b.6), B is a heteroaromatic ring which is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (IV.b), (IV.b.1), (IV.b.2), (IV.b.3), (IV.b.4), (IV.b.5), and (IV.b.6), B is a 5-6-membered heteroaromatic ring having from 1-3 ring heteroatoms, which can be the same or different, each hetero ring atom being independently selected from the group consisting of N, S, O, S(O), and S(O)₂, which heteroaromatic ring is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (IV.b), (IV.b.1), (IV.b.2), (IV.b.3), (IV.b.4), (IV.b.5), and (IV.b.6), B is an unsubstituted 6-membered heteroaromatic ring having from 1-3 ring heteroatoms, which can be the same or different, each hetero ring atom being independently selected from the group consisting of N, S, and O.

In some embodiments, in each of Formulas (IV.b), (IV.b.1), (IV.b.2), (IV.b.3), (IV.b.4), (IV.b.5), and (IV.b.6), B is a 6-membered heteroaromatic ring having from 1-3 ring heteroatoms, which can be the same or different, each hetero ring atom being independently selected from the group consisting of N, S, and O, which heteroaromatic ring is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁹, —NR²³C(N—CN)NR²⁵R²⁶ and —NR²³C(O)NR²⁵R²⁶.

In some embodiments, in each of Formulas (IV.b), (IV.b.1), (IV.b.2), (IV.b.3), (IV.b.4), (IV.b.5), and (IV.b.6), B is an unsubstituted 6-membered heteroaromatic ring having 2 ring heteroatoms, each ring heteroatom being independently selected from of N, S, and O.

In some embodiments, in each of Formulas (IV.b), (IV.b.1), (IV.b.2), (IV.b.3), (IV.b.4), (IV.b.5), and (IV.b.6), B is a 6-membered heteroaromatic ring having 2 ring heteroatoms, each ring heteroatom being independently selected from of N, S, and O, which heteroaromatic ring is substituted with one or more substituents, which can be the same or different, each substituent being independently selected from the group consisting of halogen, —CN, —NO₂, alkyl, heteroalkyl, haloalkyl, —OR¹⁹, —NR²¹R²², —C(O)R²⁴, —C(O)OR²⁰, —SR¹⁹, —S(O)R¹⁹, —SO₂R¹⁹, —OC(O)R²⁴, and —C(O)NR²⁵R²⁶.

In some embodiments, in each of Formulas (IV.b), (IV.b.1), (IV.b.2), (IV.b.3), (IV.b.4), (IV.b.5), and (IV.b.6), B is an unsubstituted 5-membered heteroaromatic ring having from 1-2 ring heteroatoms, which can be the same or different, each hetero ring atom being independently selected from the group consisting of N, S, and O.

In some embodiments, in each of Formulas (IV.b), (IV.b.1), (IV.b.2), (IV.b.3), (IV.b.4), (IV.b.5), and (IV.b.6), B is a 5-membered heteroaromatic ring having from 1-2 ring heteroatoms, which can be the same or different, each hetero ring atom being independently selected from the group consisting of N, S, and O, which heteroaromatic ring is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (IV.b), (IV.b.1), (IV.b.2), (IV.b.3), (IV.b.4), (IV.b.5), and (IV.b.6), B is an unsubstituted 5-membered heteroaromatic ring having 1 ring heteroatom selected from of N, S, and O.

In some embodiments, in each of Formulas (IV.b), (IV.b.1), (IV.b.2), (IV.b.3), (IV.b.4), (IV.b.5), and (IV.b.6), B is a 5-membered heteroaromatic ring having 1 ring heteroatom selected from of N, S, and O, which heteroaromatic ring is substituted with one or more substituents, which can be the same or different, each substituent being independently selected from the group consisting of halogen, —CN, —NO₂, alkyl, heteroalkyl, haloalkyl, —OR¹⁹, —NR²¹R²², —C(O)R²⁴, —C(O)OR²⁰, —SR¹⁹, —S(O)R¹⁹, —SO₂R¹⁹, —OC(O)R²⁴, and —C(O)NR²⁵R²⁶.

In some embodiments, in each of Formulas (IV.b), (IV.b.1), (IV.b.2), (IV.b.3), (IV.b.4), (IV.b.5), and (IV.b.6), B is a 5-membered heteroaromatic ring having S as the ring heteroatom, which heteroaromatic ring is substituted with one or more substituents, which can be the same or different, each substituent being independently selected from the group consisting of halogen, —CN, —NO₂, alkyl, heteroalkyl, haloalkyl, —OR¹⁹, —NR²¹R²², —C(O)R²⁴, —C(O)OR²⁰, —SR¹⁹, —S(O)R¹⁹, —SO₂R¹⁹, —OC(O)R²⁴, and —C(O)NR²⁵R²⁶.

In some embodiments, in each of Formulas (IV.b), (IV.b.1), (IV.b.2), (IV.b.3), (IV.b.4), (IV.b.5), and (IV.b.6), B is an unsubstituted 5-membered heteroaromatic ring having S as the ring heteroatom.

In some embodiments, in each of Formulas (IV.b), (IV.b.1), (IV.b.2), (IV.b.3), (IV.b.4), (IV.b.5), and (IV.b.6), B is selected from the group consisting of

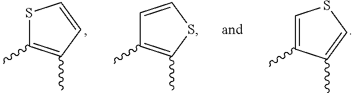

In some embodiments, in each of Formulas (IV.b), (IV.b.1), (IV.b.2), (IV.b.3), (IV.b.4), (IV.b.5), and (IV.b.6), R¹ is unsubstituted aryl.

In some embodiments, in each of Formulas (IV.b), (IV.b.1), (IV.b.2), (IV.b.3), (IV.b.4), (IV.b.5), and (IV.b.6), R¹ is unsubstituted phenyl.

In some embodiments, in each of Formulas (IV.b), (IV.b.1), (IV.b.2), (IV.b.3), (IV.b.4), (IV.b.5), and (IV.b.6), R¹ is unsubstituted naphthyl.

In some embodiments, in each of Formulas (IV.b), (IV.b.1), (IV.b.2), (IV.b.3), (IV.b.4), (IV.b.5), and (IV.b.6), R¹ is substituted aryl.

In some embodiments, in each of Formulas (IV.b), (IV.b.1), (IV.b.2), (IV.b.3), (IV.b.4), (IV.b.5), and (IV.b.6), R¹ is substituted phenyl.

In some embodiments, in each of Formulas (IV.b), (IV.b.1), (IV.b.2), (IV.b.3), (IV.b.4), (IV.b.5), and (IV.b.6), R¹ is substituted naphthyl.

In some embodiments, in each of Formulas (IV.b), (IV.b.1), (IV.b.2), (IV.b.3), (IV.b.5), and (IV.b.6), R¹ is aryl substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (IV.b), (IV.b.1), (IV.b.2), (IV.b.3), (IV.b.5), and (IV.b.6), R¹ is phenyl substituted with one or more substituents, which can be the same or different, each substituent being independently selected from the group consisting halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (IV.b), (IV.b.1), (IV.b.2), (IV.b.3), (IV.b.4), (IV.b.5), and (IV.b.6), R¹ is phenyl substituted with one to four substituents, which can be the same or different, each substituent being independently selected from the group consisting of halo, —OH, —CN, —NO₂, —NR²¹R²², and haloalkyl.

In some embodiments, in each of Formulas (IV.b), (IV.b.1), (IV.b.2), (IV.b.3), (IV.b.4), (IV.b.5), and (IV.b.6), R¹ is selected from the group consisting of:

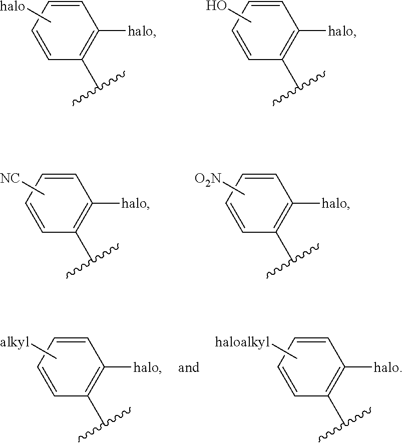

In some embodiments, in each of Formulas (IV.b), (IV.b.1), (IV.b.2), (IV.b.3), (IV.b.5), and (IV.b.6), R¹ is:

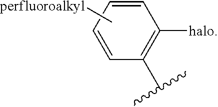

In some embodiments, in each of Formulas (IV.b), (IV.b.2), (IV.b.3), (IV.b.4), (IV.b.5), and (IV.b.6), R¹ is phenyl substituted with one to three fluoro groups.

In some embodiments, in each of Formulas (IV.b), (IV.b.1), (IV.b.2), (IV.b.3), (IV.b.4), (IV.b.5), and (IV.b.6), R¹ is phenyl substituted with two fluoro groups.

In some embodiments, in each of Formulas (IV.b), (IV.b.2), (IV.b.3), (IV.b.4), (IV.b.5), and (IV.b.6), R¹ is phenyl substituted with one fluoro group.

In some embodiments, in each of Formulas (IV.b), (IV.b.1), (IV.b.2), (IV.b.3), (IV.b.4), (IV.b.5), and (IV.b.6), R¹ is:

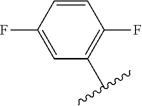

In some embodiments, in each of Formulas (IV.b), (IV.b.1), (IV.b.2), (IV.b.3), (IV.b.4), (IV.b.5), and (IV.b.6), R²⁷, R²⁸, and R²⁹ are each independently selected from the group consisting of H and alkyl.

In some embodiments, in each of Formulas (IV.b), (IV.b.1), (IV.b.2), (IV.b.3), (IV.b.4), (IV.b.5), and (IV.b.6), R² is —C(Z)R⁷.

In some embodiments, in each of Formulas (IV.b), (IV.b.1), (IV.b.2), (IV.b.3), (IV.b.4), (IV.b.5), and (IV.b.6), R² is —C(Z)NR⁹R¹⁰.

In some embodiments, in each of Formulas (IV.b), (IV.b.1), (IV.b.2), (IV.b.3), (IV.b.4), (IV.b.5), and (IV.b.6), R² is —C(Z)OR⁸.

In some embodiments, in each of Formulas (IV.b), (IV.b.1), (IV.b.2), (IV.b.3), (IV.b.4), (IV.b.5), and (IV.b.6), R² is —SO₂NR⁹R¹⁰.

In some embodiments, in each of Formulas (IV.b), (IV.b.1), (IV.b.2), (IV.b.3), (IV.b.4), (IV.b.5), and (IV.b.6), R² is alkyl.

In some embodiments, in each of Formulas (IV.b), (IV.b.1), (IV.b.2), (IV.b.3), (IV.b.4), (IV.b.5), and (IV.b.6), R² is heteroalkyl.

In some embodiments, in each of Formulas (IV.b), (IV.b.1), (IV.b.2), (IV.b.3), (IV.b.4), (IV.b.5), and (IV.b.6), R² is aryl.

In some embodiments, in each of Formulas (IV.b), (IV.b.1), (IV.b.2), (IV.b.3), (IV.b.4), (IV.b.5), and (IV.b.6), R² is heteroaryl.

In some embodiments, in each of Formulas (IV.b), (IV.b.1), (IV.b.2), (IV.b.3), (IV.b.4), (IV.b.5), and (IV.b.6), R² is cycloalkyl.

In some embodiments, in each of Formulas (IV.b), (IV.b.1), (IV.b.2), (IV.b.3), (IV.b.4), (IV.b.5), and (IV.b.6), R² is cycloalkenyl.

In some embodiments, in each of Formulas (IV.b), (IV.b.1), (IV.b.2), (IV.b.3), (IV.b.4), (IV.b.5), and (IV.b.6), R² is heterocycloalkyl.

In some embodiments, in each of Formulas (IV.b), (IV.b.1), (IV.b.2), (IV.b.3), (IV.b.4), (IV.b.5), and (IV.b.6), R² is heterocycloalkenyl.

In some embodiments, in each of Formulas (IV.b), (IV.b.1), (IV.b.2), (IV.b.3), (IV.b.4), (IV.b.5), and (IV.b.6). Z is (═O).

In some embodiments, in each of Formulas (IV.b), (IV.b.1), (IV.b.2), (IV.b.3), (IV.b.4), (IV.b.5), and (IV.b.6), Z is (═S).

In some embodiments, in each of Formulas (IV.b), (IV.b.1), (IV.b.2), (IV.b.3), (IV.b.4), (IV.b.5), and (IV.b.6), Z is (═N(R¹³)).

In some embodiments, in each of Formulas (IV.b), (IV.b.1), (IV.b.2), (IV.b.3), (IV.b.4), (IV.b.5), and (IV.b.6), Z is (═N(CN)).

In some embodiments, in each of Formulas (IV.b), (IV.b.1), (IV.b.2), (IV.b.3), (IV.b.4), (IV.b.5), and (IV.b.6), Z is (═N(OR¹⁴)).

In some embodiments, in each of Formulas (IV.b), (IV.b.1), (IV.b.2), (IV.b.3), (IV.b.4), (IV.b.5), and (IV.b.6), Z is (═N(R¹⁵)(R¹⁶)).

In some embodiments, in each of Formulas (IV.b), (IV.b.1), (IV.b.2), (IV.b.3), (IV.b.4), (IV.b.5), and (IV.b.6), Z is (═C(R¹⁷)(R¹⁸)).

In some embodiments, in each of Formulas (IV.b), (IV.b.1), (IV.b.2), (IV.b.3), (IV.b.4), (IV.b.5), and (IV.b.6), R² is —O(Z)R⁷, and Z is (═O).

In some embodiments, in each of Formulas (IV.b), (IV.b.1), (IV.b.2), (IV.b.3), (IV.b.4), (IV.b.5), and (IV.b.6), R² is —C(O)H.

In some embodiments, in each of Formulas (IV.b), (IV.b.1), (IV.b.2), (IV.b.3), (IV.b.4), (IV.b.5), and (IV.b.6), R² is —C(O)alkyl.

In some embodiments, in each of Formulas (IV.b), (IV.b.1), (IV.b.2), (IV.b.3), (IV.b.4), (IV.b.5), and (IV.b.6), R² is —C(O)CH₃.

In some embodiments, in each of Formulas (IV.b), (IV.b.1), (IV.b.2), (IV.b.3), (IV.b.4), (IV.b.5), and (IV.b.6), R² is —C(O)R⁷, wherein said R⁷ is alkyl substituted with one or more substituents, which can be the same or different, each substituent being independently selected from the group consisting of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁶, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (IV.b), (IV.b.1), (IV.b.2), (IV.b.3), (IV.b.4), (IV.b.5), and (IV.b.6), R² is —C(O)R⁷, wherein said R⁷ is alkyl substituted with one to three substituents, which can be the same or different, each substituent being independently selected from the group consisting of —OR¹⁹, and cycloalkyl.

In some embodiments, in each of Formulas (IV.b), (IV.b.1), (IV.b.2), (IV.b.3), (IV.b.4), (IV.b.5), and (IV.b.6), R² is —C(O)R⁷, wherein said R⁷ is alkyl, wherein said alkyl is substituted with alkyl and —OH.

In some embodiments, in each of Formulas (IV.b), (IV.b.1), (IV.b.4), (IV.b.5), and (IV.b.6), R² is —C(O)R⁷, wherein said R⁷ is alkyl substituted with one to three substituents, which can be the same or different, each substituent being independently selected from the group consisting of —OH, —NH₂, and cyclopropyl.

In some embodiments, in each of Formulas (IV.b), (IV.b.1), (IV.b.2), (IV.b.3), (IV.b.4), (IV.b.5), and (IV.b.6), R² is —C(O)R⁷, wherein said R⁷ is alkyl substituted with one to two substituents, which can be the same or different, each substituent being independently selected from the group consisting of —NH₂, and cyclopropyl.

In some embodiments, in each of Formulas (IV.b), (IV.b.1), (IV.b.2), (IV.b.3), (IV.b.4), (IV.b.5), and (IV.b.6), R² is —C(O)R⁷, wherein said R⁷ is alkyl substituted with —OH.

In some embodiments, in each of Formulas (IV.b), (IV.b.2), (IV.b.3), (IV.b.4), (IV.b.5), and (IV.b.6), R² is —C(O)R⁷, wherein said R⁷ is unsubstituted heterocycloalkyl.

In some embodiments, in each of Formulas (IV.b), (IV.b.1), (IV.b.2), (IV.b.3), (IV.b.5), and (IV.b.6), R² is —C(O)R⁷, wherein said R⁷ is substituted heterocycloalkyl.

In some embodiments, in each of Formulas (IV.b), (IV.b.1), (IV.b.2), (IV.b.3), (IV.b.4), (IV.b.5), and (IV.b.6), R² is —C(O)R⁷, wherein said R⁷ is heterocycloalkyl substituted with one or more substituents, which can be the same or different, each substituent being independently selected from the group consisting of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR³²R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (IV.b), (IV.b.1), (IV.b.2), (IV.b.3), (IV.b.4), (IV.b.5), and (IV.b.6), R² is —C(O)R⁷, wherein said R⁷ is selected from the group consisting of substituted piperidine, substituted piperazine, substituted morpholine, substituted pyrrolidine, and substituted azetidine.

In some embodiments, in each of Formulas (IV.b), (IV.b.2), (IV.b.3), (IV.b.5), and (IV.b.6), R² is selected from

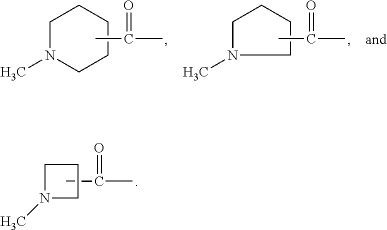

In some embodiments, in each of Formulas (IV.b), (IV.b.1), (IV.b.2), (IV.b.3), (IV.b.4), (IV.b.5), and (IV.b.6), R² is —C(O)NR⁹R¹⁰.

In some embodiments, in each of Formulas (IV.b), (IV.b.1), (IV.b.2), (IV.b.3), (IV.b.4), (IV.b.5), and (IV.b.6), R² is —C(O)NH₂.

In some embodiments, in each of Formulas (IV.b), (IV.b.1), (IV.b.2), (IV.b.3), (IV.b.4), (IV.b.5), and (IV.b.6), R² is —C(O)NR⁹R¹⁰, wherein R⁹ and R¹⁰ can be the same or different, each being independently selected from alkyl.

In some embodiments, in each of Formulas (IV.b), (IV.b.1), (IV.b.2), (IV.b.3), (IV.b.4), (IV.b.5), and (IV.b.6), R² is —C(O)NR⁹R¹⁰, wherein R⁹ is unsubstituted heterocycloalkyl and R¹⁰ is selected from the group consisting of H and alkyl.

In some embodiments, in each of Formulas (IV.b), (IV.b.1), (IV.b.2), (IV.b.3), (IV.b.4), (IV.b.5), and (IV.b.6), R² is —C(O)NR⁹R¹⁰, wherein R⁹ is substituted heterocycloalkyl and R¹⁰ is selected from the group consisting of H and alkyl.

In some embodiments, in each of Formulas (IV.b), (IV.b.1), (IV.b.2), (IV.b.3), (IV.b.4), (IV.b.5), and (IV.b.6), R² is —C(O)NR⁹R¹⁰, wherein R⁹ is heterocycloalkyl substituted with from one to three substituents, which can be the same or different, each substituent being independently selected from alkyl, and R¹⁰ is selected from the group consisting of H and alkyl.

In some embodiments, in each of Formulas (IV.b), (IV.b.1), (IV.b.2), (IV.b.3), (IV.b.4), (IV.b.5), and (IV.b.6), R² is selected from the group consisting of: alkyl, haloalkyl, heteroalkyl, heterohaloalkyl, —C(O)R⁷. —C(O)OR⁸, and —C(O)NR⁹R¹⁰.

In some embodiments, in each of Formulas (IV.b), (IV.b.1), (IV.b.2), (IV.b.3), (IV.b.4), (IV.b.5), and (IV.b.6), R² is selected from the group consisting of

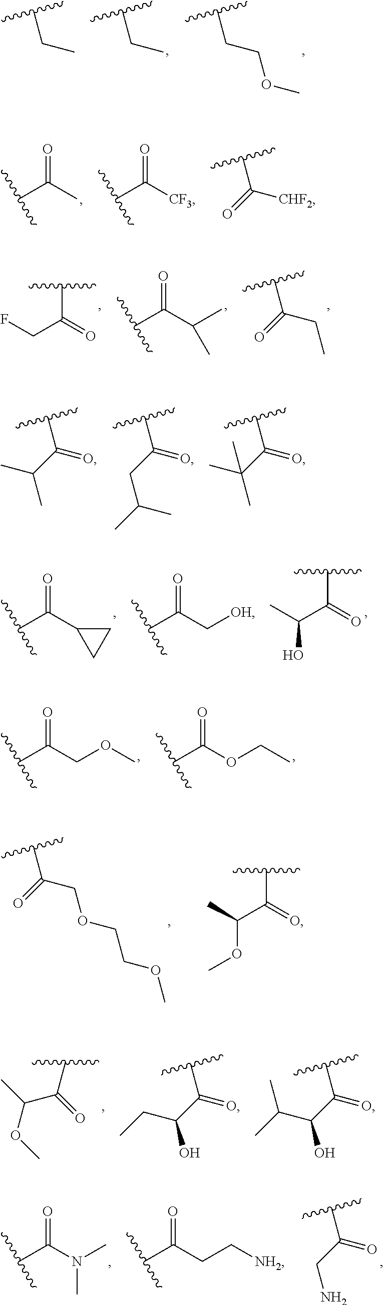

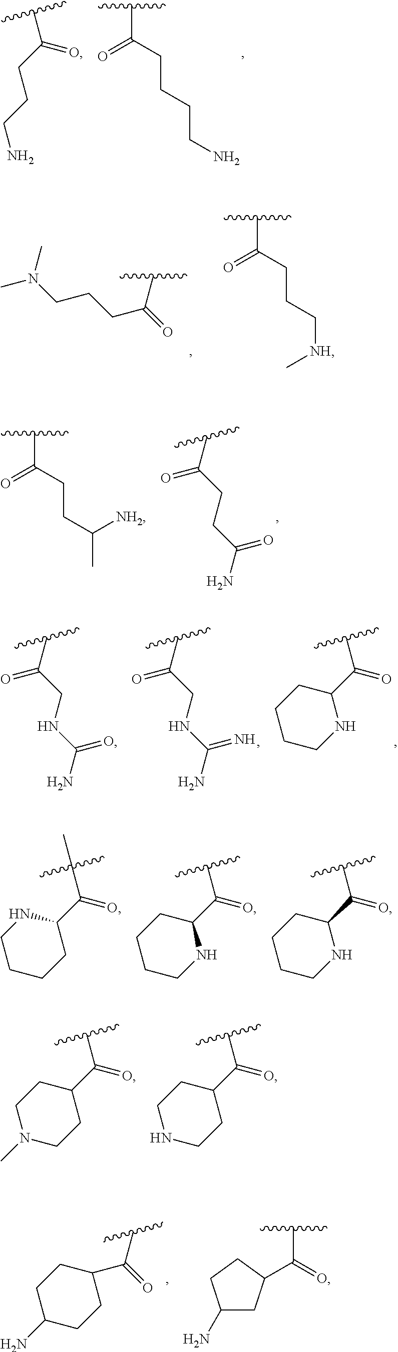

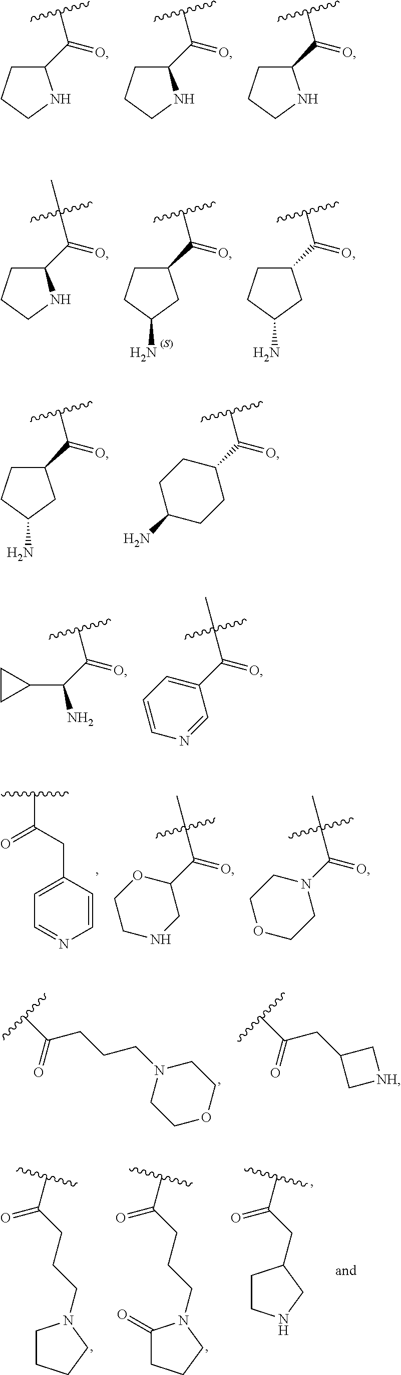

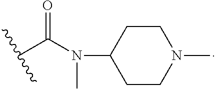

In some embodiments, in each of Formulas (IV.b), (IV.b.1), (IV.b.2), (IV.b.3), (IV.b.4), (IV.b.5), and (IV.b.6), R² is

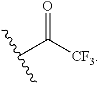

In some embodiments, in each of Formulas (IV.b), (IV.b.1), (IV.b.2), (IV.b.3), (IV.b.4), (IV.b.5), and (IV.b.6), R² is

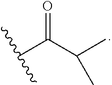

In some embodiments, in each of Formulas (Nip), (IV.b.1), (IV.b.2), (IV.b.3), (IV.b.4), (IV.b.5), and (IV.b.6), R² is

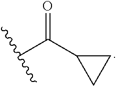

In some embodiments, in each of Formulas (IV.b), (IV.b.1), (IV.b.2), (IV.b.3), (IV.b.5), and (IV.b.6), R² is

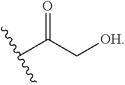

In some embodiments, in each of Formulas (IV.b.), (IV.b.1), (IV.b.2), (IV.b.3), (IV.b.4), (IV.b.5), and (IV.b.6), R² is

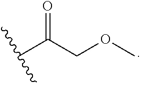

In some embodiments, in each of Formulas (IV.b), (IV.b.1), (IV.b.2), (IV.b.3), (IV.b.4), (IV.b.5), and (IV.b.6), R² is

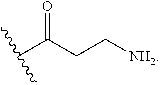

In some embodiments, in each of Formulas (IV.b), (IV.b.1), (IV.b.2), (IV.b.3), (IV.b.4), (IV.b.5), and (IV.b.6), R² is

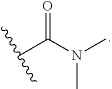

In some embodiments, in each of Formulas (IV.b), (IV.b.1), (IV.b.2), (IV.b.3), (IV.b.4), (IV.b.5), and (IV.b.6), R² is

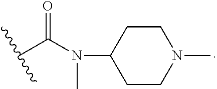

In some embodiments, in each of Formulas (IV.b), (IV.b.1), (IV.b.2), (IV.b.3), (IV.b.5), and (IV.b.6), p is 0 and R³ is not present.

In some embodiments, in each of Formulas (IV.b), (IV.b.1), (IV.b.2), (IV.b.3), (IV.b.4), (IV.b.5), and (IV.b.6), p is 1.

In some embodiments, in each of Formulas (IV.b), (IV.b.1), (IV.b.2), (IV.b.3), (IV.b.4), (IV.b.5), and (IV.b.6), p is 2.

In some embodiments, in each of Formulas (IV.b), (IV.b.1), (IV.b.2), (IV.b.3), (IV.b.4), (IV.b.5), and (IV.b.6), p is 3.

In some embodiments, in each of Formulas (IV.b), (IV.b.1), (IV.b.2), (IV.b.3), (IV.b.4), (IV.b.5), and (IV.b.6), p is 4.

In some embodiments, in each of Formulas (IV.b), (IV.b.1), (IV.b.3), (IV.b.4), (IV.b.5), and (IV.b.6), p is ≧2 and at least two groups R³ are attached to the same ring atom.

In some embodiments, in each of Formulas (IV.b), (IV.b.1), (IV.b.3), (IV.b.4), (IV.b.5), and (IV.b.6), p is 1 and R³ is independently selected from the group consisting of alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²³, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (IV.b), (IV.b.1), (IV.b.2), (IV.b.3), (IV.b.4), (IV.b.5), and (IV.b.6), p is 2, 3, or 4 and each R³ is independently selected from the group consisting of alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (IV.b), (IV.b.1), (IV.b.2), (IV.b.3), (IV.b.4), (IV.b.5), and (IV.b.6), p is 2, 3, or 4 and at least two groups R³ are bound to the same ring carbon atom, wherein each R³, which may be the same or different, is independently selected from the group consisting of alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂: —OR¹⁹, —OC(O)OR²³, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (IV.b), (IV.b.1), (IV.b.2), (IV.b.3), (IV.b.4), (IV.b.5), and (IV.b.6), p is 2, 3, or 4 and at least two groups R³ are bound to the same ring carbon atom, wherein two R³ groups, which may be the same or different, together with the carbon atom to which they are attached, form a cycloalkyl, a cycloalkenyl, a heterocycloalkyl ring containing from one to three heteroatoms selected from the group consisting of N, O, and S, or a heterocycloalkenyl ring containing from one to three heteroatoms selected from the group consisting of N, O, and S.

In one embodiment, in Formula (IV.b), p is 1, 2, 3, or 4, and each R³ is independently selected from the group consisting of alkyl, heteroalkyl, alkenyl, heteroalkenyl, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(S)R²⁴, —C(O)OR²⁰, —SR¹⁹, —S(O)R¹³, —SO₂R¹⁹, —OC(O)R²⁴, —C(O)NR²⁵R²⁶, —NR²³C(N—CN)NR²⁵R²⁶, —NR²³C(O)NR²⁵R²⁶, and —NR²³—C(NH)—NR²⁶R²⁶,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (IV.b), p is 1 and R³ is selected from the group consisting of alkyl, heteroalkyl, alkenyl, and heteroalkenyl,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (IV.b), p is 2, 3, or 4, and any two R³ groups bound to the same ring A atom are taken together to form a —C(O)— group.

In one embodiment, in Formula (IV.b), p is ≧2 and any two R³ groups bound to the same ring A atom are taken together to form a spiroheterocycloalkyl group having from 1 to 3 ring heteroatoms independently selected from the group consisting of —NH—, —NR⁶—, O, S, S(O), and S(O)₂, or spiroheterocycloalkenyl group having from 1 to 3 ring heteroatoms independently selected from the group consisting of —NH—, —NR⁶—, O, S, S(O), and S(O)₂.

In some embodiments, in each of Formulas (IV.b), (IV.b.1), (IV.b.2), (IV.b.3), (IV.b.4), (IV.b.5), and (IV.b.6), R³ is alkyl.

In some embodiments, in each of Formulas (IV.b), (IV.b.1), (IV.b.2), (IV.b.3), (IV.b.4), (IV.b.5), and (IV.b.6), R³ is heteroalkyl.

In some embodiments, in each of Formulas (IV.b), (IV.b.1), (IV.b.2), (IV.b.3), (IV.b.4), (IV.b.5), and (IV.b.6), R³ is alkenyl.

In some embodiments, in each of Formulas (IV.b), (IV.b.1), (IV.b.2), (IV.b.3), (IV.b.4), (IV.b.5), and (IV.b.6), R³ is heteroalkenyl.

In some embodiments, in each of Formulas (IV.b), (IV.b.1), (IV.b.2), (IV.b.3), (IV.b.4), (IV.b.5), and (IV.b.6), R³ is alkynyl.

In some embodiments, in each of Formulas (IV.b), (IV.b.1), (IV.b.2), (IV.b.3), (IV.b.4), (IV.b.5), and (IV.b.6), R³ is heteroalkynyl.

In some embodiments, in each of Formulas (IV.b), (IV.b.1), (IV.b.2), (IV.b.3), (IV.b.4), (IV.b.5), and (IV.b.6), R³ is aryl.

In some embodiments, in each of Formulas (IV.b), (IV.b.1), (IV.b.2), (IV.b.3), (IV.b.4), (IV.b.5), and (IV.b.6), R³ is heteroaryl.

In some embodiments, in each of Formulas (IV.b), (IV.b.1), (IV.b.2), (IV.b.3), (IV.b.4), (IV.b.5), and (IV.b.6), R³ is cycloalkyl.

In some embodiments, in each of Formulas (IV.b), (IV.b.1), (IV.b.2), (IV.b.3), (IV.b.4), (IV.b.5), and (IV.b.6), R³ is cycloalkenyl.

In some embodiments, in each of Formulas (IV.b), (IV.b.1), (IV.b.2), (IV.b.3), (IV.b.4), (IV.b.5), and (IV.b.6), R³ is heterocycloalkyl.

In some embodiments, in each of Formulas (IV.b), (IV.b.1), (IV.b.2), (IV.b.3), (IV.b.4), (IV.b.5), and (IV.b.6), R³ is heterocycloalkenyl.

In some embodiments, in each of Formulas (IV.b), (IV.b.1), (IV.b.2), (IV.b.3), (IV.b.4), (IV.b.5), and (IV.b.6), R³ is halogen.

In some embodiments, in each of Formulas (IV.b), (IV.b.1), (IV.b.2), (IV.b.3), (IV.b.4), (IV.b.5), and (IV.b.6), R³ is —CN.

In some embodiments, in each of Formulas (IV.b), (IV.b.1), (IV.b.2), (IV.b.3), (IV.b.4), (IV.b.5), and (IV.b.6), R³ is —NO₂

In some embodiments, in each of Formulas (IV.b), (IV.b.1), (IV.b.2), (IV.b.3), (IV.b.4), (IV.b.5), and (IV.b.6), R³ is —OR¹⁹.

In some embodiments, in each of Formulas (IV.b), (IV.b.1), (IV.b.2), (IV.b.3), (IV.b.4), (IV.b.5), and (IV.b.6), R³ is —OC(O)OR²⁰.

In some embodiments, in each of Formulas (IV.b), (IV.b.1), (IV.b.2), (IV.b.3), (IV.b.4), (IV.b.5), and (IV.b.6), R³ is —NR²¹R²².

In some embodiments, in each of Formulas (IV.b), (IV.b.1), (IV.b.2), (IV.b.3), (IV.b.4), (IV.b.5), and (IV.b.6), R³ is —NR²³SO₂R²⁴.

In some embodiments, in each of Formulas (IV.b), (IV.b.1), (IV.b.2), (IV.b.3), (IV.b.4), (IV.b.5), and (IV.b.6), R³ is —NR²³C(O)OR²⁰.

In some embodiments, in each of Formulas (IV.b.), (IV.b.1), (IV.b.2), (IV.b.3), (IV.b.4), (IV.b.5), and (IV.b.6), R³ is —NR²³C(O)R²⁴.

In some embodiments, in each of Formulas (IV.b), (IV.b.1), (IV.b.2), (IV.b.3), (IV.b.4), (IV.b.5), and (IV.b.6), R³ is —SO₂NR²⁵R²⁶.

In some embodiments, in each of Formulas (IV.b), (IV.b.1), (IV.b.2), (IV.b.3), (IV.b.4), (IV.b.5), and (IV.b.6), R³ is —C(O)R²⁴.

In some embodiments, in each of Formulas (IV.b), (IV.b.1), (IV.b.2), (IV.b.3), (IV.b.4), (IV.b.5), and (IV.b.6), R³ is —C(O)OR²⁰.

In some embodiments, in each of Formulas (IV.b), (IV.b.1), (IV.b.2) IV.b.3), (IV.b.4), (IV.b.5), and (IV.b.6), R³ is —SR¹⁹.

In some embodiments, in each of Formulas (IV.b), (IV.b.1), (IV.b.2), (IV.b.3), (IV.b.4), (IV.b.5), and (IV.b.6), R³ is —S(O)R¹⁹.

In some embodiments, in each of Formulas (IV.b), (IV.b.1), (IV.b.2), (IV.b.3), (IV.b.4), (IV.b.5), and (IV.b.6), R³ is —SO₂R¹⁹.

In some embodiments, in each of Formulas (IV.b), (IV.b.1), (IV.b.2), (IV.b.3), (IV.b.4), (IV.b.5), and (IV.b.6), R³ is —OC(O)R²⁴.

In some embodiments, in each of Formulas (IV.b), (IV.b.1), (IV.b.2), (IV.b.3), (IV.b.4), (IV.b.5), and (IV.b.6), R³ is —C(O)NR²⁵R²⁶.

In some embodiments, in each of Formulas (IV.b), (IV.b.1), (IV.b.2), (IV.b.3), (IV.b.4), (IV.b.5), and (IV.b.6), R³ is —NR²³C(N—CN)NR²⁵R²⁶.

In some embodiments, in each of Formulas (IV.b), (IV.b.1), (IV.b.2), (IV.b.3), (IV.b.4), (IV.b.5), and (IV.b.6), R³ is —NR²³C(O)NR²⁵R²⁶

In some embodiments, in each of Formulas (IV.b), (IV.b.1), (IV.b.2), (IV.b.3), (IV.b.4), (IV.b.5), and (IV.b.6), R³ is selected from the group consisting of: methyl, ethyl, propyl (straight or branched), butyl (straight or branched), pentyl (straight or branched), phenyl,

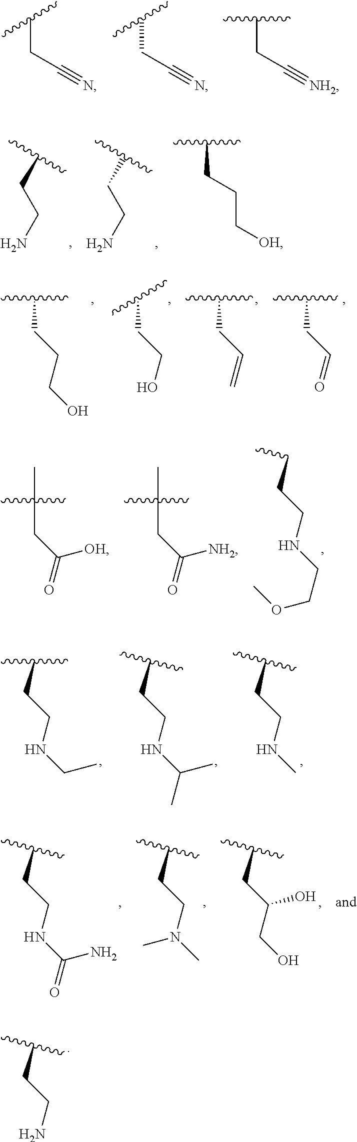

In some embodiments, in each of Formulas (IV.b), (IV.b.1), (IV.b.2), (IV.b.3), (IV.b.4), (IV.b.5), and (IV.b.6), when E is —NR⁶—, R³ is absent.

In one embodiment, the compounds of the invention have a structure shown in Formula (V.a) and include pharmaceutically acceptable salts, solvates, esters, prodrugs, or isomers of said compounds:

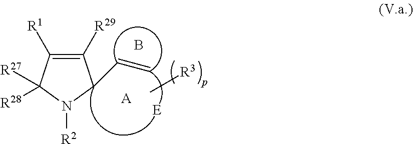

wherein R¹, R², R³, R²⁷, R²⁸, R²⁹, p, E, ring A, and ring B are selected independently of each other and wherein:
ring A (including E and the unsaturation shown) is a 7- to 8-membered cycloalkenyl or heterocycloalkenyl ring;
E is selected from the group consisting of —O—, —S—, —S(O)—, —S(O)₂—, —C(R⁴)(R⁸)—, —N(R⁶)—, —N(C(Y)R⁷)—, —N(C(Y)OR⁸)—, —N(C(Y)N(R⁹)(R¹⁰))—, —C(O)—N(R¹¹)—, —N(R¹¹)—C(O)—, —S(O)₂—N(R¹¹)—, —N(R¹¹)—S(O)₂—, —C(O)—O—, —O—C(O)—, —O—N(R⁸)—, —N(R⁶)—O—, —N(R⁶)—N(R¹²)—, —N═N—, —C(R⁷)═N—, —C(O)—C(R⁷)═N—, —C(O)—N═N—, —O—C(Y)—N(R¹¹)—, —N(R¹¹)—C(Y)—O—, —N(R¹¹)—C(Y)—N(R¹²)—, —C(Y)—N(R¹¹)—O—, —C(Y)—N(R¹¹)—N(R¹²)—, —O—N(R¹¹)—C(Y)—, and —N(R¹²)—N(R¹¹)—C(Y)—;
ring B is a substituted or unsubstituted aromatic ring;
and p, R¹, R², R³, R⁴, R⁵, R⁶, R⁷, R⁸, R⁹, R¹⁰, R¹¹, R¹², R²⁷, R²⁸, R²⁹, Y, and the optional substituents on ring B are as defined in each of the embodiments described above in Formula (I).

In one embodiment, Formula (V.a) has the general structure shown in Formula (V.a.1):

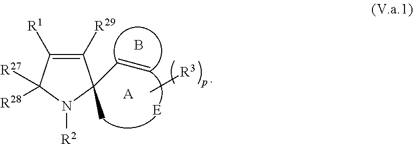

In one embodiment, Formula (V.a) has the general structure shown in Formula (V.a.2);

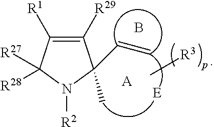

In one embodiment, Formula (V.a) has the general structure shown in Formula (V.a.3):

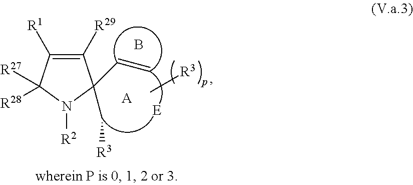

In one embodiment, Formula (V.a) has the general structure shown in Formula (V.a.4):

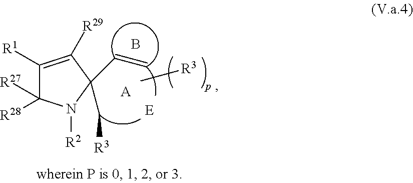

In one embodiment, Formula (V.a) has the general structure shown in Formula (V.a.5):

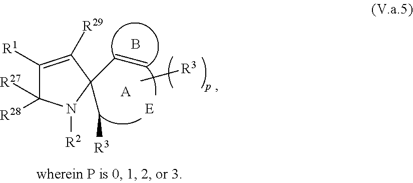

In one embodiment, Formula (V.a) has the general structure shown in Formula (V.a.6):

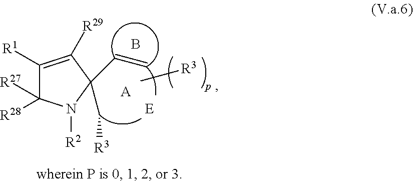

In some embodiments, in each of Formulas (V.a), (V.a.1), (V.a.2), (V.a.3), (V.a.4), (V.a.5), and (V.a.6), ring A is a cycloalkenyl ring and E is —C(R⁴)(R⁵)—.

In some embodiments, in each of Formulas (VA), (V.a.1), (V.a.2), (V.a.3), (V.a.4), (V.a.5), and (V.a.6), ring A is a heterocycloalkenyl ring and E is selected from the group consisting of —O—, —S—, —S(O)—, —S(O)₂—, and —N(R⁶)—.

In some embodiments, in each of Formulas (V.a), (V.a.1), (V.a.2), (V.a.3), (V.a.4), (V.a.5), and (V.a.6), E is selected from the group consisting of —O—, —S—, —S(O)—, —S(O)₂—, and —N(R⁶)—, wherein R⁶ is selected from the group consisting of H, alkyl, —C(O)R²⁴, and —C(S)R²⁴.

In some embodiments, in each of Formulas (V.a), (V.a.1), (V.a.2), (V.a.3), (V.a.4), (V.a.5), and (V.a.6), E is selected from the group consisting of —O— and —N(R⁶)—, wherein R⁶ is selected from the group consisting of H, alkyl, —C(O)R²⁴, and —C(S)R²⁴.

In some embodiments, in each of Formulas (V.a), (V.a.1), (V.a.2), (V.a.3), (V.a.4), (V.a.5), and (V.a.6), E is —O—.

In some embodiments, in each of Formulas (V.a), (V.a.1), (V.a.2), (V.a.3), (V.a.4), (V.a.5), and (V.a.6), E is —S—.

In some embodiments, in each of Formulas (V.a), (V.a.1), (V.a.2), (V.a.3), (V.a.4), (V.a.5), and (V.a.6), E is —S(O)—.

In some embodiments, in each of Formulas (V.a), (V.a.1), (V.a.2), (V.a.3), (V.a.4), (V.a.5), and (V.a.6). E is —S(O)₂—.

In some embodiments, in each of Formulas (V.a), (V.a.1), (V.a.2), (V.a.3), (V.a.4), (V.a.5), and (V.a.6), E is —C(R⁴)(R⁵)—.

In some embodiments, in each of Formulas (V.a), (V.a.1), (V.a.2), (V.a.3), (V.a.4), (V.a.5), and (V.a.6), E is —N(R⁶)—.

In some embodiments, in each of Formulas (V.a), (V.a.1), (V.a.2), (V.a.3), (V.a.4), (V.a.5), and (V.a.6), E is —N(C(Y)R⁷)—.

In some embodiments, in each of Formulas (V.a), (V.a.1), (V.a.2), (V.a.3), (V.a.4), (V.a.5), and (V.a.6), E is —N(C(Y)OR⁸)—.

In some embodiments, in each of Formulas (V.a), (V.a.1), (V.a.2), (V.a.3), (V.a.4), (V.a.5), and (V.a.6), E is —N(C(Y)N(R⁹)(R¹⁰))—.

In some embodiments, in each of Formulas (V.a), (V.a.1), (V.a.2), (V.a.3), (V.a.4), (V.a.5), and (V.a.6), E is —C(O)—N(R¹¹)—.

In some embodiments, in each of Formulas (V.a), (V.a.1), (V.a.2), (V.a.3), (V.a.4), (V.a.5), and (V.a.6), E is —N(R¹¹)—C(O)—.

In some embodiments, in each of Formulas (V.a), (V.a.1), (V.a.2), (V.a.3), (V.a.4), (V.a.5), and (V.a.6), E is —S(O)₂—N(R¹¹)—.

In some embodiments, in each of Formulas (V.a), (V.a.1), (V.a.2), (V.a.3), (V.a.4), (V.a.5), and (V.a.6), E is —N(R¹¹)—S(O)₂—.

In some embodiments, in each of Formulas (V.a), (V.a.1), (V.a.2), (V.a.3), (V.a.4), (V.a.5), and (V.a.6), E is —C(O)—O—.

In some embodiments, in each of Formulas (V.a), (V.a.1), (V.a.2), (V.a.3), (V.a.4), (V.a.5), and (V.a.6), E is —O—C(O)—.

In some embodiments, in each of Formulas (V.a), (V.a.1), (V.a.2), (V.a.3), (V.a.4), (V.a.5), and (V.a.6), E is —O—N(R⁶)—.

In some embodiments, in each of Formulas (V.a), (V.a.1), (V.a.2), (V.a.3), (V.a.4), (V.a.5), and (V.a.6), E is —N(R⁶)—O—.

In some embodiments, in each of Formulas (V.a), (V.a.1), (V.a.2), (V.a.3), (V.a.4), (V.a.5), and (V.a.6), E is —N(R⁶)—N(R¹²)—.

In some embodiments, in each of Formulas (V.a.), (V.a.1), (V.a.2), (V.a.3), (V.a.4), (V.a.5), and (V.a.6), E is —N═N—.

In some embodiments, in each of Formulas (V.a), (V.a.1), (V.a.2), (V.a.3), (V.a.4), (V.a.5), and (V.a.6), E is —C(R⁷)═N—.

In some embodiments, in each of Formulas (V.a), (V.a.1), (V.a.2), (V.a.3), (V.a.4), (V.a.5), and (V.a.6), E is —C(O)—C(R⁷)═N—.

In some embodiments, in each of Formulas (V.a), (V.a.1), (V.a.2), (V.a.3), (V.a.4), (V.a.5), and (V.a.6), E is —C(O)—N═N—.

In some embodiments, in each of Formulas (V.a), (V.a.1), (V.a.2), (V.a.3), (V.a.4), (V.a.5), and (V.a.6), E is —O—C(Y)—N(R¹¹)—.

In some embodiments, in each of Formulas (V.a), (V.a.1), (V.a.2), (V.a.3), (V.a.4), (V.a.5), and (V.a.6), E is —N(R¹¹)—C(Y)—O—.

In some embodiments, in each of Formulas (V.a), (V.a.1), (V.a.2), (V.a.3), (V.a.4), (V.a.5), and (V.a.6), E is —N(R¹¹)—C(Y)—N(R¹²)—.

In some embodiments, in each of Formulas (V.a), (V.a.1), (V.a.2), (V.a.3), (V.a.4), (V.a.5), and (V.a.6). E is —C(Y)—N(R¹¹)—O—.

In some embodiments, in each of Formulas (V.a), (V.a.1), (V.a.2), (V.a.3), (V.a.4), (V.a.5), and (V.a.6), E is —C(Y)—N(R¹¹)—N(R¹²)—.

In some embodiments, in each of Formulas (V.a), (V.a.1), (V.a.2), (V.a.3), (V.a.4), (V.a.5), and (V.a.6), E is —O—N(R¹¹)—C(Y)—.

In some embodiments, in each of Formulas (V.a), (V.a.1), (V.a.2), (V.a.3), (V.a.4), (V.a.5), and (V.a.6), E is —N(R¹²)—N(R¹¹)—C(Y)—.

In some embodiments, in each of Formulas (V.a), (V.a.1), (V.a.2), (V.a.3), (V.a.4), (V.a.5), and (V.a.6), Y is (═O).

In some embodiments, in each of Formulas (V.a), (V.a.1), (V.a.2), (V.a.3), (V.a.4), (V.a.5), and (V.a.6), Y is (═S).

In some embodiments, in each of Formulas (V.a), (V.a.1), (V.a.2), (V.a.3), (V.a.4), (V.a.5), and (V.a.6), Y is (═N(R¹³)).

In some embodiments, in each of Formulas (V.a), (V.a.1), (V.a.2), (V.a.3), (V.a.4), (V.a.5), and (V.a.6), Y is (═N(CN)).

In some embodiments, in each of Formulas (V.a), (V.a.1), (V.a.2), (V.a.3), (V.a.4), (V.a.5), and (V.a.6), Y is (═N(OR¹⁴)).

In some embodiments, in each of Formulas (V.a), (V.a.1), (V.a.2), (V.a.3), (V.a.4), (V.a.5), and (V.a.6), Y is (═N(R¹⁵)(R¹⁶)).

In some embodiments, in each of Formulas (V.a), (V.a.1), (V.a.2), (V.a.3), (V.a.4), (V.a.5), and (V.a.6), Y is (═C(R¹⁷)(R¹⁸)).

In some embodiments, in each of Formulas (V.a), (V.a.1), (V.a.2), (V.a.3), (V.a.4), (V.a.5), and (V.a.6), B is an unsubstituted aromatic ring.

In some embodiments, in each of Formulas (V.a), (V.a.1), (V.a.2), (V.a.3), (V.a.4), (V.a.5), and (V.a.6), B is an unsubstituted benzo ring, and Formula (IV.a.) has the general structure:

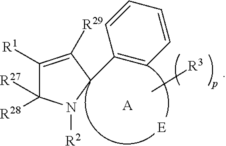

In some embodiments, in each of Formulas (V.a), (V.a.1), (V.a.2), (V.a.3), (V.a.4), (V.a.5), and (V.a.6), B is an aromatic ring which is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²³R²⁶, —NR²³C(N—CN)NR²⁵R²⁶ and —NR²³C(O)NR²⁵R²⁶.

In some embodiments, in each of Formulas (V.a), (V.a.1), (V.a.2), (V.a.3), (V.a.4), (V.a.5), and (V.a.6), B is a benzo ring which is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (V.a), (V.a.1), (V.a.2), (V.a.3), (V.a.4), (V.a.5), and (V.a.6), R¹ is unsubstituted aryl.

In some embodiments, in each of Formulas (V.a), (V.a.1), (V.a.2), (V.a.3), (V.a.4), (V.a.5), and (V.a.6), R¹ is unsubstituted phenyl.

In some embodiments, in each of Formulas (V.a), (V.a.1), (V.a.2), (V.a.3), (V.a.4), (V.a.5), and (V.a.6), R¹ is unsubstituted naphthyl.

In some embodiments, in each of Formulas (V.a), (V.a.1), (V.a.2), (V.a.3), (V.a.4), (V.a.5), and (V.a.6), R¹ is substituted aryl.

In some embodiments, in each of Formulas (V.a), (V.a.1), (V.a.2), (V.a.3), (V.a.4), (V.a.5), and (V.a.6), R¹ is substituted phenyl.

In some embodiments, in each of Formulas (V.a), (V.a.1), (V.a.2), (V.a.3), (V.a.4), (V.a.5), and (V.a.6), R¹ is substituted naphthyl.

In some embodiments, in each of Formulas (V.a), (V.a.1), (V.a.2), (V.a.3), (V.a.4), (V.a.5), and (V.a.6), R¹ is aryl substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (V.a), (V.a.1), (V.a.2), (V.a.3), (V.a.4), (V.a.5), and (V.a.6), R¹ is phenyl substituted with one or more substituents, which can be the same or different, each substituent being independently selected from the group consisting halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (V.a), (V.a.1), (V.a.2), (V.a.3), (V.a.4), (V.a.5), and (V.a.6), R¹ is phenyl substituted with one to four substituents, which can be the same or different, each substituent being independently selected from the group consisting of halo, —OH, —CN, —NO₂, —NR²¹R²², and haloalkyl.

In some embodiments, in each of Formulas (V.a), (V.a.1), (V.a.2), (V.a.3), (V.a.4), (V.a.5), and (V.a.6), R¹ is selected from the group consisting of:

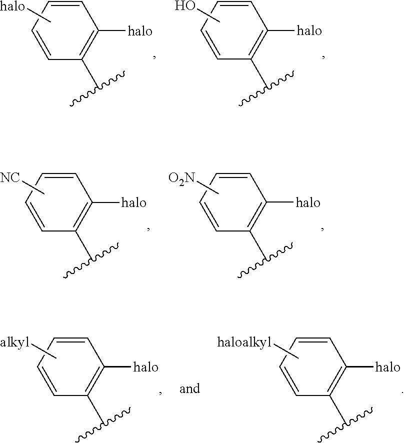

In some embodiments, in each of Formulas (V.a), (V.a.1), (V.a.2), (V.a.3), (V.a.4), (V.a.5), and (V.a.6), R¹ is:

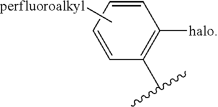

In some embodiments, in each of Formulas (V.a), (V.a.1), (V.a.2), (V.a.3), (V.a.4), (V.a.5), and (V.a.6), R¹ is phenyl substituted with one to three fluoro groups.

In some embodiments, in each of Formulas (V.a), (V.a.1), (V.a.2), (V.a.3), (V.a.4), (V.a.5), and (V.a.6), R¹ is phenyl substituted with two fluoro groups.

In some embodiments, in each of Formulas (V.a), (V.a.1), (V.a.2), (V.a.3), (V.a.4), (V.a.5), and (V.a.6), R¹ is phenyl substituted with one fluoro group.

In some embodiments, in each of Formulas (V.a), (V.a.1), (V.a.2), (V.a.3), (V.a.4), (V.a.5), and (V.a.6), R¹ is:

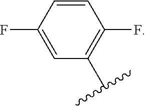

In some embodiments, in each of Formulas (V.a), (V.a.1), (V.a.2), (V.a.3), (V.a.4), (V.a.5), and (V.a.6), R²⁷, R²⁸, and R²⁹ are each independently selected from the group consisting of H and alkyl.

In some embodiments, in each of Formulas (V.a), (V.a.1), (V.a.2), (V.a.3), (V.a.4), (V.a.5), and (V.a.6), R¹ is:

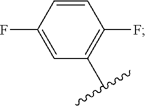

and R²⁷, R²⁸, and R²⁹ are each independently selected from the group consisting of H and alkyl.

In some embodiments, in each of Formulas (V.a), (V.a.1), (V.a.2), (V.a.3), (V.a.4), (V.a.5), and (V.a.6), R² is —C(Z)R⁷.

In some embodiments, in each of Formulas (V.a), (V.a.1), (V.a.2), (V.a.3), (V.a.4), (V.a.5), and (V.a.6), R² is —C(Z)NR⁹R¹⁰.

In some embodiments, in each of Formulas (V.a), (V.a.1), (V.a.2), (V.a.3), (V.a.4), (V.a.5), and (V.a.6), R² is —C(Z)OR⁸.

In some embodiments, in each of Formulas (V.a), (V.a.1), (V.a.2), (V.a.3), (V.a.4), (V.a.5), and (V.a.6), R² is —SO₂NR⁹R¹⁰.

In some embodiments, in each of Formulas (V.a), (V.a.1), (V.a.2), (V.a.3), (V.a.4), (V.a.5), and (V.a.6), R² is alkyl.

In some embodiments, in each of Formulas (V.a), (V.a.1), (V.a.2), (V.a.3), (V.a.4), (V.a.5), and (V.a.6), R² is heteroalkyl.

In some embodiments, in each of Formulas (V.a), (V.a.1), (V.a.2), (V.a.3), (V.a.4), (V.a.5), and (V.a.6), R² is aryl.

In some embodiments, in each of Formulas (VA, (V.a.1), (V.a.2), (V.a.3), (V.a.4), (V.a.5), and (V.a.6), R² is heteroaryl.

In some embodiments, in each of Formulas (V.a), (V.a.1), (V.a.2), (V.a.3), (V.a.4), (V.a.5), and (V.a.6), R² is cycloalkyl.

In some embodiments, in each of Formulas (V.a), (V.a.1), (V.a.2), (V.a.3), (V.a.4), (V.a.5), and (V.a.6), R² is cycloalkenyl.

In some embodiments, in each of Formulas (V.a), (V.a.1), (V.a.2), (V.a.3), (V.a.4), (V.a.5), and (V.a.6), R² is heterocycloalkyl.

In some embodiments, in each of Formulas (V.a), (V.a.1), (V.a.2), (V.a.3), (V.a.4), (V.a.5), and (V.a.6), R² is heterocycloalkenyl.

In some embodiments, in each of Formulas (V.a), (V.a.1), (V.a.2), (V.a.3), (V.a.4), (V.a.5), and (V.a.6), Z is (═O).

In some embodiments, in each of Formulas (V.a), (V.a.1), (V.a.2), (V.a.3), (V.a.4), (V.a.5), and (V.a.6), Z is (═S).

In some embodiments, in each of Formulas (V.a), (V.a.1), (V.a.2), (V.a.3), (V.a.4), (V.a.5), and (V.a.6), Z is (═N(R¹³)).

In some embodiments, in each of Formulas (V.a), (V.a.1), (V.a.2), (V.a.3), (V.a.4), (V.a.5), and (V.a.6), Z is (═N(CN)).

In some embodiments, in each of Formulas (V.a), (V.a.1), (V.a.2), (V.a.3), (V.a.4), (V.a.5), and (V.a.6), Z is (═N(OR¹⁴)).

In some embodiments, in each of Formulas (V.a), (V.a.1), (V.a.2), (V.a.3), (V.a.4), (V.a.5), and (V.a.6), Z is (═N(R¹⁵)(R¹⁶)).

In some embodiments, in each of Formulas (V.a), (V.a.1), (V.a.2), (V.a.3), (V.a.4), (V.a.5), and (V.a.6), Z is (═C(R¹⁷)(R¹⁸)).

In some embodiments, in each of Formulas (V.a), (V.a.1), (V.a.2), (V.a.3), (V.a.4), (V.a.5), and (V.a.6), R² is —C(Z)R⁷, and Z is (═O).

In some embodiments, in each of Formulas (V.a), (V.a.1), (V.a.2), (V.a.3), (V.a.4), (V.a.5), and (V.a.6), R² is —C(O)H.

In some embodiments, in each of Formulas (V.a), (V.a.1), (V.a.2), (V.a.3), (V.a.4), (V.a.5), and (V.a.6), R² is —C(O)alkyl.

In some embodiments, in each of Formulas (V.a), (V.a.1), (V.a.2), (V.a.3), (V.a.4), (V.a.5), and (V.a.6), R² is —C(O)CH₃.

In some embodiments, in each of Formulas (V.a), (V.a.1), (V.a.2), (V.a.3), (V.a.4), (V.a.5), and (V.a.6), R² is —C(O)R⁷, wherein said R⁷ is alkyl substituted with one or more substituents, which can be the same or different, each substituent being independently selected from the group consisting of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (V.a), (V.a.1), (V.a.2), (V.a.3), (V.a.4), (V.a.5), and (V.a.6), R² is —C(O)R⁷, wherein said R⁷ is alkyl substituted with one to three substituents, which can be the same or different, each substituent being independently selected from the group consisting of —OR¹⁹, —NR²¹R²², and cycloalkyl.

In some embodiments, in each of Formulas (V.a), (V.a.1), (V.a.2), (V.a.3), (V.a.4), (V.a.5), and (V.a.6), R² is —C(O)R⁷, wherein said R⁷ is alkyl, wherein said alkyl is substituted with alkyl and —OH.

In some embodiments, in each of Formulas (V.a), (V.a.1), (V.a.2), (V.a.3), (V.a.4), (V.a.5), and (V.a.6), R² is —C(O)R⁷, wherein said R⁷ is alkyl substituted with one to three substituents, which can be the same or different, each substituent being independently selected from the group consisting of —OH, —NH₂, and cyclopropyl.

In some embodiments, in each of Formulas (V.a), (V.a.1), (V.a.2), (V.a.3), (V.a.4), (V.a.5), and (V.a.6), R² is —C(O)R⁷, wherein said R⁷ is alkyl substituted with one to two substituents, which can be the same or different, each substituent being independently selected from the group consisting of —NH₂, and cyclopropyl.

In some embodiments, in each of Formulas (V.a), (V.a.1), (V.a.2), (V.a.3), (V.a.4), (V.a.5), and (V.a.6), R² is —C(O)R⁷, wherein said R⁷ is alkyl substituted with —OH.

In some embodiments, in each of Formulas (V.a), (V.a.1), (V.a.2), (V.a.3), (V.a.4), (V.a.5), and (V.a.6), R² is —C(O)R⁷, wherein said R⁷ is unsubstituted heterocycloalkyl.

In some embodiments, in each of Formulas (V.a), (V.a.1), (V.a.2), (V.a.3), (V.a.4), (V.a.5), and (V.a.6), R² is —C(O)R⁷, wherein said R⁷ is substituted heterocycloalkyl.

In some embodiments, in each of Formulas (V.a), (V.a.1), (V.a.2), (V.a.3), (V.a.4), (V.a.5), and (V.a.6), R² is —C(O)R⁷, wherein said R⁷ is heterocycloalkyl substituted with one or more substituents, which can be the same or different, each substituent being independently selected from the group consisting of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (V.a), (V.a.1), (V.a.2), (V.a.3), (V.a.4), (V.a.5), and (V.a.6), R² is —C(O)R⁷, wherein said R⁷ is selected from the group consisting of substituted piperidine, substituted piperazine, substituted morpholine, substituted pyrrolidine, and substituted azetidine.

In some embodiments, in each of Formulas (V.a), (V.a.1), (V.a.2), (V.a.3), (V.a.4), (V.a.5), and (V.a.6), R² is selected from:

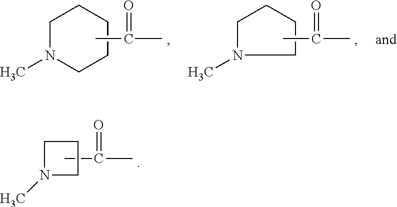

In some embodiments, in each of Formulas (V.a), (V.a.1), (V.a.2), (V.a.3), (V.a.4), (V.a.5), and (V.a.6), R² is —C(O)NR⁹R¹⁰.

In some embodiments, in each of Formulas (V.a), (V.a.1), (V.a.2), (V.a.3), (V.a.4), (V.a.5), and (V.a.6), R² is —C(O)NH₂.

In some embodiments, in each of Formulas (V.a), (V.a.1), (V.a.2), (V.a.3), (V.a.4), (V.a.5), and (V.a.6), R² is —C(O)NR⁹R¹⁰, wherein R⁹ and R¹⁰ can be the same or different, each being independently selected from alkyl.

In some embodiments, in each of Formulas (V.a), (V.a.1), (V.a.2), (V.a.3), (V.a.4), (V.a.5), and (V.a.6), R² is —C(O)NR⁹R¹⁰, wherein R⁹ is unsubstituted heterocycloalkyl and R¹⁹ is selected from the group consisting of H and alkyl.

In some embodiments, in each of Formulas (V.a), (V.a.1), (V.a.2), (V.a.3), (V.a.4), (V.a.5), and (V.a.6), R² is —C(O)NR⁹R¹⁰, wherein R⁹ is substituted heterocycloalkyl and R¹⁹ is selected from the group consisting of H and alkyl.

In some embodiments, in each of Formulas (V.a), (V.a.1), (V.a.2), (V.a.3), (V.a.4), (V.a.5), and (V.a.6), R² is —C(O)NR⁹R¹⁰, wherein R⁹ is heterocycloalkyl substituted with from one to three substituents, which can be the same or different, each substituent being independently selected from alkyl, and R¹⁰ is selected from the group consisting of H and alkyl.

In some embodiments, in each of Formulas (V.a), (V.a.1), (V.a.2), (V.a.3), (V.a.4), (V.a.5), and (V.a.6), R² is selected from the group consisting of: alkyl, haloalkyl, heteroalkyl, heterohaloalkyl, —C(O)R⁷. —C(O)OR⁸, and —C(O)NR⁹R¹⁰.

In some embodiments, in each of Formulas (V.a), (V.a.1), (V.a.2), (V.a.3), (V.a.4), (V.a.5), and (V.a.6), R² is selected from the group consisting of

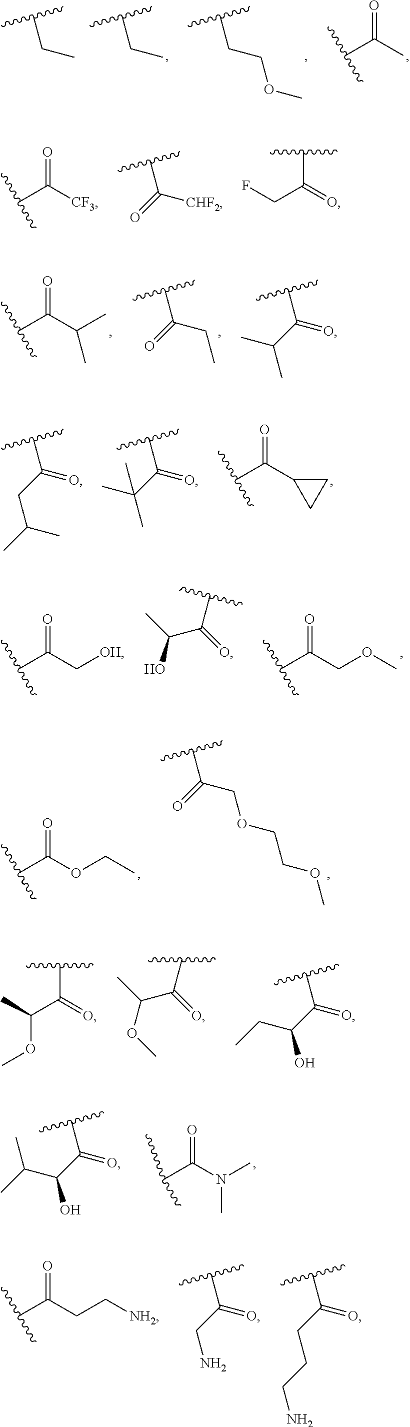

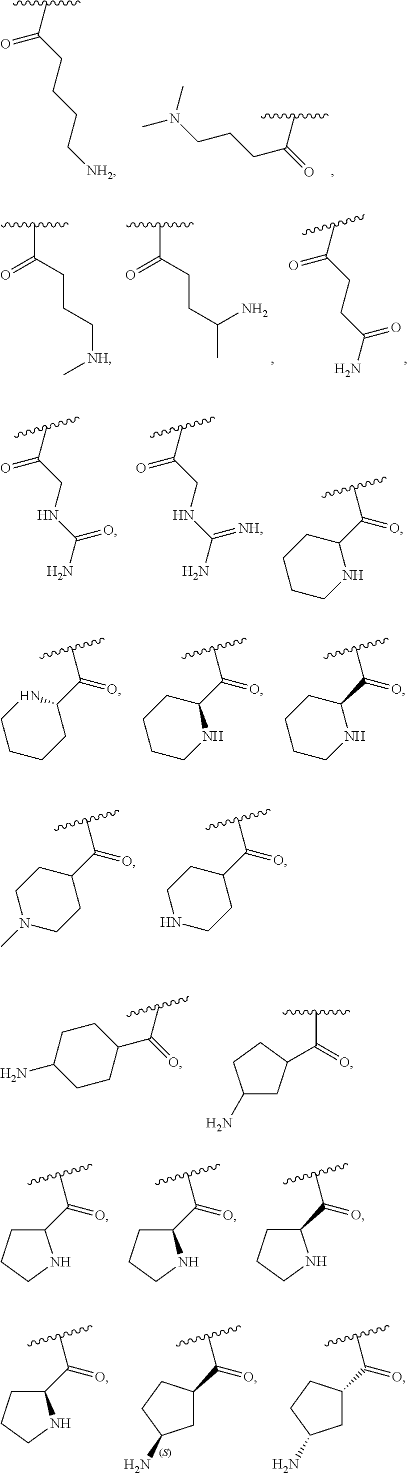

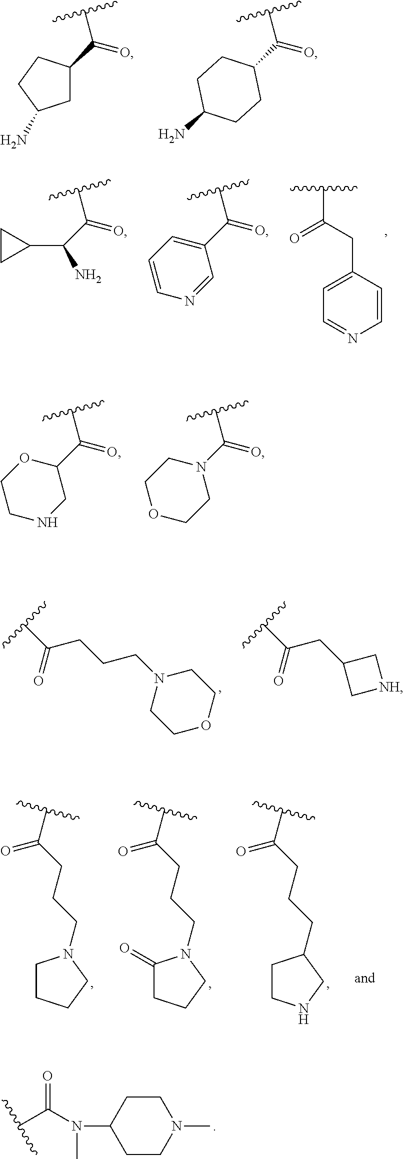

In some embodiments, in each of Formulas (V.a), (V.a.1), (V.a.2), (V.a.3), (V.a.4), (V.a.5), and (V.a.6), R² is

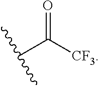

In some embodiments, in each of Formulas (V.a), (V.a.1), (V.a.2), (V.a.3), (V.a.4), (V.a.5), and (V.a.6), R² is

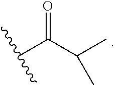

In some embodiments, in each of Formulas (V.a), (V.a.1), (V.a.2), (V.a.3), (V.a.4), (V.a.5), and (V.a.6), R² is

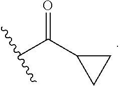

In some embodiments, in each of Formulas (V.a), (V.a.1), (V.a.2), (V.a.3), (V.a.4), (V.a.5), and (V.a.6), R² is

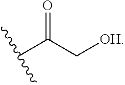

In some embodiments, in each of Formulas (V.a), (V.a.1), (V.a.2), (V.a.3), (V.a.4), (V.a.5), and (V.a.6), R² is

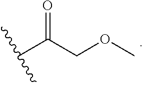

In some embodiments, in each of Formulas (V.a), (V.a.1), (V.a.2), (V.a.3), (V.a.4), (V.a.5), and (V.a.6), R² is

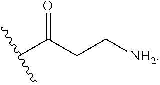

In some embodiments, in each of Formulas (V.a), (V.a.1), (V.a.2), (V.a.3), (V.a.4), (V.a.5), and (V.a.6), R² is

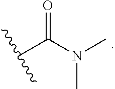

In some embodiments, in each of Formulas (V.a), (V.a.1), (V.a.2), (V.a.3), (V.a.4), (V.a.5), and (V.a.6), R² is

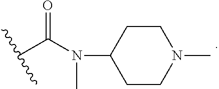

In some embodiments, in each of Formulas (V.a), (V.a.1), (V.a.2), (V.a.3), (V.a.4), (V.a.5), and (V.a.6), p is 0 and R³ is not present.

In some embodiments, in each of Formulas (V.a), (V.a.1), (V.a.2), (V.a.3), (V.a.4), (V.a.5), and (V.a.6), p is 1.

In some embodiments, in each of Formulas (V.a), (V.a.1), (V.a.2), (V.a.3), (V.a.4), (V.a.5), and (V.a.6), p is 2.

In some embodiments, in each of Formulas (V.a), (V.a.1), (V.a.2), (V.a.3), (V.a.4), (V.a.5), and (V.a.6), p is 3.

In some embodiments, in each of Formulas (V.a), (V.a.1), (V.a.2), (V.a.3), (V.a.4), (V.a.5), and (V.a.6), p is 4.

In some embodiments, in each of Formulas (V.a), (V.a.1), (V.a.2), (V.a.3), (V.a.4), (V.a.5), and (V.a.6), p is ≧2 and at least two groups R³ are attached to the same ring atom.

In some embodiments, in each of Formulas (V.a), (V.a.1), (V.a.2), (V.a.3), (V.a.4), (V.a.5), and (V.a.6), p is 1 and R³ is independently selected from the group consisting of alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (V.a), (V.a.1), (V.a.2), (V.a.3), (V.a.4), (V.a.5), and (V.a.6), p is 2, 3, or 4 and each R³ is independently selected from the group consisting of alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (V.a), (V.a.1), (V.a.2), (V.a.3), (V.a.4), (V.a.5), and (V.a.6), p is 2, 3, or 4 and at least two groups R³ are bound to the same ring carbon atom, wherein each R³, which may be the same or different, is independently selected from the group consisting of alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In some embodiments, in each of Formulas (V.a), (V.a.1), (V.a.2), (V.a.3), (V.a.4), (V.a.5), and (V.a.6), p is 2, 3, or 4 and at least two groups R³ are bound to the same ring carbon atom, wherein two R³ groups, which may be the same or different, together with the carbon atom to which they are attached, form a cycloalkyl, a cycloalkenyl, a heterocycloalkyl ring containing from one to three heteroatoms selected from the group consisting of N, O, and S, or a heterocycloalkenyl ring containing from one to three heteroatoms selected from the group consisting of N, O, and S.

In one embodiment, in Formula (V.a), p is 1, 2, 3, or 4, and each R³ is independently selected from the group consisting of alkyl, heteroalkyl, alkenyl, heteroalkenyl, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(S)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶, —NR²³C(O)NR²⁵R²⁶, and —NR²³—C(NH)—NR²⁵R²⁶,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (V.a), p is 1 and R³ is selected from the group consisting of alkyl, heteroalkyl, alkenyl, and heteroalkenyl,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (V.a), p is 2, 3, or 4, and any two R³ groups bound to the same ring A atom are taken together to form a —C(O)— group.

In one embodiment, in Formula (V.a), p is 2, 3, or 4, and any two R³ groups bound to the same ring A atom are taken together to form a spiroheterocycloalkyl group having from 1 to 3 ring heteroatoms independently selected from the group consisting of —NH—, —NR⁶—, O, S, S(O), and S(O)₂, or spiroheterocycloalkenyl group having from 1 to 3 ring heteroatoms independently selected from the group consisting of —NH—, —NR⁶—, O, S, S(O), and S(O)₂.

In some embodiments, in each of Formulas (V.a), (V.a.1), (V.a.2), (V.a.3), (V.a.4), (V.a.5), and (V.a.6), R³ is alkyl.

In some embodiments, in each of Formulas (V.a), (V.a.1), (V.a.2), (V.a.3), (V.a.4), (V.a.5), and (V.a.6), R³ is heteroalkyl.

In some embodiments, in each of Formulas (V.a), (V.a.1), (V.a.2), (V.a.3), (V.a.4), (V.a.5), and (V.a.6), R³ is alkenyl.

In some embodiments, in each of Formulas (V.a), (V.a.1), (V.a.2), (V.a.3), (V.a.4), (V.a.5), and (V.a.6), R³ is heteroalkenyl.

In some embodiments, in each of Formulas (V.a), (V.a.1), (V.a.2), (V.a.3), (V.a.4), (V.a.5), and (V.a.6), R³ is alkynyl.

In some embodiments, in each of Formulas (V.a), (V.a.1), (V.a.2), (V.a.3), (V.a.4), (V.a.5), and (V.a.6), R³ is heteroalkynyl.

In some embodiments, in each of Formulas (V.a), (V.a.1), (V.a.2), (V.a.3), (V.a.4), (V.a.5), and (V.a.6), R³ is aryl.

In some embodiments, in each of Formulas (V.a), (V.a.1), (V.a.2), (V.a.3), (V.a.4), (V.a.5), and (V.a.6), R³ is heteroaryl.

In some embodiments, in each of Formulas (V.a), (V.a.1), (V.a.2), (V.a.3), (V.a.4), (V.a.5), and (V.a.6), R³ is cycloalkyl.

In some embodiments, in each of Formulas (V.a), (V.a.1), (V.a.2), (V.a.3), (V.a.4), (V.a.5), and (V.a.6), R³ is cycloalkenyl.

In some embodiments, in each of Formulas (V.a), (V.a.1), (V.a.2), (V.a.3), (V.a.4), (V.a.5), and (V.a.6), R³ is heterocycloalkyl.

In some embodiments, in each of Formulas (V.a), (V.a.1), (V.a.2), (V.a.3), (V.a.4), (V.a.5), and (V.a.6), R³ is heterocycloalkenyl.

In some embodiments, in each of Formulas (V.a), (V.a.1), (V.a.2), (V.a.3), (V.a.4), (V.a.5), and (V.a.6), R³ is halogen.

In some embodiments, in each of Formulas (V.a), (V.a.1), (V.a.2), (V.a.3), (V.a.4), (V.a.5), and (V.a.6), R³ is —CN.

In some embodiments, in each of Formulas (V.a), (V.a.1), (V.a.2), (V.a.3), (V.a.4), (V.a.5), and (V.a.6), R³ is —NO₂.

In some embodiments, in each of Formulas (V.a), (V.a.1), (V.a.2), (V.a.3), (V.a.4), (V.a.5), and (V.a.6), R³ is —OR¹⁹.

In some embodiments, in each of Formulas (V.a), (V.a.1), (V.a.2), (V.a.3), (V.a.4), (V.a.5), and (V.a.6), R³ is —OC(O)OR²⁰.

In some embodiments, in each of Formulas (V.a), (V.a.1), (V.a.2), (V.a.3), (V.a.4), (V.a.5), and (V.a.6), R³ is —NR²¹R²².

In some embodiments, in each of Formulas (V.a), (V.a.1), (V.a.2), (V.a.3), (V.a.4), (V.a.5), and (V.a.6), R³ is —NR²³SO₂R²⁴.

In some embodiments, in each of Formulas (V.a), (V.a.1), (V.a.2), (V.a.3), (V.a.4), (V.a.5), and (V.a.6), R³ is —NR²³C(O)OR²⁰.

In some embodiments, in each of Formulas (V.a), (V.a.1), (V.a.2), (V.a.3), (V.a.4), (V.a.5), and (V.a.6), R³ is —NR²³C(O)R²⁴.

In some embodiments, in each of Formulas (V.a), (V.a.1), (V.a.2), (V.a.3), (V.a.4), (V.a.5), and (V.a.6), R³ is —SO₂NR²⁵R²⁶.

In some embodiments, in each of Formulas (V.a), (V.a.1), (V.a.2), (V.a.3), (V.a.4), (V.a.5), and (V.a.6), R³ is —C(O)R²⁴.

In some embodiments, in each of Formulas (V.a), (V.a.1), (V.a.2), (V.a.3), (V.a.4), (V.a.5), and (V.a.6), R³ is —C(O)OR²⁰.

In some embodiments, in each of Formulas (V.a), (V.a.1), (V.a.2), (V.a.3), (V.a.4), (V.a.5), and (V.a.6), R³ is —SR¹⁹.

In some embodiments, in each of Formulas (V.a), (V.a.1), (V.a.2), (V.a.3), (V.a.4), (V.a.5), and (V.a.6), R³ is —S(O)R¹⁹.

In some embodiments, in each of Formulas (V.a), (V.a.1), (V.a.2), (V.a.3), (V.a.4), (V.a.5), and (V.a.6), R³ is —SO₂R¹⁹.

In some embodiments, in each of Formulas (V.a), (V.a.1), (V.a.2), (V.a.3), (V.a.4), (V.a.5), and (V.a.6), R³ is —OC(O)R²⁴.

In some embodiments, in each of Formulas (V.a), (V.a.1), (V.a.2), (V.a.3), (V.a.4), (V.a.5), and (V.a.6), R³ is —C(O)NR²⁵R²⁶.

In some embodiments, in each of Formulas (V.a), (V.a.1), (V.a.2), (V.a.3), (V.a.4), (V.a.5), and (V.a.6), R³ is —NR²³C(N—CN)NR²⁵R²⁶.

In some embodiments, in each of Formulas (V.a), (V.a.1), (V.a.2), (V.a.3), (V.a.4), (V.a.5), and (V.a.6), R³ is —NR²³C(O)NR²⁵R²⁶

In some embodiments, in each of Formulas (V.a), (V.a.1), (V.a.2), (V.a.3), (V.a.4), (V.a.5), and (V.a.6), R³ is selected from the group consisting of: methyl, ethyl, propyl (straight or branched), butyl (straight or branched), pentyl (straight or branched), phenyl,

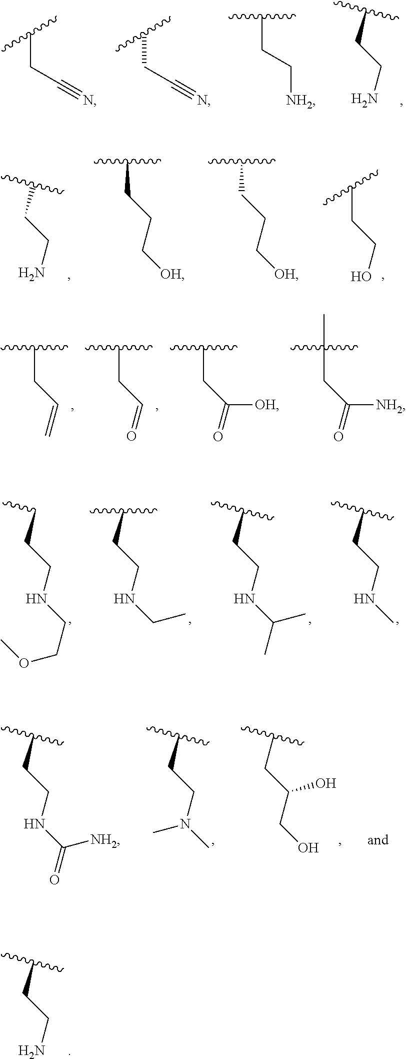

In some embodiments, in each of Formulas (V.a), (V.a.1), (V.a.2), (V.a.3), (V.a.4), (V.a.5), and (V.a.6), when E is —NR⁶—, R³ is absent.

In one embodiment, the compounds of the invention have a structure shown in Formula (V.b) and include pharmaceutically acceptable salts, solvates, esters, prodrugs, or isomers of said compounds:

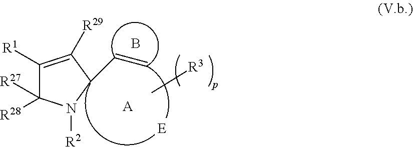

wherein R¹, R², R³, R²⁷, R²⁸, R²⁹, p, E, ring A, and ring B are selected independently of each other and wherein:
ring A (including E and the unsaturation shown) is a 7-8-membered cycloalkenyl or heterocycloalkenyl ring;
E is selected from the group consisting of —O—, —S—, —S(O)—, —S(O)₂—, —C(R⁴)(R⁵)—, —N(R⁶)—, —N(C(Y)R⁷)—, —N(C(Y)OR⁸)—, —N(C(Y)N(R⁹)(R¹⁰)—, —C(O)—N(R¹¹)—, —N(R¹¹)—C(O)—, —S(O)₂—N(R¹¹)—, —N(R¹¹)—S(O)₂—, —C(O)—O—, —O—C(O)—, —O—N(R⁶)—, —N(R⁶)—O—, —N(R⁶)—N(R¹²)—, —N═N—, —C(R⁷)═N—, —C(O)—C(R⁷)═N—, —C(O)—N═N—, —O—C(Y)—N(R¹¹)—, —N(R¹¹)—C(Y)—O—, —N(R¹¹)—C(Y)—N(R¹²)—, —C(Y)—N(R¹¹)—O—, —C(Y)—N(R¹¹)—N(R¹²)—, —O—N(R¹¹)—C(Y)—, and —N(R¹²)—N(R¹¹)—C(Y)—;
ring B is a substituted or unsubstituted heteroaromatic ring;
and p, R¹, R², R³, R⁴, R⁵, R⁶, R⁷, R⁸, R⁹, R¹⁰, R¹¹, R¹², R²⁷, R²⁸, R²⁹, Y, and the optional substituents on ring B are as defined above in Formula (I).

In one embodiment, in Formula (V.b.), ring A is a cycloalkenyl ring.

In one embodiment, in Formula (V.b.), ring A is a heterocycloalkenyl ring.

In one embodiment, in Formula (V.b.), E is —C(R⁴)(R⁶)—.

In one embodiment, in Formula (V.b.), E is selected from the group consisting of —O—, —S—, —S(O)—, —S(O)₂—, —N(R⁶)—, —N(C(Y)R⁷)—, —N(C(Y)OR⁸)—, —N(C(Y)N(R⁹)(R¹⁰))—, —C(O)—N(R¹¹)—, —N(R¹¹)—C(O)—, —S(O)₂—N(R¹¹)—, —N(R¹¹)—S(O)₂—, —C(O)—O—, —O—C(O)—, —O—N(R⁶)—, —N(R⁶)—O—, —N(R⁶)—N(R¹²)—, —N═N—, —C(R⁷)═N—, —C(O)—C(R⁷)═N—, —C(O)—N═N—, —O—C(Y)—N(R¹¹)—, —N(R¹¹)—C(Y)—O—, —N(R¹¹)—C(Y)—N(R¹²)—, —C(Y)—N(R¹¹)—O—, —C(Y)—N(R¹¹)—N(R¹²)—, —O—N(R¹¹)—C(Y)—, and —N(R¹²)—N(R¹¹)—C(Y)—.

In one embodiment, in Formula (V.b.), E is selected from the group consisting of —O—, —S—, —S(O)—, —S(O)₂—, and —N(R⁶)—.

in one embodiment, in Formula (V.b.), E is selected from the group consisting of —O—, —S—, —S(O)—, —S(O)₂—, and —N(R⁶)—, wherein R⁶ is selected from the group consisting of H, alkyl, —C(O)R²⁴, and —C(S)R²⁴.

In one embodiment, in Formula (V.b.), E is selected from the group consisting of —O— and —N(R⁶)—, wherein R⁶ is selected from the group consisting of H, alkyl, —C(O)R²⁴, and —C(S)R²⁴.

In one embodiment, in Formula (V.b.), E is —O—.

In one embodiment, in Formula (V.b.), E is —S—.

In one embodiment, in Formula (V.b.), E is —S(O)—.

In one embodiment, in Formula (V.b.), E is —S(O)₂—.

In one embodiment, in Formula (V.b.), E is —C(R⁴)(R⁵)—.

In one embodiment, in Formula (V.b.), E is —N(R⁶)—.

In one embodiment, in Formula (V.b.), E is —N(C(Y)R⁷)—.

In one embodiment, in Formula (V.b.), E is —N(C(Y)OR⁵)—.

In one embodiment, in Formula (V.b.), E is —N(C(Y)N(R⁹)(R¹⁰)—.

In one embodiment, in Formula (V.b.), E is —C(O)—N(R¹¹)—.

In one embodiment, in Formula (V.b.), E is —N(R¹¹)—C(O)—.

In one embodiment, in Formula (V.b.), E is —S(O)₂—N(R¹¹)—.

In one embodiment, in Formula (V.b.), E is —N(R¹¹)—S(O)₂—.

In one embodiment, in Formula (V.b.), E is —C(O)—O—.

In one embodiment, in Formula (V.b.), E is —O—C(O)—.

In one embodiment, in Formula (V.b.), E is —O—N(R⁶)—.

In one embodiment, in Formula (V.b.), E is —N(R⁶)—O—.

In one embodiment, in Formula (V.b.), E is —N(R⁶)—N(R¹²)—.

In one embodiment, in Formula (V.b.), E is —N═N—.

In one embodiment, in Formula (V.b.), E is —C(R⁷)═N—.

In one embodiment, in Formula (V.b.), E is —C(O)—C(R⁷)═N—.

In one embodiment, in Formula (V.b.), E is —C(O)—N═N—.

In one embodiment, in Formula (V.b.), E is —O—C(Y)—N(R¹¹)—.

In one embodiment, in Formula (V.b.), E is —N(R¹¹)—C(Y)—O—.

In one embodiment, in Formula (V.b.), E is —N(R¹¹)—C(Y)—N(R¹²)—.

In one embodiment, in Formula (V.b.), E is —C(Y)—N(R¹¹)—O—.

In one embodiment, in Formula (V.b.), E is —C(Y)—N(R¹¹)—N(R¹²)—.

In one embodiment, in Formula (V.b.), E is —O—N(R¹¹)—C(Y)—.

In one embodiment, in Formula (V.b.), E is —N(R¹²)—N(R¹¹)—C(Y)—.

In one embodiment, in Formula (V.b.), Y is (═O).

In one embodiment, in Formula (V.b.), Y is (═S).

In one embodiment, in Formula (V.b.), Y is (═N(R¹³)).

In one embodiment, in Formula (V.b.), Y is (═N(CN)).

In one embodiment, in Formula (V.b.), Y is (═N(OR¹⁴)).

In one embodiment, in Formula (V.b.), Y is (═N(R¹⁵)(R¹⁶)).

In one embodiment, in Formula (V.b.), Y is (═C(R¹⁷)(R¹⁸)).

In one embodiment, in Formula (V.b.), B is an unsubstituted heteroaromatic ring.

In one embodiment, in Formula (V.b.), B is an unsubstituted 5-6-membered heteroaromatic ring having from 1-3 ring heteroatoms, which can be the same or different, each hetero ring atom being independently selected from the group consisting of N, S, O, S(O), and S(O)₂.

In one embodiment, in Formula (V.b.), B is a heteroaromatic ring which is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (V.b.), B is a 5-6-membered heteroaromatic ring having from 1-3 ring heteroatoms, which can be the same or different, each hetero ring atom being independently selected from the group consisting of N, S, O, S(O), and S(O)₂, which heteroaromatic ring is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (V.b.), B is an unsubstituted 6-membered heteroaromatic ring having from 1-3 ring heteroatoms, which can be the same or different, each hetero ring atom being independently selected from the group consisting of N, S, and O.

In one embodiment, in Formula (V.b.), B is a 6-membered heteroaromatic ring having from 1-3 ring heteroatoms, which can be the same or different, each hetero ring atom being independently selected from the group consisting of N, S, and O, which heteroaromatic ring is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (V.b.), B is an unsubstituted 6-membered heteroaromatic ring having 2 ring heteroatoms, each ring heteroatom being independently selected from of N, S, and O.

In one embodiment, in Formula (V.b.), B is a 6-membered heteroaromatic ring having 2 ring heteroatoms, each ring heteroatom being independently selected from of N, S, and O, which heteroaromatic ring is substituted with one or more substituents, which can be the same or different, each substituent being independently selected from the group consisting of halogen, —CN, —NO₂, alkyl, heteroalkyl, haloalkyl, —OR¹⁹, —NR²¹R²², —C(O)R²⁴, —C(O)OR²⁰, —SR¹⁹, —S(O)R¹⁹, —SO₂R¹⁹, —OC(O)R²⁴, and —C(O)NR²⁵R²⁶.

In one embodiment, in Formula (V.b.), B is an unsubstituted 5-membered heteroaromatic ring having from 1-2 ring heteroatoms, which can be the same or different, each hetero ring atom being independently selected from the group consisting of N, S, and O.

In one embodiment, in Formula (V.b.), B is a 5-membered heteroaromatic ring having from 1-2 ring heteroatoms, which can be the same or different, each hetero ring atom being independently selected from the group consisting of N, S, and O, which heteroaromatic ring is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (V.b.), B is an unsubstituted 5-membered heteroaromatic ring having 1 ring heteroatom selected from of N, S, and O.

In one embodiment, in Formula (V.b.), B is a 5-membered heteroaromatic ring having 1 ring heteroatom selected from of N, S, and O, which heteroaromatic ring is substituted with one or more substituents, which can be the same or different, each substituent being independently selected from the group consisting of halogen, —CN, —NO₂, alkyl, heteroalkyl, haloalkyl, —OR¹⁹, —NR²¹R²², —C(O)R²⁴, —C(O)OR²⁰, —SR¹⁹, —S(O)R¹⁹, —SO₂R¹⁹, —OC(O)R²⁴, and —C(O)NR²⁵R²⁶.

In one embodiment, in Formula (V.b.), B is a 5-membered heteroaromatic ring having S as the ring heteroatom, which heteroaromatic ring is substituted with one or more substituents, which can be the same or different, each substituent being independently selected from the group consisting of halogen, —CN, —NO₂, alkyl, heteroalkyl, haloalkyl, —OR¹⁹, —NR²¹R²², —C(O)R²⁴, —C(O)OR²⁰, —SR¹⁹, —S(O)R¹⁹, —SO₂R¹⁹, —OC(O)R²⁴, and —C(O)NR²⁵R²⁶.

In one embodiment, in Formula (V.b.), B is an unsubstituted 5-membered heteroaromatic ring having S as the ring heteroatom.

In one embodiment, in Formula (V.b.), B is selected from the group consisting of

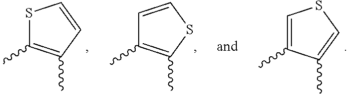

In one embodiment, in Formula (V.b.), R¹ is unsubstituted aryl.

In one embodiment, in Formula (V.b.), R¹ is unsubstituted phenyl.

In one embodiment, in Formula (V.b.), R¹ is unsubstituted naphthyl.

In one embodiment, in Formula (V.b.), R¹ is substituted aryl.

In one embodiment, in Formula (V.b.), R¹ is substituted phenyl.

In one embodiment, in Formula (V.b.), R¹ is substituted naphthyl.

In one embodiment, in Formula (V.b.), R¹ is aryl substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (V.b.), R¹ is phenyl substituted with one or more substituents, which can be the same or different, each substituent being independently selected from the group consisting halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (V.b.), R¹ is phenyl substituted with one to four substituents, which can be the same or different, each substituent being independently selected from the group consisting of halo, —OH, —CN, and haloalkyl.

In one embodiment, in Formula (V.b.), R¹ is selected from the group consisting of:

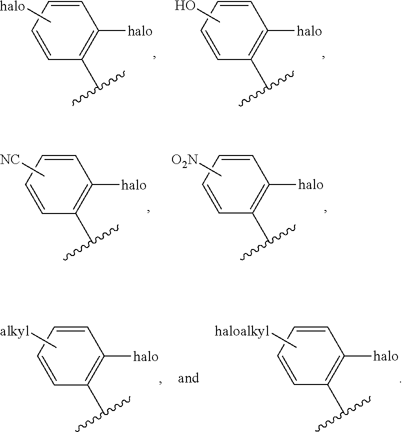

In one embodiment, in Formula (V.b.), is:

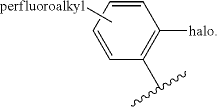

In one embodiment, in Formula (V.b.), R¹ is phenyl substituted with one to three fluoro groups.

In one embodiment, in Formula (V.b.), R¹ is phenyl substituted with two fluoro groups.

In one embodiment, in Formula (V.b.), R¹ is phenyl substituted with one fluoro group.

In one embodiment, in Formula (V.b.), is:

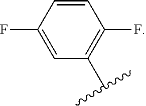

In one embodiment, in Formula (V.b.), R²⁷, R²⁸, and R²⁹ are each independently selected from the group consisting of H and alkyl.

In one embodiment, in Formula (V.b.), R¹ is:

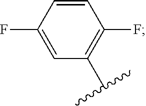

and R²⁷, R²⁸, and R²⁹ are each independently selected from the group consisting of H and alkyl.

In one embodiment, in Formula (V.b.), R² is —C(Z)R⁷.

In one embodiment, in Formula (V.b.), R² is —C(Z)NR⁹R¹⁰.

In one embodiment, in Formula (V.b.), R² is —C(Z)OR⁸.

In one embodiment, in Formula (V.b.), R² is —SO₂NR⁹R¹⁰.

In one embodiment, in Formula (V.b.), R² is alkyl.

In one embodiment, in Formula (V.b.), R² is heteroalkyl.

In one embodiment, in Formula (V.b.), R² is aryl.

In one embodiment, in Formula (V.b.), R² is heteroaryl.

In one embodiment, in Formula (V.b.), R² is cycloalkyl.

In one embodiment, in Formula (V.b.), R² is cycloalkenyl.

In one embodiment, in Formula (V.b.), R² is heterocycloalkyl.

In one embodiment, in Formula (V.b.), R² is heterocycloalkenyl.

In one embodiment, in Formula (V.b.), Z is (═O).

In one embodiment, in Formula (V.b.), Z is (═S).

In one embodiment, in Formula (V.b.), Z is (═N(R¹³)).

In one embodiment, in Formula (V.b.), Z is (═N(CN)).

In one embodiment, in Formula (V.b.), Z is (═N(OR¹⁴)).

In one embodiment, in Formula (V.b.), Z is (═N(R¹⁵)(R¹⁶)).

In one embodiment, in Formula (V.b.), Z is (═C(R¹⁷)(R¹⁸)).

In one embodiment, in Formula (V.b.), R² is —C(Z)R⁷, and Z is (═O).

In one embodiment, in Formula (V.b.), R² is —C(O)H.

In one embodiment, in Formula (V.b.), R² is —C(O)alkyl.

In one embodiment, in Formula (V.b.), R² is —C(O)CH₃.

In one embodiment, in Formula (V.b.), R² is —C(O)R⁷, wherein said R⁷ is alkyl substituted with one or more substituents, which can be the same or different, each substituent being independently selected from the group consisting of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (V.b.), R² is —C(O)R⁷, wherein said R⁷ is alkyl substituted with one to three substituents, which can be the same or different, each substituent being independently selected from the group consisting of —OR¹⁹, —NR²¹R²², and cycloalkyl.

In one embodiment, in Formula (V.b.), R² is —C(O)R⁷, wherein said R⁷ is alkyl, wherein said alkyl is substituted with alkyl and —OH.

In one embodiment, in Formula (V.b.), R² is —C(O)R⁷, wherein said R⁷ is alkyl substituted with one to three substituents, which can be the same or different, each substituent being independently selected from the group consisting of —OH, —NH₂, and cyclopropyl.

In one embodiment, in Formula (V.b.), R² is —C(O)R⁷, wherein said R⁷ is alkyl substituted with one to two substituents, which can be the same or different, each substituent being independently selected from the group consisting of —NH₂, and cyclopropyl.

In one embodiment, in Formula (V.b.), R² is —C(O)R⁷, wherein said R⁷ is alkyl substituted with —OH.

In one embodiment, in Formula (V.b.), R² is —C(O)R⁷, wherein said R⁷ is unsubstituted heterocycloalkyl.

In one embodiment, in Formula (V.b.), R² is —C(O)R⁷, wherein said R⁷ is substituted heterocycloalkyl.

In one embodiment, in Formula (V.b.), R² is —C(O)R⁷, wherein said R⁷ is heterocycloalkyl substituted with one or more substituents, which can be the same or different, each substituent being independently selected from the group consisting of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (V.b.), R² is —C(O)R⁷, wherein said R⁷ is selected from the group consisting of substituted piperidine, substituted piperazine, substituted morpholine, substituted pyrrolidine, and substituted azetidine.

In one embodiment, in Formula (V.b.), R² is selected from:

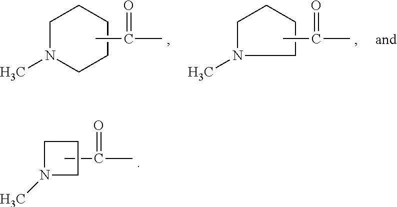

In one embodiment, in Formula (V.b.), R² is —C(O)NR⁹R¹⁰.

In one embodiment, in Formula (V.b.), R² is —C(O)NH₂.

In one embodiment, in Formula (V.b.), R² is —C(O)NR⁹R¹⁰, wherein R⁹ and R¹⁰ can be the same or different, each being independently selected from alkyl.

In one embodiment, in Formula (V.b.), R² is —C(O)NR⁹R¹⁰, wherein R⁹ is unsubstituted heterocycloalkyl and R¹⁰ is selected from the group consisting of H and alkyl.

In one embodiment, in Formula (V.b.), R² is —C(O)NR⁹R¹⁰, wherein R⁹ is substituted heterocycloalkyl and R¹⁰ is selected from the group consisting of H and alkyl.

In one embodiment, in Formula (V.b.), R² is —C(O)NR⁹R¹⁰, wherein R⁹ is heterocycloalkyl substituted with from one to three substituents, which can be the same or different, each substituent being independently selected from alkyl, and R¹⁰ is selected from the group consisting of H and alkyl.

In one embodiment, in Formula (V.b.), R² is

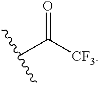

In one embodiment, in Formula (V.b.), R² is

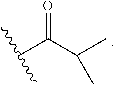

In one embodiment, in Formula (V.b.), R² is

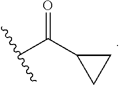

In one embodiment, in Formula (V.b.), R² is

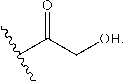

In one embodiment, in Formula (V.b.), R² is

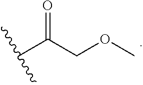

In one embodiment, in Formula (V.b.), R² is

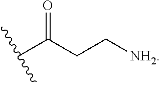

In one embodiment, in Formula (V.b.), R² is

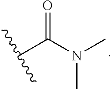

In one embodiment, in Formula (V.b.), R² is

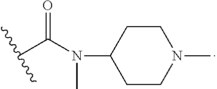

In one embodiment, in Formula (V.b.), p is 0 and R³ is not present.

In one embodiment, in Formula (V.b.), p is 1.

In one embodiment, in Formula (V.b.), p is 2.

In one embodiment, in Formula (V.b.), p is 3.

In one embodiment, in Formula (V.b.), p is 4.

In one embodiment, in Formula (V.b.), p is ≧2 and at least two groups R³ are attached to the same ring atom.

In one embodiment, in Formula (V.b.), p is 1 and R³ is independently selected from the group consisting of alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (V.b.), p is 2, 3, or 4 and each R³ is independently selected from the group consisting of alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (V.b.), p is 2, 3, or 4 and at least two groups R³ are bound to the same ring carbon atom, wherein each R³, which may be the same or different, is independently selected from the group consisting of alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, NR²³C(N—CN)NR²⁵R²⁶ and —NR²³C(O)NR²⁵R²⁶.

In one embodiment, in Formula (V.b.), p is 2, 3, or 4 and at least two groups R³ are bound to the same ring carbon atom, wherein two R³ groups, which may be the same or different, together with the carbon atom to which they are attached, form a cycloalkyl, a cycloalkenyl, a heterocycloalkyl ring containing from one to three heteroatoms selected from the group consisting of N, O, and S, or a heterocycloalkenyl ring containing from one to three heteroatoms selected from the group consisting of N, O, and S.

In one embodiment, in Formula (V.b), p is >0 and each R³ is independently selected from the group consisting of alkyl, heteroalkyl, alkenyl, heteroalkenyl, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR¹⁴R²⁶, —C(O)R²⁴, —C(S)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶, —NR²³C(O)NR²⁵R²⁶, and —NR²³—O(NH)—NR²⁵R²⁶,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (V.b), p is 1 and R³ is selected from the group consisting of alkyl, heteroalkyl, alkenyl, and heteroalkenyl,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (V.b), p is ≧2 and any two R³ groups bound to the same ring A atom are taken together to form a —C(O)— group.

In one embodiment, in Formula (V.b), p is ≧2 and any two R³ groups bound to the same ring A atom are taken together to form a spiroheterocycloalkyl group having from 1 to 3 ring heteroatoms independently selected from the group consisting of —NH—, —NR⁶—, O, S, S(O), and S(O)₂, or spiroheterocycloalkenyl group having from 1 to 3 ring heteroatoms independently selected from the group consisting of —NH—, —NR⁶—, O, S, S(O), and S(O)₂.

In one embodiment, in Formula (V.b.), R³ is alkyl.

In one embodiment, in Formula (V.b.), R³ is heteroalkyl.

In one embodiment, in Formula (V.b.), R³ is alkenyl.

In one embodiment, in Formula (V.b.), R³ is heteroalkenyl.

In one embodiment, in Formula (V.b.), R³ is alkynyl.

In one embodiment, in Formula (V.b.), R³ is heteroalkynyl.

In one embodiment, in Formula (V.b.), R³ is aryl.

In one embodiment, in Formula (V.b.), R³ is heteroaryl.

In one embodiment, in Formula (V.b.), R³ is cycloalkyl.

In one embodiment, in Formula (V.b.), R³ is cycloalkenyl.

In one embodiment, in Formula (V.b.), R³ is heterocycloalkyl.

In one embodiment, in Formula (V.b.), R³ is heterocycloalkenyl.

In one embodiment, in Formula (V.b.), R³ is halogen.

In one embodiment, in Formula (V.b.), R³ is

In one embodiment, in Formula (V.b.), R³ is —NO₂.

In one embodiment, in Formula (V.b.), R³ is —OR¹⁹.

In one embodiment, in Formula (V.b.), R³ is —OC(O)OR²⁰.

In one embodiment, in Formula (V.b.), R³ is —NR²¹R²².

In one embodiment, in Formula (V.b.), R³ is —NR²³SO₂R²⁴.

In one embodiment, in Formula (V.b.), R³ is —NR²³C(O)OR²⁰.

In one embodiment, in Formula (V.b.), R³ is —NR²³C(O)R²⁴.

In one embodiment, in Formula (V.b.), R³ is —SO₂NR²⁵R²⁶.

In one embodiment, in Formula (V.b.), R³ is —C(O)R²⁴.

In one embodiment, in Formula (V.b.), R³ is —C(O)OR²⁰.

In one embodiment, in Formula (V.b.), R³ is —SR¹⁹.

In one embodiment, in Formula (V.b.), R³ is —S(O)R¹⁹.

In one embodiment, in Formula (V.b.), R³ is —SO₂R¹⁹.

In one embodiment, in Formula (V.b.), R³ is —OC(O)R²⁴.

In one embodiment, in Formula (V.b.), R³ is —C(O)NR²⁵R²⁶.

In one embodiment, in Formula (V.b.), R³ is —NR²³C(N—CN)NR²⁵R²⁶.

In one embodiment, in Formula (V.b.), R³ is —NR²³C(O)NR²⁵R²⁶.

In one embodiment, Formula (V.b) has the general structure:

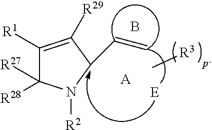

In one embodiment, Formula (V.b) has the general structure:

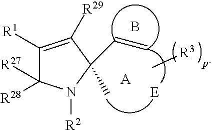

In one embodiment, Formula (V.b) has the general structure:

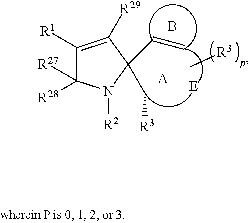

In one embodiment, Formula (V.b) has the general structure:

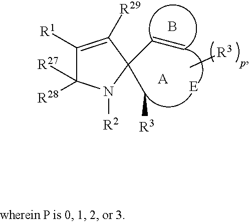

In one embodiment, Formula (V.b) has the general structure:

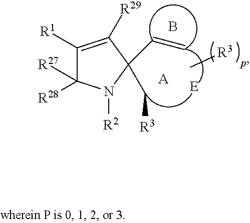

In one embodiment. Formula (V.b) has the general structure:

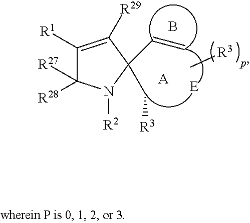

In one embodiment, the compounds of the invention have a structure shown in Formula (VI) and include pharmaceutically acceptable salts, solvates, esters, prodrugs, or isomers of said compounds:

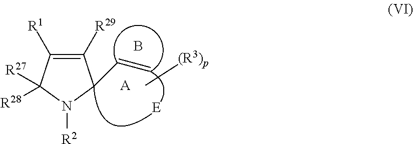

wherein R¹, R², R³, R²⁷, R²⁸, R²⁹, p, E, ring A, and ring B are selected independently of each other and wherein:
ring A (including E and the unsaturation shown) is a 4-8-membered cycloalkenyl or heterocycloalkenyl ring;
E is selected from the group consisting of —O—, —S—, —S(O)—, —S(O)₂—, —C(R⁴)(R⁵)—, —N(R⁶)—, —N(C(Y)R⁷)—, —N(C(Y)OR⁸)—, —N(C(Y)N(R⁹)(R¹⁰))—, —C(O)—N(R¹¹)—, —N(R¹¹)—C(O)—, —S(O)₂—N(R¹¹)—, —N(R¹¹)—S(O)₂—, —C(O)—O—, —O—C(O)—, —O—N(R⁶)—, —N(R⁶)—O—, —N(R⁶)—N(R¹²)—, —N═N—, —C(R⁷)═N—, —C(O)—C(R⁷)═N—, —C(O)—N═N—, —O—C(Y)—N(R¹¹)—, —N(R¹¹)—C(Y)—O—, —N(R¹¹)—C(Y)—N(R¹²)—, —C(Y)—N(R¹¹)—O—, —C(Y)—N(R¹¹)—N(R¹²)—, —O—N(R¹¹)—C(Y)—, and —N(R¹²)—N(R¹¹)—C(Y)—;
ring B is an unsubstituted or optionally independently substituted partially unsaturated alicyclic ring, or a partially unsaturated heterocyclic ring,
and p, R¹, R², R³, R⁴, R⁵, R⁶, R⁷, R⁸, R⁹, R¹⁰, R¹¹, R¹², R²⁷, R²⁸, R²⁹, Y, and the optional substituents on ring B are as defined above in Formula (I).

In one embodiment, in Formula (VI), ring A is a cycloalkenyl ring.

In one embodiment, in Formula (VI), ring A is a heterocycloalkenyl ring.

In one embodiment, in Formula (VI), ring A is a 4-membered ring.

In one embodiment, in Formula (VI), ring A is a 5-membered ring.

In one embodiment, in Formula (VI), ring A is a 6-membered ring.

In one embodiment, in Formula (VI), ring A is a 7-membered ring.

In one embodiment, in Formula (VI), ring A is an 8-membered ring.

In one embodiment, in Formula (VI), E is —C(R⁴)(R⁵)—.

In one embodiment, in Formula (VI), E is selected from the group consisting of —O—, —S—, —S(O)—, —S(O)₂—, —N(R⁶)—, —N(C(Y)R⁷)—, —N(C(Y)OR⁸)—, —N(C(Y)N(R⁹)(R¹⁰))—, —C(O)—N(R¹¹)—, —N(R¹¹)—C(O)—, —S(O)₂—N(R¹¹)—, —N(R¹¹)—S(O)₂—, —C(O)—O—, —O—C(O)—, —O—N(R⁶)—, —N(R⁶)—O—, —N(R⁶)—N(R¹²)—, —N═N—, —C(R⁷)═N—, —C(O)—C(R⁷)═N—, —C(O)—N═N—, —O—C(Y)—N(R¹¹)—, —N(R¹¹)—C(Y)—O—, —N(R¹¹)—C(Y)—N(R¹²)—, —C(Y)—N(R¹¹)—O—, —C(Y)—N(R¹¹)—N(R¹²)—, —O—N(R¹¹)—C(Y)—, and —N(R¹²)—N(R¹¹)—C(Y)—.

In one embodiment, in Formula (VI), E is selected from the group consisting of —O—, —S—, —S(O)—, —S(O)₂—, and —N(R⁶)—.

In one embodiment, in Formula (VI), E is selected from the group consisting of —O—, —S—, —S(O)—, —S(O)₂—, and —N(R⁶)—, wherein R⁶ is selected from the group consisting of H, alkyl, —C(O)R²⁴, and —C(S)R²⁴.

In one embodiment, in Formula (VI), E is selected from the group consisting of —O— and —N(R⁶)—, wherein R⁶ is selected from the group consisting of H, alkyl, —C(O)R²⁴, and —C(S)R²⁴.

In one embodiment, in Formula (VI), E is —O—.

In one embodiment, in Formula (VI), E is —S—.

In one embodiment, in Formula (VI), E is —S(O)—.

In one embodiment, in Formula (VI), E is —S(O)₂—.

In one embodiment, in Formula (VI), E is —C(R⁴)(R⁵)—.

In one embodiment, in Formula (VI), E is —N(R⁶)—.

In one embodiment, in Formula (VI), E is —N(C(Y)R⁷)—.

In one embodiment, in Formula (VI), E is —N(C(Y)OR⁸)—.

In one embodiment, in Formula (VI), E is —N(C(Y)N(R⁹)(R¹⁰))—.

In one embodiment, in Formula (VI), E is —C(O)—N(R¹¹)—.

In one embodiment, in Formula (VI), E is —N(R¹¹)—C(O)—.

In one embodiment, in Formula (VI), E is —S(O)₂—N(R¹¹)—.

In one embodiment, in Formula (VI), E is —N(R¹¹)—S(O)₂—.

In one embodiment, in Formula (VI), E is —C(O)—O—.

In one embodiment, in Formula (VI), E is —O—C(O)—.

In one embodiment, in Formula (VI), E is —O—N(R⁶)—.

In one embodiment, in Formula (VI), E is —N(R⁶)—O—.

In one embodiment, in Formula (VI), E is —N(R⁶)—N(R¹²)—.

In one embodiment, in Formula (VI), E is —N═N—.

In one embodiment, in Formula (VI), E is —C(R⁷)═N—.

In one embodiment, in Formula (VI), E is —C(O)—C(R⁷)═N—.

In one embodiment, in Formula (VI), E is —C(O)—N═N—.

In one embodiment, in Formula (VI), E is —O—C(Y)—N(R¹¹)—.

In one embodiment, in Formula (VI), E is —N(R¹¹)—C(Y)—O—.

In one embodiment, in Formula (VI), E is —N(R¹¹)—C(Y)—N(R¹²)—.

In one embodiment, in Formula (VI), E is —C(Y)—N(R¹¹)—O—.

In one embodiment, in Formula (VI), E is —C(Y)—N(R¹¹)—N(R¹²)—.

In one embodiment, in Formula (VI), E is —O—N(R¹¹)—C(Y)—.

In one embodiment, in Formula (VI), E is —N(R¹²)—N(R¹¹)—C(Y)—.

In one embodiment, in Formula (VI), Y is (═O).

In one embodiment, in Formula (VI), Y is (—S).

In one embodiment, in Formula (VI), Y is (═N(R¹³)).

In one embodiment, in Formula (VI), Y is (═N(CN)).

In one embodiment, in Formula (VI), Y is (═N(OR¹⁴)).

In one embodiment, in Formula (VI), Y is (═N(R¹⁵)(R¹⁶)).

In one embodiment, in Formula (VI), Y is (—C(R¹⁷)(R¹⁸)).

In one embodiment, in Formula (VI), ring A is a 4-membered ring and E is selected from the group consisting of —O—, —S—, —S(O)—, —S(O)₂—, —C(R⁴)(R⁵)—, —N(R⁶)—, —N(C(Y)R⁷)—, —N(C(Y)OR⁸)—, —N(C(Y)N(R⁹)(R¹⁰))—.

In one embodiment, in Formula (VI), A is a 4-membered ring and E is selected from the group consisting of —CH₂—, —CH(R⁴)—, —C(R⁴)(R⁵)—.

In one embodiment, in Formula (VI), A is a 5-membered ring and E is selected from the group consisting of —O—, —S—, —S(O)—, —S(O)₂—, —C(R⁴)(R⁵)—, —N(R⁶)—, —N(C(Y)R⁷)—, —N(C(Y)OR⁸)—, —N(C(Y)N(R⁹)(R¹⁰))—, —C(O)—N(R¹¹)—, —N(R¹¹)—C(O)—, —S(O)₂—N(R¹¹)—, —N(R¹¹)—S(O)₂—, —C(O)—O—, —O—C(O)—, —O—N(R⁶)—, —N(R⁶)—O—, —N(R⁶)—N(R¹²)—, —N═N—, and —C(R⁷)═N—.

In one embodiment, in Formula (VI), A is a 5-membered ring and E is —O—.

In one embodiment, in Formula (VI), A is a 5-membered ring and E is —S—.

In one embodiment, in Formula (VI), A is a 5-membered ring and E is —S(O)—.

In one embodiment, in Formula (VI), A is a 5-membered ring and E is —S(O)₂—.

In one embodiment, in Formula (VI), A is a 5-membered ring and E is —C(R⁴)(R⁵)—.

In one embodiment, in Formula (VI), A is a 5-membered ring and E is —N(R⁶)—.

In one embodiment, in Formula (VI), A is a 5-membered ring and E is —N(C(Y)R⁷)—.

In one embodiment, in Formula (VI), A is a 5-membered ring and E is —N(C(Y)OR⁸)—.

In one embodiment, in Formula (VI), A is a 5-membered ring and E is —N(C(Y)N(R⁹)R¹⁰))—.

In one embodiment, in Formula (VI), A is a 5-membered ring and E is —C(O)—N(R¹¹)—.

In one embodiment, in Formula (VI), A is a 5-membered ring and E is —N(R¹¹)—C(O)—.

In one embodiment, in Formula (VI), A is a 5-membered ring and E is —S(O)₂—N(R¹¹)—.

In one embodiment, in Formula (VI), A is a 5-membered ring and E is —N(R¹¹)—S(O)₂—.

In one embodiment, in Formula (VI), A is a 5-membered ring and E is —C(O)—O—.

In one embodiment, in Formula (VI), A is a 5-membered ring and E is —O—C(O)—.

In one embodiment, in Formula (VI), A is a 5-membered ring and E is —O—N(R⁶)—.

In one embodiment, in Formula (VI), A is a 5-membered ring and E is —N(R⁶)—O—.

In one embodiment, in Formula (VI), A is a 5-membered ring and E is —N(R⁶)—N(R¹²)—.

In one embodiment, in Formula (VI), A is a 5-membered ring and E is —N═N—.

In one embodiment, in Formula (VI), A is a 5-membered ring and E is —C(R⁷)═N—.

In one embodiment, in Formula (VI), A is a 6-membered ring and E is selected from the group consisting of —O—, —S—, —S(O)—, —S(O)₂—, —C(R⁴)(R⁵)—, —N(R⁶)—, —N(C(Y)R⁷)—, —N(C(Y)OR⁸)—, —N(C(Y)N(R⁹)(R¹⁰))—, —C(O)—N(R¹¹)—, —N(R¹¹)—C(O)—, —S(O)₂—N(R¹¹)—, —N(R¹¹)—S(O)₂—, —C(O)—O—, —O—C(O)—, —O—N(R⁶)—, —N(R⁶)—O—, —N(R⁶)—N(R¹²)—, —N═N—, —C(R⁷)═N—, —C(O)—C(R⁷)═N—, —C(O)—N═N—, —O—C(Y)—N(R¹¹)—, —N(R¹¹)—C(Y)—O—, —N(R¹¹)—C(Y)—N(R¹²)—, —C(Y)—N(R¹¹)—O—, —C(Y)—N(R¹¹)—N(R¹²)—, —O—N(R¹¹)—C(Y)—, and —N(R¹²)—N(R¹¹)—C(Y)—.

In one embodiment, in Formula (VI), A is a 6-membered ring and E is —O—.

In one embodiment, in Formula (VI), A is a 6-membered ring and E is —S—.

In one embodiment, in Formula (VI), A is a 6-membered ring and E is —S(O)—.

In one embodiment, in Formula (VI), A is a 6-membered ring and E is —S(O)₂—.

In one embodiment, in Formula (VI), A is a 6-membered ring and E is —C(R⁴)(R⁵)—.

In one embodiment, in Formula (VI), A is a 6-membered ring and E is —N(R⁶)—.

In one embodiment, in Formula (VI), A is a 6-membered ring and E is —N(C(Y)R⁷)—.

In one embodiment, in Formula (VI), A is a 6-membered ring and E is —N(C(Y)OR⁸)—.

In one embodiment, in Formula (VI), A is a 6-membered ring and E is —N(C(Y)N(R⁹)(R¹⁰))—.

In one embodiment, in Formula (VI), A is a 6-membered ring and E is —C(O)—N(R¹¹)—.

In one embodiment, in Formula (VI), A is a 6-membered ring and E is —N(R¹¹)—C(O)—.

In one embodiment, in Formula (VI), A is a 6-membered ring and E is —S(O)₂—N(R¹¹)—.

In one embodiment, in Formula (VI), A is a 6-membered ring and E is —N(R¹¹)—S(O)₂—.

In one embodiment, in Formula (VI), A is a 6-membered ring and E is —C(O)—O—.

In one embodiment, in Formula (VI), A is a 6-membered ring and E is —O—C(O)—.

In one embodiment, in Formula (VI), A is a 6-membered ring and E is —O—N(R⁶)—.

In one embodiment, in Formula (VI), A is a 6-membered ring and E is —N(R⁶)—O—.

In one embodiment, in Formula (VI), A is a 6-membered ring and E is —N(R⁶)—N(R¹²)—.

In one embodiment, in Formula (VI), A is a 6-membered ring and E is —N═N—.

In one embodiment, in Formula (VI), A is a 6-membered ring and E is —C(R⁷)═N—.

In one embodiment, in Formula (VI), A is a 6-membered ring and E is —C(O)—C(R⁷)═N—.

In one embodiment, in Formula (VI), A is a 6-membered ring and E is —C(O)—N═N—.

In one embodiment, in Formula (VI), A is a 6-membered ring and E is —O—C(Y)—N(R¹¹)—.

In one embodiment, in Formula (VI), A is a 6-membered ring and E is —N(R¹¹)—C(Y)—O—.

In one embodiment, in Formula (VI), A is a 6-membered ring and E is —N(R¹¹)—C(Y)—N(R¹²)—.

In one embodiment, in Formula (VI), A is a 6-membered ring and E is —C(Y)—N(R¹¹)—O—.

In one embodiment, in Formula (VI), A is a 6-membered ring and E is —C(Y)—N(R¹¹)—N(R¹²)—.

In one embodiment, in Formula (VI), A is a 6-membered ring and E is —O—N(R¹¹)—C(Y)—.

In one embodiment, in Formula (VI), A is a 6-membered ring and E is —N(R¹²)—N(R¹¹)—C(Y)—.

In one embodiment, in Formula (VI), A is a 7-membered ring and E is selected from the group consisting of —O—, —S—, —S(O)—, —S(O)₂—, —C(R⁴)(R⁵)—, —N(R⁶)—, —N(C(Y)R⁷)—, —N(C(Y)OR⁸)—, —N(C(Y)N(R⁹)(R¹⁰))—, —C(O)—N(R¹¹)—, —N(R¹¹)—C(O)—, —S(O)₂—N(R¹¹)—, —N(R¹¹)—S(O)₂—, —C(O)—O—, —O—C(O)—, —O—N(R⁶)—, —N(R⁶)—O—, —N(R⁸)—N(R¹²)—, —N═N—, —C(R⁷)═N—, —C(O)—C(R⁷)═N—, —C(O)—N═N—, —O—C(Y)—N(R¹¹)—, —N(R¹¹)—C(Y)—O—, —N(R¹¹)—C(Y)—-N(R¹²)—, —C(Y)—N(R¹¹)—O—, —C(Y)—N(R¹¹)—N(R¹²)—, —O—N(R¹¹)—C(Y)—, and —N(R¹²)—N(R¹¹)—C(Y)—.

In one embodiment, in Formula (VI), A is a 7-membered ring and E is —O—.

In one embodiment, in Formula (VI), A is a 7-membered ring and E is —S—.

In one embodiment, in Formula (VI), A is a 7-membered ring and E is —S(O)—.

In one embodiment, in Formula (VI), A is a 7-membered ring and E is —S(O)₂—.

In one embodiment, in Formula (VI), A is a 7-membered ring and E is —C(R⁴)(R⁵)—.

In one embodiment, in Formula (VI), A is a 7-membered ring and E is —N(R⁶)—.

In one embodiment, in Formula (VI), A is a 7-membered ring and E is —N(C(Y)R⁷)—.

In one embodiment, in Formula (VI), A is a 7-membered ring and E is —N(C(Y)OR⁸)—.

In one embodiment, in Formula (VI), A is a 7-membered ring and E is —N(C(Y)N(R⁹)(R¹⁰))—.

In one embodiment, in Formula (VI), A is a 7-membered ring and E is —C(O)—N(R¹¹)—.

In one embodiment, in Formula (VI), A is a 7-membered ring and E is —N(R¹¹)—C(O)—.

In one embodiment, in Formula (VI), A is a 7-membered ring and E is —S(O)₂—N(R¹¹)—.

In one embodiment, in Formula (VI), A is a 7-membered ring and E is —N(R¹¹)—S(O)₂—.

In one embodiment, in Formula (VI), A is a 7-membered ring and E is —C(O)—O—.

In one embodiment, in Formula (VI), A is a 7-membered ring and E is —O—C(O)—.

In one embodiment, in Formula (VI), A is a 7-membered ring and E is —O—N(R⁶)—.

In one embodiment, in Formula (VI), A is a 7-membered ring and E is —N(R⁶)—O—.

In one embodiment, in Formula (VI), A is a 7-membered ring and E is —N(R⁶)—N(R¹²)—.

In one embodiment, in Formula (VI), A is a 7-membered ring and E is —N═N—.

In one embodiment, in Formula (VI), A is a 7-membered ring and E is —C(R⁷)═N—.

In one embodiment, in Formula (VI), A is a 7-membered ring and E is —C(O)—C(R⁷)═N—.

In one embodiment, in Formula (VI), A is a 7-membered ring and E is —C(O)—N═N—.

In one embodiment, in Formula (VI), A is a 7-membered ring and E is —O—C(Y)—N(R¹¹)—.

In one embodiment, in Formula (VI), A is a 7-membered ring and E is —N(R¹¹)—C(Y)—O—.

In one embodiment, in Formula (VI), A is a 7-membered ring and E is —N(R¹¹)—C(Y)—N(R¹²)—.

In one embodiment, in Formula (VI), A is a 7-membered ring and E is —C(Y)—N(R¹¹)—O—.

In one embodiment, in Formula (VI), A is a 7-membered ring and E is —C(Y)—N(R¹¹)—N(R¹²)—.

In one embodiment, in Formula (VI), A is a 7-membered ring and E is —O—N(R¹¹)—C(Y)—.

In one embodiment, in Formula (VI), A is a 7-membered ring and E is —N(R¹²)—N(R¹¹)—C(Y)—.

In one embodiment, in Formula (VI), A is a 8-membered ring and E is selected from the group consisting of —O—, —S—, —S(O)—, —S(O)₂—, —C(R⁴)(R⁵)—, —N(R⁶)—, —N(C(Y)R⁷)—, —N(C(Y)OR⁸)—, —N(C(Y)N(R⁹)(R¹⁰))—, —C(O)—N(R¹¹)—, —N(R¹¹)—C(O)—, —S(O)₂—N(R¹¹)—, —N(R¹¹)—S(O)₂—, —C(O)—O—, —O—C(O)—, —O—N(R⁶)—, —N(R⁶)—O—, —N(R⁶)—N(R¹²)—, —N═N—, —C(R⁷)═N—, —C(O)—C(R⁷)═N—, —C(O)—N═N—, —O—C(Y)—N(R¹¹)—, —N(R¹¹)—C(Y)—O—, —N(R¹¹)—C(Y)—N(R¹²)—, —C(Y)—N(R¹¹)—O—, —C(Y)—N(R¹¹)—N(R¹²)—, —O—N(R¹¹)—C(Y)—, and —N(R¹²)—N(R¹¹)—C(Y)—.

In one embodiment, in Formula (VI), A is a 8-membered ring and E is —O—.

In one embodiment, in Formula (VI), A is a 8-membered ring and E is —S—.

In one embodiment, in Formula (VI), A is a 8-membered ring and E is —S(O)—.

In one embodiment, in Formula (VI), A is a 8-membered ring and E is —S(O)₂—.

In one embodiment, in Formula (VI), A is a 8-membered ring and E is —C(R⁴)(R⁵)—.

In one embodiment, in Formula (VI), A is a 8-membered ring and E is —N(R⁶)—.

In one embodiment, in Formula (VI), A is a 8-membered ring and E is —N(C(Y)R⁷)—.

In one embodiment, in Formula (VI), A is a 8-membered ring and E is —N(C(Y)OR⁸)—.

In one embodiment, in Formula (VI), A is a 8-membered ring and E is —N(C(Y)N(R⁹)(R¹⁰))—.

In one embodiment, in Formula (VI), A is a 8-membered ring and E is —C(O)—N(R¹¹)—.

In one embodiment, in Formula (VI), A is a 8-membered ring and E is —N(R¹¹)—C(O)—.

In one embodiment, in Formula (VI), A is a 8-membered ring and E is —S(O)₂—N(R¹¹)—.

In one embodiment, in Formula (VI), A is a 8-membered ring and E is —N(R¹¹)—S(O)₂—.

In one embodiment, in Formula (VI), A is a 8-membered ring and E is —C(O)—O—.

In one embodiment, in Formula (VI), A is a 8-membered ring and E is —O—C(O)—.

In one embodiment, in Formula (VI), A is a 8-membered ring and E is —O—N(R⁶)—

In one embodiment, in Formula (VI), A is a 8-membered ring and E is —N(R⁶)—O—.

In one embodiment, in Formula (VI), A is a 8-membered ring and E is —N(R⁶)—N(R¹²)—.

In one embodiment, in Formula (VI), A is a 8-membered ring and E is —N═N—.

In one embodiment, in Formula (VI), A is a 8-membered ring and E is —C(R⁷)═N—.

In one embodiment, in Formula (VI), A is a 8-membered ring and E is —C(O)—C(R⁷)═N—.

In one embodiment, in Formula (VI), A is a 8-membered ring and E is —C(O)—N═N—.

In one embodiment, in Formula (VI), A is a 8-membered ring and E is —O—C(Y)—N(R¹¹)—.

In one embodiment, in Formula (VI), A is a 8-membered ring and E is —N(R¹¹)—C(Y)—O—.

In one embodiment, in Formula (VI), A is a 8-membered ring and E is —N(R¹¹)—C(Y)—N(R¹²)—.

In one embodiment, in Formula (VI), A is a 8-membered ring and E is —C(Y)—N(R¹¹)—O—.

In one embodiment, in Formula (VI), A is a 8-membered ring and E is —C(Y)—N(R¹¹)—N(R¹²)—.

In one embodiment, in Formula (VI), A is a 8-membered ring and E is —O—N(R¹¹)—C(Y)—.

In one embodiment, in Formula (VI), A is a 8-membered ring and E is —N(R¹²)—N(R¹¹)—C(Y)—.

In one embodiment, in Formula (VI), B is a partially unsaturated alicyclic ring, which ring is unsubstituted.

In one embodiment, in Formula (VI), B is a partially unsaturated alicyclic ring which is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (VI), B is a partially unsaturated heterocyclic ring, which ring is unsubstituted.

In one embodiment, in Formula (VI), B is a partially unsaturated heterocyclic ring which is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (VI), R¹ is unsubstituted aryl.

In one embodiment, in Formula (VI), R¹ is unsubstituted phenyl.

In one embodiment, in Formula (VI), R¹ is unsubstituted naphthyl.

In one embodiment, in Formula (VI), R¹ is substituted aryl.

In one embodiment, in Formula (VI), R¹ is substituted phenyl.

In one embodiment, in Formula (VI), R¹ is substituted naphthyl.

In one embodiment, in Formula (VI), R¹ is aryl substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²³R²⁶ and —NR²³C(O)NR²⁵R²⁶.

In one embodiment, in Formula (VI), R¹ is phenyl substituted with one or more substituents, which can be the same or different, each substituent being independently selected from the group consisting halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (VI), R¹ is phenyl substituted with one to four substituents, which can be the same or different, each substituent being independently selected from the group consisting of halo, —OH, —CN, —NO₂, and —NR²¹R²², and haloalkyl.

In one embodiment, in Formula (VI), R¹ is selected from the group consisting of:

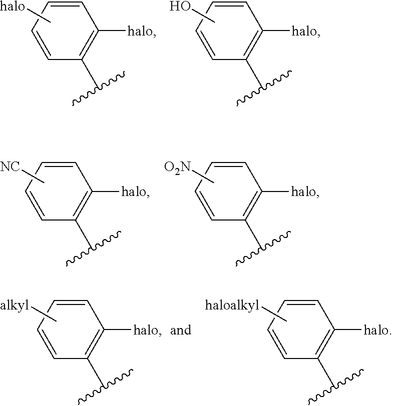

In one embodiment, in Formula (VI), R¹ is:

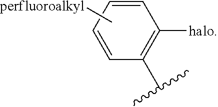

In one embodiment, in Formula (VI), R¹ is phenyl substituted with one to three fluoro groups.

In one embodiment, in Formula (VI), R¹ is phenyl substituted with two fluoro groups.

In one embodiment, in Formula (VI), R¹ is phenyl substituted with one fluoro group.

In one embodiment, in Formula (VI), R¹ is:

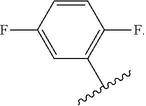

In one embodiment, in Formula (VI). R²⁷, R²⁸, and R²⁹ are each independently selected from the group consisting of H and alkyl.

In one embodiment, in Formula (VI), R¹ is:

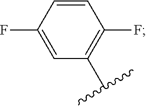

and R²⁷, R²⁸, and R²⁹ are each independently selected from the group consisting of H and alkyl.

In one embodiment, in Formula (VI), R² is —C(Z)R⁷.

In one embodiment, in Formula (VI), R² is —C(Z)NR⁹R¹⁰.

In one embodiment, in Formula (VI), R² is —C(Z)OR⁸.

In one embodiment, in Formula (VI), R² is —SO₂NR⁹R¹⁰.

In one embodiment, in Formula (VI), R² is alkyl.

In one embodiment, in Formula (VI), R² is heteroalkyl.

In one embodiment, in Formula (VI), R² is aryl.

In one embodiment, in Formula (VI), R² is heteroaryl.

In one embodiment, in Formula (VI), R² is cycloalkyl.

In one embodiment, in Formula (VI), R² is cycloalkenyl.

In one embodiment, in Formula (VI), R² is heterocycloalkyl.

In one embodiment, in Formula (VI), R² is heterocycloalkenyl.

In one embodiment, in Formula (VI), Z is (═O).

In one embodiment, in Formula (VI), Z is (═S).

In one embodiment, in Formula (VI), Z is (═N(R¹³)).

In one embodiment, in Formula (VI), Z is (═N(CN)).

In one embodiment, in Formula (VI), Z is (═N(OR¹⁴)).

In one embodiment, in Formula (VI), Z is (═N(R¹⁵)(R¹⁶)).

In one embodiment, in Formula (VI), Z is (═C(R¹⁷)(R¹⁸)).

In one embodiment, in Formula (VI), R² is —C(Z)R⁷, and Z is (═O).

In one embodiment, in Formula (VI), R² is —C(O)H.

In one embodiment, in Formula (VI), R² is —C(O)alkyl.

In one embodiment, in Formula (VI), R² is —C(O)CH₃.

In one embodiment, in Formula (VI), R² is —C(O)R⁷, wherein said R⁷ is alkyl substituted with one or more substituents, which can be the same or different, each substituent being independently selected from the group consisting of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (VI), R² is —C(O)R⁷, wherein said R⁷ is alkyl substituted with one to three substituents, which can be the same or different, each substituent being independently selected from the group consisting of —OR¹⁹, —NR²¹R²², and cycloalkyl.

In one embodiment, in Formula (VI), R² is —C(O)R⁷, wherein said R⁷ is alkyl, wherein said alkyl is substituted with alkyl and —OH.

In one embodiment, in Formula (VI), R² is —C(O)R⁷, wherein said R⁷ is alkyl substituted with one to three substituents, which can be the same or different, each substituent being independently selected from the group consisting of —OH, —NH₂, and cyclopropyl.

In one embodiment, in Formula (VI), R² is —C(O)R⁷, wherein said R⁷ is alkyl substituted with one to two substituents, which can be the same or different, each substituent being independently selected from the group consisting of —NH₂, and cyclopropyl.

In one embodiment, in Formula (VI). R² is —C(O)R⁷, wherein said R⁷ is alkyl substituted with —OH.

In one embodiment, in Formula (VI), R² is —C(O)R⁷, wherein said R⁷ is unsubstituted heterocycloalkyl.

In one embodiment, in Formula (VI), R² is —C(O)R⁷, wherein said R⁷ is substituted heterocycloalkyl.

In one embodiment, in Formula (VI), R² is —C(O)R⁷, wherein said R⁷ is heterocycloalkyl substituted with one or more substituents, which can be the same or different, each substituent being independently selected from the group consisting of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (VI), R² is —C(O)R⁷, wherein said R⁷ is selected from the group consisting of substituted piperidine, substituted piperazine, substituted morpholine, substituted pyrrolidine, and substituted azetidine.

In one embodiment, in Formula (VI), R² is selected from:

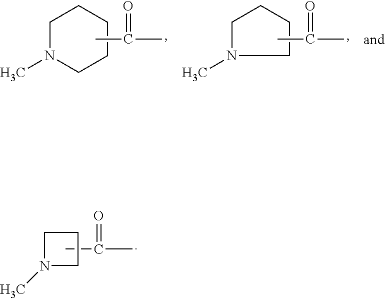

In one embodiment, in Formula (VI), R² is —C(O)NR⁹R¹⁰.

In one embodiment, in Formula (VI), R² is —C(O)NH₂.

In one embodiment, in Formula (VI), R² is —C(O)NR⁹R¹⁰, wherein R⁹ and R¹⁰ can be the same or different, each being independently selected from alkyl.

In one embodiment, in Formula (VI), R² is —C(O)NR⁹R¹⁰, wherein R⁹ is unsubstituted heterocycloalkyl and R¹⁹ is selected from the group consisting of H and alkyl.

In one embodiment, in Formula (VI), R² is —C(O)NR⁹R¹⁰, wherein R⁹ is substituted heterocycloalkyl and R¹⁰ is selected from the group consisting of H and alkyl.

In one embodiment, in Formula (VI), R² is selected from the group consisting of: alkyl, haloalkyl, heteroalkyl, heterohaloalkyl, —C(O)R⁷. —C(O)OR⁸, and —C(O)NR⁹R¹⁰.

In one embodiment, in Formula (VI), R² is selected from the group consisting of:

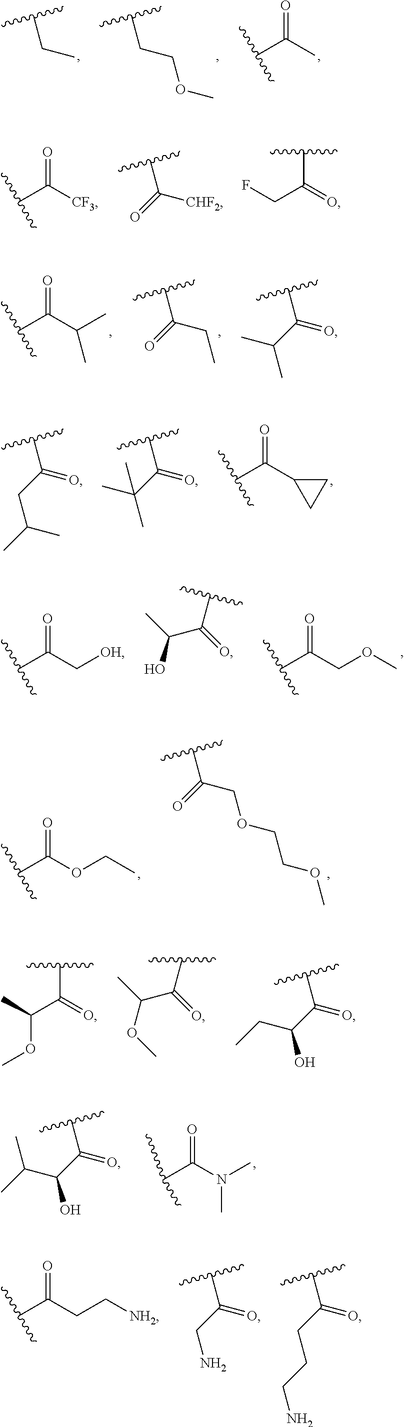

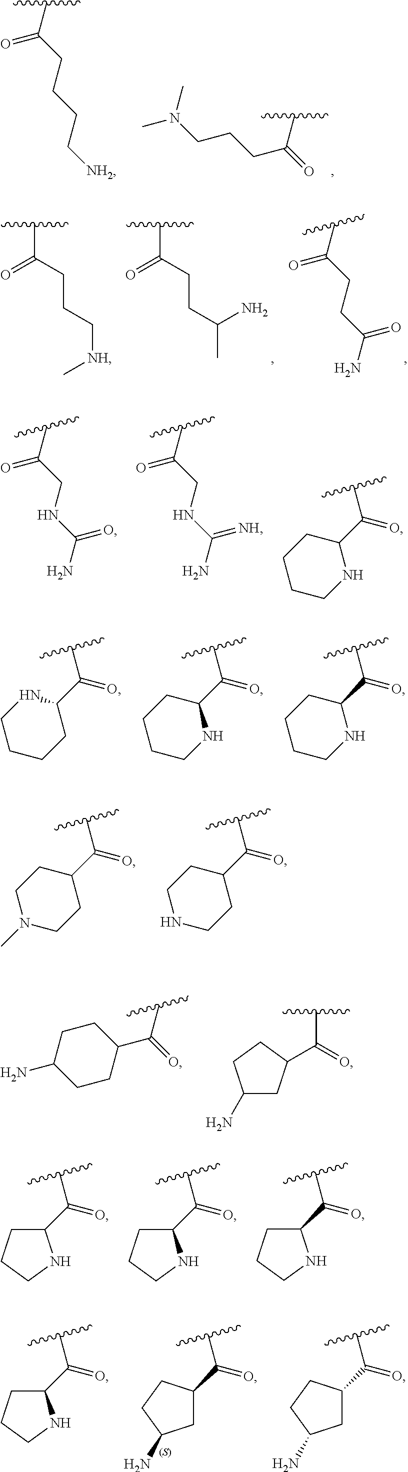

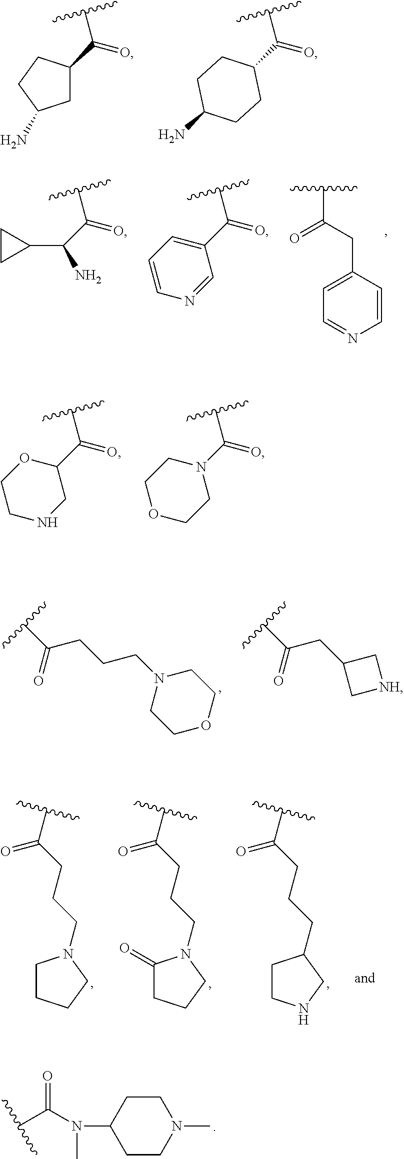

In one embodiment, in Formula (VI), R² is —C(O)NR⁹R¹⁰, wherein R⁹ is heterocycloalkyl substituted with from one to three substituents, which can be the same or different, each substituent being independently selected from alkyl, and R¹⁰ is selected from the group consisting of H and alkyl.

In one embodiment, in Formula (VI), R² is

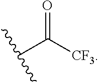

In one embodiment, in Formula (VI), R² is

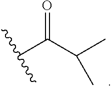

In one embodiment, in Formula (VI), R² is

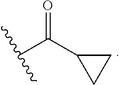

In one embodiment, in Formula (VI), R² is

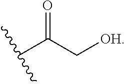

In one embodiment, in Formula (VI). R² is

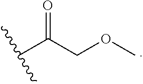

In one embodiment, in Formula (VI), R² is

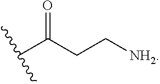

In one embodiment, in Formula (VI), R² is

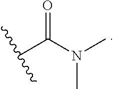

In one embodiment, in Formula (VI), R² is

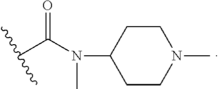

In one embodiment, in Formula (VI), p is 0 and R³ is not present.

In one embodiment, in Formula (VI), p is 1.

In one embodiment, in Formula (VI), p is 2.

In one embodiment, in Formula (VI), p is 3.

In one embodiment, in Formula (VI), p is 4.

In one embodiment, in Formula (VI), p is ≧2 and at least two groups R³ are attached to the same ring atom.

In one embodiment, in Formula (VI), p is 1 and R³ is independently selected from the group consisting of alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (VI), p is 2, 3, or 4 and each R³ is independently selected from the group consisting of alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (VI), p is 2, 3, or 4 and at least two groups R³ are bound to the same ring carbon atom, wherein each R³, which may be the same or different, is independently selected from the group consisting of alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (VI), p is 2, 3, or 4 and at least two groups R³ are bound to the same ring carbon atom, wherein two R³ groups, which may be the same or different, together with the carbon atom to which they are attached, form a cycloalkyl, a cycloalkenyl, a heterocycloalkyl ring containing from one to three heteroatoms selected from the group consisting of N, O, and S, or a heterocycloalkenyl ring containing from one to three heteroatoms selected from the group consisting of N, O, and S.

In one embodiment, in Formula (VI), p is >0 and each R³ is independently selected from the group consisting of alkyl, heteroalkyl, alkenyl, heteroalkenyl, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(S)R²⁴, C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶, —NR²³C(O)NR²⁵R²⁶, and —NR²³—C(NH)—NR²⁵R²⁶,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (VI), p is 1 and R³ is selected from the group consisting of alkyl, heteroalkyl, alkenyl, and heteroalkenyl,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

In one embodiment, in Formula (VI), p is 2, 3, or 4, and any two R³ groups bound to the same ring A atom are taken together to form a —C(O)— group.

In one embodiment, in Formula (IV), p is 2, 3, or 4, and any two R³ groups bound to the same ring A atom are taken together with the carbon atom to which they are attached to form a spirocycloalkyl, a spirocycloalkenyl, a spiroheterocycloalkyl ring containing from one to three ring heteroatoms independently selected from the group consisting of —NH—, —NR⁶—, —S—, —S(O)—, —S(O)₂—, and —O—, or a spiroheterocycloalkenyl ring containing from one to three ring heteroatoms independently selected from the group consisting of —NH—, —NR⁶—, —S—, —S(O)—, —S(O)₂—, and —O—.

In one embodiment, in Formula (IV), p is >0 and R² and R³ are taken together with the carbon atom to which they are attached to form a cycloalkyl, a cycloalkenyl, a heterocycloalkyl ring containing from one to three ring heteroatoms independently selected from the group consisting of —NH—, —NR⁶—, —S—, —S(O)—, —S(O)₂—, and —O—, or a heterocycloalkenyl ring containing from one to three ring heteroatoms independently selected from the group consisting of —NH—, —NR⁶—, —S—, —S(O)—, —S(O)₂—, and —O—.

In one embodiment, in Formula (VI), R³ is alkyl.

In one embodiment, in Formula (VI), R³ is heteroalkyl.

In one embodiment, in Formula (VI), R³ is alkenyl.

In one embodiment, in Formula (VI), R³ is heteroalkenyl.

In one embodiment, in Formula (VI), R³ is alkynyl.

In one embodiment, in Formula (VI), R³ is heteroalkynyl.

In one embodiment, in Formula (VI), R³ is aryl.

In one embodiment, in Formula (VI), R³ is heteroaryl.

In one embodiment, in Formula (VI), R³ is cycloalkyl.

In one embodiment, in Formula (VI), R³ is cycloalkenyl.

In one embodiment, in Formula (VI), R³ is heterocycloalkyl.

In one embodiment, in Formula (VI), R³ is heterocycloalkenyl.

In one embodiment, in Formula (VI), R³ is halogen.

In one embodiment, in Formula (VI), R³ is —CN.

In one embodiment, in Formula (VI), R³ is —NO₂.

In one embodiment, in Formula (VI), R³ is —OR¹⁹.

In one embodiment, in Formula (VI), R³ is —OC(O)OR²⁰.

In one embodiment, in Formula (VI), R³ is —NR²¹R²².

In one embodiment, in Formula (VI), R³ is —NR²³SO₂R²⁴.

In one embodiment, in Formula (VI), R³ is —NR²³C(O)OR²⁰.

In one embodiment, in Formula (VI), R³ is —NR²³C(O)R²⁴.

In one embodiment, in Formula (VI), R³ is —SO₂NR²⁵R²⁶.

In one embodiment, in Formula (VI), R³ is —C(O)R²⁴. In one embodiment, in Formula (VI), R³ is —C(S)R²⁴.

In one embodiment, in Formula (VI), R³ is —C(O)OR²⁰.

In one embodiment, in Formula (VI), R³ is —SR¹⁹.

In one embodiment, in Formula (VI), R³ is —S(O)R¹⁹.

In one embodiment, in Formula (VI), R³ is —SO₂R¹⁹.

In one embodiment, in Formula (VI), R³ is —OC(O)R²⁴.

In one embodiment, in Formula (VI), R³ is —C(O)NR²⁵R²⁶.

In one embodiment, in Formula (VI), R³ is —NR²³C(N—CN)NR²⁵R²⁶.

In one embodiment, in Formula (VI), R³ is —NR²³C(O)NR²⁵R²⁶.

In one embodiment, in Formula (VI), R³ is selected from the group consisting of methyl, ethyl, propyl (straight or branched), butyl (straight or branched), pentyl (straight or branched), phenyl,

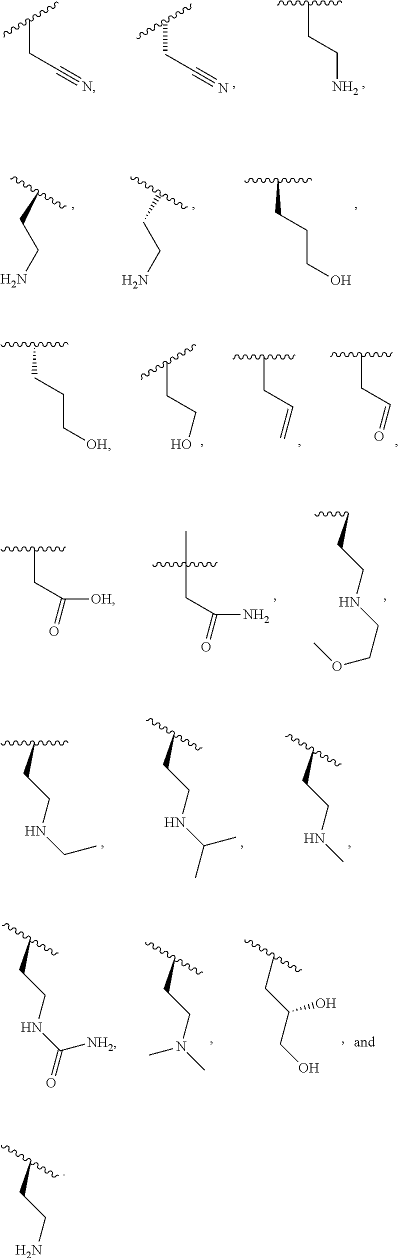

In one embodiment, in Formula (IV), when E is —NR⁶—, R³ is absent.

In one embodiment, Formula (VI) has the general structure:

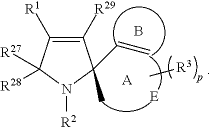

In one embodiment, Formula (VI) has the general structure:

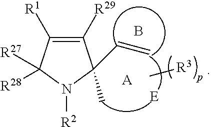

In one embodiment, Formula (VI) has the general structure:

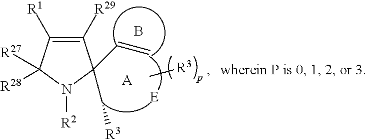

In one embodiment, Formula (VI) has the general structure:

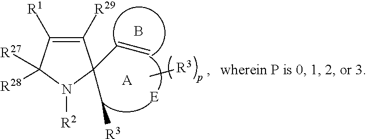

In one embodiment, Formula (VI) has the general structure:

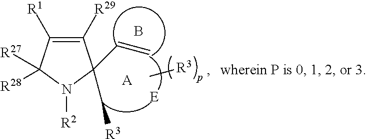

In one embodiment, Formula (VI) has the general structure:

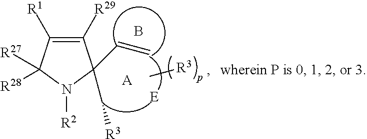

In one embodiment, the compounds of the invention have a structure shown in Table 1 below, and include pharmaceutically acceptable salts, solvates, esters, prodrugs, or isomers of said compounds,

TABLE 1

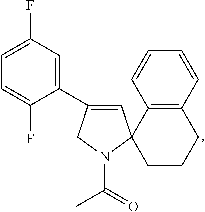

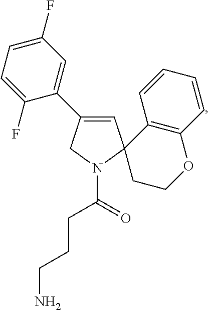

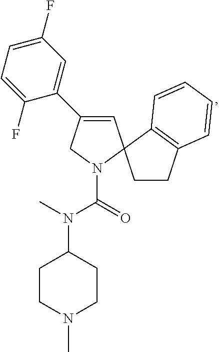

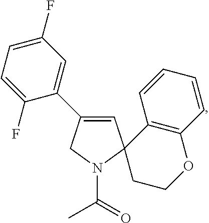

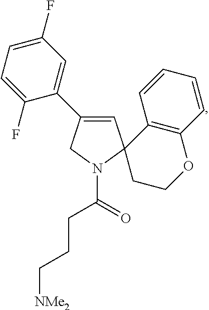

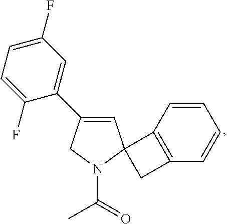

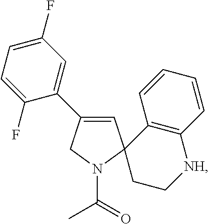

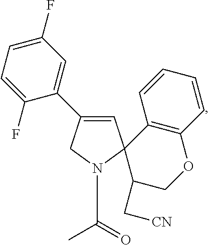

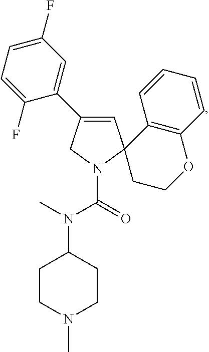

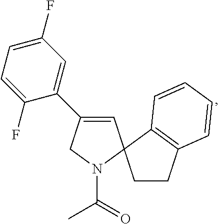

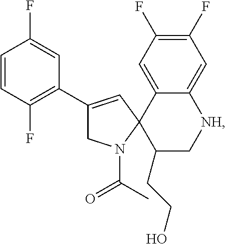

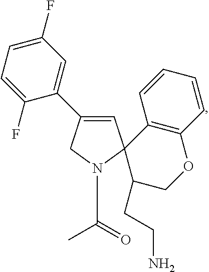

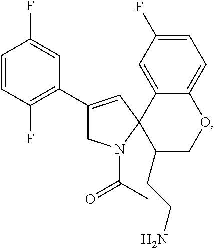

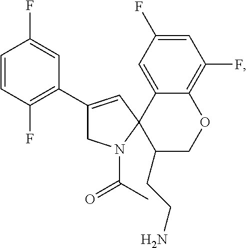

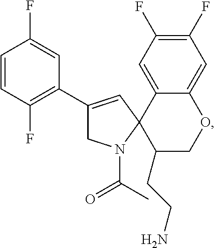

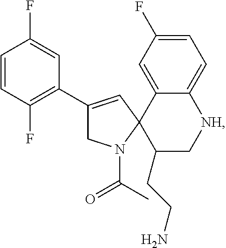

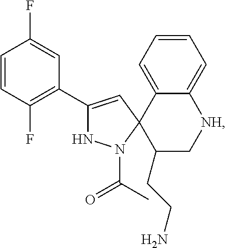

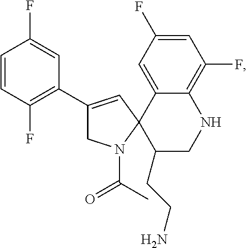

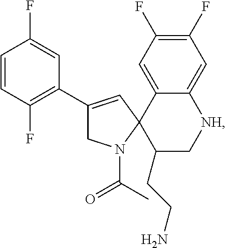

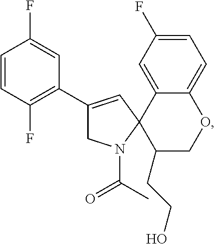

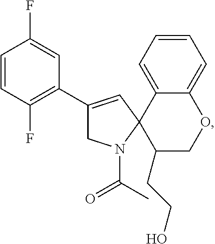

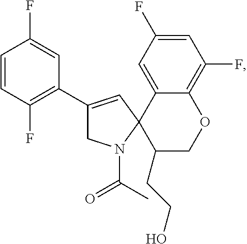

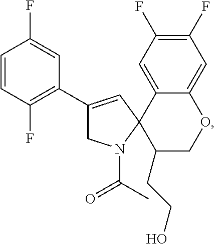

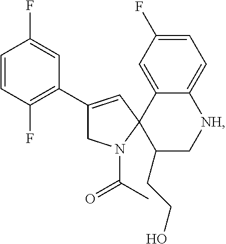

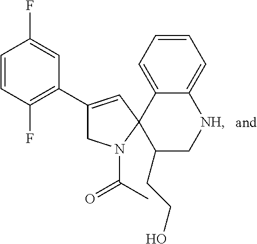

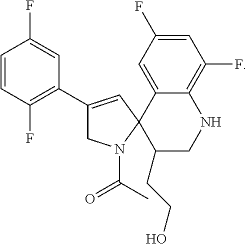

In other embodiments, the present invention provides processes for producing the compounds described in each of the various embodiments above, pharmaceutical formulations or compositions comprising one or more of such compounds, and methods of treating or preventing one or more conditions or diseases associated with KSP kinesin activity such as those discussed in detail below.

As used above, and throughout the specification, the following terms, unless otherwise indicated, shall be understood to have the following meanings:

“Subject” includes both mammals and non-mammalian animals.

“Mammal” includes humans and other mammalian animals.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties. It should be noted that any atom with unsatisfied valences in the text, schemes, examples and tables herein is assumed to have the hydrogen atom(s) to satisfy the valences.

The following definitions apply regardless of whether a term is used by itself or in combination with other terms, unless otherwise indicated. Therefore, the definition of “alkyl” applies to “alkyl” as well as the “alkyl” portions of “hydroxyalkyl”, “haloalkyl”, “alkoxy”, etc.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “lower alkyl” means a group having about 1 to about 6 carbon atoms in the chain which may be straight or branched. “Alkyl” may be unsubstituted or optionally substituted by one or more substituents as described herein. Non-limiting examples of suitable alkyl groups include methyl, ethyl, n-propyl, isopropyl and t-butyl. “Alkyl” includes “Alkylene” which refers to a difunctional group obtained by removal of a hydrogen atom from an alkyl group that is defined above. Non-limiting examples of alkylene include methylene (—CH₂—), ethylene (—CH₂CH₂—) and propylene (—C₃H₆—); which may be linear or branched.

“Heteroalkyl” means an alkyl moiety as defined above, having one or more carbon atoms, for example one, two or three carbon atoms, replaced with one or more heteroatoms, which may be the same or different, where the point of attachment to the remainder of the molecule is through a carbon atom of the heteroalkyl radical. Suitable such heteroatoms include O, S, (and S(O), S(O)₂, etc.) and N. Non-limiting examples include ethers, thioethers, amines, 2-aminoethyl, 2-dimethylaminoethyl, and the like.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. “Alkenyl” may be unsubstituted or optionally substituted by one or more substituents as described herein. Non-limiting examples of suitable alkenyl groups include ethenyl, propenyl, n-butenyl, 3-methylbut-2-enyl, n-pentenyl, octenyl and decenyl.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. “Alkynyl” may be unsubstituted or optionally substituted by one or more substituents as described herein.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl-group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl-group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

“Cycloalkylalkyl” means a cycloalkyl moiety as defined above linked via an alkyl moiety (defined above) to a parent core. Non-limiting examples of suitable cycloalkylalkyls include cyclohexylmethyl, adamantylmethyl and the like.

“Cycloalkenyl” means a non-aromatic mono or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cyclohepta-1,3-dienyl, and the like. Non-limiting example of a suitable multicyclic cycloalkenyl is norbornylenyl.

“Cycloalkenylalkyl” means a cycloalkenyl moiety as defined above linked via an alkyl moiety (defined above) to a parent core. Non-limiting examples of suitable cycloalkenylalkyls include cyclopentenylmethyl, cyclohexenylmethyl and the like.

“Halogen” means fluorine, chlorine, bromine, or iodine. Preferred are fluorine, chlorine and bromine.

“Ring system substituent” means a substituent attached to a ring system (such as an aromatic, heteroaromatic, saturated or partially unsaturated alicyclic or heterocyclic ring systems) which, for example, replaces an available hydrogen on a carbon atom or a heteroatom of the ring system. “Ring system substituents” may be referred to as such, or may be referred to as a variable or specific functional group or groups that are attached to a ring system. For example, when R² in Formula (I) is —C(O)R¹⁷ and R¹⁷ is a substituted heterocycloalkyl, the substituent attached to the heterocycloalkyl is a ring system substituent. If two or more ring system substituents are present on a given ring, such multiple substituents may be attached to the same or different available ring carbon or heteroatom. Ring system substituents may be the same or different, and are as described herein. Other non-limiting examples of ring system substituents include alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, —C(═N—CN)—NH₂, —C(═NH)—NH₂, —C(═NH)—NH(alkyl), Y₁Y₂N—, Y₁Y₂N-alkyl-, Y₁Y₂NC(O)—, Y₁Y₂NSO₂— and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system. Examples of such moiety are methylene dioxy, ethylenedioxy, —C(CH₃)₂— and the like which form moieties such as, for example:

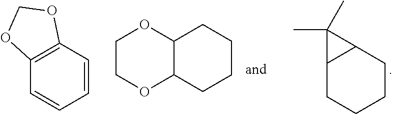

“Heteroarylalkyl” (or “heteroaryl-alkyl-”) means a heteroaryl moiety as defined above linked via an alkyl moiety (defined above) to a parent core. Non-limiting examples of suitable heteroaryls include 2-pyridinylmethyl, quinolinylmethyl and the like.

“Heterocyclyl” (or “heterocycloalkyl”) means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, azetidinyl, lactam, lactone, and the like.

“Heterocyclyl” also includes rings wherein ═O replaces two available hydrogens on the same carbon atom (i.e., heterocyclyl includes rings having a carbonyl group in the ring). An example of such moiety is pyrrolidone:

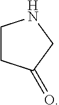

“Heterocyclylalkyl” (or “heterocycloalkylalkyl” or “heterocycloalkyl-alkyl-”) means a heterocyclyl moiety as defined above linked via an alkyl moiety (defined above) to a parent core. Non-limiting examples of suitable heterocyclylalkyls include piperidinylmethyl, piperazinylmethyl and the like.

“Heterocyclenyl” (or “heterocycloalkenyl”) means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridine, 1,2-dihydropyridyl, 1,4-dihydropyridyl, 1,2,3,6-tetrahydropyridine, 1,4,5,6-tetrahydropyrimidine, 2-pyrrolinyl, 3-pyrrolinyl, 2-imidazolinyl, 2-pyrazolinyl, dihydroimidazole, dihydrooxazole, dihydrooxadiazole, dihydrothiazole, 3,4-dihydro-2H-pyran, dihydrofuranyl, fluorodihydrofuranyl, 7-oxabicyclo[2.2.1]heptenyl, dihydrothiophenyl, dihydrothiopyranyl, and the like. “Heterocyclenyl” may also mean a single moiety (e.g., carbonyl) which simultaneously replaces two available hydrogens on the same carbon atom on a ring system. Example of such moiety is pyrrolidinone:

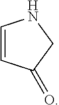

“Heterocyclenylalkyl” (or “heterocycloalkenylalkyl” or “heterococloalkenyl-alkyl-”) means a heterocyclenyl moiety as defined above linked via an alkyl moiety (defined above) to a parent core.

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring:

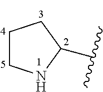

there is no —OH attached directly to carbons marked 2 and 5.

It should also be noted that tautomeric forms such as, for example, the moieties:

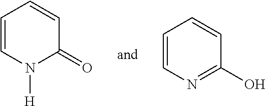

are considered equivalent in certain embodiments of this invention.

“Alkynylalkyl” means an alkynyl-alkyl-group in which the alkynyl and alkyl are as previously described. Preferred alkynylalkyls contain a lower alkynyl and a lower alkyl group. The bond to the parent moiety is through the alkyl. Non-limiting examples of suitable alkynylalkyl groups include propargylmethyl.

“Heteroaralkyl” means a heteroaryl-alkyl-group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl-group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkylsilyl” means an alkyl-Si— group in which alkyl is as previously defined and the point of attachment to the parent moiety is on Si. Preferred alkylsilyls contain lower alkyl. An example of an alkylsilyl group is trimethylsilyl (—Si(CH₃)₃).

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure”, it is meant a compound that is sufficiently robust to survive isolation to a useful degree of purity from a reaction mixture, and formulation into an efficacious therapeutic agent.

The term “optionally substituted” means optional substitution with the specified or implied groups, radicals or moieties.

The term “purified”, “in purified form” or “in isolated and purified form” for a compound refers to the physical state of said compound after being isolated from a synthetic process or natural source or combination thereof. Thus, the term “purified”, “in purified form” or “in isolated and purified form” for a compound refers to the physical state of said compound after being obtained from a purification process or processes described herein or well known to the skilled artisan, in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R², etc.) occurs more than one time in any constituent or in any one of The invention, its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

The term “pharmaceutical composition” is also intended to encompass both the bulk composition and individual dosage units comprised of more than one (e.g., two) pharmaceutically active agents such as, for example, a compound of the present invention and an additional agent selected from the lists of the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills and the like. Similarly, the herein-described method of treating a patient by administering a pharmaceutical composition of the present invention is also intended to encompass the administration of the afore-said bulk composition and individual dosage units.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g, a drug precursor) that is transformed in vivo to yield a compound of Formula (I) or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of Formula (I) or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if a compound of Formula (I) contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Formula (I) incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N- or di-N,N—(C₁-C₆)alkylaminoalkyl, —C(Y⁴)Y⁵ wherein Y⁴ is H or methyl and Y⁵ is mono-N- or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

The compounds of the invention can form salts which are also within the scope of this invention. Reference to a compound of the invention herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of any one of the invention contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the invention may be formed, for example, by reacting a compound of the invention with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al., Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the compounds of the invention include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

Compounds of The invention, and salts, solvates, esters and prodrugs thereof, may exist in their tautomeric form (for example, as an amide or imino ether). All such tautomeric forms are contemplated herein as part of the present invention.

The compounds of Formula (I) may contain asymmetric or chiral centers, and, therefore, exist in different stereoisomeric forms. It is intended that all stereoisomeric forms of the compounds of Formula (I) as well as mixtures thereof, including racemic mixtures, form part of the present invention. In addition, the present invention embraces all geometric and positional isomers. For example, if a compound of Formula (I) incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula (I) may be atropisomers (e.g., substituted biaryls) and are considered as part of this invention. Enantiomers can also be separated by use of chiral HPLC column.

It is also possible that the compounds of Formula (I) may exist in different tautomeric forms, and all such forms are embraced within the scope of the invention. Also, for example, all keto-enol and imine-enamine forms of the compounds are included in the invention.

An stereoisomers (for example, geometric isomers, optical isomers and the like) of the compounds of the invention (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of Formula (I) incorporates a double bond or a fused ring, both the cis- and trans-forms, as well as mixtures, are embraced within the scope of the invention. Also, for example, all keto-enol and imine-enamine forms of the compounds are included in the invention.) Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of Formula (I) can generally be prepared by following procedures analogous to those disclosed in the Schemes and/or in the Examples hereinbelow, by substituting an appropriate isotopically labelled reagent for a non-isotopically labelled reagent.

Polymorphic forms of the compounds of the invention, and of the salts, solvates, esters and prodrugs of the compounds of the invention, are intended to be included in the present invention.

### PREPARATIVE EXAMPLES

Generally, the compounds of the invention can be prepared by a variety of methods well known to those skilled in the art, for example, by the methods as outlined in the general scheme below and in the examples that follow. The examples should not be construed to limit the scope of the disclosure. Alternative mechanistic pathways and analogous structures will be apparent to those skilled in the art.

EC₅₀ values for the exemplified compounds appearing in the Table below are indicated according to the following ranges:

A—≦500 nM

B—>500 nM

C—>500 nM to ≦1000 nM

D—>1000 nM

The following abbreviations are used in the procedures and schemes:
ACN Acetonitrile
AcOH Acetic acid
Aq Aqueous
BOO Pert-Butoxycarbonyl
BOC-ON [2-(tert-butoxycarbonyloxyimino)-2-phenylacetonitrile]
BOC₂O BOC Anhydride
C degrees Celsius
Cpd Compound
CBZCI Benzyl chloroformate
DCM Dichloromethane
DEAD Diethyl azodicarboxylate
DIAD Diisopropylazodicarboxylate
DI EA Diisopropylethylamine
DMA N,N-Dimethylacetamide
DMAP 4-N,N-Dimethylaminopyridine
DME Dimethoxyethane
DMF Dimethylformamide
DMSO Dimethyl sulfoxide
EDCI 1-(3-Dimethylaminopropyl)-3-ethylcarbodiimide hydrochloride
EI Electron ionization
Eq Equivalents
EtOAc Ethyl acetate
EtOH Ethanol
g grams
h. hours
¹H proton
HATU N,N,N′,N′-Tetramethyl-O-(7-Azabenzotriazol-1-yl)Uronium hexafluorophosphate
Hex hexanes
HOBT 1-Hydroxybenzotriazole
HPLC High pressure liquid chromatography
KSP Kinesin spindle protein
LAH Lithium aluminum hydride
LDA Lithium diisopropylamide
LHMDS Lithium hexamethy d silylamide
M Molar
mmol milimolar
mCPBA meta-Chloroperoxybenzoic acid
Me Methyl
MeCN Acetonitrile
MeOH Methanol
min Minutes
mg Milligrams
MHZ Megahertz
mL Milliliter
MPLC Medium Pressure Liquid Chromatography
NMR Nuclear Magnetic Resonance
MS Mass Spectroscopy
NBS N-Bromosuccinimide
NIS N-Iodosuccinimide
NMM N-Methylmorpholine
NMP 1-methyl-2-pyrrolidone
ON Overnight
PCC Pyridinium Chlorochromate
PTLC Preparative thin layer chromatography
Pyr Pyridine
RT Room temperature
sgc Silica gel 60 chromatography
tBOC ten-Butoxycarbonyl
TEA Triethylamine
TFA Trifluoroacetic acid
THF Tetrahydrofuran
TLC Thin layer chromatography
t_{R} Retention time

### EXAMPLES

### General Scheme

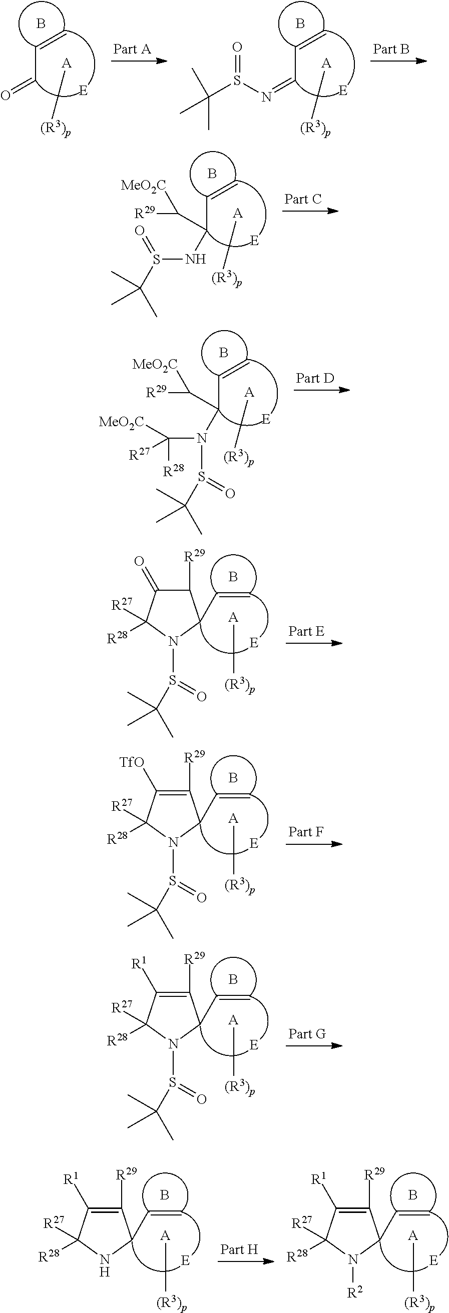

### Example 1

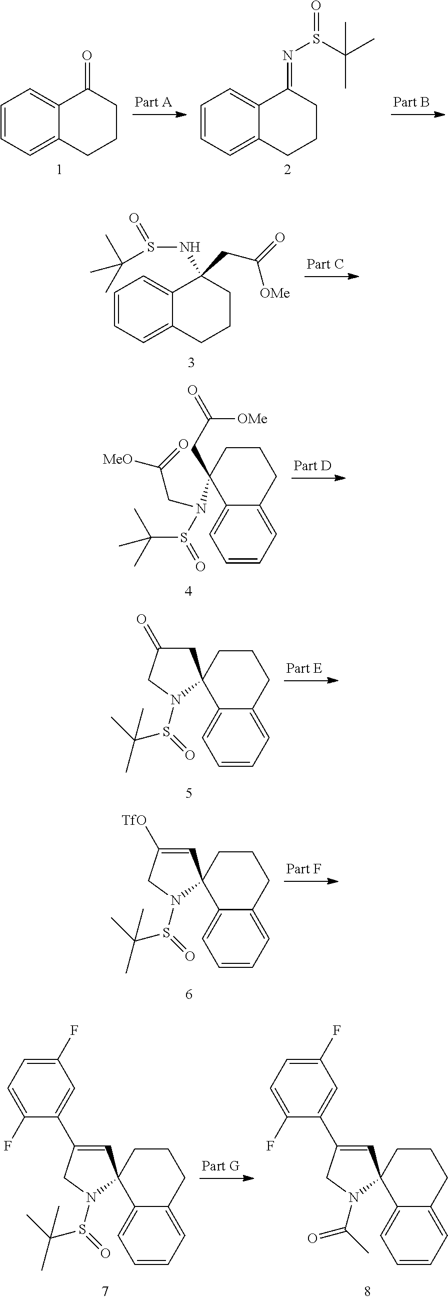

### Part A:

To a stirred solution of (R)-(+)-2-methyl-2-propanesulfinamide (8.22 g, 68 mmol) in THF (100 mL) was added Ti(OEt)₄ (31.9 g, 140 mmol) and 1-tetralone (1) (10 g, 68 mmol) then heated to 70° C. for 12 hours. The reaction mixture was cooled and concentrated in vacuo, diluted with EtOAc (80 mL) and brine (100 mL) then the mixture was filtered through a pad of celite. The organic phase was separated, dried (Na₂SO₄) and concentrated in vacuo. The residue was subjected to flash silica chromatography, gradient elution (0 to 100%) hexane/ethyl acetate to furnish compound 2 (6 g, 24 mmol, 35%). HPLC-MS t_{R}=2.02 min (UV_{254 nm}), mass calculated for formula C₁₄H₁₉NOS 249.1, observed LCMS m/z 250.1 (M+H).

### Part B:

To a solution of DIEA (2.4 mL, 17.6 mmol) in THF (35 mL) at 0° C. was added n-BuLi (11.2 mL, 1.5 M). The solution was stirred for 30 minutes then cooled to −78° C. to which was added methyl acetate (1.31 mL, 16 mmol). The solution was stirred for 40 minutes upon which was added a hexane solution of CITi(OⁱPr)₃ (33.6 mL, 1M). The mixture was stirred for a further 30 minutes to which was added compound 2 (2 g, 8 mmol) in THF (5 mL). The solution was stirred for 3 hours, quenched with saturated NH₄Cl (30 mL), filtered through a pad of celite and then washed with EtOAc (100 mL). The organic phase was separated, washed with brine (30 mL), dried (Na₂SO₄) and concentrated in vacuo. The residue was subjected to flash silica chromatography, gradient elution (0 to 100%) hexane/ethyl acetate to furnish compound 3 (2.1 g, 6.5 mmol, 81%). HPLC-MS t_{R}=1.9 min (UV_{254 nm}); mass calculated for formula C₁₇H₂₅NO₃S 323.16, observed LCMS m/z 324.1 (M+H).

### Part C:

To a solution of compound 3 (250 mg, 0.77 mmol) in CH₃CN (3 mL) is added K₂CO₃ (318 mg, 2.31 mmol) and methyl bromoacetate (176 mg, 1.16 mmol) and the mixture is heated to 80° C. for 12 hours. The mixture is filtered, concentrated in vacuo and subjected to flash silica chromatography, gradient elution (0 to 100%) hexane/ethyl acetate to furnish compound 4 (X g, XX mmol, XX %). HPLC-MS t_{R}=xx min (UV_{254 nm}), mass calculated for formula C₂₀H₂₉NO₅S 395.18 observed LCMS m/z xxx (M+H).

### Part D:

To a solution of KO^{t}Bu (168 mg, 1.5 mmol) in toluene (5 mL) at 0° C. is added a solution of compound 4 (395 mg, 1 mmol) in toluene (1 ml). The reaction is allowed to warm to room temperature and stirred for 1 hour. The mixture is quenched with saturated NH₄Cl (10 mL) and extracted with ethyl acetate (2×15 mL). The combined organics are washed with brine, dried (Na₂SO₄) and concentrated in vacuo. The residue is dissolved in DMSO (1 mL) to which is added H₂O (0.05 mL), sodium chloride (116 mg, 2 mmol) and the mixture heated to 140° C. for 2 h. The reaction is diluted with H₂O (10 mL), extracted with ethyl actetate (2×10 mL), washed with brine, dried (Na₂SO₄) and then concentrated in vacuo. The residue is subjected to flash silica chromatography, gradient elution (0 to 100%) hexane/ethyl acetate to furnish compound 5 (Xg, XX mmol, XX %). HPLC-MS t_{R}=xx min (UV_{254 nm}); mass calculated for formula C17H₂₃NO₂S 305.14, observed LCMS m/z xxx (M+H).

### Part E:

To a solution of compound 5 (305 mg, 1 mmol) in THF (15 ml) at −78° C. is added NaHMDS (1.2 mL, 1.2 mmol, 1M). The reaction is warmed to 0° C. and held for 1 hour. The reaction is re-cooled to −78° C. and a solution of N-phenylbis(trifluoro-methanesulphonimide) (427 mg, 1.2 mmol) in THF (2 mL) is slowly added. The reaction is allowed to reach room temperature, stirred for 12 hours and then quenched with saturated NH₄Cl (15 mL). The mixture is extracted with ethyl acetate (2×20 mL) and the combined organics are washed with brine (15 mL), dried (Na₂SO₄) and concentrated in vacuo. The residue is subjected to flash silica chromatography, gradient elution (0 to 100%) hexane/ethyl acetate to furnish compound 6 (X g, XX mmol, XX %). HPLC-MS t_{R}=xxx (UV_{254 nm}), mass calculated for formula C₂₃H₂₅F₂NOS 437.09, observed LCMS m/z XXX (M+H).

### Part F:

To a solution of compound 6 (437 mg, 1 mmol) in DME (8 mL) is added Na₂CO₃ (6 mL, 2M). The mixture is degassed with nitrogen followed by the addition of tetrakis (triphenylphospine) palladium (0) (57 mg, 0.05 mmol). The mixture is heated 80° C. for 2 hours, cooled to room temperature and then extracted with ethyl acetate (2×20 mL). The combined organics are washed with brine, dried (Na₂SO₄), concentrated in vacuo and the residue is subjected to flash silica chromatography, gradient elution (0 to 100%) hexane/ethyl acetate to furnish compound 7 (X g, XX mmol, XX %). HPLC-MS T_{R}=xxx min (UV_{254 nm}); mass calculated for formula C₂₃H₂₅F₂NOS 401.16, observed LCMS m/z xxx (M+H).

### Part G:

To a solution of compound 7 (401 mg, 1 mmol) in ethanol (3 mL) is added saturated ethanolic HCl (5 ml) and the reaction is stirred for 12 hours then concentrated in vacuo. The residue is re-dissolved in DCM (5 mL) to which is added triethylamine (0.5 g, 5 mmol) followed by acetyl chloride (78 mg, 1.5 mmol) at 0° C. The solution is stirred for 2 hours and then quenched with brine (5 mL). The aqueous layer is extracted with ethyl acetate (2×10 mL) and the combined organics are washed with brine, dried (Na₂SO₄), and concentrated in vacuo. The residue is subjected to flash silica chromatography, gradient elution (0 to 100%) hexane/ethyl acetate to furnish compound 8 (X g, XX mmol, x %). HPLC-MS t_{R}=xxx min (UV_{254 nm}); mass calculated for formula C₂₁H₁₉F₂NO 339.14, observed LCMS m/z xxx (M+H).

The compounds shown in Table 1 above, which may be synthesized using this procedure, are examples of the compounds of the invention.

The pharmacological properties of the compounds of this invention, including their efficacy as inhibitors of KSP activity, may be confirmed by a number of pharmacological assays. The inhibitory activity of the compounds of the invention towards KSP may be assayed by methods known in the art, for example, by using the methods as described below and in the examples above.

### KSP Biochemical Assay

KSP biochemical enzyme assays are performed in 384-well plates. All reagents are thawed on ice. Compounds were diluted in 100% DMSO to desirable concentrations. 10 mg microtubules (Cytoskeleton) are reconstituted in 10 mL tubulin buffer (80 mM PIPES pH 6.8, 1 mM EGTA, 1 mM MgCl₂, 0.005% sodium azide) plus 100 ul 2 mM paclitaxel (Cytoskeleton).

Each reaction consisted of 10 nM KSP motor domain (amino acid 15-368), 20 uM paclitaxel (Cytoskeleton), 0.18 uM microtubules, 100 uM ATP (Roche) and kinesin buffer (20 mM ACES pH 7.0, 1 mM EGTA, 1 mM MgCl₂, 25 mM KCl, 1 mM DTT). For each reaction, 19 uL of mixture containing KSP motor domain, paclitaxel, microtubules and kinesin buffer are combined with 1 uL diluted compound. The reaction is started by the addition of 5 uL ATP. The reaction is allowed to run for 1 hour at room temperature. The reaction is stopped by adding 50 ul Biomol Green (Biomol International). After an additional 30 minutes, absorbance at OD620 nm is measured using an Envision.

IC50 Determinations: Dose-response curves are plotted from inhibition data generated each in duplicate, from 8 point serial dilutions of inhibitory compounds. Concentration of compound is plotted against enzyme activity (OD reading). To generate 1050 values, the dose-response curves are then fitted to a standard sigmoidal curve and 1050 values were derived by nonlinear regression analysis.

### KSP Cellular Assay:

HCT116 colon cancer cells are grown in DMEM:F12 media with 10% heat inactivated FBS at 37 degrees with 5% CO2. Cells are plated at 7,500 cells per well in PDL coated 384-well tissue culture plates. 6 hours later media are removed and new media containing drug is added. Cells were incubated with drug for 16 hours. All further steps are performed at room temperature in the dark. Cells are fixed with 25 ul/well Prefer fixation solution plus 250 nM Hoechst dye and incubated for 30 minutes. The fixation solution is removed and cells are washed with PBS. Cells were then permeabilized with 25 ul/well 0.2% Triton-X in PBS and incubated for 10 minutes. Cells are washed with PBS and then incubated with 25 ul/well PBS containing 3% FBS for 30 minutes. Cells are then stained overnight at 4 degrees with 25 ul/well antibody solution in PBS plus 3% FBS. Antibodies used are Phos-Histone H3 (ser10)-Alexa Flur 488 Conjugate and Phos-MPM2 Texas Red Conjugate. Cells are washed with PBS and then immunofluorescence images captured with HT Pathway microscope. The percent of cells staining positive is calculated and EC₅₀ values for the compounds of the invention are determined using Excel XLfit.

EC50 Determinations: Dose-response curves are plotted from inhibition data generated each in duplicate, from 8 point serial dilutions of inhibitory compounds. Concentration of compound is plotted against enzyme activity (OD reading). To generate EC50 values, the dose-response curves are then fitted to a standard sigmoidal curve and EC50 values are derived by nonlinear regression analysis.

Methods of Use

As inhibitors of KSP activity, the compounds of the invention are contemplated as being useful in treating a wide variety of diseases, conditions, or disorders (“diseases”).

In one embodiment, the present invention provides a method of inhibiting KSP kinesin activity in a subject (e.g., cells, animals, or humans) in need thereof, comprising administering to said subject at least one compound or composition of the invention or a pharmaceutically acceptable salt, ester, isomer, tautomer, or prodrug thereof.

In one embodiment, the present invention provides a method of selectively inhibiting KSP kinesin activity in a subject (e.g., cells, animals, or humans) in need thereof, comprising administering to said subject at least one compound or composition of the invention or a pharmaceutically acceptable salt, ester, isomer, tautomer, or prodrug thereof.

In some embodiments, diseases which are amenable to treatment include those susceptible to alteration of mitosis by KSP activity inhibition. As will be appreciated by those skilled in the art, mitosis may be altered in a variety of ways, such as by increasing or decreasing the activity of a component in the mitotic pathway or by disturbing equilibrium (e.g., by inhibiting or activating certain components).

In one embodiment, the invention provides a method of treating or preventing a disease associated with KSP activity in a subject in need thereof comprising administering a therapeutically effective amount of at least one compound of the invention or a pharmaceutically acceptable salt or ester thereof to said subject.

In one embodiment, the compounds of the invention can be used to inhibit mitotic spindle formation, thus causing prolonged cell cycle arrest in mitosis. “Inhibit” in this context means decreasing or interfering with mitotic spindle formation or causing mitotic spindle dysfunction. “Mitotic spindle formation” means the organization of microtubules into bipolar structures by mitotic kinesins. “Mitotic spindle dysfunction” means mitotic arrest and monopolar spindle formation.

In one embodiment, the compounds of the invention can be useful for binding to, and/or inhibiting the activity of, KSP. In one embodiment, the KSP is human KSP. In one embodiment, such KSP activity is inhibited in vitro, in vivo (e.g., in a patient in need thereof), or ex vivo.

In other embodiments, the compounds of the invention may be used to bind to or inhibit the activity of KSP kinesins from non-human organisms. In this context, “inhibit” means increasing or decreasing spindle pole separation, causing malformation, i.e., splaying, of mitotic spindle poles, or otherwise causing morphological perturbation of the mitotic spindle.

Also included within the definition of KSP for purposes of the present invention are variants and/or fragments of KSP (see, e.g., U.S. Pat. No. 6,437,115).

The compounds of the invention can be used to treat diseases associated with or caused by aberrant cellular proliferation. Such disease states include, but are not limited to, cancer (further discussed below), hyperplasia, cardiac hypertrophy, autoimmune diseases, fungal disorders, arthritis, graft rejection, inflammatory bowel disease, immune disorders, inflammation, cellular proliferation induced after medical procedures, including, but not limited to, surgery, angioplasty, and the like. Treatment includes inhibiting cellular proliferation. It is appreciated that in some cases the cells may not be in an abnormally proliferative state and yet require treatment. For example, during wound healing, the cells may be proliferating “normally”, but inhibition of cellular proliferation may be desired. Thus, in one embodiment, the invention herein includes application to cells or subjects afflicted with or subject to impending affliction with any one of these conditions, disorders or states.

The terms “treating cancer” and “treatment of cancer” refer to administration to a mammal afflicted with a cancerous condition and to an effect that alleviates the cancerous condition by killing at least some of the cancerous cells, and also to an effect that results in the inhibition of growth and/or metastasis of the cancer.

Due to their KSP inhibitory action, the compounds, compositions and methods provided herein are useful for the treatment of a wide variety of cancers. Non-limiting examples of such cancers include solid tumors and hematological cancers, such as those of the skin, breast, brain, colon, gall bladder, thyroid, cervical carcinomas, testicles, and blood. Additional non-limiting examples of cancers suitable for treatment include:

Cardiac: sarcoma (angiosarcoma, fibrosarcoma, rhabdomyosarcoma, liposarcoma), myxoma, rhabdomyoma, fibroma, lipoma and teratoma;

Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;

Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, vinous adenoma, hamartoma, leiomyoma);

Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);

Liver: hepatoma (hepatocellular carcinoma), cholangiocarcinoma, hepatoblastoma, angiosarcoma, hepatocellular adenoma, hemangioma;

Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors;

Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma);

Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma);

Hematologic: blood (myeloid leukemia (acute and chronic), acute lymphoblastic leukemia, acute and chronic lymphocytic leukemia, myeloproliferative diseases, multiple myeloma, myelodysplastic syndrome), Hodgkin's disease, non-Hodgkin's lymphoma (malignant lymphoma), B-cell lymphoma, T-cell lymphoma, hairy cell lymphoma, Burkett's lymphoma, promyelocytic leukemia;

Skin: malignant melanoma, basal cell carcinoma, squamous cell carcinoma, Karposi's sarcoma, moles dysplastic nevi, lipoma, angioma, dermatofibroma, keloids, psoriasis;

Adrenal glands: neuroblastoma; and

Other tumors: including xenoderoma pigmentosum, keratoctanthoma and thyroid follicular cancer.

As used herein, treatment of cancer includes treatment of cancerous cells, including cells afflicted by any one of the conditions, states, or disorders described above.

The compounds of the present invention may also be useful in the chemoprevention of cancer. Chemoprevention is defined as inhibiting the development of invasive cancer by either blocking the initiating mutagenic event or by blocking the progression of pre-malignant cells that have already suffered an insult. The compounds of the present invention may also be useful in inhibiting cancer relapse.

The compounds of the present invention may also be useful in inhibiting tumor angiogenesis and metastasis.

The compounds of the present invention may also be useful as antifungal agents, by modulating the activity of the fungal members of the bimC kinesin subgroup, as is described in U.S. Pat. No. 6,284,480.

For each of the foregoing embodiments, the amount of the at least one compound of the invention administered is preferably an effective amount for the intended purpose. The phrase “effective amount” means that amount of a compound of the invention, and other pharmacological or therapeutic agents described herein, that will elicit a biological or medical response of a tissue, a cell, a population of cells (e.g., a population of aberrantly proliferating cells such as cancer cells or psioratic cells), a system, or a subject (e.g., animal or human) that is being sought by the administrator (such as a researcher, doctor or veterinarian) which includes alleviation of the symptoms of the condition or disease being treated and the prevention, slowing or halting of progression of one or more cellular proliferation diseases. “Therapeutically effective amount” means effective amount where the purpose includes a therapeutic purpose, such as in a human or non-human patient in need of treatment. The formulations or compositions, combinations and treatments of the present invention can be administered by any suitable means which produce contact of these compounds with the site of action in the targeted population of aberrantly proliferating cells or the body of the subject being treated.

Suitable dosage ranges for the various embodiments of the invention are readily determined by those skilled in the art and depend upon intended use. Suitable dose ranges include from about 0.001 to about 500 mg/kg of body weight/day of a compound of the invention or a pharmaceutically acceptable salt, ester, or prodrug (etc.) thereof. Another suitable dosage ranges from about 0.01 to about 25 mg/kg of body weight/day. For administration of pharmaceutically acceptable salts of the above compounds, the weights indicated above refer to the weight of the acid equivalent or the base equivalent of the therapeutic compound derived from the salt.

It may be preferable to administer KSP kinesin inhibitors which can specifically inhibit KSP kinesin activity at low concentrations, for example, those that cause a level of inhibition of 50% or greater at a concentration of 50 μM or less, 100 nM or less, or 50 nM or less. The administration of such compounds of the invention represents various embodiments of the present invention.

Compositions

In some embodiments, the at least one compound of the invention is administered as the neat chemical. In other embodiments, the compounds of the invention are administered as a pharmaceutical composition. Thus, pharmaceutical compositions comprising at least one compound of the invention are within the scope of the present invention. Such pharmaceutical compositions of the present invention comprise at least one compound of the invention (e.g., doses of one, two, three, or more different compounds of the invention), together with one or more acceptable carriers, and optionally other therapeutic agents. Each carrier (including, e.g., adjuvants or vehicles) must be acceptable in the sense of being compatible with the other ingredients of the composition and not injurious to the intended purpose or, in the case of therapy, the subject being treated. Accordingly, in another embodiment, this invention also provides pharmaceutical compositions comprising at least one compound of the invention, or a pharmaceutically acceptable salt, solvate, ester, prodrug, or isomer thereof and at least one pharmaceutically acceptable carrier.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

The term pharmaceutical composition is also intended to encompass both the bulk composition and individual dosage units comprised of more than one (e.g., two) pharmaceutically active agents such as, for example, a compound of the present invention and an additional agent selected from the lists of the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills and the like. Similarly, the herein-described method of treating a subject by administering a pharmaceutical composition of the present invention is also intended to encompass the administration of the afore-said bulk composition and individual dosage units.

Additionally, the compositions of the present invention may be formulated in sustained release form to provide the rate controlled release of any one or more of the components or active ingredients to optimize the therapeutic effects. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral (e.g., subcutaneous, intramuscular, introrbital, intracapsular, intraspinal, intrasternal, intravenous, etc.) administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

In one embodiment, the at least one compound or composition of the invention is formulated for subcutaneous administration.

In one embodiment, the at least one compound or composition of the invention is formulated for oral administration.

In one embodiment, the at least one compound or composition of the invention is formulated for parenteral administration.

In one embodiment, the at least one compound or composition of the invention is formulated for intravenous administration.

In one embodiment, the pharmaceutical preparation is provided in a unit dosage form. In such form, the preparation is subdivided into suitably sized unit doses containing appropriate quantities of the active component, e.g., an effective amount to achieve the desired purpose.

As stated elsewhere herein, the quantity of active compound in a unit dose of preparation may be varied or adjusted to suit intended purpose. Additional non-limiting examples of such doses range from about 1 mg to about 100 mg, alternatively from about 1 mg to about 50 mg, or alternatively from about 1 mg to about 25 mg, according to the particular application.

The actual dosage employed may be varied depending upon the requirements of the patient and the severity of the condition being treated, Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts or esters thereof will be regulated according to the judgment of the attending clinician considering such factors as age, condition and size of the patient as well as severity of the symptoms being treated. A typical recommended daily dosage regimen for oral administration can range from about 1 mg/day to about 500 mg/day, preferably 1 mg/day to 200 mg/day, in two to four divided doses.

In another embodiment, the present invention provides a kit comprising a therapeutically effective amount of at least one compound of the invention or a pharmaceutically acceptable salt or ester thereof and at least one pharmaceutically acceptable carrier, adjuvant or vehicle, and, optionally, inserts and/or labels which include instructions for use.

In another embodiment, the present invention provides a kit comprising an amount of at least one compound of the invention or a pharmaceutically acceptable salt or ester thereof and an amount of at least one additional therapeutic agent listed above, wherein the amounts of the two or more ingredients result in desired therapeutic effect.

In another embodiment, the present invention provides for: the use of at least one compound of the invention, or a pharmaceutically acceptable salt, solvate, ester or prodrug thereof, to manufacture a medicament for inhibiting KSP kinesin activity in a subject in need thereof.

In another embodiment, the present invention provides for: the use of at least one compound of the invention, or a pharmaceutically acceptable salt, solvate, ester or prodrug thereof, to manufacture a medicament for treating one or more diseases by inhibiting KSP kinesin activity in a patient in need thereof.

In another embodiment, the present invention provides for: the use of at least one compound of the invention, or a pharmaceutically acceptable salt, solvate, ester or prodrug thereof, to manufacture a medicament for treating any one of the conditions, disease, or disorders described herein.

In another embodiment, the present invention provides for: the use of a combination comprising (i) at least one compound of the invention, or a pharmaceutically acceptable salt, solvate, ester or prodrug thereof; and (ii) at least one second active ingredient described herein.

Combination Therapies

The compounds of the invention (and the compositions comprising at least one compound of the invention) are also useful in combination with one or more therapeutic agents other than a compound of the invention. Such therapeutic agents are selected according to intended purpose. Non-limiting examples of such agents include those which are effective for treating the underlying disease or condition, and/or for minimizing one or more side effects of a therapeutic agent, and/or for enhancing or altering the bioavailability of an administered therapeutic agent, etc.

Combinations of the compounds of the invention with other anti-cancer or chemotherapeutic agents are within the scope of the invention. Non-limiting examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer (or other indication) involved. The following description provides additional non-limiting examples of such combination agents. Those of ordinary skill in the art will readily be able to determine additional suitable agents.

Thus, anti-cancer agents suitable for use in combination with at least one compound of the invention (or composition comprising at least one compound of the invention) include, but are not limited to the following: estrogen receptor modulators, androgen receptor modulators, retinoid receptor modulators, cytotoxic agents, microtubule inhibitors/stabilizing agents, topoisomerase inhibitors, antisense RNA and DNA oligonucleotides, antimetabolites, antibodies coupled to cyclotoxic agents or radioisotypes, HMG-CoA reductase inhibitors, prenyltransferase inhibitors, farnesyl protein transferase inhibitors, angiogenesis inhibitors, kinase inhibitors, COX2 inhibitors, integrin blockers, PPAR agonists, and MDR inhibitors. Additional anticancer agents also include hypoxia activatable agents, proteasome inhibitors, ubiquitin inhibitors, HDM2 inhibitors, TNF activators, BUB-R inhibitors, CENP-E inhibitors, and interferons (e.g., alpha interferon). Such anti-cancer agents can be small molecules or biologics (e.g., RNA antisense and antibodies). The compounds of the invention are also useful when co-administered with radiation therapy.

“Estrogen receptor modulators” refers to compounds that interfere with or inhibit the binding of estrogen to the receptor, regardless of mechanism.

Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-ydrazone, aid SH646. Additional examples include anastrozole and letrazole.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, a difluoromethylornithine, ILX23-7553, trans-N-(4′-hydroxyphenyl) retinamide, and N-4-carboxyphenyl retinamide.

Examples of cytotoxic agents include, but are not limited to, sertenef, cachectin, ifosfamide, tasonermin, lonidamine, carboplatin, altretamine, prednimustine, dibromodulcitol, ranimustine, fotemustine, nedaplatin, oxaliplatin, temozolomide (TEMODAR™ from Schering-Plough Corporation, Kenilworth, N.J.), cyclophosphamide, heptaplatin, estramustine, improsulfan tosilate, trofosfamide, nimustine, dibrospidium chloride, pumitepa, lobaplatin, satraplatin, profiromycin, cisplatin, doxorubicin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine(chloro)platinum(II)] tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deansino-3′-morpholino-13-deoxo-10-hydroxycaminomycin, annamycin, galarubicin, elinafide, MEN10755, 4-demethoxy-3-deamino-3-aziridinyl-4-methylsulphonyl-daunombicin (see WO 00/50032), methoxtrexate, gemcitabine, and mixture thereof.

Examples of microtubule inhibitors/microtubule-stabilising agents include paclitaxel, vindesine sulfate, 3′,4′-didehydro-4′-deoxy-8′-norvincaleukoblastine, docetaxel, vincristine, vinblastin, vinorelobine, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl) benzene sulfonamide, anhydrovinblastine, N,N-dimethyl-L-valyl-L-valyl-N-methyl-L-valyl-L-prolyl-L-proline-t-butylamide, TDX258, the epothilones (see for example U.S. Pat. Nos. 6,284,781 and 6,288,237) and BMS188797.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H) propanamine, 1-amino-9-ethyl-5-fluoro-2,3-dihydro-9-hydroxy-4-methyl-1H,12H-benzo[de]pyrano[3′,4′:b,7]-indolizino[1,2b]quinoline-10,13(9H,15H)dione, lurtotecan, 7-[2-(N-isopropylamino) ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a,5aB,8aa,9b)-9-[2-[N-[2-(dimethylamino)ethyl]-N-methylamino]ethyl]-5-[4-hydroxy-3,5-dimethoxyphenyl]-5,5a,6,8,8a,9-hexohydrofuro(3′,′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino] benzo[g]isoquinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2-(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one, dimesna, and camptostar.

Examples of Antisense RNA and DNA oligonucleotides include: G3139, ODN698, RVASKRAS, GEM231, and INX3001.

Gene therapy can be used to deliver any tumor suppressing gene. Examples of such genes include, but are not limited to, p53, which can be delivered via recombinant virus-mediated gene transfer (see U.S. Pat. No. 6,069,134, for example), a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998; 5(8):1105-13), and gene therapy to interferon gamma (J Immunol 2000; 164:217-222). For an overview of genetic strategies to treating cancer, see Hail at al (Am J Hum Genet 61:785-789, 1997) and Kufe at al (Cancer Medicine, 5th Ed, pp 876-889, BC Decker, Hamilton 2000).

Examples of antimetabolites include: 5-fluorouracil, enocitabine, carmofur, tegafur, pentostatin, doxifluridine, trimetrexate, fludarabine, capecitabine, galocitabine, cytarabine ocfosfate, fosteabine sodium hydrate, raltitrexed, paititrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-fluorouracil, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine and 3-aminopyridine-2-carboxaldehyde thiosemicarbazone.

Examples of monoclonal antibody targeted therapeutic agents include those therapeutic agents which have cytotoxic agents or radioisotopes attached to a cancer cell specific or target cell specific monoclonal antibody. Examples include Bexxar.

“HMG-CoA reductase inhibitors” refers to inhibitors of 3-hydroxy-3-methylglutaryl-CoA reductase. Examples of HMG-CoA reductase inhibitors that may be used include but are not limited to lovastatin (MEVACOR; see U.S. Pat. Nos. 4,231,938, 4,294,926 and 4,319,039), simvastatin (ZOCOR®; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL®; see U.S. Pat. Nos. 5,354,772, 4,911,165, 4,929,437, 5,189,164, 5,118,853, 5,290,946 and 5,356,896) and atorvastatin (LIPITOR®; see U.S. Pat. Nos. 5,273,995, 4,681,893, 5,489,691 and 5,342,952). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefore the use of such salts, esters, open acid and lactone forms is included in the scope of this invention.

“Prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase).

Examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. Nos. 5,420,245, 5,523,430, 5,532,359, 5,510,510, 5,589,485, 5,602,098, European Patent Publ. 0 618 221, European Patent Publ. 0 675 112, European Patent Publ. 0 604181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO, 97/30053, WO 97/44350, WO 98/02436, and U.S. Pat. No. 5,532,359. For an example of the role of a prenyl-protein transferase inhibitor on angiogenesis see European of Cancer, Vol. 35, No. 9, pp. 1394-1401 (1999).

Examples of farnesyl protein transferase inhibitors include SARASAR™ (4-[2-[4-[(11R)-3,10-dibromo-8-chloro-6,11-dihydro-5H-benzo[5,6]cyclohepta[1,2-b]pyridin-11-yl-]-1-piperidinyl]-2-oxoethyl]-1-piperidinecarboxamide from Schering-Plough Corporation, Kenilworth, N.J.), tipifarnib (Zarnestre or R115777 from Janssen Pharmaceuticals), L778,123 (a farnesyl protein transferase inhibitor from Merck & Company, Whitehouse Station, N.J.), BMS 214662 (a farnesyl protein transferase inhibitor from Bristol-Myers Squibb Pharmaceuticals, Princeton, N.J.).

“Angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, interleukin-12, pentosan polysulfate, cyclooxygenase inhibitors, including nonsteroidal anti-inflammatories (NSAIDs) like aspirin and ibuprofen as well as selective cyclooxygenase-2 inhibitors like celecoxib and rofecoxib (PNAS, Vol. 89, p. 7384 (1992); JNCI, Vol. 69, p. 475 (1982); Arch. Opthalmol., Vol. 108, p. 573 (1990); Anat. Rec., Vol. 238, p. 68 (1994); FEBS Letters, Vol. 372, p. 83 (1995); Clin. Orthop. Vol. 313, p. 76 (1995); J. Mot Endocrinol., Vol. 16, p. 107 (1996); Jpn. J. Pharmacol., Vol. 75, p. 105 (1997); Cancer Res., Vol. 57, p. 1625 (1997); Cell, Vol. 93, p. 705 (1998); Intl. J. Mol. Med., Vol. 2, p. 715 (1998); J. Biol. Chem., Vol. 274, p. 9116 (1999)), steroidal anti-inflammatories (such as corticosteroids, mineralocorticoids, dexamethasone, prednisone, prednisolone, methylpred, betamethasone), carboxyamidotriazole, combretastatin A-4, squalamine, 6-O-chloroacetyl-carbonyl)-fumagillol, thalidomide, angiostatin, troponin-1, angiotensin II antagonists (see Fernandez et al., J. Lab. Clin. Med. 105:141-145 (1985)), and antibodies to VEGF (see, Nature Biotechnology, Vol. 17, pp. 963-968 (October 1999); Kim et al., Nature, 362, 841-844 (1993); WO 00/44777; and WO 00/61186).

Other examples of angiogenesis inhibitors include, but are not limited to, endostatin, ukrain, ranpirnase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]methyl]-1H-1,2,3-triazole-4-carboxamide, CM101, squalamine, combretastatin, RPI4610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-methylene]-2-indolinone (SU5416).

Other therapeutic agents that modulate or inhibit angiogenesis and may also be used in combination with the compounds of the instant invention include agents that modulate or inhibit the coagulation and fibrinolysis systems (see review in Clin. Chem. La. Med. 38:679-692 (2000)). Examples of such agents that modulate or inhibit the coagulation and fibrinolysis pathways include, but are not limited to, heparin (see Thromb. Haemost. 80:10-23 (1998)), low molecular weight heparins and carboxypeptidase U inhibitors (also known as inhibitors of active thrombin activatable fibrinolysis inhibitor [TAFla]) (see Thrombosis Res. 101:329-354 (2001)). Examples of TAFla inhibitors have been described in PCT Publication WO 03/013,526.

Examples of kinase inhibitors include: agents that inhibit cell surface receptors and signal transduction cascades downstream of those surface receptors. Such agents inhibit cell proliferation and survival. These include inhibitors of EGFR (for example gefitinib and erlotinib), antibodies to EGFR (for example C225), inhibitors of ERB-2 (for example trastuzumab), inhibitors of IGFR, inhibitors of cytokine receptors, inhibitors of MET, inhibitors of MK (for example LY294002), serine/threonine kinases (including but not limited to inhibitors of Akt such as described in WO 02/083064, WO 02/083139, WO 02/083140 and WO 02/083138), inhibitors of Raf kinase (for example BAY-43-9006), inhibitors of MEK (for example CI-1040 and PD-098059), inhibitors of mTOR (for example Wyeth CCI-779), and inhibitors of C-abl kinase (for example GLEEVEC™, Novartis Pharmaceuticals). Additional kinase inhibitors include those that inhibit proteins involved in the cell cycle. These include Aurora kinase inhibitors, CDK inhibitors (e.g., flavopiridol, CYC202, BMS387032 and polo-like kinase inhibitors.) These also include agents that interfere with cell cycle checkpoints and thereby sensitize cancer cells to DNA damaging agents. Such agents include, e.g., inhibitors of ART, ATM, Chk1 and Chk2.

The invention also encompasses combinations with NSAID's which are selective COX-2 inhibitors. For purposes of this specification NSAID's which are selective inhibitors of COX-2 are defined as those which possess a specificity for inhibiting COX-2 over COX-1 of at least 100 fold as measured by the ratio of IC50 for COX-2 over IC50 for COX-1 evaluated by cell or microsomal assays. Inhibitors of COX-2 that are particularly useful in the instant method of treatment are: 3-phenyl-4-(4-(methylsulfonyl)phenyl)-2-(5H)-furanone; and 5-chloro-3-(4-methylsulfonyl)phenyl-2-(2-methyl-5 pyridinyl)pyridine; or a pharmaceutically acceptable salt thereof.

Compounds that have been described as specific inhibitors of COX-2 and are therefore useful in the present invention include, but are not limited to, parecoxib, CELIEBREX® and BEXTRA® or a pharmaceutically acceptable salt thereof.

“integrin blockers” refers to compounds which selectively antagonize, inhibit or counteract binding of a physiological ligand to the αᵥβ₃ integrin, to compounds which selectively antagonize, inhibit or counteract binding of a physiological ligand to the αᵥβ₅ integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁ and α₆β₄ integrins.

Combinations with compounds other than anti-cancer compounds are also encompassed in the instant methods. For example, combinations of the compounds of the invention with PPAR-γ (i.e., PPAR-gamma) agonists and PPAR-δ (i.e., PPAR-delta) agonists (collectively “PPAR agonists”) are useful in the treatment of certain malingnancies. PPAR-γ and PPAR-δ are the nuclear peroxisome proliferator-activated receptors γ and δ, respectively. The expression of PPAR-γ on endothelial cells and its involvement in angiogenesis has been reported in the literature (see J. Cardiovasc. Pharmacal. 1998; 31:909-913; J. Biol. Chem. 1999; 274:9116-9121; Invest. Ophthalmol. Vis. Sci. 2000; 41:2309-2317). More recently, PPAR-γ agonists have been shown to inhibit the angiogenic response to VEGF in vitro; both troglitazone and rosiglitazone maleate inhibit the development of retinal neovascularization in mice (Arch. Ophthamol. 2001; 119:709-717). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, G1262570, PNU182716, DRF552926, 2-[(5,7-dipropyl-3-trifluoromethyl-1,2-benzisoxazol-6-yl)oxy]-2-methylpropionic acid, and 2(R)-7-(3-(2-chloro-4-(4-fluorophenoxy)phenoxy)propoxy)-2-ethylchromane-2-carboxylic acid.

The compounds of the invention can also be administered in combination with one or more inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (valspodar).

Additional anticancer agents also include hypoxia activatable agents (e.g., tirapazamine), proteasome inhibitors (e.g., lactacystin and bortezomib), ubiquitin inhibitors, HDM2 inhibitors, TNF activators, BUB-R inhibitors, CENP-E inhibitors, and interferon alpha.

The compounds of the invention can also be employed in conjunction with one or more anti-emetic agents to treat nausea or emesis, including acute, delayed, late-phase, and anticipatory emesis, which may result from the use of a compound of the present invention, alone or with radiation therapy. For the prevention or treatment of emesis, a compound of the present invention may be used in conjunction with one or more other anti-emetic agents, especially neurokinin-1 receptor antagonists, 5HT3 receptor, antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or those as described in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide or dronabinol. In one embodiment, an anti-emesis agent selected from a neurokinin-1 receptor antagonist, a 5HT3 receptor antagonist and a corticosteroid is administered as an adjuvant for the treatment or prevention of emesis that may result upon administration of the compounds of the invention.

Examples of neurokinin-1 receptor antagonists that can be used in conjunction with the compounds of the invention are described in U.S. Pat. Nos. 5,162,339, 5,232,929, 5,242,930, 5,373,003, 5,387,595, 5,459,270, 5,494,926, 5,496,833, 5,637,699, and 5,719,147, content of which are incorporated herein by reference. In an embodiment, the neurokinin-1 receptor antagonist for use in conjunction with the compounds of the present invention is selected from: 2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine, or a pharmaceutically acceptable salt thereof, which is described in U.S. Pat. No. 5,719,147.

A compound of the present invention may also be administered with one or more immunologic-enhancing drug, such as for example, levamisole, isoprinosine and Zadaxin.

As described above, the present invention includes combinations comprising an amount of at least one compound (or a composition comprising a compound) of the invention or a pharmaceutically acceptable salt or ester thereof, and an amount of one or more additional therapeutic agents listed above (administered together or sequentially) wherein the amounts of the compounds/treatments result in desired therapeutic effect.

When administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for illustration purposes, a compound of the invention and an additional therapeutic agent may be present in fixed amounts (dosage amounts) in a single dosage unit (e.g., a capsule, a tablet and the like). A commercial example of such single dosage unit containing fixed amounts of two different active compounds is VYTORIN® (available from Merck Schering-Plough Pharmaceuticals, Kenilworth, N.J.).

If formulated as a fixed dose, such combination products employ the compounds of this invention within the dosage range described herein and the other pharmaceutically active agent or treatment within its dosage range. Compounds of the invention may also be administered sequentially with known therapeutic agents when a combination formulation is inappropriate. The invention is not limited in the sequence of administration; compounds of the invention may be administered either prior to or after administration of the known therapeutic agent. Such techniques are within the skills of persons skilled in the art as well as attending physicians.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

Therefore, we claim:

1. A compound having the general structure shown in Formula (I), or a pharmaceutically acceptable salt, solvate, ester, prodrug, or isomer thereof: 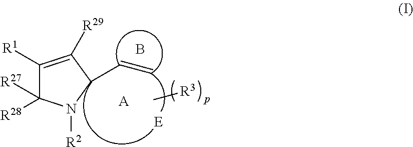
wherein R¹, R², R³, R²⁷, R²⁸, R²⁹, p, E, ring A, and ring B are selected independently of each other and wherein:
p is 0, 1, 2, 3, or 4;
ring A (including E and the unsaturation shown) is a 4-8 membered cycloalkenyl or heterocycloalkenyl ring;
E is selected from the group consisting of —O—, —S—, —S(O)—, —S(O)₂—, —C(R⁴)(R⁵)—, —N(R⁶)—, —N(C(Y)R⁷)—, —N(C(Y)OR⁸)—, —N(C(Y)N(R⁹)(R¹⁰))—, —C(O)—N(R¹¹)—, —N(R¹¹)—C(O)—, —S(O)₂—N(R¹¹)—, —N(R¹¹)—S(O)₂—, —C(O)—O—, —O—C(O)—, —O—N(R⁶)—, —N(R⁶)—O—, —N(R⁶)—N(R¹²)—, —N═N—, —C(R⁷)═N—, —C(O)—C(R⁷)═N—, —C(O)—N═N—, —O—C(Y)—N(R¹¹)—, —N(R¹¹)—C(Y)—O—, —N(R¹¹)—C(Y)—N(R¹²)—, —C(Y)—N(R¹¹)—O—, —C(Y)—N(R¹¹)—N(R¹²)—, —O—N(R¹¹)—C(Y)—, and —N(R¹²)—N(R¹)—C(Y)—,
wherein each Y is independently selected from the group consisting of (═O), (═S), (═N(R¹³)), (═N(CN)), (═N(OR¹⁴)), (═N(R¹⁵)(R¹⁶)), and (═C(R¹⁷)(R¹⁸));
ring B is an aromatic or heteroaromatic ring, or a partially unsaturated alicyclic ring, or a partially unsaturated heterocyclic ring,
wherein said ring is unsubstituted or optionally independently substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
R¹ is selected from the group consisting of aryl, heteroaryl, cycloalkyl, cycloalkenyl, heterocycloalkyl, and heterocycloalkenyl,
wherein each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl, cycloalkyl, cycloalkenyl, heterocycloalkyl, heterocycloalkenyl, azido, —OR⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
R² is selected from the group consisting of —C(Z)R⁷, —C(Z)NR⁹R¹⁰, —C(Z)OR⁸, —SO₂NR⁹R¹⁰, alkyl, heteroalkyl, aryl, heteroaryl, cycloalkyl, cycloalkenyl, heterocycloalkyl, and heterocycloalkenyl,
wherein each Z is independently selected from the group consisting of (═O), (═S), (═N(R¹³)), (═N(CN)), (═N(OR¹⁴)), (═N(R¹⁵)(R¹⁶)), and (═C(R¹⁷)(R¹⁸)), and
wherein each said alkyl, each said heteroalkyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group consisting of oxo (with the proviso that said aryl and said heteroaryl are not substituted with oxo), halogen, —CN, —NO₂, alkyl, heteroalkyl, haloalkyl, alkenyl, haloalkenyl, alkynyl, haloalkynyl, aryl, heteroaryl, aryl-alkyl, heteroaryl-alkyl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
R²⁷ (when not joined with R²⁸) is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
R²⁸ (when not joined with R²⁷) is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
or, alternatively, R²⁷ and R²⁸, together with the carbon atom to which they are attached, form a cycloalkyl, a cycloalkenyl, a heterocycloalkyl ring containing from one to three heteroatoms selected from the group consisting of N, O, and 5, or a heterocycloalkenyl ring containing from one to three heteroatoms selected from the group consisting of N, O, and S,
wherein said heterocycloalkyl ring and said heterocycloalkenyl ring are each unsubstituted or optionally independently substituted with one or more substituents, which can be the same or different, each substituent being independently selected from the group consisting of oxo, halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁴, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
R²⁹ is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
each R³ (when present) is independently selected from the group consisting of alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(S)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶, —NR²³C(O)NR²⁵R²⁶, and —NR²³—C(NH)—N(R²⁶)₂,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶,
or, alternatively, when p is 2, 3, or 4, any two R³ groups bound to the same ring carbon atom are taken together with the carbon atom to which they are attached to form a spirocycloalkyl, a spirocycloalkenyl, or a spiroheterocycloalkyl ring containing from one to three ring heteroatoms independently selected from the group consisting of —NH—, —NR⁶—, —S—, —S(O)—, —S(O)₂—, and —O—, or a spiroheterocycloalkenyl ring containing from one to three ring heteroatoms independently selected from the group consisting of —NH—, —NR⁶—, —S—, —S(O)—, —S(O)₂—, and —O—,
or, alternatively, R² and R³, together with the atom to which they are attached, are taken together with the carbon atom to which they are attached to form a cycloalkyl, a cycloalkenyl, a heterocycloalkyl ring containing from one to three ring heteroatoms independently selected from the group consisting of —NH—, —NR⁶—, —S—, —S(O)—, —S(O)₂—, and —O—, or a heterocycloalkenyl ring containing from one to three ring heteroatoms independently selected from the group consisting of —NH—, —NR⁶—, —S—, —S(O)—, —S(O)₂—, and —O—;
each R⁴ (when not joined with R⁵) is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁶, —SO₂R¹⁶, —OC(O)R²⁴, —C(O)NR²⁵R²⁶, —NR²³C(N—CN)NR²⁵R²⁶ and —NR²³C(O)NR²⁵R²⁶,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁶, —OC(O)R²⁴, —C(O)NR²⁵R²⁶, —NR²³C(N—CN)NR²⁵R²⁶ and —NR²³C(O)NR²⁵R²⁶;
each R⁵ (when not joined with R⁴) is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, halogen, —CN, —NO₂, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyi, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
or, alternatively, R⁴ and R⁵, together with the carbon atom to which they are attached, form a cycloalkyl, a cycloalkenyl, a heterocycloalkyl ring containing from one to three heteroatoms selected from the group consisting of N, O, and S, or a heterocycloalkenyl ring containing from one to three heteroatoms selected from the group consisting of N, O, and S,
wherein said heterocycloalkyl ring and said heterocycloalkenyl ring are each unsubstituted or optionally independently substituted with one or more substituents, which can be the same or different, each substituent being independently selected from the group consisting of oxo, halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
each R⁶ is independently selected from the group consisting of H, alkyl, —C(O)R²⁴, —C(O)OR²⁰, —C(S)R²⁴, heteroalkyl, alkenyl, heteroalkenyl, alkynyl, heteroalkynyl, aryl, heteroaryl, cycloalkyl, cycloalkenyl, heterocycloalkyl, heterocycloalkenyl,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or mare substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁸R²⁸, —C(O)R²⁴, —C(S)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
each R⁷ is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, hetorocycloalkenyl,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
each R⁸ is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
each R⁹ (when not joined with R¹⁰) is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
each R¹⁰ (when not joined with R⁹) is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
or, alternatively, R⁹ and R¹⁰, together with the N atom to which they are attached, form a heterocycloalkyl or a heterocycloalkenyl ring containing from one to three heteroatoms selected from the group consisting of N, O, and S.
wherein said heterocycloalkyl ring and said heterocycloalkenyl ring are each unsubstituted or optionally independently substituted with one or more substituents, which can be the same or different, each substituent being independently selected from the group consisting of oxo, halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
each R¹¹ is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
each R¹² is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²¹R²⁶, —C(O)R²⁴, —C(O)OR²⁰, —SR¹⁹, —S(O)R¹⁹, —SO₂R¹⁹, —OC(O)R²⁴, —C(O)NR²⁵R²⁵, —NR²³C(N—CN)NR²⁵R²⁶ and —NR²³C(O)NR²⁵R²⁶;
each R¹³ is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
each R¹⁴ is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
each R¹⁵ (when not joined with R¹⁶) is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
each R¹⁶ (when not joined with R¹⁵) is independently selected from the group consisting of 1-1, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
or, alternatively, R¹⁵ and R¹⁶, together with the N atom to which they are attached, form a heterocycloalkyl or a heterocycloalkenyl ring containing from one to three heteroatoms selected from the group consisting of N, O, and S,
wherein said heterocycloalkyl ring and said heterocycloalkenyl ring are each unsubstituted or optionally independently substituted with one or more substituents, which can be the same or different, each substituent being independently selected from the group consisting of oxo, halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
each R¹⁷ (when not joined with R¹⁸) is independently selected from the group consisting of H, alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, —CN, —OC(O)OR²⁰, —OR¹⁹, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
each R¹⁸ (when not joined with R¹⁷) is independently selected from the group consisting of alkyl, heteroalkyl, alkenyl, heteroalkenyl, alkynyl, heteroalkynyl, aryl, heteroaryl, cycloalkyl, cycloalkenyl, heterocycloalkyl, heterocycloalkenyl, —CN, —OC(O)OR²⁰, —OR¹⁹, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶,
wherein each said alkyl, each said heteroalkyl, each said alkenyl, each said heteroalkenyl, each said alkynyl, each said heteroalkynyl, each said aryl, each said heteroaryl, each said cycloalkyl, each said cycloalkenyl, each said heterocycloalkyl, and each said heterocycloalkenyl is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
or, alternatively, R¹⁷ and R¹⁸, together with the carbon atom to which they are attached, form a cycloalkyl, a cycloalkenyl, a heterocycloalkyl ring containing from one to three heteroatoms selected from the group consisting of N, O, and S, or a heterocycloalkenyl ring containing from one to three heteroatoms selected from the group consisting of N, O, and S,
wherein said heterocycloalkyl ring and said heterocycloalkenyl ring are each unsubstituted or optionally independently substituted with one or more substituents, which can be the same or different, each substituent being independently selected from the group consisting of oxo, halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
each R¹⁹ is independently selected from the group consisting of H, alkyl, haloalkyl, heteroalkyl, haloheteroalkyl, aryl, heteroaryl, cycloalkyl, halocycloalkyl;
each R²⁹ is independently selected from the group consisting of H, alkyl, haloalkyl, heteroalkyl, haloheteroalkyl, aryl, heteroaryl, cycloalkyl, halocycloalkyl;
each R²¹ (when not joined with R²²) is independently selected from the group consisting of H, alkyl, haloalkyl, heteroalkyl, haloheteroalkyl, aryl, heteroaryl, cycloalkyl, halocycloalkyl;
each R²² (when not joined with R²¹) is independently selected from the group consisting of H, alkyl, haloalkyl, heteroalkyl, haloheteroalkyl, aryl, heteroaryl, cycloalkyl, halocycloalkyl;
or, alternatively, R²¹ and R²², together with the N atom to which they are attached, form a heterocycloalkyl or a heterocycloalkenyl ring containing from one to three heteroatoms selected from the group consisting of N, O, and S;
each R²³ is independently selected from the group consisting of H, alkyl, haloalkyl, heteroalkyl, haloheteroalkyl, aryl, heteroaryl, cycloalkyl, halocycloalkyl;
each R²⁴ is independently selected from the group consisting of H, alkyl, haloalkyl, heteroalkyl, haloheteroalkyl, aryl, heteroaryl, cycloalkyl, halocycloalkyl;
each R²⁵ (when not joined with R²⁶) is independently selected from the group consisting of H, alkyl, haloalkyl, heteroalkyl, haloheteroalkyl, aryl, heteroaryl, cycloalkyl, halocycloalkyl; and
each R²⁶ (when not joined with R²⁵) is independently selected from the group consisting of H, alkyl, haloalkyl, heteroalkyl, haloheteroalkyl, aryl, heteroaryl, cycloalkyl, halocycloalkyl;
or, alternatively, R²⁵ and R²⁶, together with the N atom to which they are attached, form a heterocycloalkyl or a heterocycloalkenyl ring containing from one to three heteroatoms selected from the group consisting of N, O, and S.

2. A compound of claim 1, wherein ring A is a 4-7-membered cycloalkylene ring and E is —C(R⁴)(R⁵)—.

3. A compound of claim 1, wherein ring A is a 5-7-membered heterocycloalkylene ring and E is selected from the group consisting of —O—, —S—, —S(O)—, —S(O)₂—, —N(R⁶)—, —N(C(Y)R⁷)—, —N(C(Y)OR⁸)—, —N(C(Y)N(R⁹)(R¹⁰))—, —C(O)—N(R¹¹)—, —N(R¹¹)—C(O)—, —S(O)₂—N(R¹¹)—, —N(R¹¹)—S(O)₂—, —C(O)—O—, —O—C(O)—, —O—N(R⁶)—, —N(R⁶)—O—, —N(R⁶)—N(R¹²)—, —N═N—, —C(R⁷)═N—, —C(O)—C(R⁷)═N—, —C(O)—N═N—, —O—C(Y)—N(R¹¹)—, —N(R¹¹)—C(Y)—O—, —N(R¹¹)—C(Y)—N(R¹²)—, —C(Y)—N(R¹¹)—O—, —C(Y)—N(R¹¹)—N(R¹²)—, —O—N(R¹¹)—C(Y)—, and —N(R¹²)—N(R¹¹)—C(Y)—.

4. A compound of claim 1, wherein ring A is a 5-6-membered heterocycloalkylene ring and E is selected from the group consisting of —O—, —S—, —S(O)—, —S(O)₂—, —N(R⁶)—, —C(O)—N(R¹¹)—, and —N(R¹¹)—C(O)—.

5. A compound of claim 1, wherein ring A is a 5-6-membered heterocycloalkylene ring and E is selected from the group consisting of —O—, —S—, —S(O)—, —S(O)₂—, and —N(R⁶)—.

6. A compound of claim 5, wherein R⁶ is selected from the group consisting of H, alkyl, —C(O)R²⁴, —C(O)OR²⁰, and —C(S)R²⁴.

7. A compound of claim 1, wherein ring A is a 5-6-membered heterocycloalkylene ring and E is selected from the group consisting of —O— and —N(R⁶)—.

8. A compound of claim 7, wherein R⁶ is selected from the group consisting of H, alkyl, —C(O)R²⁴, —C(O)OR²⁰, and —C(S)R²⁴.

9. A compound of claim 8, wherein ring A is a 5-membered heterocycloalkylene ring.

10. A compound of claim 8, wherein ring A is a 6-membered heterocycloalkylene ring.

11. A compound of claim 1, wherein ring B is an unsubstituted aromatic ring or an aromatic ring which is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

12. A compound of claim 1, wherein ring B is an unsubstituted benzo ring or a benzo ring which is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)R²⁰, —NR²³C(O)R²⁴, —SO₂NR²⁵R²⁶, —C(O)R²⁴, —C(O)OR²⁴, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

13. A compound of claim 1, wherein ring B is an unsubstituted or substituted heteroaromatic ring or a substituted heteroaromatic ring which is substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

14. A compound of claim 13, wherein ring B is a 5-6-membered heteroaromatic ring having from 1-3 ring heteroatoms, which can be the same or different, each hetero ring atom being independently selected from the group consisting of N, S, O, S(O), and S(O)₂.

15. A compound of claim 1, wherein ring B is an unsubstituted or substituted moiety selected from the group consisting of benzo, furanyl, thiophenyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, and triazinyl.

16. A compound of claim 1, wherein R¹ is unsubstituted aryl or aryl substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

17. A compound of claim 1, wherein R¹ is phenyl substituted with one to four substituents, which can be the same or different, each substituent being independently selected from the group consisting of halo, —OH, —CN, —NO₂, —NR²¹R²², and haloalkyl.

18. A compound of claim 1, wherein R¹ is selected from the group consisting of:

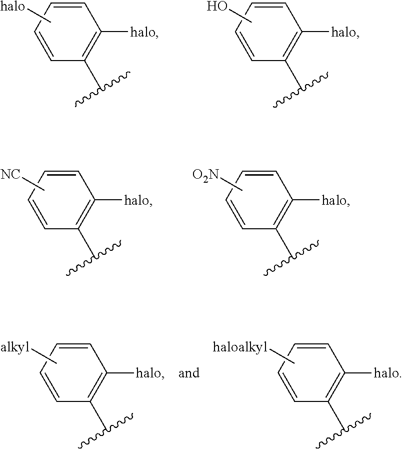

19. A compound of claim 1, wherein R¹ is a moiety selected from the group consisting of:

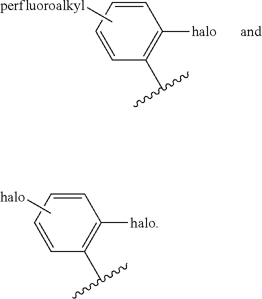

20. A compound of claim 1, wherein R¹ is: 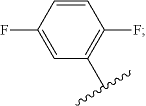
and
R²⁷, R²⁸, and R²⁹ are each independently selected from the group consisting of H and alkyl.

21. A compound of claim 1, wherein R² is selected from the group consisting of —C(O)R⁷, —C(O)NR⁹R¹⁰, and —C(O)OR³.

22. A compound of claim 1, wherein p is 0 and R³ is not present.

23. A compound of claim 1, wherein p is 1, 2, 3, or 4 and each R³ is independently selected from the group consisting of alkyl, heteroalkyl, alkenyl, heteroalkenyl, —CN, —NO₂, —OR¹⁹, —OC(O)OR²⁰, —NR²¹R²², —C(O)R²⁴, —C(S)R²⁴, —C(O)OR²⁰, and —O(O)NR²⁵R²⁶,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

24. A compound of claim 1, wherein p is 2, 3, or 4, and any two R³ groups bound to the same ring A atom are taken together with the carbon atom to which they are attached to form a spirocycloalkyl, a spirocycloalkenyl, a spiroheterocycloalkyl ring containing from one to three ring heteroatoms independently selected from the group consisting of —NH—, —NR⁶—, —S—, —S(O)—, —S(O)₂—, and —O—, or a spiroheterocycloalkenyl ring containing from one to three ring heteroatoms independently selected from the group consisting of —NH—, —NR⁶—, —S—, —S(O)—, —S(O)₂—, and —O—.

25. A compound of claim 1, wherein R² and R³ are taken together with the carbon atom to which they are attached to form a cycloalkyl, a cycloalkenyl, a heterocycloalkyl ring containing from one to three ring heteroatoms independently selected from the group consisting of —NH—, —NR⁶—, —S—, —S(O)—, —S(O)₂—, and —O—, or a heterocycloalkenyl ring containing from one to three ring heteroatoms independently selected from the group consisting of —NH—, —NR⁶—, —S—, —S(O)—, —S(O)₂—, and —O—.

26. A compound of claim 1, or a pharmaceutically acceptable salt, solvate, ester, prodrug, or isomer thereof, having the general structure shown in Formula (II): 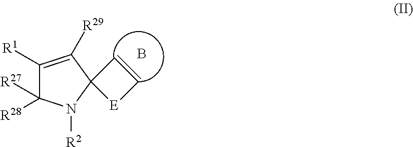
wherein R¹, R², R²⁷, R²⁸, R²⁹, E, and ring B are selected independently of each other and wherein
E is selected from the group consisting of —O—, —S—, —S(O)—, —S(O)₂—, —C(R⁴)(R⁵)—, —N(R⁶)—, —N(C(Y)R⁷)—, —N(C(Y)OR⁸)—, —N(C(Y)N(R⁹)(R¹⁰))—.

27. A compound of claim 26, wherein:
E is selected from the group consisting of —O—, —S—, —S(O)—, —S(O)₂—, —C(R⁴)(R⁵)—, and —N(R⁶)—;
ring B is an unsubstituted or substituted moiety selected from the group consisting of benzo, furanyl, thiophenyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, and triazinyl;
R¹ is phenyl substituted with one to four substituents, which can be the same or different, each substituent being independently selected from the group consisting of halo, —OH, —NR²¹R²², and haloalkyl; and
R² is selected from the group consisting of —C(O)R⁷, —C(O)NR⁹R¹⁰, and —C(O)OR⁸.

28. A compound of claim 27, wherein R¹ is: 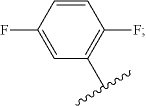
and R²⁷, R²⁸, and R²⁹ are each independently selected from the group consisting of H and alkyl.

29. A compound of claim 1, or a pharmaceutically acceptable salt, solvate, ester, prodrug, or isomer thereof, having the general structure shown in Formula (III.1): 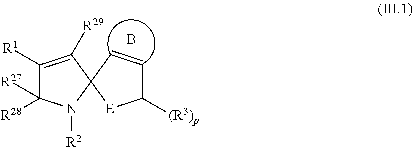
wherein R¹, R², R³, R²⁷, R²⁸, R²⁹, p, E, and ring B are selected independently of each other and wherein:
E is selected from the group consisting of —O—, —S—, —S(O)—, —S(O)₂—, —C(R⁴)(R⁵)—, —N(R⁶)—, —N(C(Y)R⁷)—, —N(C(Y)OR⁸)—, and —N(C(Y)N(R⁹)(R¹⁰))—; and
p is 0, 1, or 2.

30. A compound of claim 29, wherein:
E is selected from the group consisting of —C(R⁴)(R⁵)—, —O—, —S—, —S(O)—, —S(O)₂—, and —N(R⁶)—;
ring B is an unsubstituted or substituted aromatic ring or an unsubstituted or substituted 5-6-membered heteroaromatic ring having from 1-3 ring heteroatoms, which ring heteroatoms can be the same or different, each ring heteroatom being independently selected from the group consisting of N, S, O, S(O), and S(O)₂, said substituents on said aromatic ring or said heteroaromatic ring (when present) being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
R¹ is unsubstituted aryl or aryl substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
R² is selected from the group consisting of —C(O)R⁷, —O(O)NR⁹R¹⁰, and —C(O)OR⁸;
p is 0 or 1; and
each R³ (when present) is independently selected from the group consisting of alkyl, heteroalkyl, alkenyl, heteroalkenyl, —CN, —NO₂, —OR¹⁹, —OC(O)OR²⁰, —NR²¹R²², —C(O)R²⁴, —C(S)R²⁴, —C(O)OR²⁰, and —C(O)NR²⁵R²⁶,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁸, —NR²³C(N—CN)NR²⁵R²⁶ and —NR²³C(O)NR²⁵R²⁶.

31. A compound of claim 30, wherein:
ring B is an unsubstituted or substituted moiety selected from the group consisting of benzo, furanyl, thiophenyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, and triazinyl;
R¹ is phenyl substituted with one to four substituents, which can be the same or different, each substituent being independently selected from the group consisting of halo, —OH, —CN, —NO₂, —NR²¹R²², and haloalkyl;
R² is selected from the group consisting of —C(O)R⁷, —C(O)NR⁹R¹⁰, and —C(O)OR⁸;
p is or 1; and
each R³ (when present) is independently selected from the group consisting of alkyl, heteroalkyl, alkenyl, heteroalkenyl,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

32. A compound of claim 31, wherein R¹ is: 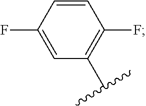
R⁶ is selected from the group consisting of H, alkyl, —C(O)R²⁴, —C(O)OR²⁰, and —C(S)R²⁴; and
R²⁷, R²⁸, and R²⁹ are each independently selected from the group consisting of H and alkyl.

33. A compound of claim 1, or a pharmaceutically acceptable salt, solvate, ester, prodrug, or isomer thereof, having the general structure shown in Formula (III.2): 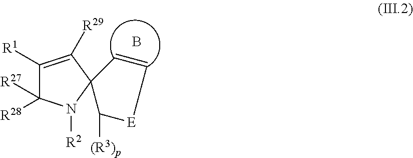
wherein R¹, R², R³, R²⁷, R²⁸, R²⁹, p, E, and ring B are selected independently of each other and wherein:
E is selected from the group consisting of —O—, —S—, —S(O)₂—, —C(R⁴)(R⁵)—, —N(R⁶)—, —N(C(Y)R⁷)—, —N(C(Y)OR⁸)—, and —N(C(Y)N(R⁹)(R¹⁰))—; and
p is 0, 1, or 2.

34. A compound of claim 33, wherein:
E is selected from the group consisting of —C(R⁴)(R⁵)—, —O—, —S—, —S(O)₂—, and —N(R⁶)—;
ring B is an unsubstituted or substituted aromatic ring or an unsubstituted or substituted 5-6-membered heteroaromatic ring having from 1-3 ring heteroatoms, which ring heteroatoms can be the same or different, each ring heteroatom being independently selected from the group consisting of N, S, O, S(O), and S(O)₂, said substituents on said aromatic ring or said heteroaromatic ring (when present) being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹—OC(O)R²⁴, —C(O)NR²⁵R²⁶, —NR²³C(N—CN)NR²⁵R²⁶ and —NR²³C(O)NR²⁵R²⁶.
R¹ is unsubstituted aryl or aryl substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, heteroaryl-alkyl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
R² is selected from the group consisting of —C(O)R⁷, —C(O)NR⁹R¹⁰, and —C(O)OR⁸;
p is 0 or 1; and
each R³ (when present) is independently selected from the group consisting of alkyl, heteroalkyl, alkenyl, heteroalkenyl, —CN, —NO₂, —OR¹⁹, —OC(O)OR²⁰, —NR²¹R²², —C(O)R²⁴, —C(S)R²⁴, —C(O)OR²⁰, and —C(O)NR²⁵R²⁶,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

35. A compound of claim 34, wherein:
ring B is an unsubstituted or substituted moiety selected from the group consisting of benzo, furanyl, thiophenyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, and triazinyl;
R¹ is phenyl substituted with one to four substituents, which can be the same or different, each substituent being independently selected from the group consisting of halo, —OH, —CN, —NO₂, —NR²¹R²², and haloalkyl;
R² is selected from the group consisting of —C(O)R⁷, —C(O)NR⁹R¹⁰, and —C(O)OR⁸;
p is 0 or 1; and
each R³ (when present) is independently selected from the group consisting of alkyl, heteroalkyl, alkenyl, heteroalkenyl,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²³, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

36. A compound of claim 35, wherein R¹ is: 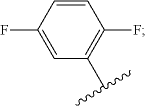
R⁶ is selected from the group consisting of H, alkyl, —C(O)R²⁴, —C(O)OR²⁰, and —C(S)R²⁴; and
R²⁷, R²⁸, and R²⁹ are each independently selected from the group consisting of H and alkyl.

37. A compound of claim 1, or a pharmaceutically acceptable salt, solvate, ester, prodrug, or isomer thereof, having the general structure shown in Formula (IV): 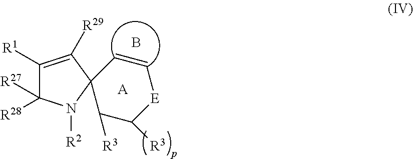
E is selected from the group consisting of —C(R⁴)(R⁵)—, —O—, —S—, —S(O)—, —S(O)₂—, and —N(R⁶)—;
ring B is an unsubstituted or substituted aromatic ring or an unsubstituted or substituted 5-6-membered heteroaromatic ring having from 1-3 ring heteroatoms, which ring heteroatoms can be the same or different, each ring heteroatom being independently selected from the group consisting of N, S, O, S(O), and S(O)₂, said substituents on said aromatic ring or said heteroaromatic ring (when present) being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl-, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
R¹ is unsubstituted aryl or aryl substituted with one or more substituents, which can be the same or different, each substituent being independently selected from the group consisting of halogen, —CN, —NO₂, alkyl, heteroalkyl, haloalkyl, alkenyl, haloalkenyl, alkynyl, haloalkynyl, aryl, heteroaryl, aryl-alkyl-, heteroaryl-alkyl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶;
R² is selected from the group consisting of —C(O)R⁷, —C(O)NR⁹R¹⁰, and —C(O)OR⁸;
p is 0, 1, or 2; and
each R³ (when present) is independently selected from the group consisting of alkyl, heteroalkyl, alkenyl, heteroalkenyl, —CN, —NO₂, —OR¹⁹, —OC(O)OR²⁰, —NR²¹R²², —C(O)R²⁴, —C(S)R²⁴, —C(O)OR²⁰, and —C(O)NR²⁵R²⁶,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

38. A compound of claim 37, wherein:
E is selected from the group consisting of —O— and —N(R⁶)—;
ring B is an unsubstituted or substituted moiety selected from the group consisting of benzo, (uranyl, thiophenyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, and triazinyl;
R¹ is phenyl substituted with one to four substituents, which can be the same or different, each substituent being independently selected from the group consisting of halo, —OH, —CN, —NO₂, —NR²¹R²², and haloalkyl;
R² is selected from the group consisting of —C(O)R⁷, —C(O)NR⁹R¹⁰, and —C(O)OR⁸;
p is 0 or 1; and
each R³ (when present) is independently selected from the group consisting of alkyl, heteroalkyl, alkenyl, heteroalkenyl,
wherein each said alkyl, each said heteroalkyl, each said alkenyl, and each said heteroalkenyl, is unsubstituted or optionally independently substituted with one or more substituents, which can be the same or different, each substituent being independently selected from the group of oxo, halogen, —CN, —NO₂, alkyl, heteroalkyl, haloalkyl, alkenyl, haloalkenyl, alkynyl, haloalkynyl, aryl, heteroaryl, cycloalkyl, cycloalkenyl, heterocycloalkyl, heterocycloalkenyl, azido, —OR¹⁹, —OC(O)OR²⁰, —NR²¹R²², —NR²³SO₂R²⁴, —NR²³C(O)OR²⁰, —NR²³C(O)R²⁴, —SO₂NR²⁵R²⁶, —C(O)R²⁴, —C(O)OR²⁰, —SR¹⁹, —S(O)R¹⁹, —SO₂R¹⁹, —OC(O)R²⁴, —C(O)NR²⁵R²⁶, —NR²³C(N—CN)NR²⁵R²⁶ and —NR²³C(O)NR²⁵R²⁶.

39. A compound of claim 38, wherein R¹ is: 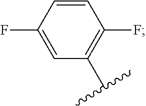
R⁶ is selected from the group consisting of H, alkyl, —C(O)R²⁴, —C(O)OR²⁰, and —C(S)R²⁴; and
R²⁷, R²⁸, and R²⁹ are each independently selected from the group consisting of H and alkyl.

40. A compound, or a pharmaceutically acceptable salt, solvate ester, prodrug, or isomer thereof, selected from the group consisting of:

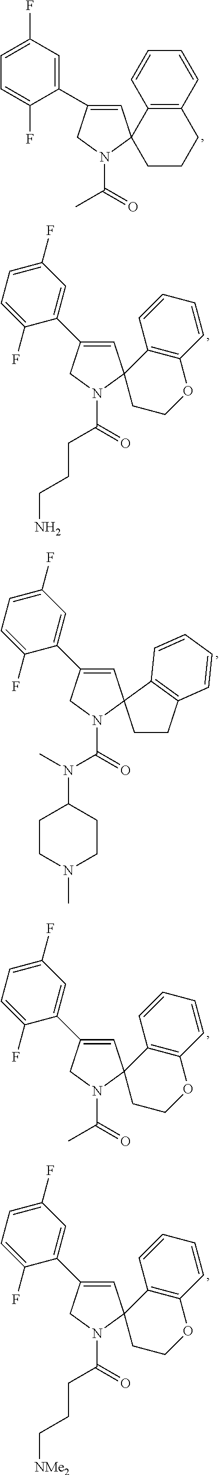

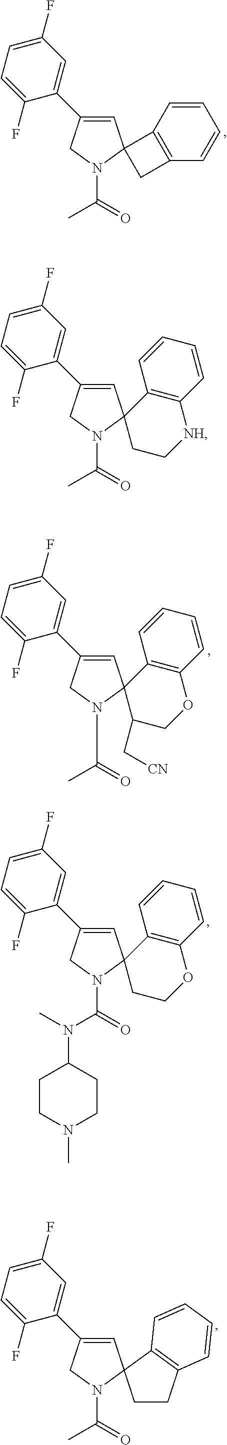

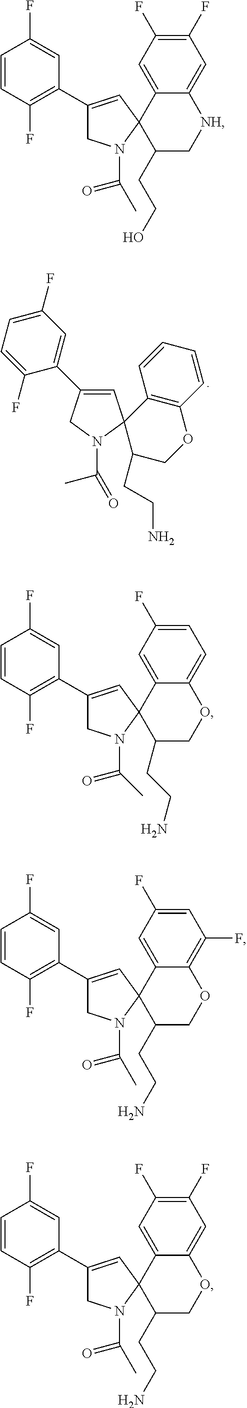

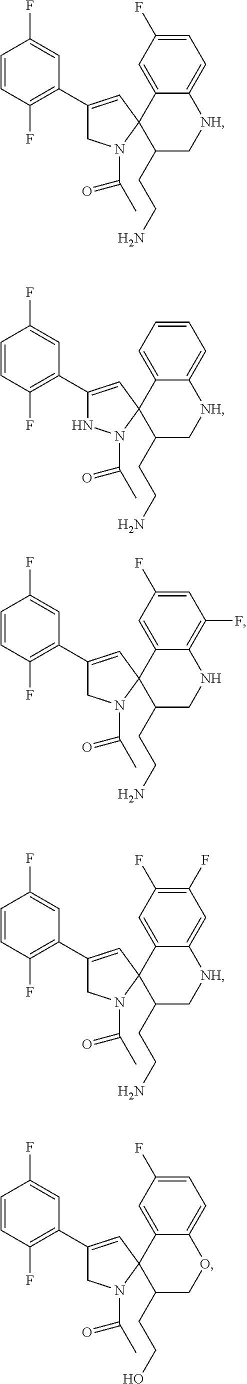

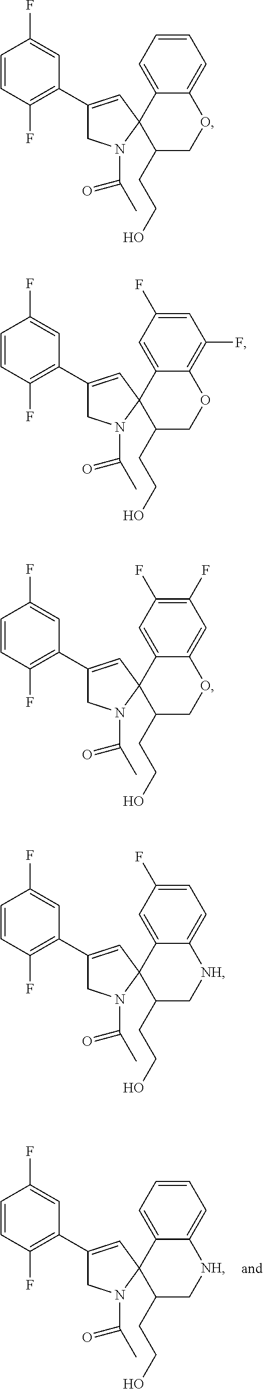

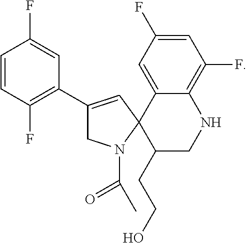

41. A compound according to any one of claims 1-40 in isolated or purified form.

42. A pharmaceutical composition comprising a therapeutically effective amount of at least one compound of any one of claims 1-40, or a pharmaceutically acceptable salt, solvate, ester, prodrug, or isomer thereof, and at least one pharmaceutically acceptable carrier.

43. A pharmaceutical composition of claim 42, further comprising at least one additional therapeutically active agent.

44. A pharmaceutical composition of claim 43, wherein said at least one additional therapeutically active agent is selected from:
estrogen receptor modulators, androgen receptor modulators, retinoid receptor modulators, cytotoxic agents, microtubule inhibitors/stabilizing agents, topoisomerase inhibitors, antisense RNA and DNA oligonucleotides, antimetabolites, antibodies coupled to cyclotoxic agents, radioisotypes, HMG-CoA reductase inhibitors, prenyltransferase inhibitors, farnesyl protein transferase inhibitors, angiogenesis inhibitors, kinase inhibitors, COX2 inhibitors, integrin blockers, PPAR agonists, MDR inhibitors, hypoxia activatable agents, proteasome inhibitors, ubiquitin inhibitors, HDM2 inhibitors, TNF activators, BUB-R inhibitors, CENP-E inhibitors, interferon, and radiation.

45. A method of inhibiting KSP kinesin activity in a subject in need thereof comprising administering to said subject an effective amount of at least one compound according to any one of claims 1-40, or a pharmaceutically acceptable salt, solvate, ester, prodrug, or isomer thereof.

46. A method of treating a disease associated with or caused by aberrant cellular proliferation in a subject in need thereof comprising administering to said subject an effective amount of at least one compound according to any one of claims 1-40, or a pharmaceutically acceptable salt, solvate, prodrug, ester, or isomer thereof.

47. A method of claim 46, wherein the said disease is selected from the group consisting of cancer, hyperplasia, cardiac hypertrophy, autoimmune diseases, fungal disorders, arthritis, graft rejection, inflammatory bowel disease, immune disorders, inflammation, tumor angiogenesis, and cellular proliferation induced by a medical procedure.

48. A method of claim 46, wherein the cellular proliferative disease is selected from solid tumor cancer and hematological cancer.

49. A method of claim 46, wherein said disease is a cancer selected from skin cancer, breast cancer, brain cancer, colon cancer, gall bladder cancer, thyroid cancer, cervics cancer, testicular cancer, and blood cancer.

50. A method of claim 46, wherein said disease is a cancer selected from: cardiac cancer, lung cancer, gastrointestinal cancer, genitourinary tract cancer, liver cancer, bone cancer, nervous system cancer, gynecological cancer, hematologic cancer, skin cancer, cancer of the adrenal gland, xenoderoma pigmentosum, keratoctanthoma, and thyroid follicular cancer.

51. The method of claim 46, wherein said cellular proliferative disease is selected from:
adenocarcinoma, Wilm's tumor (nephroblastoma), lymphoma, leukemia, squamous cell carcinoma, transitional cell carcinoma, adenocarcinoma, prostate cancer, testicular cancer,
hepatoma (hepatocellular carcinoma), cholangiocarcinoma, hepatoblastoma, angiosarcoma, hepatocellular adenoma, hemangioma;
osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors;
osteoma, hemangioma, granuloma, xanthoma, osteitis deformans, meningioma, meningiosarcoma, gliomatosis, astrocytoma, medulloblastoma, glioma, ependymoma, germinoma (pinealoma), glioblastoma multiform, oligodendroglioma, schwannoma, retinoblastoma, congenital tumors, spinal cord neurofibroma, meningioma, glioma, sarcoma;
endometrial carcinoma, cervical carcinoma, pre-tumor cervical dysplasia, serous cystadenocarcinoma, mucinous cystadenocarcinoma, unclassified carcinoma, granulosa-thecal cell tumors, Sertoli-Leydig cell tumors, dysgerminoma, malignant teratoma, squamous cell carcinoma, intraepithelial carcinoma, adenocarcinoma, fibrosarcoma, melanoma, clear cell carcinoma, squamous cell carcinoma, botryoid sarcoma (embryonal rhabdomyosarcoma), fallopian tube carcinoma;
myeloid leukemia (acute and chronic), acute lymphoblastic leukemia, acute and chronic lymphocytic leukemia, myeloproliferative diseases, multiple myeloma, myelodysplastic syndrome, Hodgkin's disease, non-Hodgkin's lymphoma (malignant lymphoma), B-cell lymphoma, T-cell lymphoma, hairy cell lymphoma, Burkett's lymphoma, promyelocytic leukemia;
malignant melanoma, basal cell carcinoma, squamous cell carcinoma, Karposi's sarcoma, moles dysplastic nevi, lipoma, angioma, dermatofibroma, keloids, psoriasis, and neuroblastoma.

52. The method of claim 51, further comprising radiation therapy.

53. The method of claim 46, further comprising administering to the subject at least one additional therapeutically active agent selected from:
estrogen receptor modulators, androgen receptor modulators, retinoid receptor modulators, cytotoxic agents, microtubule inhibitors/stabilizing agents, topoisomerase inhibitors, antisense RNA and DNA oligonucleotides, antimetabolites, antibodies coupled to cyclotoxic agents, radioisotypes, HMG-CoA reductase inhibitors, prenyltransferase inhibitors, farnesyl protein transferase inhibitors, angiogenesis inhibitors, kinase inhibitors, COX2 inhibitors, integrin blockers, PPAR agonists, MDR inhibitors, hypoxia activatable agents, proteasome inhibitors, ubiquitin inhibitors, HDM2 inhibitors, TNF activators, BUB-R inhibitors, CENP-E inhibitors, interferon, and radiation.

54. The use of at least one compound according to any one of claims 1-40, or a pharmaceutically acceptable salt, solvate, ester or prodrug thereof, for the manufacture a medicament for inhibiting KSP kinesin activity in a subject in need thereof.

55. The use of at least one compound according to any one of claims 1-40, or a pharmaceutically acceptable salt, solvate, ester or prodrug thereof, for the manufacture of a medicament for treating one or more diseases by inhibiting KSP kinesin activity in a patient in need thereof.

56. The use of a combination comprising (i) a compound according to any one of claims 1-40, or a pharmaceutically acceptable salt, solvate, ester or prodrug thereof; and (ii) at least one second compound, the second compound being selected from:
estrogen receptor modulators, androgen receptor modulators, retinoid receptor modulators, cytotoxic agents, microtubule inhibitors/stabilizing agents, topoisomerase inhibitors, antisense RNA and DNA oligonucleotides, antimetabolites, antibodies coupled to cyclotoxic agents, radioisotypes, HMG-CoA reductase inhibitors, prenyltransferase inhibitors, farnesyl protein transferase inhibitors, angiogenesis inhibitors, kinase inhibitors, COX2 inhibitors, integrin blockers, PPAR agonists, MDR inhibitors, hypoxia activatable agents, proteasome inhibitors, ubiquitin inhibitors, HDM2 inhibitors, TNF activators, BUB-R inhibitors, CENP-E inhibitors, interferon, and radiation
to manufacture a medicament for treating one or more diseases by inhibiting KSP kinesin activity in a subject in need thereof.
